(19) United States
(12) Patent Application Publication
(10) Pub. No.: US 2023/0038520 A1
(43) Pub. Date: Feb. 9, 2023

(54) THERAPEUTIC ADENO-ASSOCIATED VIRUS COMPRISING LIVER-SPECIFIC PROMOTERS FOR TREATING POMPE DISEASE AND LYSOSOMAL DISORDERS

(71) Applicant: ASKLEPIOS BIOPHARMACEUTICAL, INC., Research Triangle Park, NC (US)

(72) Inventors: Michael ROBERTS, Research Triangle Park, NC (US); Juan Manuel IGLESIAS, Research Triangle Park, NC (US); Anna TRETIAKOVA, Research Triangle Park, NC (US); Michael W. O'CALLAGHAN, Research Triangle Park (US); Achille FRANCOIS, Research Triangle Park, NC (US)

(73) Assignee: ASKLEPIOS BIOPHARMACEUTICAL, INC., Research Triangle Park, NC (US)

(21) Appl. No.: 17/778,175

(22) PCT Filed: Nov. 19, 2020
(86) PCT No.: PCT/US2020/061223

§ 371 Date: May 19, 2022

Related U.S. Application Data
(60) Provisional application No. 63/023,570, filed on May 12, 2020.
(60) Provisional application No. 62/937,556, filed on Nov. 19, 2019.
(60) Provisional application No. 62/937,583, filed on Nov. 19, 2019.

(51) Int. Cl. C12N 15/86; C12N 9/26; C07K 14/62

(52) U.S. Cl. CPC C12N 15/86; C12N 9/2408; C12Y 302/0102; C07K 14/62; C07K 2319/02; C12N 2750/14143; C12N 2830/38; C12N 2830/42

(57) ABSTRACT

Recombinant AAV (rAAV) vectors comprising a rAVV genome comprising a heterologous nucleic acid encoding a lysosomal protein, e.g., acid alpha-glucosidase (GAA) polypeptide, and optionally a signal peptide and/or optionally a targeting sequence, e.g., IGF2 targeting peptide, operatively linked to a liver-specific promoter (LSP), enabling the GAA polypeptide to be secreted from the liver and targeted to the lysosomes. Particular embodiments relate to a recombinant AAV (rAAV) vector encoding an alpha-glucosidase (GAA) polypeptide, having a liver secretory signal peptide and a IGF2 targeting peptide that binds human cation-independent mannose-6-phosphate receptor (CI-MPR) or to the IGF2 receptor, permitting proper subcellular localization of the GAA polypeptide to lysosomes. Also encompassed are cells, and methods to treat a lysosomal disease, for example, a glycogen storage disease type II (GSD II) disease and/or Pompe Disease with the rAAV vector.

### SEQUENCE LISTING

This invention claims benefit under 35 U.S.C. § 119(e) of U.S. Provisional Application 62/937,556 filed on Nov. 19, 2019, and U.S. Provisional Application 62/937,583 filed on Nov. 19, 2019, and U.S. Provisional Application 63/023,570 filed on May 12, 2020, the contents of each are incorporated herein in their entirety by reference.

### SEQUENCE LISTING

The instant application contains a Sequence Listing which has been submitted electronically in ASCII format, and is hereby incorporated by reference in its entirety. Said ASCII copy, created on Nov. 17, 2020, is named 046192-096600WOPT SL.txt and is 840,179 bytes in size.

### FIELD OF THE INVENTION

The present invention relates to adeno-associated virus (AAV) particles, virions and vectors for targeted translocation of lysosomal enzymes, such as, e.g., an alpha-glucosidase (GAA) polypeptide, and method of use for the treatment of lysosomal storage diseases and disorders, such as, e.g., Pompe disease.

### BACKGROUND

More than forty lysosomal storage diseases (LSDs) are caused, directly or indirectly, by the absence of one or more lysosomal enzymes in the lysosome Enzyme replacement therapy for LSDs is being actively pursued. Therapy generally requires that LSD proteins be taken up and delivered to the lysosomes of a variety of cell types in an M6P-dependent fashion. One possible approach involves purifying an LSD protein and modifying it to incorporate a carbohydrate moiety with M6P. This modified material may be taken up by the cells more efficiently than unmodified LSD proteins due to interaction with M6P receptors on the cell surface.

As an alternative or adjunct to enzyme therapy, the feasibility of gene therapy approaches to treat GSD-II have been investigated (Amalfitano, A., et al., (1999) Proc. Natl. Acad. Sci. USA 96:8861-8866, Ding, E., et al. (2002) Mol. Ther. 5:436-446, Fraites, T. J., et al., (2002) Mol. Ther. 5:571-578, Tsujino, S., et al. (1998) Hum. Gene Ther. 9:1609-1616).

However, viral or AAV delivery of genes, in particular lysosomal proteins and enzymes for treatment of lysosomal storage diseases has challenges. Normally, mammalian lysosomal enzymes are synthesized in the cytosol and traverse the ER where they are glycosylated with N-linked, high mannose type carbohydrate. In the Golgi, the high mannose carbohydrate is modified on lysosomal proteins by the addition of mannose-6-phosphate (M6P) which targets these proteins to the lysosome. The M6P-modified proteins are delivered to the lysosome via interaction with either of two M6P receptors. However, recombinantly produced proteins used in enzyme replacement therapy often lack the addition of the M6P which is required for targeting them to the lysosomes, therefore, often requiring high doses of recombinantly produced enzymes to be administered to a patient and/or frequent infusions.

Acid alpha-glucosidase (GAA) is a lysosomal enzyme that hydrolyzes the alpha 1-4 linkage in maltose and other linear oligosaccharides, including the outer branches of glycogen, thereby breaking down excess glycogen in the lysosome (Hirschhorn et al. (2001) in The Metabolic and Molecular Basis of Inherited Disease, Scriver, et al., eds. (2001), McGraw-Hill: New York, p. 3389-3420). Like other mammalian lysosomal enzymes, GAA is synthesized in the cytosol and traverses the ER where it is glycosylated with N-linked, high mannose type carbohydrate. In the Golgi, the high mannose carbohydrate is modified on lysosomal proteins by the addition of mannose-6-phosphate (M6P) which targets these proteins to the lysosome. The M6P-modified proteins are delivered to the lysosome via interaction with either of two M6P receptors. The most favorable form of modification is when two M6Ps are added to a high mannose carbohydrate.

Insufficient GAA activity in the lysosome results in Pompe disease, a disease also known as acid maltase deficiency (AMD), glycogen storage disease type II (GSDII), glycogenosis type II, or GAA deficiency. The diminished enzymatic activity occurs due to a variety of missense and nonsense mutations in the gene encoding GAA. Consequently, glycogen accumulates in the lysosomes of all cells in patients with Pompe disease. In particular, glycogen accumulation is most pronounced in lysosomes of cardiac and skeletal muscle, liver, and other tissues. Accumulated glycogen ultimately impairs muscle function. In the most severe form of Pompe disease, death occurs before two years of age due to cardio-respiratory failure.

There is a need for an effective treatment of Pompe disease. Enzyme replacement therapeutics for Pompe require a recombinant GAA protein to be administered and taken up by muscle and liver cells in the subject where it is subsequently transported to the lysosomes in those cells in a M6P-dependent fashion. However, while enzyme therapy has demonstrated reasonable efficacy for severe infantile GSD II, the benefit of GAA enzyme therapy is limited by the need for frequent infusions as well as the subject developing inhibitor or neutralizing antibodies against recombinant hGAA protein (Amalfitano, A., et al. (2001) Genet. In Med. 3:132-138).

Gene therapy has the potential to not only cure genetic disorders, but to also facilitate the long-term non-invasive treatment of acquired and degenerative disease using a virus. One gene therapy vector is adeno-associated virus (AAV). AAV itself is a non-pathogenic-dependent parvovirus that needs helper viruses for efficient replication. AAV has been utilized as a virus vector for gene therapy because of its safety and simplicity. AAV has a broad host and cell type tropism capable of transducing both dividing and non-dividing cells.

However, AAV delivery of the GAA polypeptide has some challenges with respect to achieving sufficient expression in the liver and/or delivery to lysosomes with patients reporting to experience glycaemia.

In particular, in human subjects, the administration of rAAV vectors encoding GAA polypeptide have resulted in a number of patients experiencing hypoglycemia or becoming hyperglycemic due to non-specific update in cells (see, e.g., Byrne et al., A study on the safety and efficacy of Reveglucosidease alfa in patients with late-onset Pompe disease; Orphanet J. of Rare diseases; 2017; 12: 144).

Accordingly, there is a need in the art for improved methods of producing lysosomal polypeptides such as GAA in vitro and in vivo, for example, to treat lysosomal polypeptide deficiencies, including modifications of GAA. Moreover, there is a need for improved secretion from the liver as well as improved targeting of GAA to the lysosomes to help reduce any side effects from overexpression of the GAA polypeptide, and reducing the risk of hypoglycemia. Further, there is a need for methods that result in systemic delivery of GAA and other lysosomal polypeptides to affected tissues and organs. In particular, there remains a need for more efficient methods for administering GAA protein to subjects and targeting GAA protein to patient lysosomes, while reducing any potential side effects.

### SUMMARY OF THE INVENTION

The technology described herein relates generally to gene therapy constructs, methods and composition, for the treatment lysosomal storage diseases and disorders, such as, for example but not limited to, Pompe Disease. More particularly, the technology relates to adeno-associated (AAV) virions configured for delivering a lysosomal enzyme, e.g., a GAA polypeptide to a subject, and more particularly for delivering a lysosomal enzyme, e.g., a GAA polypeptide to the liver of a subject where it is targeted to the lysosomes and secreted from the liver cells.

In particular, described herein are targeted viral vectors, e.g., using rAAV vectors as an exemplary example, that comprise a nucleotide sequence containing inverted terminal repeats (ITRs), a promoter, a heterologous gene, a poly-A tail and potentially other regulator elements for use to treat a lysosomal storage disease, such as those listed in Table 5A or Table 6A herein, wherein the heterologous gene is a lysosomal enzyme, such as, e.g., GAA, and wherein the vector, e.g., rAAV can be administered to a patient in a therapeutically effective dose that is delivered to the appropriate tissue and/or organ for expression of the heterologous lysosomal enzyme gene and treatment of the disease, e.g., Pompe disease.

Aspects of the present invention teach certain benefits in construction and use which give rise to the exemplary advantages described below.

Accordingly, in particular embodiments described herein are rAAV vectors that comprises a nucleotide sequence containing inverted terminal repeats (ITRs) and located between the ITRs, a liver specific promoter (LSP), a heterologous nucleic acid sequence that encodes the acid alpha-glucosidase (GAA) protein, a poly-A tail and potentially other regulator elements for use to treat Pompe Disease, and wherein the rAAV expressing GAA protein can be administered to a patient in a therapeutically effective dose that is delivered to the appropriate tissue and/or organ for expression of the heterologous gene encoding the GAA protein for the treatment of a subject with Pompe disease.

More specifically, the AAV virion or genome comprises a LSP selected from any promoter listed in Table 4 herein, or a functional variant or functional fragment thereof, or any LSP selected from SEQ ID NO: 86, 91-96, or 146-150 or a functional variant or functional fragment thereof, that enables the lysosomal protein, e.g., GAA protein to be preferentially expressed in the liver. In some embodiments, the liver-specific promoter, while preferentially expresses the hGAA protein in the liver, can also express the hGAA to some extent in another tissue of interest, e.g., the muscle, or CNS, or muscle and CNS tissues. In some embodiments, the expressed lysosomal enzyme, e.g., GAA protein can be configured as GAA-fusion protein with a targeting sequence, such as a IGF2 targeting peptide as disclosed herein that targets the GAA protein to lysosomes, and/or fused with a signal peptide (SP), the GAA protein is expressed by the rAAV genome in the liver, where it is secreted and taken up by lysosomes of mammalian cells, in particular muscle cells.

In some embodiments of the compositions and methods described herein, the rAAV vector disclosed herein comprises, in its genome: 5′ and 3′ AAV inverted terminal repeats (ITR) sequences, and located between the 5′ and 3′ ITRs, a liver specific promoter (LSP) operatively linked to a heterologous nucleic acid sequence encoding an alpha-glucosidase (GAA) polypeptide, wherein the liver-specific promoter (LSP) comprises a nucleic acid sequence selected from any promoter listed from SEQ ID NOS: 86 (CRM 0412), SEQ ID NO: 91 (SP0412) or SEQ ID NO: 92 (SP0422), SEQ ID NOS: 93 (SP0239), SEQ ID NO: 94 (SP0265, also referred to SP131_A1), SEQ ID NO: 95 (SP0240) or SEQ ID NO: 96 (SP0246), or SEQ ID NO: 146 (SP0265-UTR), SEQ ID NO: 147 (SP0239-UTR), SEQ ID NO: 148 (SP0240-UTR), SEQ ID NO: 149 (SP0246-UTR) or SEQ ID NO: 150 (SP0131-A1-UTR) or a functional fragment or variant thereof, or any LSP selected from SEQ ID NO: 270-341 or 342-430, or a functional fragment or variant thereof. In some embodiments, the GAA polypeptide is not fused to either a IGF2 targeting sequence, or signal sequence. In some embodiments, the GAA polypeptide is fused to a signal sequence as disclosed herein, and/or a IGF2 targeting sequence as disclosed herein.

In some embodiments of the compositions and methods described herein, the rAAV vector disclosed herein comprises, in its genome: 5′ and 3′ AAV inverted terminal repeats (ITR) sequences, and located between the 5′ and 3′ ITRs, a liver specific promoter (LSP) operatively linked to a heterologous nucleic acid sequence encoding a fusion polypeptide comprising (i) a secretory signal peptide, and/or an IGF2 targeting peptide; and (ii) an alpha-glucosidase (GAA) polypeptide, wherein the liver-specific promoter (LSP) is selected from any promoter listed in Table 4 herein, or a functional variant or functional fragment thereof, or any LSP selected from SEQ ID NO: 86, 91-96, or 146-150 or a functional variant or functional fragment thereof.

In some embodiments, the rAAV vector disclosed herein comprises, in its genome: 5′ and 3′ AAV inverted terminal repeats (ITR) sequences, and located between the 5′ and 3′ ITRs, a heterologous nucleic acid sequence encoding a fusion polypeptide comprising (i) a secretory signal peptide (also referred to as a leader peptide), and (ii) an alpha-glucosidase (GAA) polypeptide, wherein the heterologous nucleic acid is operatively linked to a liver-specific promoter (LSP) selected from any promoter listed in Table 4 herein, or a functional variant or functional fragment thereof, or any LSP selected from SEQ ID NO: 86, 91-96, or 146-150, or a functional variant or functional fragment thereof or any LSP selected from Table 4 herein, or a functional variant or functional fragment thereof. Exemplary leader sequences include, but are not limited to the innate GAA leader sequence, AAT sequence, IL2(1-3), IL2 leader sequence (IL2 wt), a modified IL2 leader sequence (IL2 mut), fibronectin (FN1) signal sequence, or IgG leader sequence or functional variants thereof, as disclosed herein. In some embodiments, the AAV vector comprises a Kozak sequence located between the LSP and the leader sequence.

In some embodiments, the rAAV vector disclosed herein comprises, in its genome: 5′ and 3′ AAV inverted terminal repeats (ITR) sequences, and located between the 5′ and 3′ ITRs, a heterologous nucleic acid sequence encoding a fusion polypeptide comprising (i) an IGF2 targeting peptide, and (ii) an alpha-glucosidase (GAA) polypeptide, wherein the heterologous nucleic acid is operatively linked to a liver-specific promoter (LSP) selected from any promoter listed in Table 4 herein, or a functional variant or functional fragment thereof, or any LSP selected from SEQ ID NO: 86, 91-96, or 146-150 or a functional variant or functional fragment thereof.

In a further embodiments, the rAAV vector disclosed herein comprises, in its genome: 5′ and 3′ AAV inverted terminal repeats (ITR) sequences, and located between the 5′ and 3′ ITRs, a heterologous nucleic acid sequence encoding an alpha-glucosidase (GAA) polypeptide (i.e., where the GAA polypeptide not fused to a heterologous signal peptide (or a leader sequence), or not fused to an IGF2 targeting sequence as described herein), wherein the heterologous nucleic acid is operatively linked to a liver-specific promoter (LSP) selected from any promoter listed in Table 4 herein, or a functional variant or functional fragment thereof, or any LSP selected from SEQ ID NO: 86, 91-96, or 146-150 or a functional variant or functional fragment thereof.

In some embodiments, the rAAV vector comprises a liver specific capsid, e.g., a liver specific capsid selected from XL32 and XL32.1, as disclosed in WO2019/241324, which is incorporated herein in its entirety by reference. In some embodiments, the rAAV vector is a AAVXL32 or AAVXL32.1 as disclosed in WO2019/241324, which is incorporated herein in its entirety by reference, or a AAV8 vector, or a haploid AAV vector comprising at least one AAV8 capsid protein (e.g., at least one of VP1, VP2, or VP3 is from the AAV8 serotype), and in some embodiments, the AAV vector is a haploid AAV vector comprising at least two AAV8 capsid proteins). In some embodiments, the AAV vector comprises a capsid disclosed in WO2019241324A1, or International Patent application PCT/US2019/036676, which are incorporated herein in their entirety by reference. In some embodiments, the AAV vector comprises a capsid which is encoded by a nucleic acid AAV capsid coding sequence that is at least 90% identical to a nucleotide sequence of any one of SEQ ID NOs: 1-3 as disclosed in WO2019241324A1; or (b) a nucleotide sequence encoding any one of SEQ ID NOS:4-6 as disclosed in WO2019241324A1. In some embodiments, an AAV capsid comprises an amino acid sequence at least 90% identical to any one of SEQ ID NOS:4-6 as disclosed in WO2019241324A1, along with AAV particles comprising an AAV vector genome and the AAV capsid of the invention.

In some embodiments, the rAAV vector comprises capsid proteins such that the AAV vector transduces liver cells, and in some embodiments the rAAV vector comprises the rAAV vector comprises capsid proteins such that the AAV vector transduces muscle and liver cells.

An exemplary LSP encompassed for use in the methods and compositions is SP0412 (SEQ ID NO: 91) or a functional variant thereof. In alternative embodiments, a LSP can be selected from any of SEQ ID NOS: 86 (CRM 0412), SEQ ID NO: 91 (SP0412) or SEQ ID NO: 92 (SP0422), SEQ ID NOs: 93 (SP0239), SEQ ID NO: 94 (SP0265, also referred to SP131_A1), SEQ ID NO: 95 (SP0240) or SEQ ID NO: 96 (SP0246), or SEQ ID NO: 146 (SP0265-UTR), SEQ ID NO: 147 (SP0239-UTR), SEQ ID NO: 148 (SP0240-UTR), SEQ ID NO: 149 (SP0246-UTR) or SEQ ID NO: 150 (SP0131-A1-UTR), or functional fragments or variants thereof.

In some embodiments of the compositions and methods described herein, the secretory signal peptide is selected from any of: AAT signal peptide, a fibronectin signal peptide (FN1), a GAA signal peptide, innate GAA leader sequence, AAT sequence, IL2(1-3), IL2 leader sequence (IL2 wt), a modified IL2 leader sequence (IL2 mut), or IgG leader sequence or functional variants thereof having secretory signal activity.

In some embodiments of the compositions and methods described herein, the alpha-glucosidase (GAA) polypeptide is linked to the IGF2 targeting peptide at the N-terminal end of a GAA polypeptide. In some embodiments, the IGF2 targeting peptide is linked to the N-terminal at amino acid 70 of human acid alpha-glucosidase (GAA) polypeptide (SEQ ID NO: 10) (i.e., linked to the N-terminal of residues 70-952 of human acid alpha-glucosidase (GAA) polypeptide), or a GAA polypeptide at least 85% sequence identity to amino acids 70-952 of SEQ ID NO: 10. In alternative embodiments, the IGF2 targeting peptide is linked to the N-terminal at amino acid 40 of human acid alpha-glucosidase (GAA) polypeptide (SEQ ID NO: 10) (i.e., linked to the N-terminal of residues 40-952 of human acid alpha-glucosidase (GAA) polypeptide)), or a GAA polypeptide at least 85% sequence identity to amino acids 40-952 of SEQ ID NO: 10. In some embodiments of the compositions and methods described herein, the GAA polypeptide is encoded by the wild-type GAA nucleic acid sequence (e.g., SEQ ID NO: 11 or SEQ ID NO: 72), or can be a codon optimized GAA nucleic acid sequence, e.g., for any one of increasing expression in vivo, reducing CpG islands and/or reducing innate immune response in a subject. Exemplary codon optimized GAA nucleic acid sequences include, but are not limited to SEQ ID NO; 73, SEQ ID NO: 74, SEQ ID NO: 75, SEQ ID NO: 76 and SEQ ID NO: 182.

In some embodiments of the methods and compositions disclosed herein, the recombinant AAV vector comprises a liver-specific promoter (LSP), for example but not limited to, a liver specific promoter is selected from any in Table 4 herein or functional variants thereof, or functional variants thereof. Exemplary LSP encompassed for use in the methods and compositions include SP0412 and functional variants thereof. In alternative embodiments, a LSP can comprise a nucleic acid sequence selected from any of SP0422, SP0131A1, SP0239, SP0240 or SP0246, or a functional variant thereof as disclosed herein. For Example, the liver specific promoter can comprise a nucleic acid sequence selected from any of SEQ ID NOS: 86 (CRM 0412), SEQ ID NO: 91 (SP0412) or SEQ ID NO: 92 (SP0422), or a functional variant or functional fragment thereof. In alternative embodiments, the liver specific promoter can comprise a nucleic acid sequence selected from any of SEQ ID NOs: 93 (SP0239), SEQ ID NO: 94 (SP0265 also called SP131_A1), SEQ ID NO: 95 (SP0240) or SEQ ID NO: 96 (SP0246), or SEQ ID NO: 146 (SP0265-UTR), SEQ ID NO: 147 (SP0239-UTR), SEQ ID NO: 148 (SP0240-UTR), SEQ ID NO: 149 (SP0246-UTR), or SEQ ID NO: 150 (SP0131-A1-UTR). In some embodiments of the compositions and methods disclosed herein, a liver-specific promoter, includes a liver-specific cis-regulatory element (CRE), a synthetic liver-specific cis-regulatory module (CRM) or a synthetic liver-specific promoter comprising a promoter sequence selected from any of SEQ ID NOs: 270-341 (minimal LSP, which can include a CRM) or SEQ ID NO: 342-430 (exemplary synthetic LSP), or a functional fragment or functional variant thereof, as previously disclosed in Tables 4A or 4B of provisional application 62,937,556, which is encompassed in its entirety by reference herein. These liver-specific promoter elements can include minimal liver-specific promoters (see, e.g., SEQ ID NO: 86, 270-341 or liver-specific proximal promoters (see, e.g., SEQ ID Nos: 91-96, 146-150 and 342-430). For Example, SEQ ID NOs: 86 (CRM 0412), SEQ ID NO: 91 (SP0412) or SEQ ID NO: 92 (SP0422), or a functional variant or functional fragment thereof.

In some embodiments of the methods and compositions disclosed herein, the recombinant AAV vector comprises a liver-specific promoter (LSP), for example but not limited to, a liver specific promoter is selected from any of SEQ ID NOs: 86, 91-96, 146-150, 370-430 or a functional variant or functional fragment thereof.

For example, a functional variant or a functional fragment of a liver-specific promoter disclosed in Table 4 herein, or any LSP selected from SEQ ID NO: 86, 91-96, or 146-150, or 370-430 or a functional variant or functional fragment thereof has at least about 75% sequence identity to, or at least about 80% sequence identity to, at least about 90% sequence identity to, at least about 95% sequence identity to, at least about 98% sequence identity to the original unmodified reference sequence, and also at least 35% of the promoter activity, or at least about 45% of the promoter activity, or at least about 50% of the promoter activity, or at least about 60% of the promoter activity, or at least about 75% of the promoter activity, or at least about 80% of the promoter activity, or at least about 85% of the promoter activity, or at least about 90% of the promoter activity, or at least about 95% of the promoter activity of the corresponding unmodified promoter sequence.

For example, a functional variant or a functional fragment of SEQ ID NO: 92 (SP0422) or SEQ ID NO: 91 (SP0412) has at least about 75% sequence identity to SEQ ID NO: 92 or SEQ ID NO: 91, or at least about 80% sequence identity to SEQ ID NO: 92 or SEQ ID NO: 91, at least about 90% sequence identity to SEQ ID NO: 92 or SEQ ID NO: 91, at least about 95% sequence identity to SEQ ID NO: 92 or SEQ ID NO: 91, at least about 98% sequence identity to SEQ ID NO: 92 or SEQ ID NO: 91, or the original unmodified sequence, and also at least 35% of the promoter activity, or at least about 45% of the promoter activity, or at least about 50% of the promoter activity, or at least about 60% of the promoter activity, or at least about 75% of the promoter activity, or at least about 80% of the promoter activity, or at least about 85% of the promoter activity, or at least about 90% of the promoter activity, or at least about 95% of the promoter activity of the corresponding unmodified promoter sequence of SEQ ID NO: 92 or SEQ ID NO: 91, respectively.

A functional fragment is a portion of the promoter that has at least 35%, or at least about 45%, or at least about 50%, or at least about 75%, or at least about 80%, or at least about 85%, or at least about 90% of the untrunkated promoter. In some embodiments, a functional fragment comprises a contiguous portion of the unmodified promoter sequence. While TTR (SEQ ID NO: 431) is disclosed in the Examples herein as an exemplary LSP, one of ordinary skill in the art can replace the TTR promoter (SEQ ID NO: 431) with any one or more of the liver-specific promoter listed in Table 4 herein, for example, a nucleic acid sequence comprising at least SEQ ID NO: 92 (SP0422) or SEQ ID NO: 91 (SP0412) or a functional variant or fragment of SEQ ID NO: 92 (SP0422) or SEQ ID NO: 91 (SP0412), or a nucleic acid sequence comprising any of SEQ ID NOs: 93 (SP0239), SEQ ID NO: 94 (SP131_A1), SEQ ID NO: 95 (SP0240), SEQ ID NO: 96 (SP0246), or SEQ ID NO: 146 (SP0265-UTR), SEQ ID NO: 147 (SP0239-UTR), SEQ ID NO: 148 (SP0240-UTR), SEQ ID NO: 149 (SP0246-UTR) or SEQ ID NO: 150 (SP0131-A1-UTR), or any sequence selected from SEQ ID NO: 270-341 or 342-430, or a functional variant or functional fragment thereof. In some embodiments, the LSP, while preferentially expresses the hGAA protein in the liver, can also express the hGAA to some extent in another tissue of interest, e.g., the muscle, or CNS, or muscle and CNS tissues.

In some embodiments of the methods and compositions disclosed herein, a recombinant AAV vector comprises a heterologous nucleic acid sequence that encodes a wild-type GAA polypeptide (wtGAA) or a modified GAA polypeptide, as disclosed herein, where one or more amino acids of the GAA polypeptide is modified, e.g., H199R, R223H, H201L modifications. In some embodiments of the methods and compositions disclosed herein, a recombinant AAV vector comprises a heterologous nucleic acid sequence encoding the GAA polypeptide that is the human GAA gene or a human codon optimized GAA gene (coGAA) or a modified GAA nucleic acid sequence that is codon optimized that encodes a modified GAA polypeptide comprising one or more of the modifications selected from: H199R, R223H, H201L. In all aspects of the methods and compositions as disclosed herein, a nucleic acid sequence encoding the GAA polypeptide is codon optimized for any one or more of: enhanced expression in vivo, to reduce CpG islands, or to reduce the innate immune response. In all aspects of the methods and compositions as disclosed herein, a nucleic acid sequence encoding the GAA polypeptide is codon optimized to reduce CpG islands and to reduce the innate immune response. In some embodiments, the nucleic acid sequence encoding the wild type GAA polypeptide comprises modifications as disclosed in SEQ ID NO: 182, and described herein.

Another aspect of the technology herein relates to a pharmaceutical composition comprising any of the recombinant AAV vector compositions disclosed herein, and a pharmaceutically acceptable carrier.

Another aspect of the technology herein relates to a composition comprising a nucleic acid sequence comprising in the following order: a 5′ ITR, a liver specific promoter (LSP) operatively linked to a nucleic acid sequence comprising a nucleic acid encoding a modified GAA polypeptide comprising one or more of the modifications selected from: H199R, R223H, H201L, and a 3′ ITR. In one aspect the nucleic acid sequence optionally further comprises a nucleic acid sequence encoding a leader sequence (or signal sequence) located between the LSP and the nucleic acid encoding the GAA polypeptide, where the leader sequence is selected from any of: the innate GAA leader sequence, AAT sequence, IL2(1-3), IL2 leader sequence (IL2 wt), a modified IL2 leader sequence (IL2 mut), fibronectin (FN1), or IgG leader sequence or functional variants thereof, as disclosed herein. In some embodiments, the nucleic acid sequence optionally further comprises a kozak sequence located between the LSP and the leader sequence. In some embodiments, the nucleic acid sequence optionally further comprises am IGF2 targeting peptide located between the leader sequence and the nucleic acid encoding the GAA polypeptide. In some embodiments, the nucleic acid sequence optionally further comprises a 3′ UTR located 3′ of the nucleic acid encoding the GAA polypeptide and the poly A sequence. In some embodiments, the nucleic acid sequence optionally further comprises an intron sequence 3′ of the LSP and 5′ of the nucleic acid encoding the GAA polypeptide, preferably between the LSP and the kozak sequence. Exemplary constructs for the rAAV vector or rAAV genome are shown in FIGS. 5A-5G.

Another aspect of the technology herein relates to a composition comprising a nucleic acid sequence comprising a 5′ ITR, a liver specific promoter (LSP) operatively linked to a nucleic acid sequence encoding a modified GAA polypeptide comprising one or more of the modifications selected from: H199R, R223H, H201L, a polyA sequence and a 3′ ITR sequence, where the poly A sequence can be a full length or truncated polyA signal sequence. Another aspect of the technology herein relates to a composition comprising a nucleic acid sequence comprising a 5′ ITR, a liver specific promoter (LSP) operatively linked to a nucleic acid sequence encoding a modified GAA polypeptide comprising one or more of the modifications selected from: H199R, R223H, H201L, a full-length polyA sequence, a terminal repeat sequence and a 3′ ITR sequence, where the nucleic acid lacks a AAV P5 promoter sequence.

Another aspect of the technology herein relates to a composition comprising a nucleic acid sequence comprising: a liver specific promoter (LSP) operatively linked to a nucleic acid sequence comprising, in the following order: (a) a nucleic acid encoding a secretory signal peptide, (b) a nucleic acid encoding a IGF2 targeting peptide, and (c) a nucleic acid encoding a GAA polypeptide.

Another aspect of the technology herein relates to a composition comprising a nucleic acid sequence for a recombinant adenovirus associated (rAAV) vector genome, the nucleic acid sequence comprising: (a) a 5′ and a 3′ AAV inverted terminal repeats (ITR) nucleic acid sequences, and (b) located between the 5′ and 3′ ITR sequence, a heterologous nucleic acid sequence encoding a polypeptide comprising a secretory signal peptide and an alpha-glucosidase (GAA) polypeptide, wherein the heterologous nucleic acid is operatively linked to a liver-specific promoter as described above. An exemplary liver-specific promoter is SP0412 or SP0422 or a functional variant thereof. In some embodiments, a liver-specific promoter for use in the methods and compositions as disclosed herein includes a liver-specific cis-regulatory element (CRE), a synthetic liver-specific cis-regulatory module (CRM) or a synthetic liver-specific promoter as disclosed in Table 4 herein.

In some embodiments of the methods and compositions disclosed herein, the nucleic acid sequence comprises a heterologous nucleic acid sequence encoding a GAA polypeptide, where the nucleic acid sequence is a human GAA gene or a human codon optimized GAA gene (coGAA) or a modified GAA nucleic acid sequence. In some embodiments of the methods and compositions disclosed herein, the nucleic acid sequence comprises a heterologous nucleic acid sequence that is a codon optimized (coGAA) GAA gene, for any one or more of enhanced expression in vivo, to reduce CpG islands or to reduce the innate immune response. In some embodiments of the methods and compositions disclosed herein, the nucleic acid sequence comprises a heterologous nucleic acid sequence that is a codon optimized (coGAA) GAA gene to reduce CpG islands and to reduce the innate immune response.

In some embodiments of the methods and compositions disclosed herein, the nucleic acid sequence comprises a heterologous nucleic acid sequence encoding a GAA polypeptide selected from any of SEQ ID NO: 11 (full length hGAA), SEQ ID NO: 55 (Dwight cDNA), SEQ ID NO: 56 (hGAA Δ1-66) or SEQ ID NO: 182 (modGAA, H199R, R223H), or a nucleic acid sequence encoding a GAA polypeptide having the amino acid sequence of SEQ ID NO: 170 (modGAA; H199R, R223H), SEQ ID NO: 171 (modGAA; H199R, R223H, H201L), or a nucleic acid sequence encoding a GAA polypeptide that is at least about 75%, or 80%, or 85%, or 90%, or 95%, or 98%, or 99% sequence identity to any of SEQ ID NOs: 11, 55, 56 or 182.

In some embodiments of the methods and compositions disclosed herein, the nucleic acid sequence comprises a heterologous nucleic acid sequence encoding the GAA polypeptide, where the nucleic acid encoding the GAA polypeptide is selected from any of SEQ ID NO: 74 (codon optimized 1), SEQ ID NO: 75 (codon optimized 2), and SEQ ID NO: 76 (codon optimized 3), or SEQ ID NO: 182 (modGAA, H199R, R223H), or a nucleic acid sequence at least about 75%, or 80%, or 85%, or 90%, or 95%, or 98%, or 99% sequence identity to any of SEQ ID NOs: 74, 75, 76 or 182.

Another aspect of the technology herein relates to use of the rAAV and nucleic acid compositions disclosed herein in a method to treat a disease. In particular, one aspect of the technology herein relates to use of the rAAV vector compositions and nucleic acid compositions disclosed herein, in a method to treat a subject with a glycogen storage disease type II (GSD II, Pompe Disease, Acid Maltase Deficiency) or having a deficiency in alpha-glucosidase (GAA) polypeptide, the method comprising administering any of the recombinant AAV vector, or the rAAV genome or the nucleic acid sequence disclosed herein to the subject. In some embodiments of the methods disclosed herein, the expressed GAA polypeptide is secreted from the subject's liver and there is uptake of the secreted GAA by skeletal muscle tissue, cardiac muscle tissue, diaphragm muscle tissue or a combination thereof, wherein uptake of the secreted GAA results in a reduction in lysosomal glycogen stores in the tissue(s). In some embodiments in the disclosed methods, the recombinant AAV vector, or the rAAV genome or the nucleic acid sequence is administered to the subject by any suitable administration method, for example, but not limited to, an administration method selected from any of: intramuscular, sub-cutaneous, intraspinal, intracisternal, intrathecal, intravenous administration. In some embodiments, the pharmaceutical composition disclosed herein can be used in the methods disclosed herein.

Another aspect of the technology herein relates to a cell comprising any one or more of a rAAV composition, a rAAV genome composition, or a nucleic acid composition as disclosed herein. In some embodiments, the cell is a human cell, or a non-human cell mammalian cell, or an insect cell.

Another aspect of the technology herein relates to host animal comprising any one or more of a rAAV composition, a rAAV genome composition, or a nucleic acid composition as disclosed herein. In some embodiments, the host animal is a mammal, a non-human mammal or a human.

Another aspect of the technology herein relates to host animal comprising at least one cell that comprises any one or more of a rAAV composition, a rAAV genome composition, or a nucleic acid composition as disclosed herein. In some embodiments, the host animal comprising such a modified cell is a mammal, a non-human mammal or a human.

In some embodiments, disclosed herein is a pharmaceutical formulation comprising an rAAV vectors, nucleic acid encoding a rAAV genome as disclosed herein, and a pharmaceutically acceptable carrier.

Aspects of the present invention teach certain benefits in construction and use which give rise to the exemplary advantages described below. Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

This application file contains at least one drawing executed in color. Copies of this patent application publication with color drawings will be provided by the Office upon request and payment of the necessary fee. The accompanying drawings illustrate aspects of the present invention. In such drawings:

FIG. 1 is a graph illustrating a y-axis of vector genomes per diploid genome and an x-axis of different AAV serotypes AAV3b, AAV3ST, AAV8, and AAV9, as measured in whole blood, in accordance with at least one embodiment.

FIG. 2 is a graph illustrating a y-axis of vector genomes per diploid genome and an x-axis of different AAV serotypes AAV3b, AAV3ST, AAV8, and AAV9, as measured in left, median and right liver lobes, in accordance with at least one embodiment.

FIGS. 3A-3B are exemplary plasmids for production of rAAV vectors useful in the methods and compositions as disclosed herein. FIG. 3A is an illustration of a plasmid map of pAAV-LSPhGAA plasmid for production of a rAAV vector in a producer cell line, e.g., a pro-10 cell line, in accordance with at least one embodiment, where the plasmid comprises a 5′ ITR, LSP, hGAA nucleic acid sequence, 3′ UTR, polyA sequence, and 3′ ITR, where the ITRs are from AAV2. FIG. 3B shows a more detailed map of the illustration of the plasmid map of FIG. 3A.

FIGS. 4A-4G are illustrations of exemplary nucleic acid constructs for a rAAV genome as disclosed herein that have a targeting peptide, using hGAA as the exemplary lysosomal protein being expressed. FIG. 4A shows a nucleic acid construct for a rAAV genome, comprising a 5′ ITR, a Liver specific promoter (LSP), operatively linked to a heterologous nucleic acid encoding a secretory signal peptide (SS), a targeting peptide (TP) and a human GAA (hGAA) polypeptide, and a 3′ ITR. FIG. 4B shows an exemplary nucleic acid construct for a rAAV genome as disclosed herein, comprising the same elements as FIG. 4A, and additionally comprising at least one polyA signal 3′ of the hGAA polypeptide and 5′ of the 3′-ITR. FIG. 4C shows an exemplary nucleic acid construct for a rAAV genome as disclosed herein, comprising the same elements as FIG. 4B, except comprising with an intron sequence 3′ of the promoter. FIG. 4D shows an exemplary nucleic acid construct for a rAAV genome as disclosed herein, comprising the same elements as FIG. 4C, except comprising a collagen stability (CS) sequence and/or a 3′ UTR sequence located 3′ of the hGAA polypeptide nucleic acid sequence and before the poly A sequence. FIG. 4E shows an exemplary nucleic acid construct for a rAAV genome as disclosed herein, comprising the same elements as FIG. 4D, except also comprising a nucleic acid encoding a spacer of at least 1 amino acid that is located between the nucleic acid encoding the hGAA polypeptide and the nucleic acid encoding the targeting peptide (TP), e.g., IGF2 targeting peptide. FIG. 4F shows an exemplary nucleic acid construct for a rAAV genome as disclosed herein, comprising the same elements as FIG. 4E, wherein the promoter is a liver promoter, the intron sequence is selected from a MVM or HBB2 intron sequence, the secretory signal peptide is selected from any of FN1 signal peptide (e.g., hFN1, ratFN1), a AAT signal peptide or a hGAA signal peptide; the targeting peptide is a IGF2 targeting peptide as disclosed herein, and the at least polyA sequence is selected from hGHpA or a synPA poly A sequence. FIG. 4G shows an exemplary nucleic acid construct for a rAAV genome as disclosed herein, comprising the same elements as FIG. 4F, except where the IGF2 targeting peptide is a nucleic acid sequence selected from SEQ ID NO: 2 (IGF2 Δ2-7), SEQ ID NO: 3 (IGF2 Δ1-7), or SEQ ID NO: 4 (IGF2 V43M).

FIG. 5A-5G shows an exemplary nucleic acid constructs for a rAAV genome. FIG. 5A is a schematic of exemplary rAAV genome comprising a 5′ ITR, a liver specific promoter, operatively linked to a nucleic acid encoding a hGAA polypeptide, a polyA sequence (e.g., any one or more of hGHpA, synPA, RBG or SV40 polyA sequences) and a 3′ ITR. FIG. 5B is a schematic of an exemplary rAAV genome comprising a 5′ ITR, a liver specific promoter, operatively linked to a nucleic acid encoding a signal secretory peptide (e.g., selected from any of FN1, AAT or cognate GAA signal peptide, IL2, mutIL2, IgG), a nucleic acid encoding a human GAA polypeptide and a polyA sequence and a 3′ ITR. FIG. 5C is a schematic of an exemplary rAAV genome comprising a 5′ ITR, a liver specific promoter, operatively linked to an intron sequence (e.g., MVM, SV40 or HBB2 intron sequence), a nucleic acid encoding a signal secretory peptide (e.g., selected from any of FN1, AAT or cognate GAA signal peptide, IL2, mutIL2, IgG), a nucleic acid encoding a human GAA polypeptide and a polyA sequence and a 3′ ITR. FIG. 5D is a schematic of a similar construct to FIG. 5C, which includes a collagen stability (CS) sequence or 3′ UTR located between the 3′ of the nucleic acid encoding GAA and the at least one polyA sequence (e.g., hGHpA and/or synPA polyA sequence). In some embodiments, the construct comprises both a CS sequence and a 3 UTR sequence as disclosed herein. In some embodiments, the CS sequence can be replaced by a 3′ UTR sequence as disclosed herein. In FIGS. 5A-5D, exemplary liver specific promoter can be selected from any of those disclosed in Table 4 herein, and include, but are not limited to SEQ ID NOs 86, 91-96, or 146-150, or a sequence with at least 85% sequence identity to SEQ ID NOs: 86, 91-96, or 146-150. FIG. 5E is a schematic of one embodiment of a AAV vector useful in the methods and compositions as disclosed herein for treating Pompe Disease, comprising, flanked between a 5′ ITR and a 3′ ITR sequence, the nucleic acid comprising in a 5′ to 3′ direction: a LSP promoter, a kozak sequence, a signal sequence (referred to as leader sequence in FIG. 5E), a nucleic acid encoding hGAA and a poly A sequence. In some embodiments, the leader sequence can be selected from any of: innate GAA leader sequence, IL2 leader sequence (IL2 wt), a modified IL2 leader sequence (IL2 mut) or IgG leader sequence or functional variants thereof; and the hGAA sequence can be selected from a consensus hGAA nucleic acid sequence or a hGAA nucleic acid with at least the H201L mutation, or other modifications as disclosed herein (e.g., H199R, R223H). FIG. 5F is a schematic of another embodiment of a AAV vector useful in the methods and compositions as disclosed herein for treating Pompe Disease, comprising in a 5′ to 3′ direction: a liver specific promoter, an intron sequence, a kozak sequence, a signal sequence (also referred to as a leader sequence), an IGF2 targeting peptide sequence (referred to in FIG. 5F as a “GILT”), a nucleic acid encoding hGAA, optionally a 3′ UTR sequence, and a poly A sequence, showing that different embodiments, e.g., the promoter can be selected from any LSP as disclosed herein, e.g., LSP that have different levels of expression, such as a High-expression level LSP (LSP-H), a medium expression level LSP (LSP-M) or low-expressing LSP (LSP-L), the intron sequence can be selected from HBB2, MVM, SV40 and other intron sequences, the leader sequence can be selected from any of: innate GAA leader sequence, AAT sequence (referred to as A1AT in FIG. 5F), IL2(1-3), IL2 leader sequence (IL2 wt), a modified IL2 leader sequence (IL2 mut), fibronectin (FN1, referred to as FBN in FIG. 5F), or IgG leader sequence or functional variants thereof, an IGF2 targeting peptide sequence selected from any of the IGF2 targeting peptides described herein, e.g., WT IGF2 (SEQ ID NO: 1), Δ2-7, V43M (SEQ ID NO: 9), Δ2-7V43M, or functional variants thereof, and a hGAA nucleic acid sequence that is codon optimized as disclosed herein, e.g., C1-10, which can optionally also comprise at least the H201L mutation, and/or other modifications as disclosed herein (e.g., H199R, R223H), and a polyA sequence, selected from, e.g., RBG or SV40 polyA. The LSP designated LSP-H, M-LSP and LSP-L represent liver specific promoters that predominantly and preferentially express hGAA in the liver, but can express hGAA in one or more other tissues, for example, in the muscle. Such LSPs allows for expression in the liver for systemic secretion and uptake by the muscle cells, as well as some expression in the muscle tissues.

FIG. 5G shows schematic of different embodiments of a AAV vector construct useful in the methods and compositions as disclosed herein for treating Pompe Disease, where construct 1 (top panel) shows a rAAV vector construct comprising in a 5′ to 3′ direction, a 5′ ITR, AAV P5 promoter, liver-specific promoter (LSP), hGAA nucleic acid sequence, truncated polyA sequence (t-pA), and 3′ ITR; and Construct 2 (bottom panel) shows an exemplary rAAV vector construct where the P5 AAV promoter fragment is removed, the construct comprising in a 5′ to 3′ direction, a 5′ ITR, liver-specific promoter (LSP), hGAA nucleic acid sequence, full length polyA sequence (fl-pA), a terminator sequence in the antisense orientation and 3′ ITR (in the sense orientation).

FIG. 6 shows an illustration of the Gibson cloning technique to generate rAAV genomes as disclosed herein. In particular, a triple ligation is performed to ligate 3 blocks of nucleic acid sequence together, which can then be cloned into a vector with the promoter, e.g., liver specific promoter, and 5′ and 3′ ITRs to generate the rAAV genome. The Gibson cloning methodology was used to generate the following rAAV genomes: SEQ ID NO: 57 (AAT-V43M-wtGAA (delta1-69aa)); SEQ ID NO: 58 (ratFN1-IGF2V43M-wtGAA (delta1-69aa)); SEQ ID NO: 59 (hFN1-IGF2V43M-wtGAA (delta1-69aa)); SEQ ID NO: 60 (AAT-IGF2Δ2-7-wtGAA (delta 1-69)); SEQ ID NO: 61 (FN1rat-IGFΔ2-7-wtGAA (delta 1-69)); SEQ ID NO: 62 (hFN1-IGFΔ2-7-wtGAA (delta 1-69)).

FIG. 7 shows the generation of an exemplary rAAV genome of SEQ ID NO: 57 comprising AAT-V43M-wtGAA (delta1-69aa)) using Gibson cloning of nucleic acid sequence blocks (1, 2 and 3). One of ordinary skill in the art can readily replace the TTR liver promoter with any of the liver specific promoters disclosed in Table 4 herein, including but not limited to a promoter selected from any of SEQ ID NO: 86, 91-96, 146-150, or a functional variant or functional fragment thereof. Also shown in the AAT-V43M-wtGAA (delta1-69aa)) vector is the location a 3 amino acid (3aa) spacer nucleic acid sequence (showing the exemplary 3aa sequence “G-A-P” as SEQ ID NO: 31) which is located 3′ of the nucleic acid sequence encoding the IGF2(V43M) targeting peptide and 5′ of the nucleic acid encoding wtGAA(A1-69) enzyme, and a stuffer nucleic acid sequence (referred to in FIG. 8. as a “spacer” sequence) which is located 3′ of the polyA sequence and 5′ of the 3′ITR sequence.

FIG. 8 shows the generation of a rAAV genome of SEQ ID NO: 62 comprising hFN1-IGFΔ2-7-wtGAA (delta 1-69), using Gibson cloning of nucleic acid sequence blocks (8, 2 and 3). One of ordinary skill in the art can readily replace the TTR liver promoter with any of the liver specific promoters disclosed in Table 4 herein, including but not limited to a promoter selected from any of SEQ ID NO: 86, 91-96, 146-150, or a functional variant or functional fragment thereof. Also shown in the hFN1-IGFΔ2-7-wtGAA (delta 1-69) vector is the location a 3 amino acid (3aa) spacer nucleic acid sequence (showing the exemplary 3aa sequence “G-A-P” as SEQ ID NO: 31) which is located 3′ of the nucleic acid sequence encoding the IGFΔ2-7 targeting peptide and 5′ of the nucleic acid encoding wtGAA(A1-69) enzyme, and a stuffer nucleic acid sequence (referred to in FIG. 13. as a “spacer” sequence) which is located 3′ of the polyA sequence and 5′ of the 3′ITR sequence.

FIGS. 9A-9F shows schematics of exemplary constructs of rAAV genomes expressing wild-type GAA. FIG. 9A shows a schematic of exemplary rAAV genome construct of Candidate 1_AAT_hIGF2-V43M_wtGAA_del1-69_Stuffer.V02 (SEQ ID NO: 79). FIG. 9B shows a schematic of exemplary rAAV genome construct of Candidate 2_FIBrat_hIGF2-V43M_wtGAA_del1-69_Stuffer.V02 (SEQ ID NO: 80). FIG. 9C shows a schematic of exemplary rAAV genome construct of Candidate 3_FIBhum_hIGF2-V43M_wtGAA_del1-69_Stuffer.V02 (SEQ ID NO: 81) FIG. 9D shows a schematic of exemplary rAAV genome construct of Candidate 4_AAT_GILT_wtGAA_del1-69__Stuffer.V02 (SEQ ID NO: 82). FIG. 9E shows a schematic of exemplary rAAV genome construct of Candidate 5_FIBrat_GILT_wtGAA_del1-69_Stuffer.V02 (SEQ ID NO: 83). FIG. 9F shows a schematic of exemplary rAAV genome construct of Candidate 6_FIBhum_GILT_wtGAA_del1-69_Stuffer.V02 (SEQ ID NO: 84). One of ordinary skill in the art can readily replace the TTR liver promoter shown in FIGS. 9A-9F for any LPS, e.g., any liver specific promoters disclosed in Table 4 herein, including but not limited to a promoter selected from any of SEQ ID NO: 86, 91-96, or 146-150. Moreover, TTR promoter can be replaced with a LSP that can express the hGAA polypeptide preferentially in the liver and also in at least one other tissue of interest, e.g., the muscle, or CNS, and in some embodiments, the TTR promoter can be replaced with a LSP that can express the hGAA polypeptide preferentially in the liver and the muscle and CNS tissues. In some embodiments, the expressed lysosomal enzyme, e.g., GAA protein can be configured as GAA-fusion protein with a targeting sequence, such as a IGF2 targeting peptide as disclosed herein that targets the GAA protein to lysosomes, and/or fused with a signal peptide (SP), the GAA protein is expressed by the rAAV genome in the liver, where it is secreted and taken up by lysosomes of mammalian cells, in particular muscle cells.

As these are exemplary constructs for illustration purposes only, one can also readily substitute the wtGAA sequence with a codon optimized sequence as disclosed herein, or a GAA sequence that has been modified to reduce CpG islands and/or to reduce innate immunity as disclosed herein (see FIG. 11B).

FIG. 10 shows the mean in vivo luciferase expression in mice driven by exemplary liver-specific promoters SP0244 and SP0239. The expression level is shown as the mean bioluminescence intensity total flux (in photons per second). Error bars are standard error of the mean. When animals are injected with saline only (n=10), no luciferase bioluminescence is detected. When animals are injected with a construct comprising luciferase operably linked to the LP1 promoter (n=9), luciferase bioluminescence is detected. To test the activity of exemplary liver-specific promoters, animals are injected with an equivalent construct comprising luciferase operably inked to the SP0244 promoter (n=8) and the SP0239 promoter (n=10). Promoters SP0244 and SP0239 showed higher luciferase expression in vivo than the control LP1.

FIGS. 11A-11D shows exemplary modifications to the nucleic acid sequence encoding the GAA polypeptide, and the nucleic acid construct to optimize for GAA protein expression by AAV in vivo. FIG. 11A shows a schematic of the wild type GAA (wtGAA) nucleotide sequence operatively linked to a liver specific promoter as disclosed herein, e.g., a LSP of Table 4, with alternative reading frames shown by the arrows, and three CpG islands. FIG. 11B shows a schematic similar to FIG. 11A that shows in more detail modifications to the nucleic acid sequence encoding GAA to remove the CpG islands. FIG. 11C shows modifications to the wtGAA nucleic acid sequence of SEQ ID NO: 182 that has been modified to (i) reduce the alternative reading frames, (ii) the number of CpG islands and (iii) to include modifications for an optimal Kozak sequence. FIG. 11D is another schematic to show modifications in the nucleic acid sequence encoding the GAA polypeptide to reduce the alternative reading frames, the number of CpG islands and to modifications for an optimal Kozak sequence.

FIG. 12 shows schematics of exemplary rAAV constructs comprising LSP for expressing GAA under liver specific promoters. The LSP can be selected from any of the liver specific promoters disclosed in Table 4 herein, with or without a stuffer sequence.

FIGS. 13A-13B show GAA expression from construct comprising liver specific promoters SP0412 and SP0422 in Huh 7 cells and HEPG2 cells. FIG. 13A shows a western blot of GAA expression from construct comprising the liver specific promoter SP0412 (SEQ ID NO: 91) and SP0422 (SEQ ID NO: 92) in Huh 7 cells. FIG. 13A shows that expression of hGAA using promoters 412 (SEQ ID NO: 91) and 422 (SEQ ID NO: 92) leads to significantly higher expression of hGAA in Huh7 cells as compared to the expression using the LP1 promoter (SEQ ID NO: 432) which is referred to as “LSP SS”. FIG. 13B shows a western blot of GAA expression from construct comprising the liver specific promoter SP0412 (SEQ ID NO: 91) and SP0422 (SEQ ID NO: 92) in HEPG2 cells. GAA polypeptide was expressed from rAAV generated using the following plasmids: LSP NEW (SEQ ID NO: 160), 412 NEW (SEQ ID NO: 159), TTR NEW (SEQ ID NO: 155), LSP ss (AAV with LP-1), 412 TTR, 422 Stuffer (SEQ ID NO: 158), 422 TTR, 412 Stuffer (SEQ ID NO: 156). FIG. 13B shows that expression of hGAA using promoters 412 (SEQ ID NO: 91) and 422 (SEQ ID NO: 92) leads to significantly higher expression of hGAA in HepG2 cells as compared to the expression using the LP1 promoter (SEQ ID NO: 432) which is referred to as “LSP SS”.

The above described figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

The disclosure described herein generally relates to recombinant AAV (rAAV) vectors and constructs for rAAV genomes for gene therapy for delivering a lysosomal protein, such as a GAA polypeptide to a subject. In particular, the technology described herein relates in general to a rAAV vector, or a rAAV genome for producing a lysosomal protein, e.g., GAA polypeptide that is expressed in the liver and effectively targeted to the lysosomes of mammalian cells, for example, human cardiac and skeletal muscle cells. For example, the technology relates to a rAAV vector for transducing liver cells, where the transduced liver cells secrete the GAA polypeptide, and the secreted GAA polypeptide is targeted to lysosomes in skeletal muscle tissue, cardiac muscle tissue, diaphragm muscle tissue or a combination thereof

Accordingly, one aspect of the technology described herein provides a rAAV vector comprising a rAAV genome that can be used to produce a lysosomal protein, e.g., GAA or modified GAA, that is more effectively secreted from cells, e.g., liver cells, and then targeted to the lysosomes of mammalian cells, for example, human cardiac and skeletal muscle cells.

In particular, in some embodiments, the lysosomal protein, e.g., GAA polypeptide is expressed by itself. In some embodiments, the lysosomal protein is expressed as a fusion protein comprising at least a signal peptide that promotes secretion of the lysosomal protein, e.g., GAA polypeptide from the liver. In some embodiments, the GAA polypeptide, or modified GAA, is expressed as a fusion protein comprising at least a signal peptide that promotes secretion of the GAA polypeptide from the liver, and also a targeting sequence, that allows effective targeting to lysosomes in mammalian cells, e.g., muscle cells, for example, human cardiac and skeletal muscle cells. In some embodiments, the targeting peptide is a IGF2 targeting peptide a described herein.

One aspect of the technology described herein relates to a rAAV vector that comprises a nucleotide sequence containing inverted terminal repeats (ITRs), a liver specific promoter, a heterologous gene, a poly-A tail and potentially other regulator elements for use to treat a disease, such as Pompe Disease, and further, for the treatment of Pompe Disease, wherein the heterologous gene is a GAA and wherein the rAAV GAA can be administered to a patient in a therapeutically effective dose that is delivered to the appropriate tissue and/or organ for expression of the heterologous gene and treatment of the disease.

One aspect of the technology described herein relates to a rAAV vector that comprises in its genome the following in a 5′ to 3′ direction: 5′- and 3′-AAV inverted terminal repeats (ITR) sequences, and located between the 5′ and 3′ ITRs, a heterologous nucleic acid sequence encoding an alpha-glucosidase (GAA) polypeptide, wherein the heterologous nucleic acid is operatively linked to a liver specific promoter, for example, a liver specific promoter disclosed in Table 4 herein, or a functional variant thereof. Another aspect of the technology described herein relates to a rAAV vector that comprises in its genome the following in a 5′ to 3′ direction: 5′- and 3′-AAV inverted terminal repeats (ITR) sequences, and located between the 5′ and 3′ ITRs, a heterologous nucleic acid sequence encoding a secretory signal peptide (SS), a nucleic acid sequence encoding an alpha-glucosidase (GAA) polypeptide, wherein the heterologous nucleic acid is operatively linked to a liver specific promoter, for example, a liver specific promoter disclosed in Table 4 herein, or a functional variant thereof.

One aspect of the technology described herein relates to a rAAV vector that comprises in its genome the following in a 5′ to 3′ direction: 5′- and 3′-AAV inverted terminal repeats (ITR) sequences, and located between the 5′ and 3′ ITRs, a heterologous nucleic acid sequence encoding a fusion polypeptide comprising (i) a secretory signal peptide (SS), (ii) an IGF2 targeting peptide; and (iii) an alpha-glucosidase (GAA) polypeptide, wherein the heterologous nucleic acid is operatively linked to a liver specific promoter, for example, a liver specific promoter disclosed in Table 4 herein, or a functional variant thereof.

In all aspects of all embodiments of the technology described herein, the liver specific promoter expresses the lysosomal protein, e.g., hGAA polypeptide preferentially in the liver. In all aspects of all embodiments of the technology described herein, the liver specific promoter expresses the lysosomal protein e.g., hGAA polypeptide preferentially in the liver and at least one other tissue of interest, e.g., the muscle, or CNS, and in some embodiments, the LSP can be replaced with a LSP that can express the hGAA polypeptide preferentially in the liver and the muscle and CNS tissues. In all aspects of all embodiments of the technology described herein, in some embodiments where the AAV vector comprises at least one capsid protein targeting the muscle, the liver specific promoter can be replaced with another promoter, e.g., a muscle promoter.

In some embodiments of the methods and compositions as disclosed herein, the secretory signal peptide is selected from any of: AAT signal peptide, a fibronectin signal peptide (FN1), a GAA signal peptide, or an active fragment thereof having secretory signal activity.

In some embodiments, the a rAAV vector described herein is from any serotype. In some embodiments, the rAAV vector is a AAV3b serotype, including, but not limited to, an AAV3b265D virion, an AAV3b265D549A virion, an AAV3b549A virion, an AAV3bQ263Y virion, or an AAV3bSASTG virion (i.e., a virion comprising a AAV3b capsid comprising Q263A/T265 mutations). In some embodiments, the rAAV vector comprises a liver specific capsid, e.g., a liver specific capsid selected from XL32 and XL32.1, as disclosed in WO2019/241324, which is incorporated herein in its entirety by reference. In some embodiments, the rAAV vector is a AAVXL32 or AAVXL32.1 as disclosed in WO2019/241324, which is incorporated herein in its entirety by reference. In some embodiments, the rAAV vector is a rAAV8 vector, or a haploid rAAV vector comprising at least one capsid protein from AAV8 (i.e., any one or more of VP1, VP2 or VP3 is from AAV8 or a chimeric protein thereof). In some embodiments, the AAV vector comprises a capsid disclosed in WO2019241324A 1, or International Patent application PCT/US2019/036676, which are incorporated herein in their entirety by reference. In some embodiments, the AAV vector comprises a capsid which is encoded by a nucleic acid AAV capsid coding sequence that is at least 90% identical to a nucleotide sequence of any one of SEQ ID NOs: 1-3 as disclosed in WO2019241324A1; or (b) a nucleotide sequence encoding any one of SEQ ID NOS:4-6 as disclosed in WO2019241324A1. In some embodiments, an AAV capsid comprises an amino acid sequence at least 90% identical to any one of SEQ ID NOS:4-6 as disclosed in WO2019241324A1, along with AAV particles comprising an AAV vector genome and the AAV capsid of the invention. In some embodiments, the rAAV vector comprises capsid proteins such that the AAV vector transduces liver cells, and in some embodiments the rAAV vector comprises the rAAV vector comprises capsid proteins such that the AAV vector transduces muscle and liver cells. In such embodiments, where the rAAV comprises capsid proteins that enable transduction of muscle cells, the LSP can be replaced with another promoter, e.g., a muscle promoter, or promoter that expresses a protein in liver cells and muscle cells.

### I. Definitions

The following terms are used in the description herein and the appended claims:

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Furthermore, the term “about,” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461,463 (CCPA 1976) (emphasis in the original); see also MPEP § 2111.03. Thus, the term “consisting essentially of” when used in a claim of this invention is not intended to be interpreted to be equivalent to “comprising.” Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate further, if, for example, the specification indicates that a particular amino acid can be selected from A, G, I, Land/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed (e.g., by negative proviso). For example, in particular embodiments the amino acid is not A, G or I; is not A; is not G or V; etc. as if each such possible disclaimer is expressly set forth herein.

The term “parvovirus” as used herein encompasses the family Parvoviridae, including autonomously replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, Muscovy duck parvovirus, B19 virus, and any other autonomous parvovirus now known or later discovered. Other autonomous parvoviruses are known to those skilled in the art. See, e.g., BERNARD N. FIELDS et al., VIROLOGY, volume 2, chapter 69 (4th ed., Lippincott-Raven Publishers).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, ovine AAV, and any other AAV now known or later discovered. See, e.g., BERNARD N. FIELDS et al., VIROLOGY, volume 2, chapter 69 (4th ed., Lippincott-Raven Publishers). A number of relatively new AAV serotypes and clades have been identified (see, e.g., Gao et al., (2004) J. Virology 78:6381-6388; Moris et al., (2004) Virology 33-:375-383); and also Table 1 as disclosed in U.S. Provisional Application 62,937,556, filed on Nov. 19, 2019 and Table 1 in International Applications WO2020/102645, and WO2020/102667, each of which is incorporated herein in their entirety.

The genomic sequences of various serotypes of AAV and the autonomous parvoviruses, as well as the sequences of the native inverted terminal repeats (ITRs), Rep proteins, and capsid subunits are known in the art. Such sequences may be found in the literature or in public databases such as GenBank. See, e.g., GenBank Accession Numbers NC_002077, NC_001401, NC_001729, NC_001863, NC 001829, NC 001862, NC 000883, NC_001701, NC 001510, NC_006152, NC_006261, AF063497, U89790, AF043303, AF028705, AF028704, J02275, J01901, J02275, X01457, AF288061, AH009962, AY028226, AY028223, NC 001358, NC_001540, AF513851, AF513852, AY530579; the disclosures of which are incorporated by reference herein for teaching parvovirus and AAV nucleic acid and amino acid sequences. See also, e.g., Srivistava et al., (1983) J Virology 45:555; Chiarini et al., (1998) J. Virology 71:6823; Chiarini et al., (1999) J. Virology 73:1309; Bantel-Schaal et al., (1999) J. Virology 73:939; Xiao et al., (1999) J. Virology 73:3994; Muramatsu et al., (1996) Virology 221:208; Shade et al., (1986) J. Viral. 58:921; Gao et al., (2002) Proc. Nat. Acad. Sci. USA 99:11854; Morris et al., (2004) Virology 33-:375-383; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1 and Table 5 disclosed in U.S. Pat. No. 62,937,556, filed on Nov. 19, 2019 or Table 1 as disclosed in International Applications WO2020/102645, and WO2020/102667, each of which is incorporated herein in their entirety. The capsid structures of autonomous parvoviruses and AAV are described in more detail in BERNARD N. FIELDS et al., VIROLOGY, volume 2, chapters 69 & 70 (4th ed., Lippincott-Raven Publishers). See also, description of the crystal structure of AAV2 (Xie et al., (2002) Proc. Nat. Acad. Sci. 99:10405-10), AAV4 (Padron et al., (2005) J. Viral. 79: 5047-58), AAV5 (Walters et al., (2004) J. Viral. 78: 3361-71) and CPV (Xie et al., (1996) J Mal. Biol. 6:497-520 and Tsao et al., (1991) Science 251: 1456-64).

The term “tropism” as used herein refers to preferential entry of the virus into certain cells or tissues, optionally followed by expression (e.g., transcription and, optionally, translation) of a sequence(s) carried by the viral genome in the cell, e.g., for a recombinant virus, expression of a heterologous nucleic acid(s) of interest.

As used here, “systemic tropism” and “systemic transduction” (and equivalent terms) indicate that the virus capsid or virus vector of the invention exhibits tropism for and/or transduces tissues throughout the body (e.g., brain, lung, skeletal muscle, heart, liver, kidney and/or pancreas). In embodiments of the invention, systemic transduction of the central nervous system (e.g., brain, neuronal cells, etc.) is observed. In other embodiments, systemic transduction of cardiac muscle tissues is achieved.

As used herein, “selective tropism” or “specific tropism” means delivery of virus vectors to and/or specific transduction of certain target cells and/or certain tissues.

Unless indicated otherwise, “efficient transduction” or “efficient tropism,” or similar terms, can be determined by reference to a suitable control (e.g., at least about 50%, 60%, 70%, 80%, 85%, 90%, 95%, 100%, 125%, 150%, 175%, 200%, 250%, 300%, 350%, 400%, 500% or more of the transduction or tropism, respectively, of the control). In particular embodiments, the virus vector efficiently transduces or has efficient tropism for liver cells and muscle cells. Suitable controls will depend on a variety of factors including the desired tropism and/or transduction profile.

Similarly, it can be determined if a virus “does not efficiently transduce” or “does not have efficient tropism” for a target tissue, or similar terms, by reference to a suitable control. In particular embodiments, the virus vector does not efficiently transduce (i.e., has does not have efficient tropism) for kidney, gonads and/or germ cells. In particular embodiments, transduction (e.g., undesirable transduction) of tissue(s) (e.g., kidney) is 20% or less, 10% or less, 5% or less, 1% or less, 0.1% or less of the level of transduction of the desired target tissue(s) (e.g., liver, skeletal muscle, diaphragm muscle, cardiac muscle and/or cells of the central nervous system).

In some embodiments of this invention, an AAV particle comprising a capsid of this invention can demonstrate multiple phenotypes of efficient transduction of 30 certain tissues/cells and very low levels of transduction (e.g., reduced transduction) for certain tissues/cells, the transduction of which is not desirable.

As used herein, the term “polypeptide” encompasses both peptides and proteins, unless indicated otherwise.

A “polynucleotide” is a sequence of nucleotide bases, and may be RNA, DNA or DNA-RNA hybrid sequences (including both naturally occurring and non-naturally occurring nucleotides), but in representative embodiments are either single or double stranded DNA sequences.

The terms “heterologous nucleotide sequence” and “heterologous nucleic acid molecule” are used interchangeably herein and refer to a nucleic acid sequence that is not naturally occurring in the virus. Generally, the heterologous nucleic acid molecule or heterologous nucleotide sequence comprises an open reading frame that encodes a polypeptide and/or nontranslated RNA of interest (e.g., for delivery to a cell and/or subject).

A “chimeric nucleic acid” comprises two or more nucleic acid sequences covalently linked together to encode a fusion polypeptide. The nucleic acids may be DNA, RNA, or a hybrid thereof.

The term “fusion polypeptide” comprises two or more polypeptides covalently linked together, typically by peptide bonding.

As used herein, an “isolated” polynucleotide (e.g., an “isolated DNA” or an “isolated RNA”) means a polynucleotide at least partially separated from at least some of the other components of the naturally occurring organism or virus, for example; the cell or viral structural components or other polypeptides or nucleic acids commonly found associated with the polynucleotide. In representative embodiments an “isolated” nucleotide is enriched by at least about 10-fold, 100′-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

A population of virions can be generated by any of the methods described herein. In one embodiment, the population is at least 101 virions. In one embodiment, the population is at least 102 virions, at least 103, virions, at least 104 virions, at least 105 virions, at least 106 virions, at least 107 virions, at least 108 virions, at least 109 virions, at least 1010 virions, at least 1011 virions, at least 1012 virions, at least 1013 virions, at least 1014 virions, at least 1015 virions, at least 1016 virions, or at least 1017 virions. A population of virions can be heterogeneous or can be homogeneous (e.g., substantially homogeneous or completely homogeneous).

A “substantially homogeneous population” as the term is used herein, refers to a population of virions that are mostly identical, with few to no contaminant virions (those that are not identical) therein. A substantially homogeneous population is at least 90% of identical virions (e.g., the desired virion), and can be at least 91%, at least 92%, at least 93%, at least 94%, at least 95%, at least 96%, at least 97%, at least 98%, at least 99%, at least 99.5%, at least 99.9% of identical virions.

A population of virions that is completely homogeneous contains only identical virions.

As used herein, by “isolate” or “purify” (or grammatical equivalents) a virus vector or virus particle or population of virus particles, it is meant that the virus vector or virus particle or population of virus particles is at least partially separated from at least some of the other components in the starting material. In representative embodiments an “isolated” or “purified” virus vector or virus particle or population of virus particles is enriched by at least about 10-fold, 100-fold, 1000-fold, 10,000-fold or more as compared with the starting material.

Unless indicated otherwise, “efficient transduction” or “efficient tropism,” or similar terms, can be determined by reference to a suitable control (e.g., at least about 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300%, 350%, 400%, 500% or more of the transduction or tropism, respectively, of the control). In particular embodiments, the virus vector efficiently transduces or has efficient tropism for neuronal cells and cardiomyocytes. Suitable controls will depend on a variety of factors including the desired tropism and/or transduction profile.

A “therapeutic polypeptide” is a polypeptide that can alleviate, reduce, prevent, delay and/or stabilize symptoms that result from an absence or defect in a protein in a cell or subject and/or is a polypeptide that otherwise confers a benefit to a subject, e.g., enzyme replacement to reduce or eliminate symptoms of a disease, or improvement in transplant survivability or induction of an immune response.

The terms “heterologous nucleotide sequence” and “heterologous nucleic acid molecule” are used interchangeably herein and refer to a nucleic acid sequence that is not naturally occurring in the virus. Generally, the heterologous nucleic acid molecule or heterologous nucleotide sequence comprises an open reading frame that encodes a polypeptide and/or nontranslated RNA of interest (e.g., for delivery to a cell and/or subject), for example the GAA polypeptide.

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone.

An “rAAV vector genome” or “rAAV genome” is an AAV genome (i.e., vDNA) that comprises one or more heterologous nucleic acid sequences. rAAV vectors generally require only the inverted terminal repeat(s) (TR(s)) in cis to generate virus. All other viral sequences are dispensable and may be supplied in trans (Muzyczka, (1992) Curr. Topics Microbial. Immunol. 158:97). Typically, the rAAV vector genome will only retain the one or more TR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention the rAAV vector genome comprises at least one ITR sequence (e.g., AAV TR sequence), optionally two ITRs (e.g., two AAV TRs), which typically will be at the 5′ and 3′ ends of the vector genome and flank the heterologous nucleic acid, but need not be contiguous thereto. The TRs can be the same or different from each other.

The term “terminal repeat” or “TR” includes any viral terminal repeat or synthetic sequence that forms a hairpin structure and functions as an inverted terminal repeat (i.e., an ITR that mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

An “AAV terminal repeat” or “AAV TR,” including an “AAV inverted terminal repeat” or “AAV ITR” may be from any AAV, including but not limited to serotypes 1, 2, 3, 4, 5, 6, 7, 8, 9, 10, 11 or 12 or any other AAV now known or later discovered. An AAV terminal repeat need not have the native terminal repeat sequence (e.g., a native AAV TR or AAV ITR sequence may be altered by insertion, deletion, truncation and/or missense mutations), as long as the terminal repeat mediates the desired functions, e.g., replication, virus packaging, integration, and/or provirus rescue, and the like.

AAV proteins VP1, VP2 and VP3 are capsid proteins that interact together to form an AAV capsid of an icosahedral symmetry. VP1.5 is an AAV capsid protein described in US Publication No. 2014/0037585.

The virus vectors of the invention can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al., (2000) Molecular Therapy 2:619.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the invention.

Further, the viral capsid or genomic elements can contain other modifications, including insertions, deletions and/or substitutions.

A “chimeric” capsid protein as used herein means an AAV capsid protein (e.g., any one or more of VP1, VP2 or VP3) that has been modified by substitutions in one or more (e.g., 2, 3, 4, 5, 6, 7, 8, 9, 10, etc.) amino acid residues in the amino acid sequence of the capsid protein relative to wild type, as well as insertions and/or deletions of one or more (e.g., 2, 3, 4, 5, 6, 7, 8, 9, 10, etc.) amino acid residues in the amino acid sequence relative to wild type. In some embodiments, complete or partial domains, functional regions, epitopes, etc., from one AAV serotype can replace the corresponding wild type domain, functional region, epitope, etc. of a different AAV serotype, in any combination, to produce a chimeric capsid protein of this invention. Production of a chimeric capsid protein can be carried out according to protocols well known in the art and a significant number of chimeric capsid proteins are described in the literature as well as herein that can be included in the capsid of this invention.

As used herein, the term “haploid AAV” shall mean that AAV as described in International Application WO2018/170310, or US Application US2018/037149, which are incorporated herein in their entirety by reference. In some embodiments, a population of virions is a haploid AAV population where a virion particle can be constructed wherein at least one viral protein from the group consisting of AAV capsid proteins, VP1, VP2 and VP3, is different from at least one of the other viral proteins, required to form the virion particle capable of encapsulating an AAV genome. For each viral protein present (VP1, VP2, and/or VP3), that protein is the same type (e.g., all AAV2 VP1). In one instance, at least one of the viral proteins is a chimeric viral protein and at least one of the other two viral proteins is not a chimeric. In one embodiment VP1 and VP2 are chimeric and only VP3 is non-chimeric. For example, only the viral particle composed of VP1/VP2 from the chimeric AAV2/8 (the N-terminus of AAV2 and the C-terminus of AAV8) paired with only VP3 from AAV2; or only the chimeric VP1/VP2 28m-2P3 (the N-terminal from AAV8 and the C-terminal from AAV2 without mutation of VP3 start codon) paired with only VP3 from AAV2. In another embodiment only VP3 is chimeric and VP1 and VP2 are non-chimeric. In another embodiment at least one of the viral proteins is from a completely different serotype. For example, only the chimeric VP1/VP2 28m-2P3 paired with VP3 from only AAV3. In another example, no chimeric is present.

The term a “hybrid” AAV vector or parvovirus refers to a rAAV vector where the viral TRs or ITRs and viral capsid are from different parvoviruses. Hybrid vectors are described in international patent publication WO 00/28004 and Chao et al., (2000) Molecular Therapy 2:619. For example, a hybrid AAV vector typically comprises the adenovirus 5′ and 3′ cis ITR sequences sufficient for adenovirus replication and packaging (i.e., the adenovirus terminal repeats and PAC sequence).

The term “polyploid AAV” refers to a AAV vector which is composed of capsids from two or more AAV serotypes, e.g., and can take advantages from individual serotypes for higher transduction but not in certain embodiments eliminate the tropism from the parents.

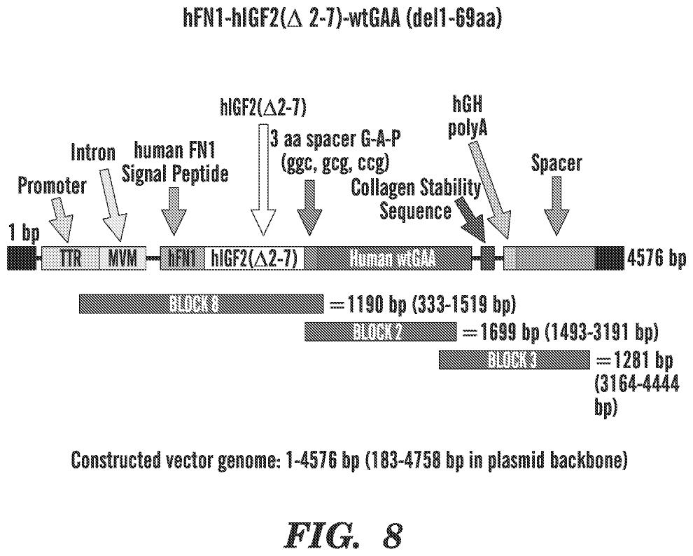

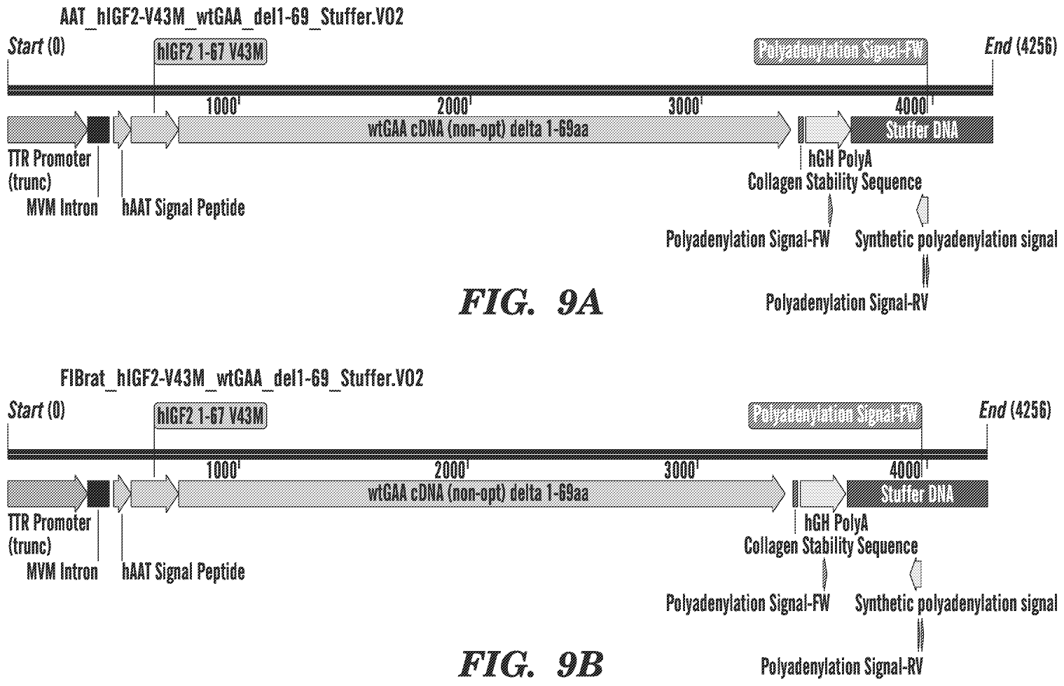

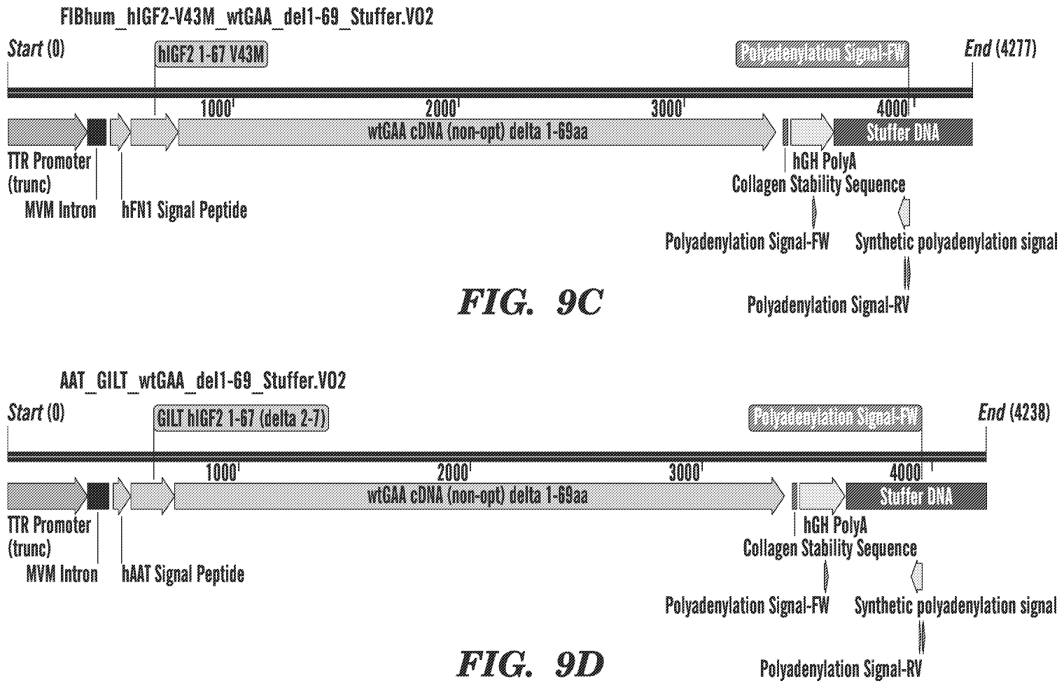

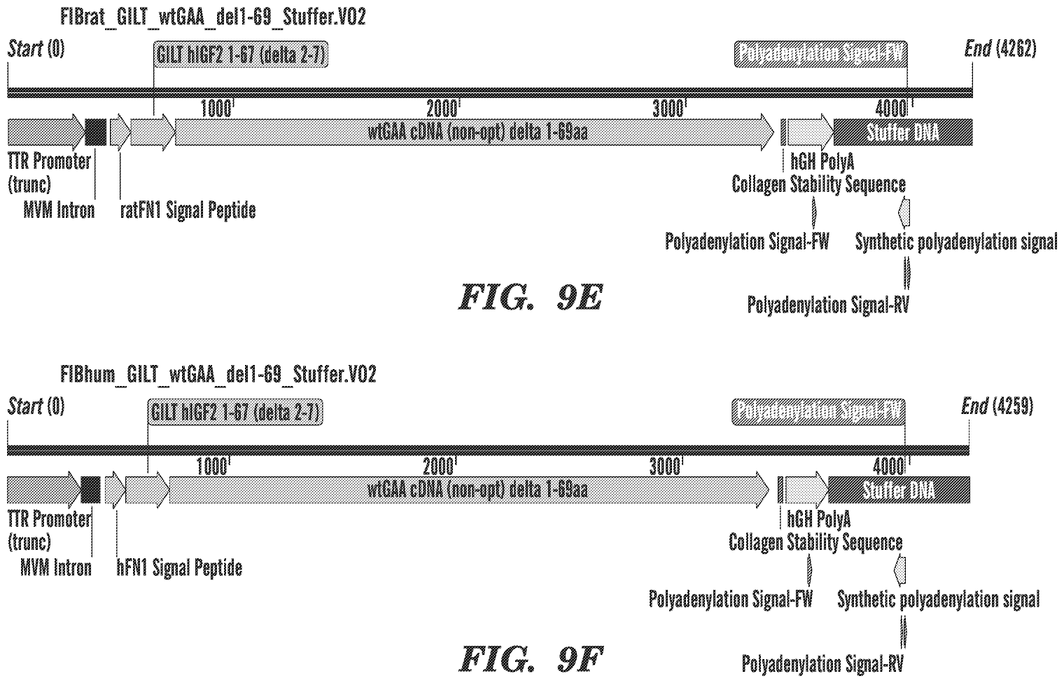

The term “GAA” or “GAA polypeptide,” as used herein, encompasses mature (˜76 or ˜67 kDa) and precursor (e.g., ˜ 10 kDa) GAA as well as modified (e.g., truncated or mutated by insertion(s), deletion(s) and/or substitution(s)) GAA proteins or fragments thereof that retain biological function (i.e., have at least one biological activity of the native GAA protein, e.g., can hydrolyze glycogen, as defined above) and GAA variants (e.g., GAA II as described by Kunita et al., (1997) Biochemica et Biophysica Acta 1362:269; GAA polymorphisms and SNPs are described by Hirschhorn, R. and Reuser, A. J. (2001) in The Metabolic and Molecular Basis for Inherited Disease (Scriver, C. R., Beaudet. A. L., Sly, W. S. & Valle, D. Eds.), pp. 3389-3419, McGraw-Hill, New York, see pages 3403-3405; each incorporated herein by reference in its entirety). Any GAA coding sequence known in the art may be used, for example, see the coding sequences of FIGS. 8 and 9; GenBank Accession number NM_00152 and Hoefsloot et al., (1988) EMBO J. 7:1697 and Van Hove et al., (1996) Proc. Natl. Acad. Sci. USA 93:65 (human), GenBank Accession number NM_008064 (mouse), and Kunita et al., (1997) Biochemica et Biophysics Acta 1362:269 (quail); the disclosures of which are incorporated herein by reference for their teachings of GAA coding and noncoding sequences.

The terms “cation-independent mannose-6-phosphate receptor (CI-MPR),” “M6P/IGF-II receptor,” “CI-MPR/IGF-II receptor,” “IGF-II receptor” or “IGF2 Receptor,” or abbreviations thereof, are used interchangeably herein, referring to the cellular receptor which binds both M6P and IGF-II.

The term “targeting peptide” is also referred to as a “targeting sequence” as used herein is intended to refer to a peptide that targets a particular subcellular compartment, for example, a mammalian lysosome. A targeting peptide encompassed for use herein is a lysosome targeting peptide that is mannose-6-phosphate-independent. An exemplary targeting sequence is an IGF2 targeting peptide as disclosed herein.

The term “IGF2 sequence” is used in conjunction with “IGF2 targeting sequence” or “IGF2 leader sequence” and “IGF2 targeting peptide” are used interchangeably herein and refer to a sequence of the IGF2 polypeptide that binds to the CI-MBR on the surface of the cell. In particular, the IGF2 sequence is a peptide that comprises a part of the IGF2 uptake sequence of SEQ ID NO: 5, or comprises a modification in amino acid of SEQ ID NO:5. An IGF2 targeting peptide refers to a peptide sequence that binds to a receptor domain consisting essentially of repeats 11-12, repeat 11 or amino acids 1508-1566 of the human cation-independent mannose-6-phosphate receptor (CI-MPR or CA-M6P receptor).

The term “leader sequence” is used interchangeably herein with the term “secretory signal sequence” or “signal sequence” or “signal peptide” or variations thereof, and intended to refer to amino acid sequences that function to enhance (as defined above) secretion of an operably linked polypeptide, (e.g., a GAA peptide or IGF2-GAA fusion protein) from the cell as compared with the level of secretion seen with the native polypeptide. As defined above, by “enhanced” secretion, it is meant that the relative proportion of lysosomal polypeptide synthesized by the cell that is secreted from the cell is increased; it is not necessary that the absolute amount of secreted protein is also increased. In particular embodiments of the invention, essentially all (i.e., at least 95%, 97%, 98%, 99% or more) of the GAA-polypeptide is secreted. It is not necessary, however, that essentially all or even most of the GAA polypeptide is secreted, as long as the level of secretion is enhanced as compared with the native GAA polypeptide. Exemplary leader sequences include, but are not limited to the innate GAA leader sequence (also referred to cognate GAA leader sequence), AAT sequence, IL2(1-3), IL2 leader sequence (IL2 wt), a modified IL2 leader sequence (IL2 mut), fibronectin (FN1, also referred to as FBN), or IgG leader sequence or functional variants thereof, as disclosed herein.

As used herein, the term “amino acid” encompasses any naturally occurring amino acid, modified forms thereof, and synthetic amino acids. Naturally occurring, levorotatory (L-) amino acids are disclosed in Table 2 of US Publication 2018/0371496, which is incorporated herein in its entirety. Alternatively, the amino acid can be a modified amino acid residue (nonlimiting examples are shown in Table 4 of US Publication of US Publication 2018/0371496) and/or can be an amino acid that is modified by post-translation modification (e.g., acetylation, amidation, formylation, hydroxylation, methylation, phosphorylation or sulfatation). Further, the non-naturally occurring amino acid can be an “unnatural” amino acid as described by Wang et al., Annu Rev Biophys Biomol Struct. 35:225-49 (2006). These unnatural amino acids can advantageously be used to chemically link molecules of interest to the AAV capsid protein.

To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed (e.g., by negative proviso). For example, in particular embodiments the amino acid is not A, G or I; is not A; is not G or V; etc. as if each such possible disclaimer is expressly set forth herein.

The term “cis-regulatory element” or “CRE”, is a term well-known to the skilled person, and means a nucleic acid sequence such as an enhancer, promoter, insulator, or silencer, that can regulate or modulate the transcription of a neighboring gene (i.e. in cis). CREs are found in the vicinity of the genes that they regulate. CREs typically regulate gene transcription by binding to transcription factors (TFs), i.e. they include TF binding site (TFBS). A single TF may bind to many CREs, and hence control the expression of many genes (pleiotropy). CREs are usually, but not always, located upstream of the transcription start site (TSS) of the gene that they regulate. “Enhancers” are CREs that enhance (i.e. upregulate) the transcription of genes that they are operably associated with, and can be found upstream, downstream, and even within the introns of the gene that they regulate. Multiple enhancers can act in a coordinated fashion to regulate transcription of one gene. “Silencers” in this context relates to CREs that bind TFs called repressors, which act to prevent or downregulate transcription of a gene. The term “silencer” can also refer to a region in the 3′ untranslated region of messenger RNA, that bind proteins which suppress translation of that mRNA molecule, but this usage is distinct from its use in describing a CRE. Generally, the CREs of the present invention are liver-specific enhancers (often referred to as liver-specific CREs, or liver-specific CRE enhancers, or suchlike). In the present context, it is preferred that the CRE is located 1500 nucleotides or less from the transcription start site (TSS), more preferably 1000 nucleotides or less from the TSS, more preferably 500 nucleotides or less from the TSS, and suitably 250, 200, 150, or 100 nucleotides or less from the TSS. CREs of the present invention are preferably comparatively short in length, preferably 100 nucleotides or less in length, for example they may be 90, 80, 70, 60 nucleotides or less in length.

The term “cis-regulatory module” or “CRM” means a functional module made up of two or more CREs; in the present invention the CREs are typically liver-specific enhancers. Thus, in the present application a CRM typically comprises a plurality of liver-specific enhancer CREs. Typically, the multiple CREs within the CRM act together (e.g. additively or synergistically) to enhance the transcription of a gene that the CRM is operably associated with. There is conservable scope to shuffle (i.e. reorder), invert (i.e. reverse orientation), and alter spacing in CREs within a CRM. Accordingly, functional variants of CRMs of the present invention include variants of the referenced CRMs wherein CREs within them have been shuffled and/or inverted, and/or the spacing between CREs has been altered.

As used herein, the phrase “promoter” refers to a region of DNA that generally is located upstream of a nucleic acid sequence to be transcribed that is needed for transcription to occur, i.e. which initiates transcription. Promoters permit the proper activation or repression of transcription of a coding sequence under their control. A promoter typically contains specific sequences that are recognized and bound by plurality of TFs. TFs bind to the promoter sequences and result in the recruitment of RNA polymerase, an enzyme that synthesizes RNA from the coding region of the gene. A great many promoters are known in the art.

The term “synthetic promoter” as used herein relates to a promoter that does not occur in nature. In the present context it typically comprises a synthetic CRE and/or CRM of the present invention operably linked to a minimal (or core) promoter or liver-specific proximal promoter. The CREs and/or CRMs of the present invention serve to enhance liver-specific transcription of a gene operably linked to the promoter. Parts of the synthetic promoter may be naturally occurring (e.g. the minimal promoter or one or more CREs in the promoter), but the synthetic promoter as a complete entity is not naturally occurring.

As used herein, “minimal promoter” (also known as the “core promoter”) refers to a short DNA segment which is inactive or largely inactive by itself, but can mediate transcription when combined with other transcription regulatory elements. Minimum promoter sequence can be derived from various different sources, including prokaryotic and eukaryotic genes. Examples of minimal promoters are discussed above, and include the dopamine beta-hydroxylase gene minimum promoter, cytomegalovirus (CMV) immediate early gene minimum promoter (CMV-MP), and the herpes thymidine kinase minimal promoter (MinTK). A minimal promoter typically comprises the transcription start site (TSS) and elements directly upstream, a binding site for RNA polymerase II, and general transcription factor binding sites (often a TATA box).

As used herein, “proximal promoter” relates to the minimal promoter plus the proximal sequence upstream of the gene that tends to contain primary regulatory elements. It often extends approximately 250 base pairs upstream of the TSS, and includes specific TFBS. In the present case, the proximal promoter is suitably a naturally occurring liver-specific proximal promoter that can be combined with one or more CREs or CRMs of the present invention. However, the proximal promoter can be synthetic.

A “functional variant” of a cis-regulatory element (CRE), cis-regulatory module (CRM), promoter or other nucleic acid sequence in the context of the present invention is a variant of a reference sequence that retains the ability to function in the same way as the reference sequence, e.g. as a liver-specific cis-regulatory enhancer element, liver-specific cis-regulatory module or liver-specific promoter. Alternative terms for such functional variants include “biological equivalents” or “equivalents”.

It will be appreciated that the ability of a given cis-regulatory element to function as a liver-specific enhancer is determined principally by the ability of the sequence to bind the same liver-specific transcription factors (TFs) that bind to the reference sequence. Accordingly, in most cases, a functional variant of a cis-regulatory element will contain TFBS for the same TFs as the reference cis-regulatory element. It is preferred, but not essential, that the transcription factor binding site (TFBS) of a functional variant are in the same relative positions (i.e. order) as the reference cis-regulatory element. It is also preferred, but not essential, that the TFBS of a functional variant are in the same orientation as the reference sequence (it will be noted that TFBS can in some cases be present in reverse orientation, e.g. as the reverse complement vis-à-vis the sequence in the reference sequence). It is also preferred, but not essential, that the TFBS of a functional variant are on the same strand as the reference sequence. Thus, in preferred embodiments, the functional variant comprises TFBS for the same TFs, in the same order, in the same orientation and on the same strand as the reference sequence. It will also be appreciated that the sequences lying between TFBS (referred to in some cases as spacer sequences, or suchlike) are of less consequence to the function of the cis-regulatory element. Such sequences can typically be varied considerably, and their lengths can be altered. However, in preferred embodiments the spacing (i.e. the distance between adjacent TFBS) is substantially the same (e.g. it does not vary by more than 20, preferably by not more than 10%, more preferably it is the same) in a functional variant as it is in the reference sequence. It will be apparent that in some cases a functional variant of a cis-regulatory enhancer element can be present in the reverse orientation, e.g. it can be the reverse complement of a cis-regulatory enhancer element as described above, or a variant thereof.

Levels of sequence identity between a functional variant and the reference sequence can also be an indicator or retained functionality. High levels of sequence identity in the TFBS of the cis-regulatory element is of generally higher importance than sequence identity in the spacer sequences (where there is little or no requirement for any conservation of sequence). However, it will be appreciated that even within the TFBS, a considerable degree of sequence variation can be accommodated, given that the sequence of a functional TFBS does not need to exactly match the consensus sequence.

The ability of one or more TFs to bind to a TFBS in a given functional variant can determined by any relevant means known in the art, including, but not limited to, electromobility shift assays (EMSA), binding assays, chromatin immunoprecipitation (ChIP), and ChIP-sequencing (ChIP-seq). In a preferred embodiment the ability of one or more TFs to bind a given functional variant is determined by EMSA. Methods of performing EMSA are well-known in the art. Suitable approaches are described in Sambrook et al. cited above. Many relevant articles describing this procedure are available, e.g. Hellman and Fried, Nat Protoc. 2007; 2(8): 1849-1861.

The terms “liver-specific” or “liver-specific expression” when in reference to a promoter refers to the ability of a cis-regulatory element, cis-regulatory module or promoter to enhance or drive expression of a gene in the liver (or in liver-derived cells) in a preferential or predominant manner as compared to other tissues (e.g. spleen, muscle, heart, lung, and brain). Expression of the gene can be in the form of mRNA or protein. In some embodiments, liver-specific expression is such that there is negligible expression in other (i.e. non-liver) tissues or cells, i.e. expression is highly liver-specific. In some embodiments, while a liver-specific promoter drives expression preferentially in the liver, it can also drive expression of the gene in another tissue of interest at a lower level, e.g., muscle.

The ability of a cis-regulatory element to function as a liver-specific cis-regulatory enhancer element can be readily assessed by the skilled person. The skilled person can thus easily determine whether any variant of the specific cis-regulatory elements recited above remains functional (i.e. it is a functional variant as defined above). For example, any given cis-regulatory element to be assessed can be operably linked to a minimal promoter (e.g. positioned upstream of CMV-MP) and the ability of the cis-regulatory element to drive liver-specific expression of a gene (typically a reporter gene) is measured. Alternatively, a variant of a cis-regulatory enhancer element can be substituted into a synthetic liver-specific promoter in place of a reference cis-regulatory enhancer element, and the effects on liver-specific expression driven by said modified promoter can be determined and compared to the unmodified form. Similarly, the ability of a cis-regulatory module or promoter to drive liver-specific expression can be readily assessed by the skilled person (e.g. as described in the examples below). Expression levels of a gene driven by a variant of a reference promoter can be compared to the expression levels driven by the reference sequence. In some embodiments, where liver-specific expression levels driven by a variant promoter are at least 50%, at least 60%, at least 70%, at least 80%, at least 90%, or at least 100% of the expression levels driven by the reference promoter, it can be said that the variant remains functional. Suitable nucleic acid constructs and reporter assays to assess liver-specific expression enhancement can easily be constructed, and the examples set out below give suitable methodologies.

Liver-specificity can be identified wherein the expression of a gene (e.g. a therapeutic or reporter gene) occurs preferentially or predominantly in liver-derived cells. Preferential or predominant expression can be defined, for example, where the level of expression is significantly greater in liver-derived cells than in other types of cells (i.e. non-liver-derived cells). For example, expression in liver-derived cells is suitably at least 5-fold higher than non-liver cells, preferably at least 10-fold higher than non-liver cells, and it may be 50-fold higher or more in some cases. For convenience, liver-specific expression can suitably be demonstrated via a comparison of expression levels in a hepatic cell line (e.g. liver-derived cell line such as Huh7 and/or HepG2 cells) or liver primary cells, compared with expression levels in a kidney-derived cell line (e.g. HEK-293), a cervical tissue-derived cell line (e.g. HeLa) and/or a lung-derived cell line (e.g. A549).

The synthetic liver-specific promoters of the present invention are preferably suitable for promoting expression in the liver of a subject, e.g. driving liver-specific expression of a transgene, preferably a therapeutic transgene. In some embodiments, the liver-specific promoters of the invention are suitable for promoting liver-specific transgene expression at a level at least 1.5-fold greater than the LP1 promoter of SEQ ID NO: 432, preferably 2-fold greater than the LP1 promoter, more preferably 3-fold greater than the LP1 promoter, and yet more preferably 5-fold greater than the LP1 promoter (SEQ ID NO: 432). Such expression is suitably determined in liver-derived cells, e.g. in Huh7, and/or HepG2 cells or primary liver cells (suitably primary human hepatocytes). In some embodiments, the synthetic liver-specific promoters of the present invention are suitable for promoting gene expression at a level of at least 1.5-fold less than an LP1 promoter (SEQ ID NO: 432) in non-liver-derived cells (e.g. HEK-293, HeLa, and/or A549 cells).

Preferred synthetic liver-specific promoters of the present invention are suitable for promoting liver-specific transgene expression and have an activity in liver cells which is at least 15%, 20%, 30%, 40%, 50%, 60%, 70%, 80%, 90%, 100%, 125%, 150%, 175%, 200%, 250%, 300%, 350% or 400% of the activity of the TBG promoter (SEQ ID NO: 435).

The synthetic liver-specific promoters of the present invention are preferably suitable for promoting liver-specific expression at a level at least 1.5-fold greater than a CMV-IE promoter of SEQ ID NO: 433 in liver-derived cells, preferably at least 2-fold greater than a CMV promoter in liver-derived cells (e.g. HEK-293, HeLa, and/or A549 cells). The synthetic liver specific promoters disclosed herein can be LSP-H, LSP-M and LSP-L promoters, referring to high, medium and low expression in the liver, and in some embodiments, the LSP-H, LSP-M and LSP-L can preferentially or predominantly express a protein in the liver, but can also express the protein on one or more other tissues, for example, in the muscle and/or brain. Such LSP-H, LSP-M and LSP-L promoters disclosed herein can preferentially express at least 90%, or at least 80%, or at least 70% or at least 60%, or at least 50% of a protein in the liver, and also express at least 10%, or at least 20%, or at least 30%, or at least 40% or at least 50% in another tissue, for example, in muscle tissue. In some embodiments, a LSP-H, LSP-M and LSP-L promoter useful in the method and compositions as disclosed herein, for example for the treatment of Pompe or a lysosomal disease drives or enhances gene expression in a preferential or predominant manner in the liver, but can also express at least some of the protein in muscle tissue.

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250).

The term “synthetic” as used herein means a nucleic acid molecule that does not occur in nature. Synthetic nucleic acid expression constructs of the present invention are produced artificially, typically by recombinant technologies. Such synthetic nucleic acids may contain naturally occurring sequences (e.g. promoter, enhancer, intron, and other such regulatory sequences), but these are present in a non-naturally occurring context. For example, a synthetic gene (or portion of a gene) typically contains one or more nucleic acid sequences that are not contiguous in nature (chimeric sequences), and/or may encompass substitutions, insertions, and deletions and combinations thereof.

A “spacer sequence” or “spacer” as used herein is a nucleic acid sequence that separates two functional nucleic acid sequences. It can have essentially any sequence, provided it does not prevent the functional nucleic acid sequence (e.g. cis-regulatory element) from functioning as desired (e.g. this could happen if it includes a silencer sequence, prevents binding of the desired transcription factor, or suchlike). Typically, it is non-functional, as in it is present only to space adjacent functional nucleic acid sequences from one another.

The term “pharmaceutically acceptable” as used herein is consistent with the art and means compatible with the other ingredients of the pharmaceutical composition and not deleterious to the recipient thereof.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is substantially less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some preventative benefit is provided to the subject.

The phrase a “therapeutically effective amount” and like phrases mean a dose or plasma concentration in a subject that provides the desired specific pharmacological effect, e.g. to express a therapeutic gene in the liver, and secretion into the plasma. It is emphasized that a therapeutically effective amount may not always be effective in treating the conditions described herein, even though such dosage is deemed to be a therapeutically effective amount by those of skill in the art. The therapeutically effective amount may vary based on the route of administration and dosage form, the age and weight of the subject, and/or the disease or condition being treated.

The terms “individual,” “subject,” and “patient” are used interchangeably, and refer to any individual subject with a disease or condition in need of treatment. For the purposes of the present disclosure, the subject may be a primate, preferably a human, or another mammal, such as a dog, cat, horse, pig, goat, or bovine, and the like.

Additional patents incorporated for reference herein that are related to, disclose or describe an AAV or an aspect of an AAV, including the DNA vector that includes the gene of interest to be expressed are: U.S. Pat. Nos. 6,491,907; 7,229,823; 7,790,154; 7,201898; 7,071,172; 7,892,809; 7,867,484; 8,889,641; 9,169,494; 9,169,492; 9,441,206; 9,409,953; and, 9,447,433; 9,592,247; and, 9,737,618.

### II. rAAV Genome Elements

As disclosed herein, one aspect of the technology relates to a rAAV vector comprising a capsid, and within its capsid, a nucleotide sequence referred to as the “rAAV vector genome”. The rAAV vector genome (also referred to as “rAAV genome) includes multiple elements, including, but not limited to two inverted terminal repeats (ITRs, e.g., the 5′-ITR and the 3′-ITR), and located between the ITRs are additional elements, including a promoter, a heterologous gene and a poly-A tail.

In some embodiments, the rAAV genome disclosed herein comprises a 5′ ITR and 3′ ITR sequence, and located between the 5′ITR and the 3′ ITR, a promoter, e.g., a liver specific promoter sequence as disclosed herein, which operatively linked to a heterologous nucleic acid encoding a nucleic acid encoding an alpha-glucosidase (GAA) polypeptide, where the heterologous nucleic acid sequence can optionally further comprise one or more of the following elements: an intron sequence, a nucleic acid encoding a secretory signal peptide, a nucleic acid encoding an IGF2 targeting peptide, and a poly A sequence.

In some embodiments, the rAAV genome disclosed herein comprises a 5′ ITR and 3′ ITR sequence, and located between the 5′ITR and the 3′ ITR, a promoter operatively linked to a heterologous nucleic acid encoding a secretory peptide and nucleic acid encoding an alpha-glucosidase (GAA) polypeptide (i.e., the heterologous nucleic acid encodes a GAA fusion polypeptide comprising a signal peptide-GAA polypeptide), where the rAAV genome optionally further comprises one or more of: an intron sequence, a collagen stability (CS) sequence, a polyA tail and a nucleic acid encoding a spacer of at least 1 amino acid. In some embodiments, the rAAV genome disclosed herein comprises a 5′ ITR and 3′ ITR sequence, and located between the 5′ITR and the 3′ ITR, a liver specific promoter as disclosed herein operatively linked to a heterologous nucleic acid encoding a secretory peptide (e.g., FN1, AAT or GAA signal peptides) and nucleic acid encoding an alpha-glucosidase (GAA) polypeptide, where the rAAV genome optionally further comprises one or more of: an intron sequence (e.g., MVM or HBB2 intron sequence), a collagen stability (CS) sequence, a polyA tail and a nucleic acid encoding a spacer of at least 1 amino acid.

In some embodiments, the rAAV genome disclosed herein comprises a 5′ ITR and 3′ ITR sequence, and located between the 5′ITR and the 3′ ITR, a promoter operatively linked to a heterologous nucleic acid encoding a secretory peptide, a targeting peptide and a GAA polypeptide (i.e., the heterologous nucleic acid encodes a GAA fusion polypeptide comprising a signal peptide-targeting sequence-GAA polypeptide), where targeting peptide is a IGF2 targeting peptide as described herein, and where the rAAV genome can optionally further comprise one or more of: an intron sequence, a collagen stability (CS) sequence, a polyA tail and a nucleic acid encoding a spacer of at least 1 amino acid.

Each of the elements in the rAAV genome are discussed herein.

### A. Alpha-Glucosidase (GAA) Polypeptide

Alpha-glucosidase (GAA) polypeptide is a member of family 31 of glycoside hydrolyases. Human GAA is synthesized as a 110 kDal precursor (Wisselaar et al. (1993) J. Biol. Chem. 268(3): 2223-31). The mature form of the enzyme is a mixture of monomers of 70 and 76 kDal (Wisselaar et al. (1993) J. Biol. Chem. 268(3): 2223-31). The precursor enzyme has seven potential glycosylation sites and four of these are retained in the mature enzyme (Wisselaar et al. (1993) J. Biol. Chem. 268(3): 2223-31). The proteolytic cleavage events which produce the mature enzyme occur in late endosomes or in the lysosome (Wisselaar et al. (1993) J. Biol. Chem. 268(3): 2223-31).

The rAAV vector genome can encode a GAA polypeptide can include, for example, amino acid residues 40-952 or 70-952 of human GAA, or a smaller portion, such as amino acid residues 40-790 or 70-790.

In one embodiment, the GAA polypeptide can be fused to an IGF2 targeting sequence. In some embodiments, a IGF2 targeting sequence is fused to amino acid 40, or amino acid 70, or to an amino acid within one or two positions of amino acid 40 or 70 of human GAA polypeptide. In some embodiments, the IGF2 targeting peptide as disclosed herein is a ligand for an extracellular receptor, for example, the IGF2 targeting peptide binds to human cation-independent mannose-6-phosphate receptor (CI-MPR) or the IGF2 receptor.

The C-terminal 160 amino acids are absent from the mature 70 and 76 kDal GAA polypeptide species. However, certain Pompe alleles resulting in the complete loss of GAA activity map to this region, for example Val949Asp (Becker et al. (1998) J. Hum. Genet. 62:991). The phenotype of this mutant indicates that the C-terminal portion of the protein, although not part of the 70 or 76 kDal species, plays an important role in the function of the protein. It has also been reported that the C-terminal portion of the protein, although cleaved from the rest of the protein during processing, remains associated with the major species (Moreland et al. (Nov. 1, 2004) J. Biol. Chem., Manuscript 404008200). Accordingly, the C-terminal residues could play a direct role in the catalytic activity of the protein, and/or may be involved in promoting proper folding of the N-terminal portions of the protein.

The native GAA gene encodes a precursor polypeptide which possesses a signal sequence and an adjacent putative trans-membrane domain, a trefoil domain (PFAM PF00088) which is a cysteine-rich domain of about 45 amino acids containing 3 disulfide linkages (Thim (1989) FEBS Lett. 250:85), the domain defined by the mature 70/76 kDal polypeptide, and the C-terminal domain. It has been reported that both the trefoil domain and the C-terminal domain are required for the production of functional GAA, and that it is possible that the C-terminal domain interacts with the trefoil domain during protein folding perhaps facilitating appropriate disulfide bond formation in the trefoil domain.

The GAA polypeptide is described in U.S. Pat. Nos. 5,962,313 and 6,537,785, which are incorporated herein in their entireties by reference. One of ordinary skill in the art can appreciate particular positions of GAA to which a secretory signal peptide (SS) or alternatively, the targeting peptide (e.g., IGF2 targeting peptide) can be fused. Accordingly, in one aspect the invention relates to a GAA fusion protein, where the SP or IGF2 targeting peptide is fused to amino acid 40, 68, 69, 70, 71, 72, 779, 787, 789, 790, 791, 792, 793, or 796 of human GAA of SEQ ID NO: 10, or a modified GAA protein of SEQ ID NO: 170-174, or a portion thereof.

In some embodiments of the methods and compositions as disclosed herein, the human GAA protein expressed by the AAV comprises amino acids of SEQ ID NO: 10, or fragments or variants thereof, for example a human GAA protein beginning at residue 40, 68, 69, 70, 71, 72, 779, 787, 789, 790, 791, 792, 793, or 796 of SEQ ID NO: 10. In some embodiments of the methods and compositions as disclosed herein, the human GAA protein expressed by the AAV comprises amino acids of SEQ ID NO: 10, or a protein at least 60%, or 70%, or 80%, 85% or 90% or 95%, or 98%, or 99% identical to SEQ ID NO: 10. In some embodiments of the methods and compositions as disclosed herein, the human GAA protein expressed by the AAV comprises amino acids is a human GAA protein beginning at residue 40, 68, 69, 70, 71, 72, 779, 787, 789, 790, 791, 792, 793, or 796 of SEQ ID NO: 10, or a protein at least 60%, or 70%, or 80%, 85% or 90% or 95%, or 98%, or 99% identical thereto. In some embodiments, the human GAA protein expressed by the AAV comprises amino acids of beginning at residue 40, 68, 69, 70, 71, 72, 779, 787, 789, 790, 791, 792, 793, or 796 of any of SEQ ID NO: 170 (modGAA; H199R, R223H) or SEQ ID NO: 171 (modGAA; H199R, R223H, H201L) or a protein at least 60%, or 70%, or 80%, 85% or 90% or 95%, or 98%, or 99% identical thereto.

In some embodiments, one of ordinary skill in the art can appreciate particular positions of GAA to which a secretory signal peptide (SS) or alternatively, the targeting peptide (e.g., IGF2 targeting peptide) can be fused. For example, International Patent application WO2018046774A1, which is incorporated herein in its entirety, discloses truncated GAA polypeptides to which the secretory signal peptide (SS) or alternatively, the targeting peptide (e.g., IGF2 targeting peptide) can be attached. The signal peptide or IGF2 targeting peptide can be attached to any truncated GAA polypeptide or truncated modified GAA polypeptide, starting amino acids of GAA truncated proteins as disclosed in U.S. Provisional Application 62,937,556, filed on Nov. 19, 2019 and International Application WO 2020/102667, filed Nov. 15, 2019. which is incorporated herein in its entirety by reference.

In some embodiments, the GAA-fusion polypeptides encoded by the rAAV genome as described herein can include, for example, amino acid residues 40-952 or residues 70-952 of human GAA, or a smaller portion, such as amino acid residues 40-790 or 70-790. In one embodiment, a secretory signal peptide (SS) or targeting peptide, e.g., IGF2 targeting peptide is fused to amino acid 40, or to amino acid 70, or to an amino acid within one or two positions of amino acid 40 or 70.

In some embodiments, the fusion protein comprising the secretory signal peptide (SS) and GAA polypeptide and optionally an IGF2 targeting peptide (i.e., a SS-GAA fusion polypeptide, or a SS-IGF2-GAA fusion protein) comprises amino acid residues 40-952 or residues 70-952 of human acid alpha-glucosidase (GAA) (SEQ ID NO: 10). In some embodiments, the N-terminal of the GAA polypeptide is attached to the C-terminus of the SS and in some embodiments, the N-terminal of the GAA polypeptide is attached to the C-terminus of the IGF2 targeting peptide, and the N-terminus of the IGF2 targeting peptide is attached to the C-terminus of the secretory signal peptide.

(i) Modified GAA (modGAA)

In some embodiments, the GAA protein comprises a H201L variant, as disclosed in US2014/0186326, and Moreland et al., Gene, 2012; 491 (25-30), which are both incorporated herein in their entirety by reference. In particular, the histidine (His) at amino acid position 201 is changed to a leucine (L) residue to enables rapid processing of the 76 kD GAA pre-protein into the mature 70 kD GAA protein.

In particular, in some embodiments, a fusion protein as disclosed herein comprises a GAA polypeptide of SEQ ID NO: 10, with a modification of amino acids that results in increased hydrophobicity at or near the N-terminal 70-kDa processing site. In some instances, the GAA peptide is modified at one or more amino acids corresponding to positions 190-209 of SEQ ID NO: 10. In further embodiments, the polypeptide is modified at one or more amino acids corresponding to positions 195-209 of SEQ ID NO: 10. In further embodiments, the modification is at one or more amino acids corresponding to amino acid positions 200-204 of SEQ ID NO: 10. In certain embodiments, the modification is at the amino acid corresponding to position 201 of SEQ ID NO: 10. In further embodiments, the modification is substitution of one or more amino acids with a more hydrophobic amino acid. In other embodiments, the modification is insertion of one or more hydrophobic amino acids. In even further embodiments, the hydrophobic amino acid is chosen from leucine and tyrosine, or a conservative amino acid of leucine or tyrosine.

In certain embodiments, GAA is modified to increase its hydrophobicity at or near the N-terminal 70-kDa processing site by substituting at least one amino acid with a more hydrophobic amino acid. In some embodiments, the substitution may be made within 5 amino acids upstream or downstream of the N-terminal 70-kDa processing site. In certain examples, the amino acid substitution may be made at an amino acid corresponding to position 195 to 209 of SEQ ID NO: 10. In other instances, the amino acid substitution may be made at an amino acid corresponding to position 200 to 204 of SEQ ID NO: 10. In further embodiments, the modified human GAA contains a hydrophobic amino acid at the position corresponding to amino acid position 201 of SEQ ID NO: 10. In some embodiments, GAA is modified by inserting one or more hydrophobic amino acids at or near the N-terminal 70-kDa processing site. Additional modifications include deletion of one or more amino acids at or near the N-terminal 70-kDa processing site.

In certain embodiments, a modified human GAA is provided containing a hydrophobic amino acid (natural or synthetic) at more than one position at the N-terminal 70-kDa processing site, or within 5 amino acids of the N-terminal 70-kDa processing site. In one embodiment, one of the modified amino acids is at the position corresponding to amino acid 201 of SEQ ID NO: 10.

In various embodiments the hydrophobic amino acid is chosen from valine, leucine, isoleucine, methionine, phenylalanine, tryptophan, tyrosine, cysteine or alanine. In further embodiments, the hydrophobic amino acid is leucine or tyrosine. In some embodiments, the modified human GAA contains a synthetic or non-natural amino acid that exhibits hydrophobic properties. Generally, the substituted amino acid is more hydrophobic than the wild-type amino acid, and thus increases the hydrophobicity at or near the N-terminal 70 kDa processing site.

In one exemplary embodiment, the modified GAA has a leucine at the position corresponding to amino acid 201 of SEQ ID NO: 10. In another embodiment, the modified GAA has a tyrosine at the position corresponding to amino acid 201 of SEQ ID NO: 10.

In some embodiments, the modified human GAA protein comprises a polypeptide with a His (H) to Arginine (R) (H199R) modification at amino acid position 199 of SEQ ID NO: 10 (GAA(H199R), or a modification of an arginine (R) to a histidine (H) (R223H) at amino acid position 223 of SEQ ID NO: 10 (GAA(R223H). In some embodiments, the modified human GAA protein comprises a polypeptide with a His (H) to Arginine (R) (H199R) modification at amino acid position 199 of SEQ ID NO: 10, and a modification of an arginine (R) to a histidine (H) (R223H) at amino acid position 223 of SEQ ID NO: 10 (GAA(H199R-R223H). In some embodiments, the modified human GAA protein comprises SEQ ID NO: 170 or a variant of at least 80%, 90%, 95%, or 99% homology to at least 500, 550, 600, 650, 700, 750, 800, 850, or 900 amino acids of SEQ ID NO: 170, having at least one modification of H199R or R223H, or both. In some embodiments, the cognate leader sequence of GAA (i.e., SEQ ID NO: 175 or amino acids 1-27 of SEQ ID NO: 170) is replaced with an IGF2 targeting peptide as disclosed herein, or a leader sequence of SEQ ID NO: 176, or an IL2 wild type leader peptide (SEQ ID NO: 178), modified IL2 leader peptide (SEQ ID NO: 180) or leader peptides at least 90% sequence identity to SEQ ID Nos 176, 178 or 180.

In some embodiments, the modified human GAA protein comprises SEQ ID NO: 171 or a variant of at least 80%, 90%, 95%, or 99% homology to at least 500, 550, 600, 650, 700, 750, 800, 850, or 900 amino acids of SEQ ID NO: 171, comprising at least the modification of H210L. In some embodiments, the cognate leader sequence of GAA (i.e., SEQ ID NO: 175 or amino acids 1-27 of SEQ ID NO: 171) is replaced with an IGF2 targeting peptide as disclosed herein, or a leader sequence of SEQ ID NO: 176, or an IL2 wild type leader peptide (SEQ ID NO: 178), modified IL2 leader peptide (SEQ ID NO: 180) or leader peptides at least 90% sequence identity to SEQ ID Nos 176, 178 or 180

In some embodiments, the modified human GAA protein comprises a polypeptide with at least one modification selected from: H199R, R223H, or H201L of SEQ ID NO: 10, or a variant of at least 80%, 90%, 95%, or 99% homology to at least 500, 550, 600, 650, 700, 750, 800, 850, or 900 amino acids of SEQ ID NO: 10 having at least one of these modification. In some embodiments, the modified human GAA protein comprises a polypeptide comprises at least two modifications selected from: H199R, R223H, or H201L of SEQ ID NO: 10, or a variant of at least 80%, 90%, 95%, or 99% homology to at least 500, 550, 600, 650, 700, 750, 800, 850, or 900 amino acids of SEQ ID NO: 10 having at least two of these modification. In some embodiments, the modified human GAA protein comprises a polypeptide with three modifications H199R, R223H, and H201L of SEQ ID NO: 10 (GAA-H199R-H201L-R223H), or a variant of at least 80%, 90%, 95%, or 99% homology to at least 500, 550, 600, 650, 700, 750, 800, 850, or 900 amino acids of SEQ ID NO: 10 having these three modifications.

In certain embodiments, modified human GAAs are provided having at least 80%, 90%, 95%, or 99% homology to at least 500, 550, 600, 650, 700, 750, 800, 850, or 900 amino acids of SEQ ID NO: 10, and wherein the modified human GAA has at least one amino acid in the N-terminal 70-kDa processing site substituted with a more hydrophobic amino acid.

In some embodiments, at least 50% of the modified human GAA is processed to a 70-kDa form in the lysosome within 20, 30, or 40 hours. In still further embodiments, substantially all of the modified human GAA is processed to a 70-kDa form in the lysosome within 55, 65, or 75 hours.

In certain embodiments, a modified human GAA of the invention can be identified by its more rapid proteolytic processing to a mature 70-kDa form, or a corresponding variant thereof. In other embodiments, a modified human GAA as described herein can be identified by the production of an 82-kDa intermediate polypeptide that is not produced during proteolytic processing of native human GAA. In further embodiments, a modified human GAA can be identified by the absence of a 76-kDa intermediate polypeptide that is produced during proteolytic processing of unmodified human GAA.

In certain embodiments, the polypeptide has at least 80% identity to at least 500 amino acids of SEQ ID NO: 10 or SEQ ID NO: 170-171. In some instances, the polypeptide has at least 90% identity to at least 500 amino acids of SEQ ID NO: 10 or SEQ ID NO: 170-171. In other instances, the polypeptide has at least 95% identity to at least 500 amino acids of SEQ ID NO: 10 or SEQ ID NO: 170-171.

In certain embodiments, the GAA polypeptide with a modification at amino acid 201 to a hydrophobic residue, e.g., for example a H201L modification, exhibits more rapid lysosomal protease processing when compared to an unmodified human acid alpha-glucosidase protein. In some embodiments, at least 50% of the GAA pre-polypeptide is proteolytically processed to a 70-kDa mature GAA form within 20 hours of expression. In other embodiments, substantially all the GAA pre-polypeptide is proteolytically processed to a 70-kDa mature GAA form within 55 hours of expression.

In some embodiments, the cognate GAA leader peptide of amino acids 1-27 of SEQ ID NO: 10 (i.e., MGVRHPPCSHRLLAVCALVSLATAALL, SEQ ID NO: 175) is replaced with a different signal peptide (leader peptide). For example, the cognate leader peptide of GAA (SEQ ID NO: 175) can be replaced with any of: (i) an IgG1 leader peptide (referred to herein as a “201 leader peptide” or “2011p” having an amino acid sequence of: MEFGLSWVFLVALLKGVQCE (SEQ ID NO: 176) encoded by nucleic acid sequence SEQ ID NO: 177, (ii) wtIL21p: MYRMQLLSCIALSLALVTNS (SEQ ID NO: 178) encoded by nucleic acid sequence SEQ ID NO: 179, or (iii) mutIL21p: MYRMQLLLLIALSLALVTNS (SEQ ID NO: 180) encoded by nucleic acid sequence SEQ ID NO: 181. In some embodiments, the cognate GAA leader peptide (SEQ ID NO: 175) remains present, and an additional signal peptide is added, e.g., any one or more of signal peptides AAT, FN1, an IgG1 leader peptide (referred to herein as a “201 leader peptide” or “2011p” having an amino acid sequence of: MEFGLSWVFLVALLKGVQCE (SEQ ID NO: 176) encoded by nucleic acid sequence SEQ ID NO: 177, (ii) wtIL21p: MYRMQLLSCIALSLALVTNS (SEQ ID NO: 178) encoded by nucleic acid sequence SEQ ID NO: 179, or (iii) mutIL21p: MYRMQLLLLIALSLALVTNS (SEQ ID NO: 180) encoded by nucleic acid sequence SEQ ID NO: 181

In some embodiments, GAA is modified to add or remove glycosylation sites such as N-linked glycosylation sites, O-linked glycosylation sites or both. In certain embodiments, the addition or removal of glycosylation sites are achieved by N-terminal deletions, C-terminal deletions, internal deletions, random point mutagenesis, or, site directed mutagenesis. In some embodiments, the exemplary GAA modification involve addition of one or more Asparagine (Asn) residue/s or, one or more mutation to yield Asparagine (Asn) residue/s or, deletion of one or more Asparagine (Asn) residue/s. In certain embodiments, all or some of the N-linked, and/or, O-linked glycosylation sites present in GAA are mutated. In some embodiments, GAA modifications will yield information pertaining to the biological activity, physical structure and/or substrate binding potential of GAA.

(ii) Nucleic Acid Encoding GAA

In some embodiments, the rAAV genome comprises a heterologous nucleic acid sequence encoding the entire GAA polypeptide (e.g., the N-terminal/catalytic and the C-terminal domain), that is not fused to a heterologous signal sequence or a targeting peptide.

In some embodiments, the rAAV genome comprises a heterologous nucleic acid sequence encoding a secretory signal peptide or IGF2 targeting peptide fused in frame to the 3′ terminus of a GAA nucleic acid sequence that encodes the entire GAA polypeptide (e.g., the N-terminal/catalytic and the C-terminal domain). For example, heterologous nucleic acid sequence encoding a secretory signal peptide, or IGF2 targeting peptide is fused in frame to the 3′ terminus of a GAA nucleic acid sequence that encodes the 70 kDa and 76 kDa GAA polypeptides, such both polypeptides are expressed from the rAAV genome when the rAAV vector transduces a mammalian cell. In some embodiments, expression of the GAA nucleic acid can be driven by two promoters in the rAAV genome or by one promoter driving expression of a bicistronic construct.

In some embodiments of the methods and compositions as disclosed herein, the rAAV vector comprises a nucleic acid sequence encoding a GAA protein is a wild type GAA nucleic acid sequence, e.g., SEQ ID NO: 11 or SEQ ID NO: 72 or SEQ ID NO: 182. In some embodiments of the methods and compositions as disclosed herein, the rAAV vector comprises a nucleic acid sequence encoding a GAA protein which is a codon optimized GAA nucleic acid sequence, for any one or more of (i) enhanced expression in vivo, (ii) to reduce CpG islands, (iii).to reduce the innate immune response. Exemplary codon optimized GAA nucleic sequences encompassed for use in the methods and rAAV compositions as disclosed herein can be selected from any of: SEQ ID NO: 73, SEQ ID NO: 74, SEQ ID NO: 75, SEQ ID NO: 76 or SEQ ID NO: 182, or a nucleic acid sequence having at least 60%, or 70%, or 80%, 85% or 90% or 95%, or 98%, or 99% sequence identity to SEQ ID NO: 73, SEQ ID NO: 74, SEQ ID NO: 75, SEQ ID NO: 76 or SEQ ID NO: 182.

In addition, in some embodiments, the GAA nucleic acid sequences encompassed for use in the methods and rAAV compositions as disclosed herein are further modified with at least one or more of the following modifications: (i) removal of at least one, or two or in some embodiments, all alternative reading frames, (ii) removal of one or more CpGs islands, (iii) modification of the Kozak sequence, (iv) modification of a translational terminator sequence, and (v) removal of a spacer between promoter and Kozak sequence.

For example, in some embodiments, the rAAV composition comprises a hGAA nucleotide sequence of SEQ ID NO: 182, or a nucleic acid sequence having at least 60%, or 70%, or 80%, 85% or 90% or 95%, or 98%, or 99% sequence identity to SEQ ID NO: 182, where SEQ ID NO: 182 comprises the following elements shown in Table 1A, as compared to the wild type nucleic acid sequence for GAA;

TABLE 1A
elements of modGAA nucleic acid sequence.

| | Base pair (nucleotide numbers |
|---|---|
| modification | based on SEQ ID NO: 440) |
| cognate leader peptide | 954-1034 |
| | (81 bp) |
| GAT to GAC for Asp (T154C) | 1152-1154 |
| AST to AAC for Asn (T1901C) | 1899-1901 |
| GAT to GAC for Asp (T1910C) | 1909-1910 |
| GAT to GAC for Asp (T1967C) | 1965-1967 |
| GTT to GTG to remove CpG | 2022-2024 |
| (T2024G) | |
| CAA to CAG for gln (A2156G) | 2154-2156 |
| CAC to GAT to remove CpG | 2163-2165 |
| (C2165T) | |
| GTA to GTG for Val (A2393G) | 2391-2393 |
| AAT to AAC for Asn (T2561C) | 2259-2561 |
| GTC to GTG to remove CpG | 3399-3401 |
| (C3401G) | |
| GAT to GAC for Asp (T3536C) | 3534-3536 |
| Replacement of non optimal | 3810-3821 |
| stop | (12 bp) |

In some in some embodiments, the rAAV composition comprises a hGAA nucleotide sequence of SEQ ID NO: 182, or a nucleic acid sequence having at least 60%, or 70%, or 80%, 85% or 90% or 95%, or 98%, or 99% sequence identity to SEQ ID NO: 182, where the hGAA nucleotide sequence has been modified to with a series of point mutations that eliminate 3 potentially pro-inflammatory CpG motifs and a number of alternative reading frames (ARFs), where SEQ ID NO: 182 comprises the following point mutations as shown in Table 1B, as compared to the wildtype nucleic acid sequence for GAA, where numbering in table 1B assumes “A” in the GAA start codon ATG is the first nucleotide.

TABLE 1B

| NT# | Purpose | Original NT | New NT |
|---|---|---|---|
| 201 | Remove ORF | T | C |
| 949 | Remove ORF | T | C |
| 957 | Remove ORF | T | C |
| 1014 | Remove ORF | T | C |
| 1071 | Destroy CpG | T | G |
| 1203 | Remove ORF | A | G |
| 1212 | Destroy CpG | C | T |
| 1440 | Remove ORF | A | G |
| 1608 | Remove ORF | T | C |
| 2448 | Destroy CpG | C | G |
| 2583 | Remove ORF | T | C |

In some embodiments, the nucleic acid sequence encoding the cognate leader peptide in SEQ ID NO: 182 (e.g., nucleotides 1-81 of SEQ ID NO: 182) can be replaced by nucleic acid sequences encoding any of 2011p, wtIL21p or mutIL21p. Accordingly, in some embodiments, the nucleic residues 1-81 of SEQ ID NO: 182 (encoding the cognate leader peptide of GAA) can be replaced by nucleic acid sequences of SEQ ID NO: 177 (2011p), SEQ ID NO: 179 (wtIL21p) or SEQ ID NO: 181 (mutIL21p), or a nucleic acid sequence having at least 60%, or 70%, or 80%, 85% or 90% or 95%, or 98%, or 99% sequence identity to SEQ ID NOS: 177, 179 or 181.

In some embodiments, the rAAV vector or rAAV genome comprises a heterologous nucleic acid sequence encoding a GAA polypeptide comprising SEQ ID NO: 170 (GAA polypeptide with a cognate GAA signal sequence and H199R, R223H modifications), or SEQ ID NO: 171 (GAA polypeptide with a cognate GAA signal sequence and H199R, H201L, R223H modifications). The GAA polypeptide of SEQ ID NO: 170 is encoded by the nucleic acid sequence of SEQ ID NO: 182. Accordingly, in some embodiments, the rAAV vector comprises a nucleic acid of SEQ ID NO: 182 encoding a modified GAA polypeptide comprising H199R, R223H modifications. The GAA polypeptide of SEQ ID NO: 171 is encoded by the nucleic acid sequence of SEQ ID NO: 182 where basepairs (bp) 667-669 of SEQ ID NO: 182 are changed from CAC to any of: UUA, UUG, CUU, CUC CUA, CUG (resulting in a Histadine (H) to Leucine (L) amino acid change); or where bp 668 of SEQ ID NO: 182 is changed from A to U. Accordingly, in some embodiments, the rAAV vector comprises a nucleic acid of SEQ ID NO: 182, where bp 667-669 of SEQ ID NO: 182 are changed from CAC to any of: UUA, UUG, CUU, CUC CUA, CUG (which changes the amino acid from Histidine (H) to leucine (L)); or where bp 668 of SEQ ID NO: 182 is changed from A to U, which encodes a modified GAA polypeptide comprising H199R, H201L and R223H modifications.

In some embodiments, the rAAV vector or rAAV genome comprises a heterologous nucleic acid sequence encoding a GAA polypeptide selected from any of: SEQ ID NO: 172 (GAA polypeptide where cognate signal peptide is replaced with a IgG signal sequence and H199R, R223H modifications), or SEQ ID NO: 173 (GAA polypeptide where cognate signal peptide is replaced with a wtIL2 signal sequence and H199R, R223H modifications), SEQ ID NO: 174 (GAA polypeptide where cognate signal peptide is replaced with a mutIL3 signal sequence and H199R, R223H modifications).

In some embodiments, the rAAV vector or rAAV genome comprises a heterologous nucleic acid sequence comprising SEQ ID NO: 182 where bp 1-81 of SEQ ID NO: 182 is replaced with the nucleic acid of SEQ ID NO: 177 (IgG signal sequence), which encodes a GAA polypeptide of SEQ ID NO: 172 (IgG leader-GAA with H199R, R223H modifications). In some embodiments, the rAAV vector comprises a heterologous nucleic acid sequence comprising SEQ ID NO: 182, where bp 668 of SEQ ID NO: 182 is changed from A to U and where bp 1-81 of SEQ ID NO: 182 is replaced with the nucleic acid of SEQ ID NO: 177 (IgG signal peptide), which encodes a GAA polypeptide of SEQ ID NO: 172 (IgG leader-GAA with H199R, H201L and R223H modifications).

In some embodiments, the rAAV vector or rAAV genome comprises a heterologous nucleic acid sequence comprising SEQ ID NO: 182 where bp 1-81 of SEQ ID NO: 182 is replaced with the nucleic acid of SEQ ID NO: 179 (wt IL2 signal peptide), which encodes a GAA polypeptide of SEQ ID NO: 173 (wt IL2 signal peptide-GAA with H199R, R223H modifications). In some embodiments, the rAAV vector comprises a heterologous nucleic acid sequence comprising SEQ ID NO: 182, where bp 668 of SEQ ID NO: 182 is changed from A to U and where bp 1-81 of SEQ ID NO: 182 is replaced with the nucleic acid of SEQ ID NO: 179 (wt IL2 signal peptide), which encodes a GAA polypeptide of SEQ ID NO: 173 (wt IL2 signal peptide-GAA with H199R, H201L and R223H modifications).

In some embodiments, the rAAV vector comprises a heterologous nucleic acid sequence comprising SEQ ID NO: 182 where bp 1-81 of SEQ ID NO: 182 is replaced with the nucleic acid of SEQ ID NO: 181 (mutIL2 signal peptide), which encodes a GAA polypeptide of SEQ ID NO: 174 (mutIL2 signal peptide-GAA with H199R, R223H modifications). In some embodiments, the rAAV vector comprises a heterologous nucleic acid sequence comprising SEQ ID NO: 182, where bp 668 of SEQ ID NO: 182 is changed from A to U and where bp 1-81 of SEQ ID NO: 182 is replaced with the nucleic acid of SEQ ID NO: 181 (mut IL2 signal peptide), which encodes a GAA polypeptide of SEQ ID NO: 174 (mut IL2 signal peptide-GAA with H199R, H201L, and R223H modifications).

The C-terminal domain of GAA functions in trans in conjunction with the 70/76 kDal species to generate active GAA. The boundary between the catalytic domain and the C-terminal domain appears to be at about amino acid residue 791, based on its presence in a short region of less than 18 amino acids that is absent from most members of the family 31 hydrolyases and which contains 4 consecutive proline residues in GAA. It has been reported that the C-terminal domain associated with the mature species begins at amino acid residue 792 (Moreland et al. (Nov. 1, 2004) J. Biol. Chem., Manuscript 404008200). Accordingly, in some embodiments, the GAA nucleic acid sequence that encodes the entire GAA polypeptide, with the exception of the C-terminal domain. Thus, in such an embodiment, the rAAV vector can be used to transduce a mammalian cell that expresses the C-terminal domain of GAA as a separate polypeptide.

### B. Secretory Signal Peptide

Native GAA signal peptide is not cleaved in the ER thereby causing native GAA polypeptide to be membrane bound in the ER (Tsuji et al. (1987) Biochem. Int. 15(5):945-952). Disruption of the membrane association of GAA can be accomplished by replacing the endogenous GAA signal peptide (and optionally adjacent sequences) with an alternate signal peptide for GAA.

Accordingly, in representative embodiments, the rAAV vector and rAAV genome as disclosed herein further comprises a heterologous nucleic acid encoding a GAA polypeptide to be transferred to a target cell, attached to a heterologous nucleic acid sequence that encodes a secretory signal peptide in the place of the endogenous GAA signal peptide. The heterologous nucleic acid is operatively associated with the segment encoding the secretory signal peptide, such that upon transcription and translation a fusion polypeptide is produced containing the secretory signal sequence operably associated with (e.g., directing the secretion of) the GAA polypeptide.

In some embodiments, the AAV vector encodes a GAA polypeptide that comprises the endogenous GAA signal peptide (e.g., amino acids 1-27 of SEQ ID NO: 10 (also referred to as “innate GAA” or “cognate GAA” signal peptide). In some embodiments, the AAV vector encodes a GAA polypeptide that comprises the endogenous GAA signal peptide (e.g., amino acids 1-27 of SEQ ID NO: 10 (also referred to as “innate GAA” or “cognate GAA” signal peptide) and an additional heterologous (non native) signal sequence. In some embodiments, the GAA polypeptide that lacks the endogenous signal peptide of amino acids 1-27 of GAA is fused to a secretory signal. In some embodiments of the compositions and methods described herein, the secretory signal serves a general purpose of assisting the secretion of the GAA polypeptide, or a fusion polypeptide, e.g., the IGF2 targeting peptide-GAA fusion polypeptide from the liver cells into the blood, where it can travel and be targeted to the lysosomes of mammalian cells, for example, human cardiac and skeletal muscle cells, as described herein. In some embodiments, a heterologous secretory signal is selected from any of: a AAT signal peptide, a fibronectin signal peptide (FN1), a GAA signal peptide, or an active fragment of AAT, FN1 or GAA signal peptide having secretory signal activity.

In some embodiments, the secretory signal peptide is heterologous to (i.e., foreign or exogenous to) the polypeptide of interest. For example, a heterologous secretory signal peptide is a fibronectin secretory signal peptide, the polypeptide of interest is not fibronectin. In some embodiments, the secretory signal peptide is selected from any of: AAT signal peptide, a fibronectin signal peptide (FN1), or an active fragment of AAT, FN1 or GAA signal peptide having secretory signal activity. In alternative embodiments, the secretory signal peptide is not heterologous to GAA, i.e., the signal peptide is the GAA signal peptide (i.e., residues 1-27 of the native GAA polypeptide).

In some embodiments, the cognate GAA signal peptide of amino acids 1-27 of SEQ ID NO: 10 (i.e., MGVRHPPCSHRLLAVCALVSLATAALL, SEQ ID NO: 175) is replaced with a different or heterologous leader peptide. For example, the cognate leader peptide of GAA (SEQ ID NO: 175) can be replaced with any of the heterologous signal peptides selected from: (i) an IgG1 leader peptide (referred to herein as a “201 leader peptide” or “2011p” having an amino acid sequence of: MEFGLSWVFLVALLKGVQCE (SEQ ID NO: 176) encoded by nucleic acid sequence SEQ ID NO: 177, (ii) wtIL21p: MYRMQLLSCIALSLALVTNS (SEQ ID NO: 178) encoded by nucleic acid sequence SEQ ID NO: 179, or (iii) mutIL21p: MYRMQLLLLIALSLALVTNS (SEQ ID NO: 180) encoded by nucleic acid sequence SEQ ID NO: 181, or a leader peptide having at least 90% sequence identity to any of SEQ ID NOs 176, 178 or 180.

In general, the secretory signal peptide will be at the amino-terminus (N-terminus) of the fusion polypeptide (i.e., the nucleic acid segment encoding the secretory signal peptide is 5′ to the heterologous nucleic acid encoding the GAA peptide or GAA-fusion peptide in the rAAV vector or rAAV genome as disclosed herein). Alternatively, the secretory signal may be at the carboxyl-terminus or embedded within the GAA polypeptide or GAA fusion polypeptide (e.g., IGF2-GAA fusion polypeptide), as long as the secretory signal is operatively associated therewith and directs secretion of the GAA polypeptide or GAA fusion polypeptide of interest (either with or without cleavage of the signal peptide from the GAA polypeptide) from the cell.

The secretory signal is operatively associated with the GAA polypeptide or GAA fusion polypeptide is targeted to the secretory pathway. Alternatively stated, the secretory signal is operatively associated with the GAA polypeptide such that the GAA-polypeptide or GAA fusion polypeptide is secreted from the cell at a higher level (i.e., a greater quantity) than in the absence of the secretory signal peptide. In general, typically at least about 20%, 30%, 40%, 50%, 70%, 80%, 85%, 90%, 95% or more of the GAA-polypeptide or IGF2-GAA fusion polypeptide (alone and/or fused with the signal peptide) is secreted from the cell when a signal peptide is attached as compared to in the absence of the attachment of a secretory signal peptide. In other embodiments, essentially all of the detectable polypeptide (alone and/or in the form of the fusion polypeptide) is secreted from the cell.

By the phrase “secreted from the cell”, the polypeptide may be secreted into any compartment (e.g., fluid or space) outside of the cell including but not limited to: the interstitial space, blood, lymph, cerebrospinal fluid, kidney tubules, airway passages (e.g., alveoli, bronchioles, bronchia, nasal passages, etc.), the gastrointestinal tract (e.g., esophagus, stomach, small intestine, colon, etc.), vitreous fluid in the eye, and the cochlear endolymph, and the like.

In one embodiment, the rAAV genome comprises a heterologous nucleic acid that encodes a secretory signal peptide (SP) fused to the GAA-fusion polypeptide, where the GAA-fusion polypeptide comprises a targeting peptide (e.g., IGF2 targeting peptide) fused to a GAA polypeptide. As used herein GAA also refers to the modified GAA described above. Accordingly, the signal peptide disclosed herein increases the efficacy of secretion of the GAA polypeptide or IGF2-GAA fusion polypeptide from the cell transduced with the rAAV vector or comprising the rAAV genome as described herein

Accordingly, in some embodiments, the rAAV genome disclosed herein comprises a 5′ ITR and 3′ ITR sequence, and located between the 5′ITR and the 3′ ITR, a promoter operatively linked to a heterologous nucleic acid encoding a secretory peptide and nucleic acid encoding an alpha-glucosidase (GAA) polypeptide (i.e., the heterologous nucleic acid encodes a GAA fusion polypeptide comprising a signal peptide-GAA polypeptide).

In alternative embodiments, the rAAV genome disclosed herein comprises a 5′ ITR and 3′ ITR sequence, and located between the 5′ITR and the 3′ ITR, a promoter operatively linked to a heterologous nucleic acid encoding a secretory peptide and nucleic acid encoding an alpha-glucosidase (GAA) fusion polypeptide, where the fusion protein comprises IGF2 targeting peptide and a GAA polypeptide (i.e., the heterologous nucleic acid encodes a GAA fusion polypeptide comprising a signal peptide-IGF2-GAA polypeptide).

Generally, secretory signal peptides are cleaved within the endoplasmic reticulum and, in some embodiments, the secretory signal peptide is cleaved from the GAA polypeptide prior to secretion. It is not necessary, however, that the secretory signal peptide is cleaved as long as secretion of the GAA polypeptide or IGF2-GAA fusion polypeptide from the cell is enhanced and the GAA polypeptide is functional. Thus, in some embodiments, the secretory signal peptide is partially or entirely retained.

In some embodiments, the rAAV genome, or an isolated nucleic acid as disclosed herein comprises a nucleic acid encoding a chimeric polypeptide comprising a GAA polypeptide operably linked to a secretory signal peptide, and the chimeric polypeptide is expressed and produced from a cell transduced with the rAAV vector and the GAA polypeptide is secreted from the cell. The GAA polypeptide or GAA fusion polypeptide (e.g., IGF2-GAA fusion polypeptide) can be secreted after cleavage of all or part of the secretory signal peptide. Alternatively, the GAA polypeptide or GAA fusion polypeptide (e.g., IGF2-GAA fusion polypeptide) can retain the secretory signal peptide (i.e., the secretory signal is not cleaved). Thus, in this context, the “GAA polypeptide or GAA fusion polypeptide” can be a chimeric polypeptide comprising the secretory peptide.

The secretory signal sequences of the invention are not limited to any particular length as long as they direct the polypeptide of interest to the secretory pathway. In representative embodiments, the signal peptide is at least about 6, 8, 10 12, 15, 20, 25, 30 or 35 amino acids in length up to a length of about 40, 50, 60, 75, or 100 amino acids or longer.

Secretory signal peptide encoded by the rAAV genome and in the rAAV vector as disclosed herein can comprise, consist essentially of or consist of a naturally occurring secretory signal sequence or a modification thereof. Numerous secreted proteins and sequences that direct secretion from the cell are known in the art, are disclosed in U.S. Pat. No. 9,873,868, which is incorporated herein in its entirety by reference. Exemplary secreted proteins (and their secretory signals) include but are not limited to: erythropoietin, coagulation Factor IX, cystatin, lactotransferrin, plasma protease C1 inhibitor, apolipoproteins (e.g., APO A, C, E), MCP-1, α-2-HS-glycoprotein, α-1-microgolubilin, complement (e.g., C1Q, C3), vitronectin, lymphotoxin-α, azurocidin, VIP, metalloproteinase inhibitor 2, glypican-1, pancreatic hormone, clusterin, hepatocyte growth factor, insulin, α-1-antichymotrypsin, growth hormone, type IV collagenase, guanylin, properdin, proenkephalin A, inhibin p (e.g., A chain), prealbumin, angiocenin, lutropin (e.g., R chain), insulin-like growth factor binding protein 1 and 2, proactivator polypeptide, fibrinogen (e.g., R chain), gastric triacylglycerol lipase, midkine, neutrophil defensins 1, 2, and 3, α-1-antitrypsin, matrix gla-protein, α-tryptase, bile-salt-activated lipase, chymotrypsinogen B, elastin, IG lambda chain V region, platelet factor 4 variant, chromogranin A, WNT-1 proto-oncogene protein, oncostatin M, β-neoendorphin-dynorphin, von Willebrand factor, plasma serine protease inhibitor, serum amyloid A protein, nidogen, fibronectin, rennin, osteonectin, histatin 3, phospholipase A2, cartilage matrix Protein, GM-CSF, matrilysin, neuroendocrine protein 7B2, placental protein 11, gelsolin, M-CSF, transcobalamin I, lactase-phlorizin hydrolase, elastase 2B, pepsinogen A, MIP 1-β, prolactin, trypsinogen II, gastrin-releasing peptide II, atrial natriuretic factor, secreted alkaline phosphatase, pancreatic α-amylase, secretogranin I, β-casein, serotransferrin, tissue factor pathway inhibitor, follitropin β-chain, coagulation factor XII, growth hormone-releasing factor, prostate seminal plasma protein, interleukins (e.g., 2, 3, 4, 5, 9, 11), inhibin (e.g., alpha chain), angiotensinogen, thyroglobulin, IG heavy or light chains, plasminogen activator inhibitor-1, lysozyme C, plasminogen activator, antileukoproteinase 1, statherin, fibulin-1, isoform B, uromodulin, thyroxine-binding globulin, axonin-1, endometrial α-2 globulin, interferon (e.g., alpha, beta, gamma), β-2-microglobulin, procholecystokinin, progastricsin, prostatic acid phosphatase, bone sialoprotein II, colipase, Alzheimer's amyloid A4 protein, PDGF (e.g., A or B chain), coagulation factor V, triacylglycerol lipase, haptoglobuin-2, corticosteroid-binding globulin, triacylglycerol lipase, prorelaxin H2, follistatin 1 and 2, platelet glycoprotein IX, GCSF, VEGF, heparin cofactor II, antithrombin-III, leukemia inhibitory factor, interstitial collagenase, pleiotrophin, small inducible cytokine A1, melanin-concentrating hormone, angiotensin-converting enzyme, pancreatic trypsin inhibitor, coagulation factor VIII, α-fetoprotein, α-lactalbumin, senogelin II, kappa casein, glucagon, thyrotropin beta chain, transcobalamin II, thrombospondin 1, parathyroid hormone, vasopressin copeptin, tissue factor, motilin, MPIF-1, kininogen, neuroendocrine convertase 2, stem cell factor procollagen al chain, plasma kallikrein keratinocyte growth factor, as well as any other secreted hormone, growth factor, cytokine, enzyme, coagulation factor, milk protein, immunoglobulin chain, and the like.

In some embodiments, other secretory signal peptides encoded by the rAAV genome and in the rAAV vector as disclosed herein can be selected from, but are not limited to, the secretory signal sequences from prepro-cathepsin L (e.g., GenBank Accession Nos. KHRTL, NP_037288; NP_034114, AAB81616, AAA39984, P07154, CAA68691; the disclosures of which are incorporated by reference in their entireties herein) and prepro-alpha 2 type collagen (e.g., GenBank Accession Nos. CAA98969, CAA26320, CGHU2S, NP_000080, BAA25383, P08123; the disclosures of which are incorporated by reference in their entireties herein) as well as allelic variations, modifications and functional fragments thereof (as discussed above with respect to the fibronectin secretory signal sequence). Exemplary secretory signal sequences include for preprocathepsin L (Rattus norvegicus, MTPLLLLAVLCLGTALA [SEQ ID NO: 27]; Accession No. CAA68691) and for prepro-alpha 2 type collagen (Homo sapiens, MLSFVDTRTLLLLAVTLCLATC [SEQ ID NO: 28]; Accession No. CAA98969). Also encompassed are longer amino acid sequences comprising the full-length secretory signal sequence from preprocathepsin L and prepro-alpha 2 type collagen or functional fragments thereof (as discussed above with respect to the fibronectin secretory signal sequence).

In some embodiments, the secretory signal peptide is derived in part or in whole from a secreted polypeptide that is produced by liver cells. In some embodiments, a secretory signal peptide can further be in whole or in part synthetic or artificial. Synthetic or artificial secretory signal peptides are known in the art, see e.g., Barash et al., “Human secretory signal peptide description by hidden Markov model and generation of a strong artificial signal peptide for secreted protein expression,” Biochem. Biophys. Res. Comm. 294:835-42 (2002); the disclosure of which is incorporated herein in its entirety. In particular embodiments, the secretory signal peptide comprises, consists essentially of, or consists of the artificial secretory signal: MWWRLWWLLLLLLLLWPMVWA (SEQ ID NO: 29) or variations thereof having 1, 2, 3, 4, or 5 amino acid substitutions (optionally, conservative amino acid substitutions, conservative amino acid substitutions are known in the art).

Exemplary signal peptides for use in the methods and compositions as disclosed herein can be selected from any signal peptide disclosed in Table 2, or functional variants thereof. Exemplary signal peptides are Fibronectin (FN1), or AAT. In some embodiments of the methods and compositions disclosed herein, the rAAV vector composition comprises the nucleic acid encoding a secretory signal peptide, e.g., encoding a secretory signal peptide selected from an AAT signal peptide (e.g., SEQ ID NO: 17), a fibronectin signal peptide (FN1) (e.g., SEQ ID NO: 18-21), a GAA signal peptide, an hIGF2 signal peptide (e.g., SEQ ID NO: 22) or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NOs: 17-22.

In some embodiments of the methods and compositions as disclosed herein, the nucleic acid encoding the secretory signal is selected from any of SEQ ID NO: 17, 81-21, 22-26, or a nucleic acid sequence at least about 75%, or 80%, or 85%, or 90%, or 95%, or 98%, or 99% sequence identity to any of SEQ ID NOs: 17 or 22-26.

In some embodiments, one can readily substitute a FN1 or AAT signal peptide with any signal peptide, including signal peptides for over liver expressed proteins, or signal peptides disclosed in U.S. Pat. No. 62,937,556, filed on Nov. 19, 2019, or PCT/US19/61653 filed on Nov. 15, 2019.

Fibronectin Secretory Signal Peptide:

In some embodiments, the secretory signal peptide is a fibronectin secretory signal peptide, which term includes modifications of naturally occurring sequences (as described in more detail below).

In some embodiments, the secretory signal peptide is a fibronectin signal peptide, e.g., a signal sequence of human fibronectin or a signal sequence from rat fibronectin. Fibronectin (FN1) signal sequences and modified FN1 signal peptides encompassed for use in the rAAV genome and rAAV vectors described herein are disclosed in U.S. Pat. No. 7,071,172, which is incorporated herein in its entirety by reference, and in Table 3 of provisional application 62/937,556, filed on Nov. 19, 2019. Examples of exemplary fibronectin secretory signal sequences include, but are not limited to those listed in Table 1 of U.S. Pat. No. 7,071,172, which is incorporated herein in its entirety by reference.

TABLE 2
Exemplary Fibronectin (FN1) secretory signal peptides

| Species | Secretory Signal sequence | Nucleic acid sequence |
|---|---|---|
| H. Sapiens | MLRGPGPGLLLLAVQCLGTAV | ATG CTT AGG GGT CCG GGG CCC GGG CTG |
| | PSTGA (SEQ ID NO: 20) | CTG CTG CTG GCC GTC CAG TGC CTG GGG |
| | | ACA GCG GTG CCC TCC ACG GGA GCC |
| | | (SEQ ID NO: 25) |
| R. | MLRGPGPGRLLLLAVLCLGTSV | 5′-ATGCTCAGGGGTCCGGGACCCGGGCGGCT |
| Norvegicus | RCTETGKSKR (SEQ ID NO: 18) | GCTGCTGCTAGCAGTCCTGTGCCTGGGGAC |
| | | ATCGGTGCGCTGCACCGAAACCGGGAAGA |
| | | GCAAGAGG-3 (SEQ ID NO: 23) |
| | | (nucleotides 208-303) |
| R. | MLRGPGPGRLLLLAVLCLGTSV | 5′-ATG CTC AGG GGT CCG GGA CCC GGG |
| Norvegicus | RCTETGKSKR ↑ LALQIV | CGG CTG CTG CTG CTA GCA GTC CTG TGC |
| | (SEQ ID NO: 19) | CTG GGG ACA TCG GTG CGC TGC |
| | | ACC GAA ACC GGG AAG AGC AAG AGG ↑ |
| | | CAGGCTCAGCAAATCGTG-3′. (SEQ ID |
| | | NO: 24) (↑ denotes the cleavage site) |
| X. laevis | MRRGALTGLLLVLCLSVVLRA | ATG CGC CGG GGG GCC CTG ACC GGG CTG |
| | APSATSKKRR (SEQ ID NO: 21) | CTC CTG GTC CTG TGC CTG AGT GTT GTG |
| | | CTA CGT GCA GCC CCC TCT GCA ACA AGC |
| | | AAG AAG CGC AGG (SEQ ID NO: 26) |

An exemplary nucleotide sequence encoding the fibronectin secretory signal sequence of Rattus norvegicus is found at GenBank accession number X15906 (the disclosure of which is incorporated herein by reference). As yet another illustrative sequence, the nucleotide sequence encoding the secretory signal peptide of human fibronectin 1, transcript variant 1 (Accession No. NM_002026, nucleotides 268-345; the disclosure of Accession No. NM_002026 is incorporated herein by reference in its entirety). Another exemplary secretory signal sequence is encoded by the nucleotide sequence encoding the secretory signal peptide of the Xenopus laevis fibronectin protein (Accession No. M77820, nucleotides 98-190; the disclosure of Accession No. M77820 incorporated herein by reference in its entirety).

In another embodiment, the fibronectin signal sequence (FN1, nucleotides 208-303, 5′-ATG CTC AGG GGT CCG GGA CCC GGG CGG CTG CTG CTG CTA GCA GTC CTG TGC CTG GGG ACA TCG GTG CGC TGC ACC GAA ACC GGG AAG AGC AAG AGG-3′, SEQ ID NO: 23) was derived from the rat fibronectin mRNA sequence (Genbank accession 4X15906) and codes for the following peptide signal sequence: Met Leu Arg Gly Pro Gly Pro Gly Arg Leu Leu Leu Leu Ala Val Leu Cys Leu Gly Thr Ser Val Arg Cys Thr Glu Thr Gly Lys Ser Lys Arg (SEQ ID NO: 18). In some embodiments of the methods and compositions disclosed herein, a recombinant AAV vector comprises a heterologous nucleic acid sequence that encodes a secretory signal peptide which is a fibronectin signal peptide (FN 1) or an active fragment thereof having secretory signal activity (e.g., a FN 1 signal peptide has the sequence of any of SEQ ID NO: 18-21, or an amino acid sequence at having at least about 75%, or 80%, or 85%, or 90%, or 95%, or 98%, or 99% sequence identity to any of SEQ ID NOs: 18-21), and the heterologous nucleic acid sequence encodes a IGF2 targeting peptide selected from any of: SEQ ID NO: 5, SEQ ID NO: 6, SEQ ID NO: 7, SEQ ID NO: 8 or SEQ ID NO: 9, or a IGF2 peptide having at least about 75%, or 80%, or 85%, or 90%, or 95%, or 98%, or 99% sequence identity to SEQ ID NOs: 5-9. In some embodiments of the methods and compositions disclosed herein, a recombinant AAV vector comprises a heterologous nucleic acid sequence that encodes a secretory signal peptide is AAT signal peptide or an active fragment thereof having secretory signal activity, (e.g., a AAT signal peptide has the sequence of SEQ ID NO: 17, or an amino acid sequence at having at least about 75%, or 80%, or 85%, or 90%, or 95%, or 98%, or 99% sequence identity to SEQ ID NO: 17), and the heterologous nucleic acid sequence encodes a IGF2 targeting peptide selected from any of: SEQ ID NO: 5, SEQ ID NO: 6, SEQ ID NO: 7, SEQ ID NO: 8 or SEQ ID NO: 9, or a IGF2 peptide having at least about 75%, or 80%, or 85%, or 90%, or 95%, or 98%, or 99% sequence identity to SEQ ID NOs: 5-9.

Those skilled in the art will appreciate that the secretory signal sequence may encode one, two, three, four, five or all six or more of the amino acids at the C-terminal side of the peptidase cleavage site (identified by an T) (see e.g., SEQ ID NO: 19 and 24 in Table 2). Those skilled in the art will appreciate that additional amino acids (e.g., 1, 2, 3, 4, 5, 6 or more amino acids) on the carboxy-terminal side of the cleavage site may be included in the secretory signal sequence.

In some embodiments of the methods and compositions disclosed herein, a recombinant AAV vector comprises, or consist of, located between the 5′ ITR and the 3′ ITR, a heterologous nucleic acid sequence that encodes a secretory signal peptide and nucleic acid encoding a hGAA polypeptide, where the nucleic acid sequence that encodes the signal sequence is selected from any of: an AAT signal peptide (e.g., SEQ ID NO: 17), a fibronectin signal peptide (FN1) (e.g., SEQ ID NO: 18-21), a cognate GAA signal peptide (SEQ ID NO: 175), an hIGF2 signal peptide (e.g., SEQ ID NO: 22), a IgG1 leader peptide (SEQ ID NO: 177), wtIL2 leader peptide (SEQ ID NO: 179), mutant IL2 leader peptide (SEQ ID NO: 181) or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NOs: 17-22, 175, 177, 179 or 181, and where the nucleic acid encoding the signal peptide is located 5′ of a nucleic acid encoding a hGAA polypeptide as disclosed herein, and where the nucleic acid encoding the signal sequence and the hGAA polypeptide are operatively linked to any LSP disclosed herein in Table 4, or a functional variant thereof.

In embodiments of the invention, the functional fragment has at least about 50%, 70%, 80%, 90% or more secretory signal activity as compared with the sequences specifically disclosed herein or even has a greater level of secretory signal activity.

Peptidase Cleavage Sites

In some embodiments, one or more exogenous peptidase cleavage site may be inserted into the secretory signal peptide—GAA fusion polypeptide, e.g., between the secretory signal peptide and the GAA polypeptide. In particular embodiments, an autoprotease (e.g., the foot and mouth disease virus 2A autoprotease) is inserted between the secretory signal peptide and the GAA polypeptide or IGF2-GAA fusion polypeptide. In other embodiments, a protease recognition site that can be controlled by addition of exogenous protease is employed (e.g., Lys-Arg recognition site for trypsin, the Lys-Arg recognition site of the Aspergillus KEX2-like protease, the recognition site for a metalloprotease, the recognition site for a serine protease, and the like). Modification of the GAA polypeptide to delete or inactivate native protease sites is encompassed herein and disclosed in U.S. Provisional Application 62,937,556, filed on Nov. 19, 2019 and International Application PCT/US19/61653, filed Nov. 15, 2019.

### C. IGF2 Targeting Peptide Sequence

In one embodiment, the rAAV genome comprises a heterologous nucleic acid that encodes a targeting peptide (TP) fused to the GAA polypeptide. In some embodiments, the targeting peptide is a ligand for an extracellular receptor, wherein the targeting peptide binds an extracellular domain of a receptor on the surface of a target cell and, upon internalization of the receptor, permits localization of the polypeptide in a human lysosome. In one embodiment, the targeting peptide includes a urokinase-type plasminogen receptor moiety capable of binding the cation-independent mannose-6-phosphate receptor. In some embodiments, the targeting peptide incorporates one or more amino acid sequences of a IGF2 targeting peptide.

In some embodiments, the IGF2 targeting peptide as disclosed herein comprises at least part of a ligand for an extracellular receptor, for example, the IGF2 targeting peptide binds to human cation-independent mannose-6-phosphate receptor (CI-MPR) or the IGF2 receptor.

IGF2 is also known by alias; chromosome 11 open reading frame 43, insulin-like growth factor 2, IGF-II, FLJ44734; IGF2, somatomedin A and preptin. The mRNA of wild-type human IGF2 sequence is corresponds to: GCTTACCGCCCCAGTGAGACCCTGTGCGGCGGGGAGCTGGTGGACACCCTCCAGTTCGTC TGTGGGGACCGCGGCTTCTACTTCAGCAGGCCCGCAAGCCGTGTGAGCCGTCGCAGCCGT GGCATCGTTGAGGAGTGCTGTTTCCGCAGCTGTGACCTGGCCCTCCTGGAGACGTACTGT GCTACCCCCGCCAAGTCCGAG (SEQ ID NO: 1). The full length IGF2 protein (including the IGF2 targeting sequence) is encoded by the nucleic acid sequence of NM_000612.6, and encodes the full length IGF2 protein NP_000603.1.

The mature human IGF2 targeting peptide is shown below:

(SEQ ID NO: 5)
A Y R P S E T L C G G E L V D T L Q F V C G D R G
F Y F S R P A S R V S R R S R G I V E E C C F R S
C D L A L L E T Y C A T P A K S E

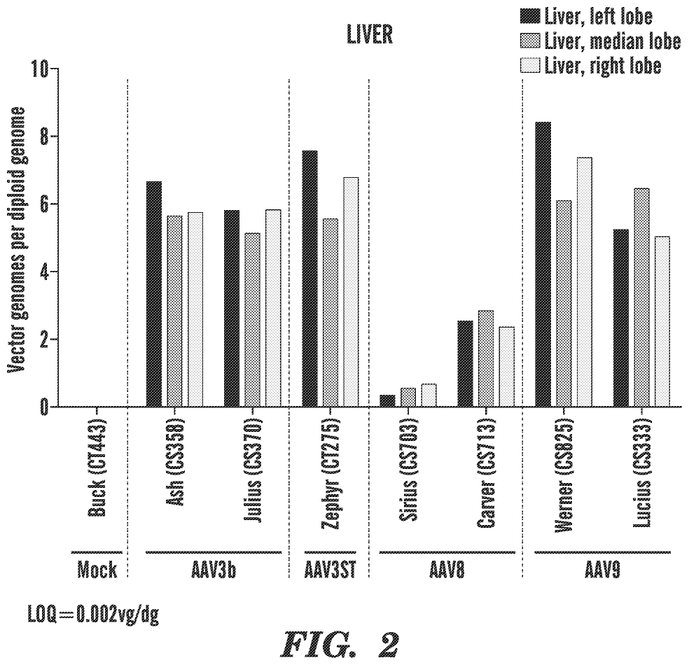

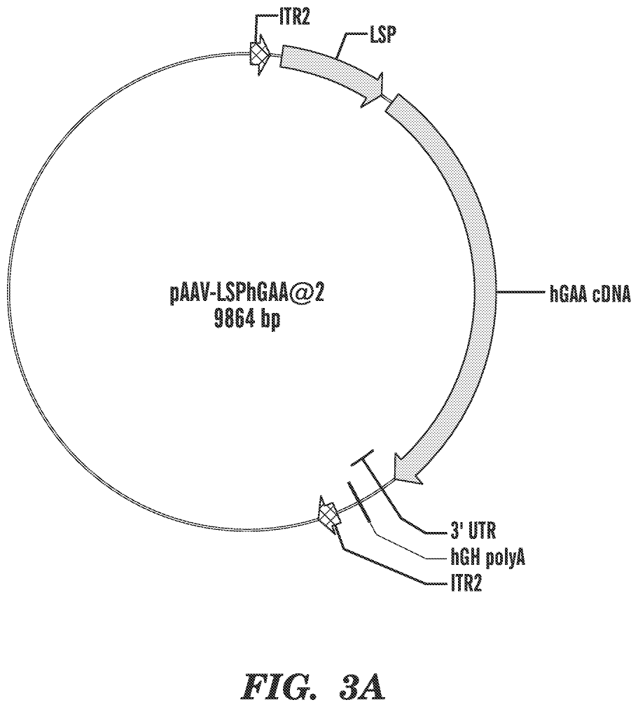

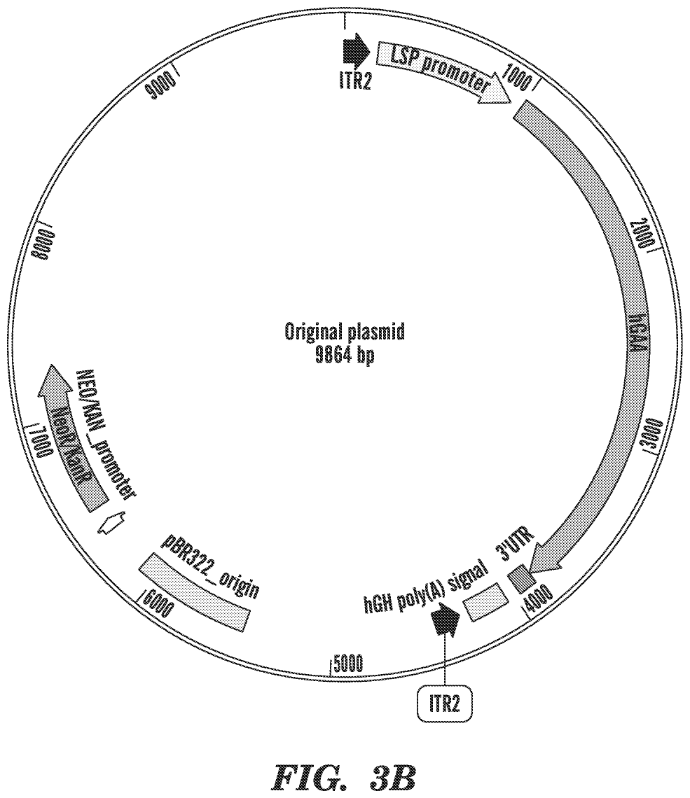

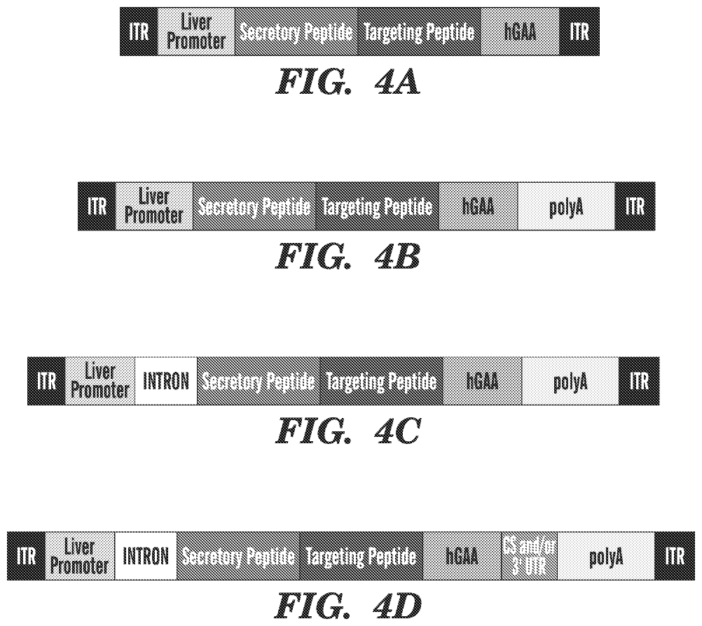

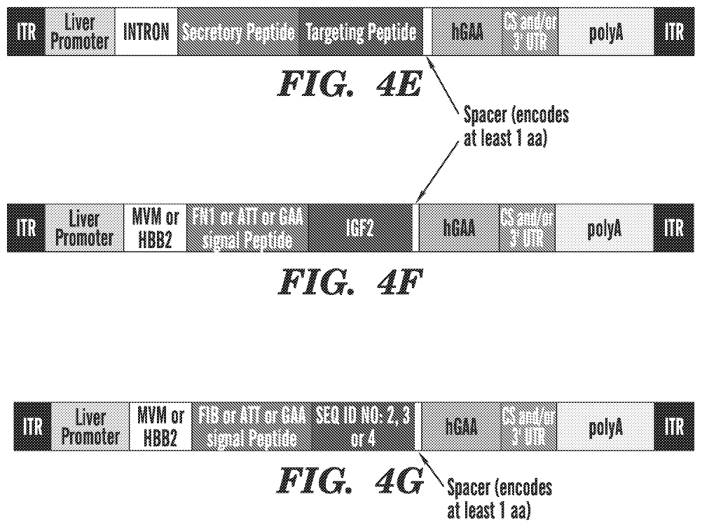

The coding sequence of human IGF2 is also disclosed in U.S. Pat. No. 8,492,388 (see e.g., FIG. 2) which is incorporated herein in its entirety by reference. IGF2 protein is synthesized as a pre-pro-protein with a 24 amino acid signal peptide at the amino terminus and a 89 amino acid carboxy terminal region both of which are removed post-translationally, reviewed in O'Dell et al. (1998) Int. J. Biochem Cell Biol. 30(7):767-71. The mature protein is 67 amino acids. A Leishmania codon optimized version of the mature IGF2 is disclosed in U.S. Pat. No. 8,492,388 (see, e.g., FIG. 3 of U.S. Pat. No. 8,492,388) (Langford et al. (1992) Exp. Parasitol. 74(3):360-1). Additional cassettes containing a deletion of amino acids 1-7 or 2-7 of the mature polypeptide (A1-7), alteration of residue 27 from tyrosine to leucine (Y27L) or both mutations (A1-7,Y27L or Δ2-7,Y27L) were made to produce IGF-2 cassettes with specificity for only the desired receptor as described below. Accordingly, in some embodiments, the IGF2 targeting sequence can be selected from any of: wildtype, Y27L, Δ1-7, Δ2-7 and Y27L-A1-7, Y27L-Δ2-7, V43M, Y27L-V43M, Y27L-A1-7-V43M, Y27L-Δ2-7-V43M IGF2 variants are encompassed for use herein.

Exemplary IGF2 targeting peptide for use in the methods and compositions herein are disclosed in U.S. Provisional Application 62,937,556, filed on Nov. 19, 2019 and International Application PCT/US19/61653, filed Nov. 15, 2019, and International application PCT/US19/61701, filed Nov. 15, 2019, each of which are incorporated herein in their entirety by reference.

In some embodiments, an IGF2 targeting peptide for use in the methods and compositions herein can have a modification of any one or more of: E6R, F26S, Y27L, V43L, F48T, R495, S50I, A54R, L55R, K65R, as disclosed in US application 2019/0343968, which is incorporated herein in its entirety. In some embodiments, the IGF2 targeting peptide has a modification of V43M in addition to one or more modifications selected from: E6R, F26S, Y27L, V43L, F48T, R495, S50I, A54R, L55R and K65R. In some embodiments, the IGF2 targeting peptide has a Δ1-7 or Δ2-7 modification in addition to one or more modifications selected from: E6R, F26S, Y27L, V43L, F48T, R495, S50I, A54R, L55R and K65R. In some embodiments, the IGF2 targeting peptide has a Δ1-7 or Δ2-7 modification, a V43M modification, and one or more modifications selected from: E6R, F26S, Y27L, V43L, F48T, R495, S50I, A54R, L55R and K65R.

In particular embodiments, the IGF2 targeting peptide comprises a modification at valine 43, where valine is modified to a met (V43M), such that translation initiation starts at amino acid 43. A IGF2 targeting peptide with a modification of V43M encompassed for use herein as a targeting peptide or IGF2 targeting peptide binds the cation-independent mannose-6-phosphate receptor. In alterative embodiments, the IGF2 targeting peptide is delta 1-42 of IGF2 with V43 changed to an Met (i.e., IGF2-A1-42 (SEQ ID NO: 8) or IGF2-V43M (SEQ ID NO:9).

In some embodiments, the rAAV genome comprises a nucleic acid encoding an IGF2-GAA fusion protein, where the nucleic acid encoding the mature IGF2 targeting peptide (SEQ ID NO: 5) or a IGF2 targeting peptide variant (e.g., SEQ ID NO: 6 (IGF2-Δ2-7); SEQ ID NO: 7 (IGF2-A1-7); SEQ ID NO: 8 (IGF2-Δ1-42), SEQ ID NO: 9 (IGF2-V43M)) or sequences having at least 85%, or 90% or 95% sequence identity to SEQ ID NO: 5-9, is fused to the 5′ end of nucleic acid encoding the GAA protein, fusion proteins (e.g., IGF2-GAA fusion polypeptides) are created that can be taken up by a variety of cell types and transported to the lysosome. Alternatively, a nucleic acid encoding a precursor IGF2 polypeptide can be fused to the 3′ end of a GAA gene; the precursor includes a carboxy-terminal portion that is cleaved in mammalian cells to yield the mature IGF2 polypeptide, but the IGF2 targeting peptide is preferably omitted (or moved to the 5′ end of the GAA gene). This method has numerous advantages over methods involving glycosylation including simplicity and cost effectiveness, because once the protein is isolated, no further modifications need be made.

In some embodiments, the IGF2 targeting peptide encompassed for use herein is described U.S. Pat. Nos. 7,785,856 and 9,873,868 which are each incorporated herein in their entirety by reference.

### (i) Deletion Mutants of IGF2:

In some embodiments, the IGF2 targeting peptide is a modified or truncated IGF2 targeting peptide (also referred to as a deletion mutant of IGF2), as disclosed in International Application PCT/US19/61701, filed Nov. 15, 2019, which is incorporated herein in its entirety by reference. For example, in some embodiments, the IGF2 targeting peptide comprises a V43M modification and also any deletion of one or more amino acids from amino acid 1-42. For example, in some embodiments of the methods and compositions as disclosed herein, the IGF2 targeting peptide comprises V43M and further comprises one or more deletions selected from any of: Δ1-3, Δ1-4, Δ1-5, Δ1-6, Δ1-8, Δ1-9, Δ1-10, Δ1-11, Δ1-12, Δ1-13, Δ1-14, Δ1-15, Δ1-16, Δ1-17, Δ1-18, Δ1-19, Δ1-20, Δ1-21, Δ1-22, Δ1-23, Δ1-24, Δ1-25, Δ1-26, Δ1-27, Δ1-28, Δ1-29, Δ1-30, Δ1-31, Δ1-32, Δ1-33, Δ1-34, Δ1-35, Δ1-36, Δ1-37, Δ1-38, Δ1-39, Δ1-40, Δ1-41 or Δ1-42 of SEQ ID NO: 5 and wherein residue 43 of SEQ ID NO: 5 is a methionine (V43M). In some embodiments of the methods and compositions as disclosed herein, the IGF2 targeting peptide comprises V43M and further comprises a Δ1-7 deletion (IGF2-A1-7,V43M).

In some embodiments of the methods and compositions as disclosed herein, the lysosomal IGF2 targeting peptide further comprises one or more modifications selected from any of: Δ2-3, Δ2-4, Δ2-5, Δ2-6, Δ2-8, Δ2-9, Δ2-10, Δ2-11, Δ2-12, Δ2-13, Δ2-14, Δ2-15, Δ2-16, Δ2-17, Δ2-18, Δ2-19, Δ2- 20, Δ2-21, Δ2-22, Δ2-23, Δ2-24, Δ2-25, Δ2-26, Δ2-27, Δ2-28, Δ2-29, Δ2-30, Δ2-31, Δ2-32, Δ2-33, Δ2-34, Δ2-35, Δ2-36, Δ2-37, Δ2-38, Δ2-39, Δ2-40, Δ2-41 or Δ2-42 of SEQ ID NO: 5 and wherein residue 43 of SEQ ID NO: 5 is a methionine (V43M). In some embodiments of the methods and compositions as disclosed herein, the IGF2 targeting peptide comprises V43M and further comprises a Δ2-7 deletion (IGF2-Δ2-7,V43M).

In some embodiments, a IGF2 targeting peptide for fusion to a GAA-polypeptide can comprise amino acids 8-28 and 41-61 of IGF2. In some embodiments, these stretches of amino acids can be joined directly or separated by a linker. Alternatively, amino acids 8-28 and 41-61 can be provided on separate polypeptide chains. In some embodiments, amino acids 8-28 of IGF2, or a conservative substitution variant thereof, could be fused to GAA polypeptide to express a IGF2-GAA fusion protein from the rAVV vector, and a separate rAAV vector could express IGF2 amino acids 41-61, or a conservative substitution variant thereof.

In order to facilitate proper presentation and folding of the IGF2 targeting peptide, longer portions of IGF2 proteins can be used. For example, an IGF2 targeting peptide including amino acid residues 1-67, 1-87, or the entire precursor form can be used.

In some embodiments, the IGF2 targeting peptide is a nucleic acid sequence that encodes an IGF2 targeting peptide of any of the following: residue 1 followed by residues 8-67 of wild-type mature human insulin-like growth factor II (IGF2) of SEQ ID NO: 5 (i.e., SEQ ID NO: 6; i.e., IGF2-delta 2-7); residues 8-67 of wild-type mature human insulin-like growth factor II (IGF2) of SEQ ID NO: 5 (i.e., SEQ ID NO: 7; IGF2-delta 1-7) or residues 43-67 of wild-type mature human insulin-like growth factor II (IGF2) of SEQ ID NO: 5 (i.e., IGF2-V43M (SEQ ID NO: 9) or IGF-delta 1-42 (SEQ ID NO: 8).

In some embodiments of the methods and compositions as disclosed herein, the IGF2 targeting peptide is a nucleic acid sequence selected from any nucleic acid sequence comprising any of: SEQ ID NO: 2 (i.e., IGF2-delta 2-7); SEQ ID NO: 3 (i.e., IGF2-delta 1-7) or SEQ ID NO: 4 (i.e., IGF2-V43M) or a sequence at least sequence at least 85%, 90%, 95%, 96%, 97%, 98% or 99% sequence identity thereto.

In some embodiments of the methods and compositions as disclosed herein, the IGF2(V43M) sequence is a nucleic acid sequence encoding a IGF2(V43M) sequence of any of SEQ ID NO: 65 (IGF2-Δ2-7V43M) or an amino acid sequence having at least 85%, or 90%, or 95% or 96%, or 97%, or 98% or 99% or 100% identity to SEQ ID NO: 65, or SEQ ID NO: 66 (IGFΔ1-7V43M) or an amino acid sequence having at least 85%, or 90%, or 95% or 96%, or 97%, or 98% or 99% or 100% identity to SEQ ID NO: 66.

TABLE 3
Exemplary nucleic acid sequences encoding IGF2 targeting peptide:

| IGF2 targeting | |
|---|---|
| peptide | Sequence |
| IGF2-delta 2-7 | GCT\|CTGTGCGGCGGGGAGCTGGTGGACACCCTCCAGTTCGTCTGTGGGGA |
| (IGFΔ2-7) | CCGCGGCTTCTACTTCAGCAGGCCCGCAAGCCGTGTGAGCCGTCGCAGCC |
| | GTGGCATCGTTGAGGAGTGCTGTTTCCGCAGCTGTGACCTGGCCCTCCTG |
| | GAGACGTACTGTGCTACCCCCGCCAAGTCCGAG) (SEQ ID NO: 2) |
| IGF2-delta 1-7 | CTGTGCGGCGGGGAGCTGGTGGACACCCTCCAGTTCGTCTGTGGGGACCG |
| (IGFΔ1-7) | CGGCTTCTACTTCAGCAGGCCCGCAAGCCGTGTGAGCCGTCGCAGCCGTG |
| | GCATCGTTGAGGAGTGCTGTTTCCGCAGCTGTGACCTGGCCCTCCTGGAG |
| | ACGTACTGTGCTACCCCCGCCAAGTCCGAG (SEQ ID NO: 3) |
| IGF2-V43M | GCTTACCGCCCCAGTGAGACCCTGTGCGGCGGGGAGCTGGTGGACACCCT |
| | CCAGTTCGTCTGTGGGGACCGCGGCTTCTACTTCAGCAGGCCCGCAAGCC |
| | GTGTGAGCCGTCGCAGCCGTGGCATCATGGAGGAGTGCTGTTTCCGCAGC |
| | TGTGACCTGGCCCTCCTGGAGACGTACTGTGCTACCCCCGCCAAGTCCGA |
| | G (SEQ ID NO: 4) |

In some embodiments, in order to facilitate proper presentation and folding of the IGF2 targeting peptide, longer portions of IGF2 proteins can be used. For example, an IGF2 targeting peptide including amino acid residues 1-67, 1-87, or the entire precursor form can be used.

In some embodiments of the methods and compositions disclosed herein, the recombinant AAV comprises a heterologous nucleic acid sequence encoding a signal peptide-GAA (SP-GAA) fusion polypeptide further comprises a IGF2 targeting peptide located between the secretory signal peptide (SP) and the an alpha-glucosidase (GAA) polypeptide.

In some embodiments of the methods and compositions disclosed herein, the recombinant AAV vector comprises a heterologous nucleic acid sequence that encodes a IGF2 targeting peptide which binds human cation-independent mannose-6-phosphate receptor (CI-MPR) or the IGF2 receptor, for example, the heterologous nucleic acid sequence encodes a IGF2 targeting peptide having the amino acid sequence of SEQ ID NO: 5 or comprises at least one amino modification in SEQ ID NO: 5 that binds to the IGF2 receptor. In some embodiments, the recombinant AAV vector comprises a heterologous nucleic acid sequence that encodes a IGF2 targeting peptide that has at least one amino modification in SEQ ID NO: 5 is a V43M amino acid modification (SEQ ID NO: 8 or SEQ ID NO: 9) or Δ2-7 (SEQ ID NO: 6) or Δ1-7 (SEQ ID NO: 7), or is a IGF2 peptide having at least about 75%, or 80%, or 85%, or 90%, or 95%, or 98%, or 99% sequence identity to SEQ ID NOs: 5-9.

In some embodiments of the methods and compositions disclosed herein, the nucleic acid encoding a IGF2 targeting peptide is selected from any of SEQ ID NO: 2 (IGF2-Δ2-7), SEQ ID NO: 3 (IGF2-Δ1-7), or SEQ ID NO: 4 (IGF2 V43M), or a nucleic acid sequence at least about 75%, or 80%, or 85%, or 90%, or 95%, or 98%, or 99% sequence identity to any of SEQ ID NOs: 2, 3 or 4.

In some embodiments of the compositions and methods described herein, the IGF2 targeting peptide is a nucleic acid sequence that encodes any of: residue 1 followed by residues 8-67 of wild-type mature human insulin-like growth factor II (IGF2) of SEQ ID NO: 5 (i.e., IGF2-delta 2-7 or IGF2-Δ2-7; which corresponds to SEQ ID NO: 6); residues 8-67 of wild-type mature human insulin-like growth factor II (IGF2) of SEQ ID NO: 5 (i.e., IGF2-delta 1-7 or IGF2A1-7, which corresponds to SEQ ID NO: 7;) or residues 43-67 of wild-type mature human insulin-like growth factor II (IGF2) of SEQ ID NO: 5 (i.e., IGF2 delta 1-42 or IGF2A1-42, which corresponds to SEQ ID NO: 8). In some embodiments of the compositions and methods described herein, the IGF2 targeting peptide is a nucleic acid sequence that has a modification of amino acid residue 43, for example residue 43 is modified to a start codon, for example IGF2-V43M (corresponding to SEQ ID NO: 9).

In some embodiments of the compositions and methods described herein, the IGF2 targeting peptide is a nucleic acid sequence comprising any of: SEQ ID NO: 2 (i.e., IGF2-delta 2-7); SEQ ID NO: 3 (i.e., IGF2-delta 1-7) or SEQ ID NO: 4 (i.e., IGF2-V43M).

In some embodiments of the compositions and methods described herein, the fusion protein comprising the GAA polypeptide and a IGF2 targeting peptide comprises amino acid residues 40-952 or residues 70-952 of human acid alpha-glucosidase (GAA) polypeptide (SEQ ID NO: 10) that is attached to an IGF2 targeting peptide that comprises residue 1 followed by residues 8-67 of wild-type mature human insulin-like growth factor II (IGF2) (SEQ ID NO: 5), (that is—residues 2-7 of mature human IGF2 (SEQ ID NO:5) are not present), wherein the IGF2 targeting peptide is linked to amino acid residue 70 of human GAA (SEQ ID NO: 10).

In some embodiments of the compositions and methods described herein, the fusion protein comprising the GAA polypeptide and a IGF2 targeting peptide comprises amino acid residues 40-952 or residues 70-952 of human acid alpha-glucosidase (GAA) polypeptide (SEQ ID NO: 10) that is attached to an IGF2 targeting peptide that comprises residues 8-67 of wild-type mature human insulin-like growth factor II (IGF2) (SEQ ID NO: 5), (that is—residues 1-7 of mature human IGF2 (i.e., Y R P S E T; SEQ ID NO: 63) are not present), wherein the IGF2 targeting peptide is linked to amino acid residue 70 of human GAA (SEQ ID NO: 10).

In some embodiments of the compositions and methods described herein, the fusion protein comprising the GAA polypeptide and a IGF2 targeting peptide comprises amino acid residues 40-952 or residues 70-952 of human acid alpha-glucosidase (GAA) (SEQ ID NO: 10) that is attached to a modified IGF2 targeting peptide that comprises residues 43-67 of wild-type mature human insulin-like growth factor II (IGF2) (SEQ ID NO: 5), (where residues 1-42 of mature human IGF2 (SEQ ID NO: 5) are not present), and where the IGF2 targeting peptide is linked to amino acid residue 70 of human GAA (SEQ ID NO: 10).

In some embodiments of the methods and compositions disclosed herein, a recombinant AAV vector comprises a heterologous nucleic acid sequence that encodes an IGF2 peptide, where the IGF2 peptide sequence is SEQ ID NO: 8 or SEQ ID NO: 9, or a IGF2 peptide having at least about 75%, or 80%, or 85%, or 90%, or 95%, or 98%, or 99% sequence identity to SEQ ID NO: 8 or 9.

### (ii) Modified IGF2 Targeting Peptides and IGF2 Homologues

In some embodiments, the nucleic acid encoding IGF2 can be modified to diminish their affinity for IGFBPs, and/or decreasing affinity for binding to IGF-I receptor, thereby increasing targeting to the lysosomes and increasing the bioavailability of the fused GAA-polypeptide.

IGF2 targeting peptide preferably specifically targets and binds to the M6P receptor. Particularly useful are IGF2 targeting peptides which have mutations in the IGF2 polypeptide that result in a protein that binds the CI-MPR/M6P receptor with high affinity while no longer binding the other two receptors with appreciable affinity.

IGF2(V43M) targeting peptide is preferably targeted specifically to the M6P receptor. Particularly useful are IGF2(V43M) targeting peptides which have mutations in the IGF2 polypeptide that result in a protein that binds the CI-MPR/M6P receptor with high affinity while no longer binding the other two receptors with appreciable affinity.

The IGF2(V43M) targeting peptide can also be modified to minimize binding to serum IGF-binding proteins (IGFBPs) (Baxter (2000) Am. J. Physiol Endocrinol Metab. 278(6):967-76) and to IGF-I receptor, in order to avoid sequestration of IGF2 constructs. A number of studies have localized residues in IGF-1 and IGF2 necessary for binding to IGF-binding proteins. Constructs with mutations at these residues can be screened for retention of high affinity binding to the M6P/IGF2 receptor and for reduced affinity for IGF-binding proteins. For example, replacing Phe 26 of IGF2 with Ser is reported to reduce affinity of IGF2 for IGFBP-1 and -6 with no effect on binding to the M6P/IGF2 receptor (Bach et al. (1993) J. Biol. Chem. 268(13):9246-54). Other substitutions, such as Ser for Phe 19 and Lys for Glu 9, can also be advantageous. The analogous mutations, separately or in combination, in a region of IGF-I that is highly conserved with IGF2 result in large decreases in IGF-BP binding (Magee et al. (1999) Biochemistry 38(48): 15863-70).

The IGF2 targeting peptide can also be modified to minimize binding to serum IGF-binding proteins (IGFBPs) and to IGF-I receptor, in order to avoid sequestration of IGF2 constructs.

In some embodiments, a IGF2 targeting peptide is modified to be furin resistant, i.e., resistant to degradation by furin protease, which recognizes Arg-X-X-Arg cleavage sites. Such IGF2 targeting peptides are disclosed in US application 22012/0213762 which is incorporated herein in its entirety by reference. In some embodiments, a furin resistant IGF2 targeting peptide for use in a rAAV genome as described herein contains a mutation within a region corresponding to amino acids 30-40 (e.g., 31-40, 32-40, 33-40, 34-40, 30-39, 31-39, 32-39, 34-37, 32-39, 33-39, 34-39, 35-39, 36-39, 37-40, 34-40) of SEQ ID NO: 5 (wt IGF2 targeting peptide) can be substituted with any other amino acid or deleted. For example, substitutions at position 34 may affect furin recognition of the first cleavage site. Insertion of one or more additional amino acids within each recognition site may abolish one or both furin cleavage sites. Deletion of one or more of the residues in the degenerate positions may also abolish both furin cleavage sites.

In some embodiments, a furin-resistant IGF2 targeting peptide contains amino acid substitutions at positions corresponding to Arg37 (R37) or Arg40 (R40) of SEQ ID NO:5. In some embodiments, a furin-resistant IGF2 targeting peptide contains a Lys (K) or Ala (A) substitution at positions Arg37 or Arg40 of SEQ ID NO: 5. Other substitutions are possible, including combinations of Lys and/or Ala mutations at both positions 37 and 40, or substitutions of amino acids other than Lys (K) or Ala (A). In some embodiments, the IGF2 targeting peptide encompassed for use in the rAVV genome as disclosed herein is IGFΔ2-7-K37, or IGFΔ2-7-K40 or IGFΔ1-7-K37 or IGFΔ1-7-K40, indicating that the IGF2 targeting peptides has a deletion of aa 2-7 or 1-7 and a modification of a Arg (R) residue at position 37 to a lysine (i.e., R37K modification) or R40K respectively. In some embodiments, the IGF2 targeting peptide encompassed for use in the rAVV genome as disclosed herein is IGFΔ2-7-K37-K40, or IGFΔ1-7-R37K-R40K indicating that the IGF2 targeting peptides has a deletion of residues 2-7 or residues 1-7 and a modification of a R residue at position 37 and position 40 to lysinines (R37K and R40K). In some embodiments, the IGF2 targeting peptide encompassed for use in the rAVV genome as disclosed herein is selected from any of: IGFΔ2-7-R37A, or IGFΔ2-7-R40A or IGFΔ1-7-R37A or IGFΔ1-7-R40A, IGFΔ2-7-R37A-R40A, or IGFΔ1-7-R37A-R40A. Exemplary constructs for the IGF2 targeting peptide encompassed for use in the rAVV genome as disclosed herein are disclosed in US application 2012/0213762, which is incorporated herein in its entirety by reference.

In some embodiments, the furin-resistant IGF2 targeting peptide suitable for the invention may contain additional mutations. For example, up to 30% or more of the residues of SEQ ID NO: 5 may be changed (e.g., up to 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or more residues may be changed). Thus, a furin-resistant IGF2 mutein suitable for the invention may have an amino acid sequence at least 70%, including at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99%, identical to SEQ ID NO: 5.

Moreover, use of a IGF2 targeting peptide as disclosed herein is also referred to in the art as Glycosylation Independent Lysosomal Targeting (GILT) because the IGF2 targeting peptide replaces M6P as the moiety targeting the lysosomes. Details of the GILT technology are described in U.S. Application Publication Nos. 2003/0082176, 2004/0006008, 2004/0005309, 2003/0072761, 2005/0281805, 2005/0244400, and international publications WO 03/032913, WO 03/032727, WO 02/087510, WO 03/102583, WO 2005/078077, the disclosures of all of which are hereby incorporated by reference.

Other modifications to the amino acid sequence of the IGF2 targeting peptide for use in the methods and compositions as disclosed herein are disclosed in US provisional application 62,937,556, filed on Nov. 19, 2019 and in PCT application PCT/US19/61653, filed Nov. 15, 2019, both of which are incorporated herein in their entirety by reference.

IGF2 binds to the IGF2/M6P and IGF-I receptors with relatively high affinity and binds with lower affinity to the insulin receptor. Substitution of IGF2 residues 48-50 (Phe Arg Ser) with the corresponding residues from insulin, (Thr Ser Ile), or substitution of residues 54-55 (Ala Leu) with the corresponding residues from IGF-I (Arg Arg) result in diminished binding to the IGF2/M6P receptor but retention of binding to the IGF-I and insulin receptors (Sakano et al. (1991) J. Biol. Chem. 266(31):20626-35).

IGF2 binds to repeat 11 of the cation-independent M6P receptor. Indeed, a minireceptor in which only repeat 11 is fused to the transmembrane and cytoplasmic domains of the cation-independent M6P receptor is capable of binding IGF2 (with an affinity approximately one tenth the affinity of the full length receptor) and mediating internalization of IGF2 and its delivery to lysosomes (Grimme et al. (2000) J. Biol. Chem. 275(43):33697-33703). The structure of domain 11 of the M6P receptor is known (Protein Data Base entries 1GP0 and 1GP3; Brown et al. (2002) EMBO J. 21(5):1054-1062). The putative IGF2 binding site is a hydrophobic pocket believed to interact with hydrophobic amino acids of IGF2; candidate amino acids of IGF2 include leucine 8, phenylalanine 48, alanine 54, and leucine 55. Although repeat 11 is sufficient for IGF2 binding, constructs including larger portions of the cation-independent M6P receptor (e.g. repeats 10-13, or 1-15) generally bind IGF2 with greater affinity and with increased pH dependence (see, for example, Linnell et al. (2001) J. Biol. Chem. 276(26):23986-23991).

Substitution of IGF2 residues Tyr 27 with Leu, or Ser 26 with Phe diminishes the affinity of IGF2 for the IGF-I receptor by 94-, 56-, and 4-fold respectively (Torres et al. (1995) J. Mol. Biol. 248(2):385-401). Deletion of residues 1-7 of human IGF2 resulted in a 30-fold decrease in affinity for the human IGF-I receptor and a concomitant 12-fold increase in affinity for the rat IGF2 receptor (Hashimoto et al. (1995) J. Biol. Chem. 270(30):18013-8). Truncation of the C-terminus of IGF2 (residues 62-67) also appear to lower the affinity of IGF2 for the IGF-I receptor by 5 fold (Roth et al. (1991) Biochem. Biophys. Res. Commun. 181(2):907-14).

Substitution of IGF2 residue phenylalanine 26 with serine reduces binding to IGFBPs 1-5 by 5-75 fold (Bach et al. (1993) J. Biol. Chem. 268(13):9246-54). Replacement of IGF2 residues 48-50 with threonine-serine-isoleucine reduces binding by more than 100 fold to most of the IGFBPs (Bach et al. (1993) J. Biol. Chem. 268(13):9246-54); these residues are, however, also important for binding to the cation-independent mannose-6-phosphate receptor. The Y27L substitution that disrupts binding to the IGF-I receptor interferes with formation of the ternary complex with IGFBP3 and acid labile subunit (Hashimoto et al. (1997) J. Biol. Chem. 272(44):27936-42); this ternary complex accounts for most of the IGF2 in the circulation (Yu et al. (1999) J. Clin. Lab Anal. 13(4):166-72). Deletion of the first six residues of IGF2 also interferes with IGFBP binding (Luthi et al. (1992) Eur. J. Biochem. 205(2):483-90).

Studies on IGF-I interaction with IGFBPs revealed additionally that substitution of serine for phenylalanine 16 did not affect secondary structure but decreased IGFBP binding by between 40 and 300 fold (Magee et al. (1999) Biochemistry 38(48):15863-70). Changing glutamate 9 to lysine also resulted in a significant decrease in IGFBP binding. Furthermore, the double mutant lysine 9/serine 16 exhibited the lowest affinity for IGFBPs. The conservation of sequence between this region of IGF-I and IGF2 suggests that a similar effect will be observed when the analogous mutations are made in IGF2 (glutamate 12 lysine/phenylalanine 19 serine).

In some embodiments, the IGF2(V43M) sequence comprises at least amino acids 48-55; at least amino acids 8-28 and 41-61; or at least amino acids 8-87, or a sequence variant thereof (e.g. R68A) or truncated form thereof (e.g. C-terminally truncated from position 62) that binds the cation-independent mannose-6-phosphate receptor.

In another embodiment of the invention, the rAAV genome encoding the targeting peptide (e.g., IGF2 targeting peptide) is inserted into the native GAA coding sequence at the junction of the mature 70/76 kDal polypeptide and the C-terminal domain, for example at position 791. This creates a single chimeric polypeptide. In some embodiments, a protease cleavage site may be inserted just downstream of the targeting peptide (e.g., IGF2 targeting peptide).

In one embodiment, a targeting peptide, e.g., IGF2 targeting peptide as defined herein, is fused directly to the N- or C-terminus of the GAA polypeptide. In another embodiment, a IGF2 targeting peptide is fused to the N- or C-terminus of the GAA polypeptide by a spacer. In one specific embodiment, a IGF2 targeting peptide is fused to the GAA polypeptide by a spacer of 10-25 amino acids. In another embodiment, a IGF2 targeting peptide is fused to the GAA polypeptide by a spacer including glycine residues.

In some embodiments, a IGF2 targeting peptide is fused to the GAA polypeptide by a spacer of at least 1, 2, or 3 amino acids. In some embodiments, the spacer comprises amino acids GAP or Gly-Ala-Pro (SEQ ID NO: 31), or an amino acid sequence at least 50% identical thereto. In some embodiments, the spacer is GGG or GA or AP, or GP or variants thereof. In some embodiments, the spacer is encoded by nucleic acids GGC GCG CCG (SEQ ID NO: 30).

In some embodiments, a IGF2 targeting peptide is fused to the GAA polypeptide by a spacer including a helical structure. In another specific embodiment, a IGF2 targeting peptide is fused to the GAA polypeptide by a spacer at least 50% identical to the sequence GGGTVGDDDDK (SEQ ID NO: 35). In some embodiments of the methods and compositions as disclosed herein, the spacer is SEQ ID NO: 31 (encoded by nucleic acids of SEQ ID NO: 30). In some embodiments of the methods and compositions as disclosed herein, the spacer is selected from any of: SEQ ID NO: 31, SEQ ID NO: 32, SEQ ID NO: 33, SEQ ID NO: 34 or SEQ ID NO: 35, or a sequence at least sequence at least 85%, 90%, 95%, 96%, 97%, 98% or 99% sequence identity thereto.

(iii) Alternative Targeting Peptides that Bind to the Cation-Independent M6P Receptor (CI-MPR).

In some embodiments, the targeting peptide is a lysosomal targeting peptide or protein, or other moiety other than the IGF2 targeting peptide disclosed herein that binds to the cation independent M6P/IGF2 receptor (CI-MPR) in a mannose-6-phosphate-independent manner. The CI-MPR also contains binding sites for at least three distinct ligands that can be used as targeting peptides. As disclosed herein, IGF2 ligand binds to CI-MPR with a dissociation constant of about 14 nM at or about pH 7.4, primarily through interactions with repeat 11. The CI-MPR is capable of binding high molecular weight O-glycosylated IGF2 forms. Accordingly, in some embodiments, the IGF2 targeting peptide can be post-transcriptionally modified to comprises O-glycosylation.

In an alternative embodiment, the targeting peptide that binds to CI-MPR is retinoic acid. Retinoic acid binds to the receptor with a dissociation constant of 2.5 nM. Affinity photolabeling of the cation-independent M6P receptor with retinoic acid does not interfere with IGF2 or M6P binding to the receptor, indicating that retinoic acid binds to a distinct site on the receptor. Binding of retinoic acid to the receptor alters the intracellular distribution of the receptor with a greater accumulation of the receptor in cytoplasmic vesicles and also enhances uptake of M6P modified β-glucuronidase. Retinoic acid has a photoactivatable moiety that can be used to link it to a therapeutic agent without interfering with its ability to bind to the cation-independent M6P receptor.

The urokinase-type plasminogen receptor (uPAR) also binds CI-MPR with a dissociation constant of 9 μM. uPAR is a GPI-anchored receptor on the surface of most cell types where it functions as an adhesion molecule and in the proteolytic activation of plasminogen and TGF-3. Binding of uPAR to the CI-M6P receptor targets it to the lysosome, thereby modulating its activity. Thus, fusing the extracellular domain of uPAR, or a portion thereof competent to bind the cation-independent M6P receptor, to a therapeutic agent permits targeting of the agent to a lysosome.

### D. Spacer and Fusion Junction of the GAA Polypeptide

Where GAA is expressed as a fusion protein with a secretory signal peptide (e.g., SS-GAA fusion polypeptide) or with a targeting peptide (i.e., SS-IGF2-GAA polypeptide double fusion polypeptide), the signal peptide or IGF2 targeting peptide can be fused directly to the GAA polypeptide or can be separated from the GAA polypeptide by a linker. An amino acid linker (also referred to herein as a “spacer”) incorporates one or more amino acids other than that appearing at that position in the natural protein. Spacers can be generally designed to be flexible or to interpose a structure, such as an a-helix, between the two protein moieties.

Accordingly, in some embodiments of the methods and compositions disclosed herein, a recombinant AAV vector comprises a heterologous nucleic acid sequence encoding an IGF2-GAA fusion polypeptide, wherein the IGF2-GAA fusion protein further comprises a spacer comprising a nucleotide sequence of at least 1 amino acid in length, which is located N-terminal to the GAA polypeptide, and C-terminal to the IGF2 targeting peptide. In some embodiments of the methods and compositions disclosed herein, a recombinant AAV vector comprises a heterologous nucleic acid sequence that comprises a nucleic acid encoding a spacer of at least 1 amino acids located between the nucleic acid encoding the IGF2 targeting peptide and the nucleic acid encoding the GAA polypeptide.

In one embodiment, the IGF2 targeting peptide is fused directly to the N- or C-terminus of the GAA polypeptide. In another embodiment, a IGF2 targeting peptide is fused to the N- or C-terminus of the GAA polypeptide by a spacer. In one specific embodiment, a IGF2 targeting peptide is fused to the GAA polypeptide by a spacer of 10-25 amino acids. In another specific embodiment, a IGF2 targeting peptide is fused to the GAA polypeptide by a spacer including glycine residues. In another specific embodiment, a IGF2 targeting peptide is fused to the GAA polypeptide by a spacer including a helical structure. In another specific embodiment, a IGF2 targeting peptide is fused to the GAA polypeptide by a spacer at least 50% identical to the sequence GGGTVGDDDDK (SEQ ID NO: 35).

In some embodiments, a spacer or linker can be relatively short, e.g., at least 1, 2, 3, 4 or 5 amino acids, or such as the sequence Gly-Ala-Pro (SEQ ID NO: 31) or Gly-Gly-Gly-Gly-Gly-Pro (SEQ ID NO: 32), or can be longer, such as, for example, 5-10 amino acids in length or 10-25 amino acids in length. For example, flexible repeating linkers of 3-4 copies of the sequence (GGGGS (SEQ ID NO:33)) and a-helical repeating linkers of 2-5 copies of the sequence (EAAAK (SEQ ID NO:34)) have been described (Arai et al. (2004) Proteins: Structure, Function and Bioinformatics 57:829-838).

The use of another linker, GGGTVGDDDDK (SEQ ID NO: 35), in the context of an IGF2 fusion protein has also been reported (DiFalco et al. (1997) Biochem. J. 326:407-413) and is encompassed for use. Linkers incorporating an a-helical portion of a human serum protein can be used to minimize immunogenicity of the linker region.

In some embodiments, the spacer is encoded by nucleic acids GGC GCG CCG (SEQ ID NO: 30) which encodes the amino acid spacer comprising amino acids GAP or Gly-Ala-Pro (SEQ ID NO: 31).

The site of a fusion junction in the GAA polypeptide to fuse with either the signal peptide (to generate a SS-GAA fusion protein) or with the targeting peptide (e.g., to generate a SP-IGF2-GAA double fusion polypeptide) should be selected with care to promote proper folding and activity of each polypeptide in the fusion protein and to prevent premature separation of a signal peptide from a GAA polypeptide.

In some embodiments, a IGF2 targeting peptide is fused to the GAA polypeptide by a spacer including a helical structure. In another specific embodiment, a IGF2 targeting peptide is fused to the GAA polypeptide by a spacer at least 50% identical to the sequence GGGTVGDDDDK (SEQ ID NO: 35). In some embodiments of the methods and compositions as disclosed herein, the spacer is SEQ ID NO: 31 (encoded by nucleic acids of SEQ ID NO: 30). In some embodiments of the methods and compositions as disclosed herein, the spacer is selected from any of: SEQ ID NO: 31, SEQ ID NO: 32, SEQ ID NO: 33, SEQ ID NO: 34 or SEQ ID NO: 35.

Four exemplary strategies for creating a IGF2-GAA fusion protein can be generated, which are disclosed in provisional application 62,937,556, filed on Nov. 19, 2019, and in PCT/US19/61653, filed Nov. 15, 2019, which are incorporated herein in their entirety by reference.

In some embodiments, a targeting peptide (e.g., a IGF2 targeting peptide) can be fused, directly or by a spacer, to amino acid 40 or amino acid 70 of GAA, a position permitting expression of the protein, catalytic activity of the GAA protein, and proper targeting by the IGF2 targeting peptide as described herein in the Examples. Alternatively, a targeting peptide (e.g., a IGF2 targeting peptide) can be fused at or near the cleavage site separating the C-terminal domain of GAA from the mature polypeptide. This permits synthesis of a GAA protein with an internal targeting peptide (e.g., a IGF2 targeting peptide), which optionally can be cleaved to liberate the mature polypeptide or the C-terminal domain from the targeting domain, depending on placement of cleavage sites. Alternatively, the mature polypeptide can be synthesized as a fusion protein at about position 791 without incorporating C-terminal sequences in the open reading frame of the expression construct.

In order to facilitate folding of the IGF2 targeting peptide, GAA amino acid residues adjacent to the fusion junction can be modified. For example, since it is possible that GAA cysteine residues may interfere with proper folding of the targeting peptide (e.g., a IGF2 targeting peptide), the terminal GAA cysteine 952 can be deleted or substituted with serine to accommodate a C-terminal targeting peptide (e.g., a IGF2 targeting peptide). The targeting peptide (e.g., a IGF2 targeting peptide) can also be fused immediately preceding the final Cys952. The penultimate cys938 can be changed to proline in conjunction with a mutation of the final Cys952 to serine.

### E. CS Sequence

In some embodiments of the methods and compositions disclosed herein, a recombinant AAV vector comprises a heterologous nucleic acid sequence that further comprises at collagen stability (CS) sequence located 3′ of the nucleic acid encoding the GAA polypeptide and 5′ of the 3′ ITR sequence. In some embodiments, the rAAV genome disclosed herein comprises a heterologous nucleic acid sequence that can optionally comprise a Collagen stability sequence (CS or CSS), which is positioned 3′ of the GAA gene and 5′ of a polyA signal. In some embodiments, the CS sequence can be replaced by a 3′ UTR sequence as disclosed herein.

Exemplary collagen stability sequences include CCCAGCCCACTTTTCCCCAA (SEQ ID NO: 65) or a sequence at least 85%, 90%, 95%, 96%, 97%, 98% or 99% sequence identity thereto. An exemplary collagen stability sequence can have an amino acid sequence of P S P L F P (SEQ ID NO: 66) or an amino acid sequence having at least 85%, 90%, 95%, 96%, 97%, 98% or 99% sequence identity thereto. CS sequences are disclosed in Holick and Liebhaber, Proc. Nat. Acad. Sci. 94: 2410-2414, 1997 (See, e.g. FIG. 3, p. 5205), which is incorporated herein its entirety by reference.

### F. Promoters

In some embodiments, to achieve appropriate levels of GAA expression, the rAAV genotype comprises a liver specific promoter (LSP). A LSP enables expression of the operatively linked gene in the liver, and can in some embodiments, be and inducible LSP. In an embodiment, a LSP is located upstream 5′ and is operatively linked to the heterologous nucleic acid sequence encoding the GAA protein. Exemplary liver-specific promoters are disclosed herein, and include for example, the LSP comprising SEQ ID NO: 86, 91-96 or 146-150, or functional variant or functional fragment thereof, or any LSP listed in Table 4 herein, or a functional fragment or functional variants thereof. In some embodiments of the compositions and methods disclosed herein, a liver-specific promoter includes a liver-specific cis-regulatory element (CRE), a synthetic liver-specific cis-regulatory module (CRM) or a synthetic liver-specific promoter is selected from any of SEQ ID NO: 270-341 (minimal LSP with CRM) or SEQ ID NO: 342-430 (synthetic liver specific proximal promoters) disclosed in Table 4 herein). In some embodiments, an rAAV vector genome can include one or more constitutive promoters, such as viral promoters or promoters from mammalian genes that are generally active in promoting transcription.

### (i) Synthetic Liver-Specific Promoters

In some embodiments of the methods and compositions as disclosed herein, the promoter is a liver specific promoter, and can be selected from promoters including, but not limited to, those listed in Table 4 disclosed herein or functional variants thereof, and or any selected from Tables 4A and 4B of U.S. provisional application 62,937,556, filed on Nov. 19, 2019, or functional variants thereof.

While transthyretin promoter (TTR) (SEQ ID NO: 431) and SP0412 (SEQ ID NO: 91) and SP0422 (SEQ ID NO: 92) are used as an exemplary liver specific promoters (see Examples 1, 12 and 13) in the specification and Examples, one of ordinary skill in the art can readily replace TTR with any liver specific promoter as disclosed herein in Table 4 or functional variants thereof, and or any selected from Tables 4A and 4B of U.S. provisional application 62,937,556, filed on Nov. 19, 2019, or functional variants thereof. A liver-specific promoter can comprise a liver-specific cis-regulatory element (CRE), a synthetic liver-specific cis-regulatory module (CRM) or a synthetic liver-specific promoter as disclosed herein, in Tables 4A and 4B of U.S. provisional application 62,937,556, filed on Nov. 19, 2019, or functional variants thereof.

Table 4 shows exemplary liver-specific promoters. The relatively small size of liver-specific promoters disclosed herein is advantageous because it takes up the minimal amount of the payload of the vector. This is particularly important when a LSP is used in a vector with limited capacity, such as an AAV-based vector.

Table 4: Exemplary LSP identified by SEQ ID NOs for use in the methods and compositions as disclosed herein

TABLE 4
Exemplary LSP

| SEQ ID | |
|---|---|
| NO: | Name of LSP |
| 270 | CRM_SP0107 |
| 271 | CRM_SP0109 |
| 273 | CRM_SP0111 |
| 274 | CRM_SP0112 |
| 275 | CRM_SP0113 |
| 276 | CRM_SP0115 |
| 277 | CRM_SP0116 |
| 278 | CRM_SP0121 |
| 279 | CRM_SP0124 |
| 280 | CRM_SP0127 |
| | (CRM_LVR_127) |
| 281 | CRM_SP0127_A1 |
| | (CRM_LVR_127_A1) |
| 282 | CRM_SP0127V1 |
| | (CRM_LVR_127_V1) |
| 283 | CRM_SP0127V2 |
| | (CRM_LVR_127_V2) |
| 284 | CRM_SP0128 |
| 285 | CRM_SP0131 |
| | (CRM_LVR_131) |
| 286 | CRM_SP0132 |
| | (CRM_LVR_132) |
| 287 | CRM_SP0133 |
| | (CRM_LVR_133) |
| 288 | CRM_SP0155 |
| 289 | CRM_SP0158 |
| 290 | CRM_SP0163 |
| 291 | CRM_SP0236 |
| 292 | CRM_SP0239 |
| 293 | CRM_SP0240 |
| 294 | CRM_SP0241 |
| 295 | CRM_SP0242 |
| 296 | CRM_SP0243 |
| 297 | CRM_SP0244 |
| 298 | CRM_SP0246 |
| 299 | CRM_SP0247 |
| 300 | CRM_SP0248 |
| 301 | CRM_SP0249 |
| 302 | CRM_SP0250 |
| 303 | CRM_SP0251 |
| 304 | CRM_SP0252 |
| 305 | CRM_SP0253 |
| 306 | CRM_SP0254 |
| 307 | CRM_SP0255 |
| 308 | CRM_SP0256 |
| 309 | CRM_SP0257 |
| 310 | CRM_SP0258 |
| 311 | CRM_SP0259 |
| 312 | CRM SP0264 |
| 313 | CRM_SP0265 |
| | (CRM_LVR_131_A1) |
| 314 | CRM_SP0266 |
| | (CRM_LVR_131_V1) |
| 315 | CRM_SP0267 |
| | (CRM_LVR_131_V2) |
| 316 | CRM_SP0268 |
| | (CRM_LVR_132_A1) |
| 317 | CRM_SP0269 |
| | (CRM_LVR_132_V1) |
| 318 | CRM_SP0270 |
| | (CRM_LVR_132_V2) |
| 319 | CRM_SP0271 |
| | (CRM_LVR_133_A1) |
| 320 | CRM_SP0272 |
| | (CRM LVR_133_V1) |
| 321 | CRM_SP0273 |
| | (CRM_LVR_133_V2) |
| 322 | CRM_SP0368 |
| 323 | CRM_SP0373 |
| 324 | CRM_SP0378 |
| 325 | CRM_SP0379 |
| 326 | CRM_SP0380 |
| 327 | CRM_SP0381 |
| 328 | CRM_SP0384 |
| 329 | CRM_SP0388 |
| 330 | CRM_SP0396 |
| 331 | CRM_SP0397 |
| 332 | CRM_SP0398 |
| 333 | CRM_SP0399 |
| 334 | CRM_SP0403 |
| 335 | CRM_SP0404 |
| 336 | CRM_SP0405 |
| 337 | CRM_SP0406 |
| 338 | CRM_SP0407 |
| 339 | CRM_SP0409 |
| 340 | CRM_SP0411 |
| 86 | CRM_SP0412 |
| 341 | CRM_SP0413 |
| 342 | SP0107 |
| 343 | SP0109 |
| 344 | SP0111 |
| 345 | SP0112 |
| 346 | SP0113 |
| 347 | SP0115 |
| 348 | SP0116 |
| 349 | SP0121 |
| 350 | SP0124 |
| 351 | SP0127 |
| | (LVR_SP127) |
| 352 | SP0127A1 |
| | (LVR_SP127_A1) |
| 353 | SP0127V1 |
| | (LVR_SP127_V1) |
| 354 | SP0127V2 |
| | (LVR_SP127_V2) |
| 355 | SP0128 |
| 356 | SP0131 |
| | (LVR_SP131) |
| 357 | SP0132 |
| | (LVR_SP132) |
| 358 | SP0133 |
| | (LVR_SP133) |
| 359 | SP0155 |
| 360 | SP0158 |
| 361 | SP0163 |
| 362 | SP0236 |
| 93 | SP0239 |
| 95 | SP0240 |
| 363 | SP0241 |
| 364 | SP0242 |
| 365 | SP0243 |
| 366 | SP0244 |
| 96 | SP0246 |
| 367 | SP0247 |
| 368 | SP0248 |
| 369 | SP0249 |
| 370 | SP0250 |
| 371 | SP0251 |
| 372 | SP0252 |
| 373 | SP0253 |
| 374 | SP0254 |
| 375 | SP0255 |
| 376 | SP0256 |
| 377 | SP0257 |
| 378 | SP0258 |
| 379 | SP0259 |
| 380 | SP0264 |
| 94 | SP0265 |
| | (LVR_SP131_A1) |
| 381 | SP0266 |
| | (LVR_SP131_V1) |
| 382 | SP0267 |
| | (LVR_SP131_V2) |
| 383 | SP0268 |
| | (LVR_132_A1) |
| 384 | SP0269 |
| | (LVR_132_V1) |
| 385 | SP0270 |
| | (LVR_132_V2) |
| 386 | SP0271 |
| | (LVR_133_A1) |
| 387 | SP0272 |
| | (LVR_133_V1) |
| 388 | SP0273 |
| | (LVR_133_V2) |
| 389 | SP0256 |
| 390 | SP0257 |
| 391 | SP0258 |
| 392 | SP0259 |
| 393 | SP0264 |
| 394 | SP0265 |
| | (LVR_SP131_A1) |
| 395 | SP0266 |
| | (LVR_SP131_V1) |
| 396 | SP0267 |
| | (LVR_SP131_V2) |
| 397 | SP0268 |
| | (LVR_132_A1) |
| 398 | SP0269 |
| | (LVR_132_V1) |
| 399 | SP0270 |
| | (LVR_132_V2) |
| 400 | SP0271 |
| | (LVR_133_A1) |
| 401 | SP0272 |
| | (LVR_133_V1) |
| 402 | SP0273 |
| | (LVR_133_V2) |
| 403 | SP0368 |
| 404 | SP0373 |
| 405 | SP0378 |
| 406 | SP0379 |
| 407 | SP0380 |
| 408 | SP0381 |
| 409 | SP0272 |
| | (LVR_133_V1) |
| 410 | SP0273 |
| | (LVR_133_V2) |
| 411 | SP0368 |
| 412 | SP0373 |
| 413 | SP0378 |
| 414 | SP0379 |
| 415 | SP0380 |
| 416 | SP0381 |
| 417 | SP0384 |
| 418 | SP0388 |
| 419 | SP0396 |
| 420 | SP0397 |
| 421 | SP0398 |
| 422 | SP0399 |
| 423 | SP0403 |
| 424 | SP0404 |
| 425 | SP0405 |
| 426 | SP0406 |
| 427 | SP0407 |
| 428 | SP0409 |
| 429 | SP0411 |
| 91 | SP0412 |
| 92 | SP0422 |
| 146 | SP0265-UTR |
| 147 | SP0239-UTR |
| 148 | SP0240-UTR |
| 149 | SP0246-UTR |
| 150 | SP0131-A1-UTR |
| 430 | SP0413 |
| 431 | TTR promoter |
| 432 | LP1 |
| 433 | CMV-IE |
| 434 | CBA |
| 435 | TBG promoter |

### (ii) Functional Variants of Liver-Specific Promoters

In some embodiments, the synthetic liver-specific promoter useful in the methods and compositions as disclosed herein is a bi-specific, or tri-specific promoter as defined herein. As an illustrative example, a liver bi-specific promoter is active in the liver and one other tissue, for example, the muscle. Additionally, another illustrative example of a liver bi-specific promoter is active in the liver and one other tissue, e.g., the brain. As an illustrative example of a liver tri-specific promoter is active in the liver and two other tissues, for example, the muscle and brain. Additionally, another illustrative example of a liver tri-specific promoter is active in the liver and two other tissues, such as, e.g., the kidney and muscle.

In some embodiments, a synthetic liver specific promoter that is at least 50%, 60%, 70%, 80%, 90% or 95% identical to any of SEQ ID NO: 86, 91-96, 146-150, 270-430 comprises a source regulatory nucleic acid sequence which is preferentially active in liver, and is also active to a lesser extent (e.g., <50%, or about 49-40%, or about 39-30%, or about 29-20% or about 19-10% or <10% of total expression) in a second type of cell or tissue, e.g., muscle or CNS.

In some embodiments, the promoter is a synthetic liver-specific promoter comprising a combination of the cis-regulatory elements (CREs) CRE0051 (SEQ ID NO: 97) and CRE0042 (SEQ ID NO: 104), or functional variants thereof. Functional variants thereof may have a sequence that is at least 60%, 65%, 70%, 75%, 80%, 85%, 90%, 95%, 96%, 97%, 98%, or 99% identical thereto. Typically, the CREs are operably linked to a promoter element. In some preferred embodiments, the liver-specific promoter comprises said CREs, or functional variants thereof, in the order CRE0051 (SEQ ID NO: 97), CRE0042 (SEQ ID NO: 104), and then the promoter element (order is given in an upstream to downstream direction, as is conventional in the art).

The promoter element can be any suitable proximal promoter or minimal promoter. In some embodiments, the promoter element is a minimal promoter. Where the promoter is a proximal promoter, it is generally preferred that the proximal promoter is liver-specific.

In some preferred embodiments, the promoter element is CRE0059 (SEQ ID NO: 110), or a functional variant thereof. CRE0059 is a proximal promoter, as is discussed further below.

Thus, in one embodiment the promoter comprises the following regulatory elements: CRE0051 (SEQ ID NO: 97), CRE0042 (SEQ ID NO: 104) and CRE0059 (SEQ ID NO: 110), or functional variants thereof.

Functional variants of CRE0051 (SEQ ID NO: 97) are regulatory elements with sequences which vary from CRE0051, but which substantially retain activity as liver-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0051 can be viewed as a CRE which, when substituted in place of CRE0051 in a promoter, substantially retains its activity. For example, a liver-promoter which comprises a functional variant of CRE0051 substituted in place of CRE0051 preferably retains 80% of its activity, more preferably 90% of its activity, more preferably 95% of its activity, and yet more preferably 100% of its activity. For example, considering promoter SP0412 (SEQ ID NO: 91) as an example, CRE0051 in SP0412 can be replaced with a functional variant of CRE0051, and the promoter substantially retains its activity. Retention of activity can be assessed by comparing expression of a suitable reporter under the control of the reference promoter with an otherwise identical promoter comprising the substituted CRE under equivalent conditions.

In some embodiments the functional variant of CRE0051 comprises transcription factor binding sites (TFBS) for the same liver-specific TFs as CRE0051. The liver-specific TFBS present in CRE0051, listed in the order in which they are present, are: HNF1 (SEQ ID NO: 98), HNF4 (SEQ ID NO: 99), HNF3 (SEQ ID NO: 100), HNF1′ (SEQ ID NO: 101) and HNF3′ (SEQ ID NO: 102), see Table 5. The functional variant of CRE0051 thus preferably comprises all of these TFBS. Preferably, they are present in the same order that they are present in CRE0051, i.e. in the order HNF1 (SEQ ID NO: 98), HNF4 (SEQ ID NO: 99), HNF3 (SEQ ID NO: 100), HNF1′ (SEQ ID NO: 101) and HNF3′ (SEQ ID NO: 102). When the cis-regulatory element is associated with a promoter and gene, this order is preferably considered in an upstream to downstream direction (i.e. in the direction from distal from the transcription start site (TSS) to proximal to the TSS). Spacer sequences may be provided between adjacent TFBS. In some embodiments the TFBS may suitably overlap, provided they remain functional, i.e. overlapping sequences are both able to bind their respective TFs to the extent required to regulate expression.

In some embodiments the functional variant of CRE0051 (SEQ ID NO: 97) comprises the following TFBS sequences: GTTAATTTTTAAA (HNF1) (SEQ ID NO: 98), GTGGCCCTTGG (HNF4) (SEQ ID NO: 99), TGTTTGC (HNF3) (SEQ ID NO: 100), TGGTTAATAATCTCA (HNF1′) (SEQ ID NO: 101) then ACAAACA (HNF3) (SEQ ID NO: 102), sequences complementary thereto, or functional variants of these TFBS sequences that maintain the ability to bind to their respective TF. These may be present in the same order as CRE0051, i.e. the order in which they are set out above. It is well-known in the art that there is sequence variability associated with TFBS, and that for a given TFBS there is typically a consensus sequence, from which some degree of deviation is typically present. Further information about the variation that occurs in a TFBS can be illustrated using a positional weight matrix (PWM), which represents the frequency with which a given nucleotide is typically found at a given location in the consensus sequence. Details of TF consensus sequences and associated PWMs can be found in, for example, the Jaspar or Transfac databases (http://jaspar.genereg.net/ and http://gene-regulation.com/pub/databases.html). This information allows the skilled person to modify the sequence in any given TFBS of a CRE in a manner which retains, and in some cases even increases, CRE functionality.

In some embodiments, the functional variant of CRE0051 comprises the sequence:

GTTAATTTTTAAA-Na-GTGGCCCTTGG-Nb-TGTTTGC-Nc-TGGTTAATAATCTCA-Nd-ACAAACA (SEQ ID NO: 103), or a sequence that is at least 70%, 80%, 90%, 95% or 99% identical thereto, wherein Na, Nb, Nc, and Nd represent optional spacer sequences. When present, Na optionally has a length of from 10 to 26 nucleotides, preferably from 14 to 22 nucleotides, and more preferably 18 nucleotides. When present, Nb optionally has a length of from 8 to 22 nucleotides, preferably from 12 to 20 nucleotides, more preferably 16 nucleotides. When present, Nc optionally has a length of from 1 to 10 nucleotides, preferably 1 to 5 nucleotides, and more preferably 2 nucleotides. When present, Nd suitably has a length of from 1 to 13 nucleotides, preferably from 2 to 9 nucleotides in length, and more preferably 5 nucleotides in length.

In some embodiments, the CRE consists of SEQ ID No: 98-102 or a functional variant thereof.

It will be noted that the CRE or functional variant thereof can be provided on either strand of a double stranded polynucleotide and can be provided in either orientation. As such, complementary and reverse complementary sequences of SEQ ID NO: 97-102 or a functional variant thereof fall within the scope of the invention. Single stranded nucleic acids comprising the sequence according to SEQ ID NO: 97 or 103 or a functional variant thereof also fall within the scope of the invention.

In some embodiments, the CRE comprising or consisting of CRE0051 (SEQ ID NO: 97), or a functional variant thereof, has a length of 200 or fewer nucleotides, 150 or fewer nucleotides, 125 or fewer nucleotides, or 100 or fewer nucleotides.

In some embodiments, the CRE comprising or consisting of CRE0042 (SEQ ID NO: 104) or a functional variant thereof, has a length of 200 or fewer nucleotides, 150 or fewer nucleotides, 125 or fewer nucleotides, or 100 or fewer nucleotides. Functional variants thereof may have a sequence that is at least 60%, 65%, 70%, 75%, 80%, 85%, 90%, 95%, 96%, 97%, 98%, or 99% identical thereto.

Functional variants of CRE0042 (SEQ ID NO: 104) are regulatory elements with sequences which vary from CRE0042, but which substantially retain their activity as liver-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0042 (SEQ ID NO: 104) can be viewed as a CRE which, when substituted in place of CRE0042 in a promoter, substantially retains its activity. For example, a promoter which comprises a functional variant of CRE0042 substituted in place of CRE0042 preferably retains 80% of its activity, more preferably 90% of its activity, more preferably 95% of its activity, and yet more preferably 100% of its activity (compared to the reference promoter comprising CRE0042 (SEQ ID NO: 104)). For example, considering promoter SP0412 as an example, CRE0042 (SEQ ID NO: 104) in SP412 (SEQ ID NO: 91) can be replaced with a functional variant of CRE0042, and the promoter substantially retains its activity. Retention of activity can be assessed by comparing expression of a suitable reporter under the control of the reference promoter with an otherwise identical promoter comprising the substituted CRE under equivalent conditions.

In some embodiments it is preferred that the functional variant of CRE0042 (SEQ ID NO: 104) comprises TFBS for the same liver-specific TFs as CRE0042. The liver-specific TFBS present in CRE0042, listed in the order in which they are present, are: HNF-3 (SEQ ID NO: 106), C/EBP (SEQ ID NO: 107), HNF-4 (SEQ ID NO: 108) and C/EBP′ (SEQ ID NO: 109). The functional variant of CRE0042 thus preferably comprises all of these TFBS. Preferably, they are present in the same order that they are present in CRE0042, i.e. in the order HNF-3, C/EBP, HNF-4 and then C/EBP. When the cis-regulatory element is associated with a promoter and gene, this order is preferably considered in an upstream to downstream direction (i.e. in the direction from distal from the transcription start site (TSS) to proximal to the TSS). Spacer sequences may be provided between adjacent TFBS. In some embodiments the TFBS may suitably overlap, provided they remain functional, i.e. overlapping sequences are both able to bind their respective TFs.

In some embodiments the functional variant of CRE042 (SEQ ID NO: 104) comprises the following TFBS sequences: GTTCAAACATG (HNF-3) (SEQ ID NO: 106), CTAATACTCTG (C/EBP) (SEQ ID NO: 107), TGCAAGGGTCAT (HNF-4) (SEQ ID NO: 108), and TTACTCAACA (C/EBP) (SEQ ID NO: 109) and sequences complementary thereto, or functional variants of these TFBS sequences that maintain the ability to bind to their respective TF. These may be present in the same order as CRE0042, i.e. the order in which they are set out above. As discussed above, it is well-known in the art that there is sequence variability associated with TFBS, and that for a given TFBS there is typically a consensus sequence, from which some degree of deviation is typically present.

In some embodiments of the invention, the functional variant of CRE0042 comprises the sequence:

GTTCAAACATG-Na-CTAATACTCTG-Nb-TGCAAGGGTCAT-Nc-TTACTCAACA (SEQ ID NO: 105) or a sequence that is at least 70%, 80%, 90%, 95% or 99% identical thereto, wherein Na, Nb and Nc represent optional spacer sequences. When present, Na optionally has a length of from 1 to 10 nucleotides, preferably from 1 to 5 nucleotides, and more preferably 2 nucleotides. When present, Nb optionally has a length of from 1 to 10 nucleotides, preferably from 2 to 6 nucleotides, and more preferably 4 nucleotides. When present, Nc optionally has a length of from 8 to 23 nucleotides, preferably from 10 to 20 nucleotides, and more preferably 15 nucleotides.

In some embodiments of the invention the cis-regulatory enhancer element consists of CRE0042 (SEQ ID NO: 104) or a functional variant thereof.

It will be noted that the CRE or functional variant thereof can be provided on either strand of a double stranded polynucleotide and can be provided in either orientation. As such, complementary and reverse complementary sequences of SEQ ID NO: 104 or 105 or a functional variant thereof fall within the scope of the invention. Single stranded nucleic acids comprising the sequence according to SEQ ID NO: 104 or 105 or a functional variant thereof also fall within the scope of the invention.

In some embodiments, the CRE comprising or consisting of CRE0042 (SEQ ID NO: 104), or a functional variant thereof, has a length of 200 or fewer nucleotides, 150 or fewer nucleotides, 125 or fewer nucleotides, 100 or fewer nucleotides, or 80 or fewer nucleotides.

In some embodiments, the CRE comprising or consisting of CRE0059 (SEQ ID NO: 110) or a functional variant thereof, has a length of 200 or fewer nucleotides, 150 or fewer nucleotides, 125 or fewer nucleotides, or 100 or fewer nucleotides. Functional variants thereof may have a sequence that is at least 60%, 65%, 70%, 75%, 80%, 85%, 90%, 95%, 96%, 97%, 98%, or 99% identical thereto.

As discussed above, functional variants of CRE0059 (SEQ ID NO: 110) substantially retain the ability of CRE00059 to act as a liver-specific promoter element. For example, when a functional variant of CRE0059 is substituted into liver-specific promoter SP0412, the modified promoter retains at least 80% of its activity, more preferably at least 90% of its activity, more preferably at least 95% of its activity, and yet more preferably 100% of the activity of SP0412 (SEQ ID NO: 91). Suitably the functional variant of CRE0059 comprises a sequence which has at least 70%, 80%, 90%, 95% or 99% identity to SEQ ID NO: 110.

CRE0059 is a proximal promoter and comprises a TFBS for a liver-specific TF, namely HNF1, upstream of the TSS. The functional variant of CRE0059 thus preferably comprises a TFBS for HNF1 upstream of the TSS.

In some embodiments, a functional variant of CRE0059 comprises a sequence which is at least 70% identical to SEQ ID NO: 110 (preferably at least 80%, 90%, 95% or 99% identical to SEQ ID NO: 110), which contains a TFBS for HNF1 (SEQ ID NO: 111), and which contains a TSS sequence (referred to as pl@SERPINA1 or pl@AFP) which is at least 80%, 90%, 95% or completely identical to SEQ ID NO: 112 downstream of said TFBS for HNF1.

In some embodiments, a functional variant of CRE0059 comprises a sequence which has at least 70%, 80%, 90%, 95% or 99% identity to SEQ ID NO: 110, and which further comprises a TFBS comprising SEQ ID NO: 111 for HNF1 at or near position 24-36; and which comprises the TSS sequence which is at least 80%, 90%, 95% or completely identical to SEQ ID NO: 112 at or near position 73-93, positions being numbered with reference to SEQ ID NO: 110. At or near in the present context suitably means within 10, 5, 4, 3, 2, or 1 nucleotide of the recited position with reference to SEQ ID NO: 110. Suitable TFBS sequences are SEQ ID NOS 111 and SEQ ID NO: 112, but alternative TFBS sequences can be used.

In some embodiments, a promoter element comprising or consisting of CRE0059 (SEQ ID NO: 110) or a functional variant thereof has a length of 200 or fewer nucleotides, 150 or fewer nucleotides, 125 or fewer nucleotides, 110 or fewer nucleotides, or 95 or fewer nucleotides.

In some embodiments the liver-specific promoter useful in the methods and compositions as disclosed herein comprises or consists of SEQ ID NO: 91, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 91 is referred to as SP0412. The SP0412 promoter is particularly preferred in some embodiments. This promoter has been found to be powerful and is also very short, which is advantageous in some circumstances.

a. SP0265 (Also Known as SP131A1) and Variants Thereof

In some embodiments, the promoter is a synthetic liver-specific promoter comprising a combination of the CREs CRE0051 (SEQ ID NO: 97), CRE0058 (SEQ ID NO: 113), CRE0065 (SEQ ID NO: 117), and CRE0066 (SEQ ID NO: 122), or functional variants thereof. Typically, the CREs are operably linked to a promoter element. In some preferred embodiments, the liver-specific promoter comprises said CREs, or functional variants thereof, in the order CRE0051, CRE0058, CRE0065, CRE0066, and then the promoter element (in an upstream to downstream direction).

The promoter element can be any suitable proximal or minimal promoter. In some preferred embodiments, the promoter element is a minimal promoter. Where the promoter is a proximal promoter, it is generally preferred that the proximal promoter is liver-specific.

In some preferred embodiments, the promoter element is CRE0052 (also referred to as G6PC) (SEQ ID NO: 126). CRE0052 is a minimal promoter (also referred to as a core promoter).

In some embodiments, the liver-specific promoter comprises the following regulatory elements (or functional variants thereof): CRE0051, CRE0058, CRE0065, CRE0066 then CRE0052 (SEQ ID NO: 126).

The sequence of CRE0051 (SEQ ID NO: 97) and variants thereof are set out above.

In some embodiments, the CRE comprising or consisting of CRE0058 (SEQ ID NO: 113), or a functional variant thereof, has a length of 200 or fewer nucleotides, 150 or fewer nucleotides, 125 or fewer nucleotides, 100 or fewer nucleotides, or 80 or fewer nucleotides. Functional variants thereof may have a sequence that is at least 60%, 65%, 70%, 75%, 80%, 85%, 90%, 95%, 96%, 97%, 98%, or 99% identical thereto.

Functional variants of CRE0058 (SEQ ID NO: 113) are regulatory elements with sequences which vary from CRE0058, but which substantially retain their activity as liver-specific CREs. It will be appreciated by the skilled person that it is possible to vary the sequence of a CRE while retaining its ability to bind to the requisite TFs and enhance expression. A functional variant can comprise substitutions, deletions and/or insertions compared to a reference CRE, provided they do not render the CRE non-functional.

In some embodiments, a functional variant of CRE0058 (SEQ ID NO: 113) can be viewed as a CRE which, when substituted in place of CRE0058 in a promoter, substantially retains its activity. For example, a promoter which comprises a functional variant of CRE0058 substituted in place of CRE0058 preferably retains 80% of its activity, more preferably 90% of its activity, more preferably 95% of its activity, and yet more preferably 100% of its activity (compared to the reference promoter comprising CRE0058 (SEQ ID NO: 113)). For example, considering promoter SP0265 (SEQ ID NO: 94) as an example, CRE0058 in SP0265 can be replaced with a functional variant of CRE0058, and the promoter substantially retains its activity. Retention of activity can be assessed by comparing expression of a suitable reporter under the control of the reference promoter with an otherwise identical promoter comprising the substituted CRE under equivalent conditions.

In some embodiments it is preferred that the functional variant of CRE0058 (SEQ ID NO: 113) comprises transcription factor binding sites (TFBS) for the same liver-specific transcription factors (TF) as CRE0058. The liver-specific TFBS present in CRE0058, listed in the order in which they are present, are: HNF4 (SEQ ID NO: 115) and c/EBP (SEQ ID NO: 116). The functional variant of CRE0058 thus preferably comprises all of these TFBS. Preferably, they are present in the same order that they are present in CRE0058, i.e. in the order HNF4 then c/EBP. When the CRE is associated with a promoter and gene, this order is preferably considered in an upstream to downstream direction (i.e. in the direction from distal from the transcription start site (TSS) to proximal to the TSS). Spacer sequences may be provided between adjacent TFBS. In some embodiments the TFBS may suitably overlap, provided they remain functional, i.e. overlapping sequences are both able to bind their respective TFs.

In some embodiments, the functional variant of CRE0058 (SEQ ID NO: 113) comprises the following TFBS sequences: CGCCCTTTGGACC (HNF4) (SEQ ID NO: 115) and GACCTTTTGCAATCCTGG (c/EBP) (SEQ ID NO: 116), sequences complementary thereto, or functional variants of these TFBS sequences that maintain the ability to bind to their respective TF. These may be present in the same order as CRE0058, i.e. the order in which they are set out above. As discussed above, it is well-known in the art that there is sequence variability associated with TFBS, and that for a given TFBS there is typically a consensus sequence, from which some degree of deviation is typically present.

In some embodiments, the functional variant of CRE0058 comprises the sequence: GCGCCCTTTGGACCTTTTGCAATCCTGG (SEQ ID NO: 114), or a sequence that is at least 70%, 80%, 90%, 95% or 99% identical thereto.

In some embodiments, the CRE consists of SEQ ID NO: 113 or 114 or a functional variant thereof.

It will be noted that the CRE or functional variant thereof can be provided on either strand of a double stranded polynucleotide and can be provided in either orientation. As such, complementary and reverse complementary sequences of SEQ ID NO: 113 or 114 or a functional variant thereof fall within the scope of the invention. Single stranded nucleic acids comprising the sequence according to SEQ ID NO: 113 or 114, or a functional variant thereof, also fall within the scope of the invention.

In some embodiments, the CRE comprising or consisting of CRE0058, or a functional variant thereof, has a length of 120 or fewer nucleotides, 80 or fewer nucleotides, 60 or fewer nucleotides, or 40 or fewer nucleotides.

In some embodiments, the CRE comprising or consisting of CRE0065 (SEQ ID NO: 117), or a functional variant thereof, has a length of 200 or fewer nucleotides, 150 or fewer nucleotides, 125 or fewer nucleotides, 100 or fewer nucleotides, or 80 or fewer nucleotides. Functional variants thereof may have a sequence that is at least 60%, 65%, 70%, 75%, 80%, 85%, 90%, 95%, 96%, 97%, 98%, or 99% identical thereto.

Functional variants of CRE0065 (SEQ ID NO: 117) are regulatory elements with sequences which vary from CRE0065, but which substantially retain their activity as liver-specific CREs. It will be appreciated by the skilled person that it is possible to vary the sequence of a CRE while retaining its ability to bind to the requisite TFs and enhance expression. A functional variant can comprise substitutions, deletions and/or insertions compared to a reference CRE, provided they do not render the CRE non-functional.

In some embodiments, a functional variant of CRE0065 can be viewed as a CRE which, when substituted in place of CRE0065 in a promoter, substantially retains its activity. For example, a promoter which comprises a functional variant of CRE0065 substituted in place of CRE0065 preferably retains 80% of its activity, more preferably 90% of its activity, more preferably 95% of its activity, and yet more preferably 100% of its activity (compared to the reference promoter comprising CRE0065). For example, considering promoter SP0265 (SEQ ID NO: 94) as an example, CRE0065 in SP0265 can be replaced with a functional variant of CRE0065, and the promoter substantially retains its activity. Retention of activity can be assessed by comparing expression of a suitable reporter under the control of the reference promoter with an otherwise identical promoter comprising the substituted CRE under equivalent conditions.

In some embodiments it is preferred that the functional variant of CRE0065 comprises TFBS for the same liver-specific TFs as CRE0065. The liver-specific TFBS present in CRE0065, listed in the order in which they are present, are: RXR Alpha (SEQ ID NO: 119), HNF3 (SEQ ID NO: 120) and HNF3 (SEQ ID NO: 121). The functional variant of CRE0065 thus preferably comprises all of these TFBS. Preferably, they are present in the same order that they are present in CRE0065, i.e. in the order RXR Alpha, HNF3 then HNF3. When the cis-regulatory element is associated with a promoter and gene, this order is preferably considered in an upstream to downstream direction (i.e. in the direction from distal from the transcription start site (TSS) to proximal to the TSS). Spacer sequences may be provided between adjacent TFBS. In some embodiments the TFBS may suitably overlap, provided they remain functional, i.e. overlapping sequences are both able to bind their respective TFs.

In some embodiments, the functional variant of CRE0065 comprises the following TFBS sequences: ACTGAACCCTTGACCCCTGCCCT (RXR Alpha) (SEQ ID NO: 119), CTGTTTGCCC (HNF3) (SEQ ID NO: 120), and CTATTTGCCC (HNF3) (SEQ ID NO: 121), sequences complementary thereto, or functional variants of these TFBS sequences that maintain the ability to bind to their respective TF. These may be present in the same order as CRE0065, i.e. the order in which they are set out above. As discussed above, it is well-known in the art that there is sequence variability associated with TFBS, and that for a given TFBS there is typically a consensus sequence, from which some degree of deviation is typically present.

In some embodiments, the functional variant of CRE0065 comprises the sequence:

(SEQ ID NO: 118)
ACTGAACCCTTGACCCCT-Na-CTGTTTGCCC-Nb-TATTTGCCC

or a sequence that is at least 70%, 80%, 90%, 95% or 99% identical thereto, wherein Na and Nb represent optional spacer sequences. When present, Na optionally has a length of from 14 to 30 nucleotides, preferably from 18 to 26 nucleotides, and more preferably 22 nucleotides. When present, Nb optionally has a length of from 1 to 10 nucleotides, preferably from 2 to 6 nucleotides, and more preferably 4 nucleotides. In some embodiments, the CRE consists of SEQ ID NO: 117 or 118, or a functional variant thereof.

It will be noted that the CRE or functional variant thereof can be provided on either strand of a double stranded polynucleotide and can be provided in either orientation. As such, complementary and reverse complementary sequences of SEQ ID NO: 117 or 118 or a functional variant thereof fall within the scope of the invention. Single stranded nucleic acids comprising the sequence according to SEQ ID NO: 117 or 118 or a functional variant thereof also fall within the scope of the invention.

In some preferred embodiments, the CRE comprising or consisting of CRE0065, or a functional variant thereof, has a length of 200 or fewer nucleotides, 150 or fewer nucleotides, 125 or fewer nucleotides, 90 or fewer nucleotides, or 72 or fewer nucleotides.

In some embodiments, the CRE comprising or consisting of CRE0066 (SEQ ID NO: 122), or a functional variant thereof, has a length of 200 or fewer nucleotides, 150 or fewer nucleotides, 125 or fewer nucleotides, 100 or fewer nucleotides, or 80 or fewer nucleotides Functional variants thereof may have a sequence that is at least 60%, 65%, 70%, 75%, 80%, 85%, 90%, 95%, 96%, 97%, 98%, or 99% identical thereto.

Functional variants of CRE0066 (SEQ ID NO: 122) are regulatory elements with sequences which vary from CRE0066, but which substantially retain their activity as liver-specific CREs. It will be appreciated by the skilled person that it is possible to vary the sequence of a CRE while retaining its ability to bind to the requisite TFs and enhance expression. A functional variant can comprise substitutions, deletions and/or insertions compared to a reference CRE, provided they do not render the CRE non-functional.

In some embodiments, a functional variant of CRE0066 can be viewed as a CRE which, when substituted in place of CRE0066 in a promoter, substantially retains its activity. For example, a promoter which comprises a functional variant of CRE0066 substituted in place of CRE0066 preferably retains 80% of its activity, more preferably 90% of its activity, more preferably 95% of its activity, and yet more preferably 100% of its activity (compared to the reference promoter comprising CRE0066 (SEQ ID NO: 122). For example, considering promoter SP0265 (SEQ ID NO: 94) as an example, CRE0066 in SP0265 can be replaced with a functional variant of CRE0066, and the promoter substantially retains its activity. Retention of activity can be assessed by comparing expression of a suitable reporter under the control of the reference promoter with an otherwise identical promoter comprising the substituted CRE under equivalent conditions.

In some embodiments, it is preferred that the functional variant of CRE0066 comprises transcription factor binding sites (TFBS) for the same liver-specific transcription factors (TF) as CRE0066. The liver-specific TFBS present in CRE0066, listed in the order in which they are present, are: HNF4G (SEQ ID NO: 124) and FOS::JUN (SEQ ID NO: 125). The functional variant of CRE0066 thus preferably comprises all of these TFBS. Preferably, they are present in the same order that they are present in CRE0066, i.e. in the order HNF4G then FOS::JUN. When the cis-regulatory element is associated with a promoter and gene, this order is preferably considered in an upstream to downstream direction (i.e. in the direction from distal from the transcription start site (TSS) to proximal to the TSS). Spacer sequences may be provided between adjacent TFBS. In some embodiments the TFBS may suitably overlap, provided they remain functional, i.e. overlapping sequences are both able to bind their respective TFs.

In some embodiments, the functional variant of CRE0066 (SEQ ID NO: 122) comprises the following TFBS sequences: GCAGGGCAAAGTGCA (HNF4G) (SEQ ID NO: 124) and GATGACTCAG (FOS::JUN) (SEQ ID NO: 125), sequences complementary thereto, or functional variants of these TFBS sequences that maintain the ability to bind to their respective TF. These may be present in the same order as CRE0066, i.e. the order in which they are set out above. As discussed above, it is well-known in the art that there is sequence variability associated with TFBS, and that for a given TFBS there is typically a consensus sequence, from which some degree of deviation is typically present.

In some embodiments, the functional variant of CRE0066 (SEQ ID NO: 122) comprises the sequence: GCAGGGCAAAGTGCA-Na-GATGACTCAG (SEQ ID NO: 123) or a sequence that is at least 70%, 80%, 90%, 95% or 99% identical thereto, wherein Na represents an optional spacer sequence. When present, Na optionally has a length of from 10 to 28 nucleotides, preferably from 14 to 24 nucleotides, and more preferably 19 nucleotides.

In some embodiments, the CRE consists of CRE0066 or a functional variant thereof.

It will be noted that the CRE or functional variant thereof can be provided on either strand of a double stranded polynucleotide and can be provided in either orientation. As such, complementary and reverse complementary sequences of SEQ ID NO: 122 or 123 or a functional variant thereof fall within the scope of the invention. Single stranded nucleic acids comprising the sequence according to SEQ ID NO: 122 or 123, or a functional variant thereof, also fall within the scope of the invention.

In some preferred embodiments, the CRE comprising or consisting of CRE0066 or a functional variant thereof has a length of 200 or fewer nucleotides, 150 or fewer nucleotides, 125 or fewer nucleotides, 100 or fewer nucleotides, or 87 or fewer nucleotides.

In some embodiments, the promoter comprises the promoter element CRE0052 (also referred to as G6PC) (SEQ ID NO: 126) or a functional variant or functional fragment thereof. Functional variants thereof may have a sequence that is at least 60%, 65%, 70%, 75%, 80%, 85%, 90%, 95%, 96%, 97%, 98%, or 99% identical thereto.

Functional variants of CRE0052 (SEQ ID NO: 126) substantially retain the ability of CRE0052 to act as a liver-specific promoter element. For example, when a functional variant of CRE0052 is substituted into liver-specific promoter SP0265, the modified promoter retains at least 80% of its activity, more preferably at least 90% of its activity, more preferably at least 95% of its activity, and yet more preferably 100% of the activity of SP0265.

In one embodiment the liver-specific promoter comprises SEQ ID NO: 94, or a functional variant thereof. In some embodiments, functional variants may have a sequence that is at least 60%, 65%, 70%, 75%, 80%, 85%, 90%, 95%, 96%, 97%, 98%, or 99% identical to SEQ ID NO: 94. The promoter having a sequence according to SEQ ID NO: 94 is referred to as SP0265 (also known as SP131A1 or LVR 131_A1). A promoter comprising or consisting of SEQ ID NO: 94 is particularly preferred in some embodiments.

In some embodiments, the liver-specific promoter is SEQ ID NO: 94 and comprises the following components: CRE0051 (SEQ ID NO: 97); CRE0058 (SEQ ID NO: 113); CRE0065 (SEQ ID NO: 117), CRE0066 (SEQ ID NO: 122), CRE0052 (SEQ ID NO: 126); or functional variants of SEQ ID NO: 97, 113, 117, 122 or 126 which may have a sequence that is at least 60%, 65%, 70%, 75%, 80%, 85%, 90%, 95%, 96%, 97%, 98%, or 99% identical thereto.

b. SP0239 and Variants Thereof

In some embodiments, the promoter is a synthetic liver-specific promoter comprising the following CREs: CRE0018 (SEQ ID NO: 151), CRE0051 (SEQ ID NO: 97), CRE0058 (SEQ ID NO: 113), CRE0065 (SEQ ID NO: 117) and CRE0066 (SEQ ID NO: 122), or functional variants thereof. Typically, the CREs are operably linked to a promoter element. In some preferred embodiments, the liver-specific promoter comprises said CREs, or functional variants thereof, in the order CRE0018, CRE0051, CRE0058, CRE0065, CRE0066, and then the promoter element (in an upstream to downstream direction).

The promoter element can be any suitable proximal or minimal promoter. In some preferred embodiments the promoter element is CRE0052 (also referred to as G6PC). CRE0052 is a minimal promoter (also referred to as a core promoter).

In some embodiments the liver-specific promoter comprises the following elements (or functional variants thereof): CRE0018, CRE0051, CRE0058, CRE0065, CRE0066 and then CRE0052.

The sequences of CRE0051, CRE0058, CRE0065, and CRE0066 and the promoter element CRE0052, and functional variants thereof, are set out above.

CRE0018 has the sequence of SEQ ID NO: 151 or a functional variant or functional fragment thereof. Functional variants thereof may have a sequence that is at least 60%, 65%, 70%, 75%, 80%, 85%, 90%, 95%, 96%, 97%, 98%, or 99% identical thereto.

Functional variants of CRE0018 (SEQ ID NO: 151) are regulatory elements with sequences which vary from CRE0018, but which substantially retain their activity as liver-specific CREs. It will be appreciated by the skilled person that it is possible to vary the sequence of a CRE while retaining its ability to bind to the requisite TFs and enhance expression. A functional variant can comprise substitutions, deletions and/or insertions compared to a reference CRE, provided they do not render the CRE substantially non-functional.

In some embodiments, a functional variant of CRE0018 can be viewed as a CRE which, when substituted in place of CRE0018 in a promoter, substantially retains its activity. For example, a promoter which comprises a functional variant of CRE0018 substituted in place of CRE0018 preferably retains 80% of its activity, more preferably 90% of its activity, more preferably 95% of its activity, and yet more preferably 100% of its activity (compared to the reference promoter comprising CRE0018). For example, considering promoter SP0239 as an example, CRE0018 in SP0239 in can be replaced with a functional variant of CRE0018, and the promoter substantially retains its activity. Retention of activity can be assessed by comparing expression of a suitable reporter under the control of the reference promoter with an otherwise identical promoter comprising the substituted CRE under equivalent conditions.

In some embodiments, the functional variant of CRE0018 comprises TFBS for the same liver-specific TFs as CRE0018. The liver-specific TFBS present in CRE0018, listed in the order in which they are present, are: IRF (SEQ ID NO: 129), NF1 (SEQ ID NO: 130), HNF3 (SEQ ID NO: 131), HBLF (SEQ ID NO: 132), RXRa (SEQ ID NO: 133), EF-C(SEQ ID NO: 134), NF1 (SEQ ID NO: 135), and c/EBP (SEQ ID NO: 136). The functional variant of CRE0018 thus preferably comprises all of these TFBS. Preferably, they are present in the same order that they are present in CRE0018, i.e. in the order IRF, NF1, HNF3, HBLF, RXRa, EF-C, NF1, and then c/EBP. When the CRE is associated with a promoter and gene, this order is preferably considered in an upstream to downstream direction (i.e. in the direction from distal from the transcription start site (TSS) to proximal to the TSS). Spacer sequences may be provided between adjacent TFBS. In some embodiments the TFBS may suitably overlap, provided they remain functional, i.e. overlapping sequences are both able to bind their respective TFs.

In some embodiments the functional variant of CRE0018 comprises the following TFBS sequences: CTTTCACTTTC (IRF) (SEQ ID NO: 129), TCGCCAA (NF1) (SEQ ID NO: 130), TGTGTAAACA (HNF3) (SEQ ID NO: 131), TGTAAACAATA (HBLF) (SEQ ID NO: 132), CTGAACCTTTACCC (RXRa) (SEQ ID NO: 133), GTTGCCCGGCAAC (EF-C) (SEQ ID NO: 134), CAGGTCTGTGCCAAG (NF1) (SEQ ID NO: 135), TGCCAAGTGTTTG (c/EBP) (SEQ ID NO: 136), sequences complementary thereto, or functional variants of these TFBS sequences that maintain the ability to bind to their respective TF of SEQ ID NO: 129-136. These may be present in the same order as CRE0018, i.e. the order in which they are set out above. As discussed above, it is well-known in the art that there is sequence variability associated with TFBS, and that for a given TFBS there is typically a consensus sequence, from which some degree of deviation is typically present.

In some embodiments of the invention, the functional variant of CRE0018 comprises the sequence: CTTTCACTTTCTCGCCAA-Na-TGTGTAAACAATA-Nb-CTGAACCTTTACCC-Nc-GTTGCCCGGCAAC-Nd-CAGGTCTGTGCCAAGTGTTTG (SEQ ID NO: 128), or a sequence that is at least 70%, 80%, 90%, 95% or 99% identical thereto, wherein Na, Nb, Nc, and Nd represent optional spacer sequences. When present, Na optionally has a length of from 10 to 20 nucleotides, preferably from 13 to 17 nucleotides, and more preferably 15 nucleotides. When present, Nb optionally has a length of from 1 to 10 nucleotides, preferably from 1 to 5 nucleotides, more preferably 1 nucleotide. When present, Nc optionally has a length of from 1 to 10 nucleotides, preferably 1 to 5 nucleotides, and more preferably 1 nucleotide. When present, Nd suitably has a length of from 1 to 10 nucleotides, preferably from 2 to 8 nucleotides in length, and more preferably 3 nucleotides in length.

In some embodiments of the invention the CRE consists of SEQ ID NO: 127 or 128 or a functional variant thereof.

It will be noted that the CRE or functional variant thereof can be provided on either strand of a double stranded polynucleotide and can be provided in either orientation. As such, complementary and reverse complementary sequences of SEQ ID NO: 128 or 129 or a functional variant thereof fall within the scope of the invention. Single stranded nucleic acids comprising the sequence according to SEQ ID NOS: 128 or 129 or a functional variant thereof also fall within the scope of the invention.

In some embodiments, the CRE comprising or consisting of CRE0018 (SEQ ID NO: 151), or a functional variant thereof, has a length of 200 or fewer nucleotides, 150 or fewer nucleotides, 125 or fewer nucleotides, or 103 or fewer nucleotides.

In one embodiment the liver-specific promoter comprises or consist of: SEQ ID NO: 93, or a functional variant thereof. The promoter having a sequence according to SEQ ID NO: 93 is referred to as SP0239. Functional variants of SP0239 can have a sequence that is at least 60%, 65%, 70%, 75%, 80%, 85%, 90%, 95%, 96%, 97%, 98%, or 99% identical thereto.

Accordingly, in some embodiments, the liver-specific promoter is SP0239 (SEQ ID NO: 93) and comprises the following components: CRE0018 (SEQ ID NO: 151), CRE0051 (SEQ ID NO: 97), CRE0058 (SEQ ID NO: 113), CRE0065 (SEQ ID NO: 117) and CRE0066 (SEQ ID NO: 122, and CRE0052 (SEQ ID NO: 126); or functional variants may have a sequence that is at least 60%, 65%, 70%, 75%, 80%, 85%, 90%, 95%, 96%, 97%, 98%, or 99% identical thereto.

c. SP0240 and Variants Thereof

In some embodiments, the promoter is a synthetic liver-specific promoter comprising CRE0018 operably linked to a promoter element. In some preferred embodiments, the liver-specific promoter comprises CRE0018, or immediately upstream of the promoter element.

The promoter element can be any suitable proximal or minimal promoter. In some preferred embodiments the promoter element is CRE0006 (SEQ ID NO: 137). CRE0006 is a liver-specific proximal promoter.

In some embodiments the liver-specific promoter comprises the following elements (or functional variants thereof): CRE0018 and then CRE0006.

The sequence of CRE0018 and variants thereof are set out above.

CRE0006 is a proximal promoter and comprises TFBS for liver-specific TFs upstream of the TSS. The liver-specific TFBS present in CRE0006, listed in order, are HNF4 (SEQ ID NO: 138), RXRa (SEQ ID NO: 139), HNF4 (SEQ ID NO: 140), c/EBP (SEQ ID NO: 141), and HNF3 (SEQ ID NO: 142), and optionally pl@VTN (SEQ ID NO: 143). The functional variant of CRE0006 thus preferably comprises these TFBS. Preferably, they are present in the same order that they are present in CRE0006, i.e. in the order HNF4, c/EBP, HNF3, and HNF3. In some embodiments the TFBS overlap, provided they remain functional, i.e. overlapping sequences are both able to bind their respective TFs.

pl@VTN (SEQ ID NO: 143), represents the transcription start site (TSS) in CRE0006, as determined by Cap Analysis of Gene Expression (CAGE).

In some embodiments, a functional variant of CRE0006 comprises a sequence which is at least 70% identical to SEQ ID NO: 137 (preferably at least 80%, 90%, 95% or 99% identical to SEQ ID NO: 25), which contains TFBS for HNF4, RXRa, HNF4, c/EBP, and HNF3, and preferably which contains a TSS sequence which is at least 80%, 90%, 95% or completely identical to TFBS for HNF4, RXRa, HNF4, c/EBP, and HNF3 downstream of said TFBS.

In some embodiments, a functional variant of CRE0006 comprises a sequence which has at least 70%, 80%, 90%, 95% or 99% identity to SEQ ID NO: 137, and which further comprises the following TFBS: HNF4 (SEQ ID NO: 138) at or near position 25-37; RXRa (SEQ ID NO: 139) at or near position 73-83; HNF4 (SEQ ID NO: 140) at or near position 74-86; c/EBP (SEQ ID NO: 141) at or near position 123-136; and HNF3 (SEQ ID NO: 142) at or near position 129-137; and which comprises a TSS sequence which is at least 80%, 90%, 95% or completely identical to SEQ ID NO: 143 at or near position 166-196, positions being numbered with reference to SEQ ID NO: 137. At or near in the present context suitably means within 10, 5, 4, 3, 2, or 1 nucleotide of the recited position with reference to SEQ ID NO: 137. Suitable TFBS sequences are SEQ ID Nos: 138-142, but alternative TFBS sequences can be used.

In one embodiment the liver-specific promoter comprises or consist of SEQ ID NO: 95, or a functional variant thereof. The promoter having a sequence according to SEQ ID NO: 95 is referred to as SP0240. Functional variants of SP0240 can have a sequence that is at least 60%, 65%, 70%, 75%, 80%, 85%, 90%, 95%, 96%, 97%, 98%, or 99% identical thereto.

d. SP0246 and Variants Thereof

In some embodiments, the promoter is a synthetic liver-specific promoter comprising the following CREs: CRE0051, CRE0058, and CRE0065, or functional variants thereof. Typically, the CREs are operably linked to a promoter element. In some preferred embodiments, the liver-specific promoter comprises said CREs, or functional variants thereof, in the order CRE0051, CRE0058, and CRE0065, and then the promoter element (in an upstream to downstream direction).

The promoter element can be any suitable proximal or minimal promoter. In some preferred embodiments the promoter element is CRE0052 (also referred to as G6PC). CRE0052 is a minimal promoter (also referred to as a core promoter).

In some embodiments the liver-specific promoter comprises the following elements (or functional variants thereof): CRE0051, CRE0058, CRE0065, and then CRE0052. The sequences of CRE0051, CRE0058, CRE0065 and the promoter element CRE0052, and functional variants thereof, are set out above.

In one embodiment the liver-specific promoter comprises or consist of SEQ ID NO: 96, or a functional variant thereof. The promoter having a sequence according to SEQ ID NO: 96 is referred to as SP0246. Functional variants of SP0246 can have a sequence that is at least 60%, 65%, 70%, 75%, 80%, 85%, 90%, 95%, 96%, 97%, 98%, or 99% identical to SEQ ID NO: 96.

e. SP0131 and Variants Thereof

In some embodiments, the promoter is a synthetic liver-specific promoter comprising the following CREs: CRE0058, CRE0065 and CRE0066, or functional variants thereof. Typically, the CREs are operably linked to a promoter element. In some preferred embodiments, the liver-specific promoter comprises said CREs, or functional variants thereof, in the order CRE0058, CRE0065, CRE0066 and then the promoter element (in an upstream to downstream direction).

The promoter element can be any suitable proximal or minimal promoter. In some preferred embodiments the promoter element is CRE0052 (also referred to as G6PC). CRE0052 is a minimal promoter (also referred to as a core promoter).

The sequences of CRE0058, CRE0065, and CRE0066 and the promoter element CRE0052, and functional variants thereof, are set out above.

SEQ ID NO: 141, or a functional variant thereof. The promoter having a sequence according to SEQ ID NO: 141 is referred to as SP0131. Functional variants of SP0131 can have a sequence that is at least 60%, 65%, 70%, 75%, 80%, 85%, 90%, 95%, 96%, 97%, 98%, or 99% identical thereto.

f. Composite Promoters:

In some embodiments, the liver-specific promoters as set out above are operably linked to one or more additional regulatory sequences. An additional regulatory sequence can, for example, enhance expression compared to the liver-specific promoter which is not operably linked the additional regulatory sequence. Generally, it is preferred that the additional regulatory sequence does not substantively reduce the specificity of the liver-specific promoter.

For example, the liver-specific promoter can be operably linked to a sequence encoding a UTR (e.g. a 5′ and/or 3′ UTR), an intron, or such.

In some embodiments, the liver-specific promoter is operably linked to sequence encoding a UTR, e.g. a 5′ UTR. A 5′ UTR can contain various elements that can regulate gene expression. The 5′ UTR in a natural gene begins at the transcription start site and ends one nucleotide before the start codon of the coding region. It should be noted that 5′ UTRs as referred to herein may be an entire naturally occurring 5′ UTR or it may be a portion of a naturally occurring 5′ UTR. The 5′UTR can also be partially or entirely synthetic. In eukaryotes, 5′ UTRs have a median length of approximately 150 nt, but in some cases they can be considerably longer. Regulatory sequences that can be found in 5′ UTRs include, but are not limited to:
Binding sites for proteins, that may affect the mRNA's stability or translation; Riboswitches;
Sequences that promote or inhibit translation initiation; and
Introns within 5′ UTRs have been linked to regulation of gene expression and mRNA export.

In some embodiments, a liver-specific promoter as set out above is operably linked to a sequence encoding a 5′ UTR derived from the CMV major immediate gene (CMV-IE gene). For example, the 5′ UTR from the CMV-IE gene suitably comprises the CMV-IE gene exon 1 and the CMV-IE gene exon 1, or portions thereof. In some cases, the promoter element may be modified in view of the linkage to the 5′UTR, for example sequences downstream of the transcription start site (TSS) in the promoter element can be removed (e.g. replaced with the 5′ UTR).

The CMV-IE 5′UTR is described in Simari, et al., Molecular Medicine 4: 700-706, 1998 “Requirements for Enhanced Transgene Expression by Untranslated Sequences from the Human Cytomegalovirus Immediate-Early Gene”, which is incorporated herein by reference. Variants of the CMV-IE 5′ UTR sequences discussed in Simari, et al. are also set out in WO2002/031137, incorporated by reference, and the regulatory sequences disclosed therein can also be used. Other UTRs that can be used in combination with a promoter are known in the art, e.g. in Leppek, K., Das, R. & Barna, M. “Functional 5′ UTR mRNA structures in eukaryotic translation regulation and how to find them”. Nat Rev Mol Cell Biol 19, 158-174 (2018), incorporated by reference.

In some embodiments the sequence encoding the 5′ UTR comprises SEQ ID NO: 145, or a functional variant thereof. In some embodiments, functional variants may have a sequence that is at least 60%, 65%, 70%, 75%, 80%, 85%, 90%, 95%, 96%, 97%, 98%, or 99% identical thereto. SEQ ID NO: 145 encodes a CMV-IE 5′ UTR.

In some embodiments the 5′ UTR comprises a nucleic acid motif that functions as the protein translation initiation site, e.g. sequences that define a Kozak sequence in the mRNA produced. For example, in some embodiments, the sequence encoding the 5′ UTR comprises the sequence motif GCCACC (SEQ ID NO: 153) at or near its 3′ end. Other Kozak sequences or other protein translation initiation sites can be used, as is known in the art (e.g. Marilyn Kozak, “Point Mutations Define a Sequence Flanking the AUG Initiator Codon That Modulates Translation by Eukaryotic Ribosomes” Cell, Vol. 44, 283-292, Jan. 31, 1986; Marilyn Kozak “At Least Six Nucleotides Preceding the AUG Initiator Codon Enhance Translation in Mammalian Cells” J. Mol. Rid. (1987) 196, 947-950; Marilyn Kozak “An analysis of 5-noncoding sequences from 699 vertebrate messenger RNAs” Nucleic Acids Research. Vol. 15 (20) 1987, all of which are incorporated herein by reference). The protein translation initiation site (e.g. Kozak sequence) is preferably positioned immediately adjacent to the start codon.

In some embodiments the sequence encoding the 5′ UTR comprises SEQ ID NO: 438, or a functional variant thereof. In some embodiments, functional variants may have a sequence that is at least 60%, 65%, 70%, 75%, 80%, 85%, 90%, 95%, 96%, 97%, 98%, or 99% identical thereto. This 5′ UTR comprises six nucleotides of SEQ ID NO: 153 which define a Kozak sequence at the 3′ end of the CMV-IE 5′ UTR.

In some embodiments, the SP0412 promoter, or variants thereof, as discussed above is linked to a sequence encoding a 5′ UTR to provide a composite promoter/5′ UTR regulatory construct. Herein, such composite promoter/5′ UTR constructs may be referred to simply as “composite promoters”, or in some cases simply “promoters” for brevity.

In some embodiments, the composite promoter comprises or consists of SEQ ID NO: 92, or a functional variant thereof. In some embodiments, functional variants may have a sequence that is at least 60%, 65%, 70%, 75%, 80%, 85%, 90%, 95%, 96%, 97%, 98%, or 99% identical to SEQ ID NO: 92.

This composite promoter comprises SP0412 operably linked to a sequence encoding the 5′ UTR from the CMV-IE gene (SEQ ID NO: 145) and the GCCACC (SEQ ID NO: 153) Kozak sequence discussed above. This (composite) promoter is referred to as SP0422 (SEQ ID NO: 92). SP0422 is a preferred liver specific promoter in some embodiments. As discussed above, the 5′ UTR suitably comprises a nucleic acid motif that functions as the protein translation initiation site, e.g. sequences that define a Kozak sequence. In the sequence above, the 5′ UTR comprises the sequence motif GCCACC (SEQ ID NO: 153) at its 3′ end, but this sequence motif can be omitted or alternative sequences can be used.

In some embodiments, the SP0265 promoter, or variants thereof, as discussed above is linked to a sequence encoding a 5′ UTR to provide a composite promoter (SP0236-5UTR).

In some embodiments, the composite promoter comprises or consists of SEQ ID NO: 146, or a functional variant thereof. In some embodiments, functional variants may have a sequence that is at least 60%, 65%, 70%, 75%, 80%, 85%, 90%, 95%, 96%, 97%, 98%, or 99% identical to SEQ ID NO: 146.

This composite promoter comprises SP0265 (SEQ ID NO: 94) operably linked to the 5′ UTR from the CMV-IE gene (SEQ ID NO: 145) and the GCCACC (SEQ ID NO: 153) Kozak sequence. This (composite) promoter is referred to as SP0420. In this promoter, a short sequence downstream of the TSS in the CRE0052 promoter element have been replaced with sequences from the 5′ UTR from the CMV-IE. Thus, this promoter actually comprises a minor variant of SP0265 with a modification to CRE0052 whereby some sequence has been removed. SP0420 is preferred in some embodiments. As discussed above, the 5′ UTR suitably comprises a nucleic acid motif that functions as the protein translation initiation site, e.g. sequences that define a Kozak sequence. In the sequence above, the 5′ UTR comprises the sequence motif GCCACC (SEQ ID NO: 153) at its 3′ end, but this sequence motif can be omitted or alternative sequences can be used.

In some embodiments, the SP0239 promoter, or variants thereof, as discussed above is linked to a sequence encoding a 5′ UTR to provide a composite promoter (SP0239-UTR).

In some embodiments, the composite promoter comprises or consists of SEQ ID NO: 147, or a functional variant thereof. In some embodiments, functional variants may have a sequence that is at least 60%, 65%, 70%, 75%, 80%, 85%, 90%, 95%, 96%, 97%, 98%, or 99% identical to SEQ ID NO: 147.

This composite promoter/5′ UTR construct comprises SP0239 operably linked to the 5′ UTR from the CMV-IE gene and the GCCACC (SEQ ID NO: 153) Kozak sequence. This (composite) promoter is referred to as SP0421. Again, in this promoter, a short sequence downstream of the TSS in the CRE0052 promoter element have been replaced with sequences from the 5′ UTR from the CMV-IE. Thus, this promoter actually comprises a minor variant of SP0239 with a modification to CRE0052 whereby some sequence has been removed. SP0421 is preferred in some embodiments. As discussed above, the 5′ UTR suitably comprises a nucleic acid motif that functions as the protein translation initiation site, e.g. sequences that define a Kozak sequence. In the sequence above, the 5′ UTR comprises the sequence motif GCCACC (SEQ ID NO: 153) at its 3′ end, but this sequence motif can be omitted or alternative sequences can be used.

In some embodiments, the SP0240 promoter, or variants thereof, as discussed above is linked to a sequence encoding a 5′ UTR to provide a composite promoter.

In some embodiments, the composite promoter comprises or consists of SEQ ID NO: 148, or a functional variant thereof. In some embodiments, functional variants may have a sequence that is at least 60%, 65%, 70%, 75%, 80%, 85%, 90%, 95%, 96%, 97%, 98%, or 99% identical thereto.

This composite promoter/5′ UTR construct comprises SP0240 operably linked to the 5′ UTR from the CMV-IE gene and the GCCACC (SEQ ID NO: 153) Kozak sequence. This (composite) promoter is referred to as SP0240-UTR. Again, in this promoter, a short sequence downstream of the TSS in the CRE0006 promoter element have been replaced with sequences from the 5′ UTR from the CMV-IE. Thus, this promoter actually comprises a minor variant of SP0240 with a modification to CRE0006 whereby some sequence has been removed. SP0240-UTR is preferred in some embodiments. As discussed above, the 5′ UTR suitably comprises a nucleic acid motif that functions as the protein translation initiation site, e.g. sequences that define a Kozak sequence. In the sequence above, the 5′ UTR comprises the sequence motif GCCACC (SEQ ID NO: 153) at its 3′ end, but this sequence motif can be omitted or alternative sequences can be used.

In some embodiments, the SP0246 promoter, or variants thereof, as discussed above is linked to a sequence encoding a 5′ UTR to provide a composite promoter.

In some embodiments, the composite promoter comprises or consists of SEQ ID NO: 149 (SP0246-UTR), or a functional variant thereof. In some embodiments, functional variants may have a sequence that is at least 60%, 65%, 70%, 75%, 80%, 85%, 90%, 95%, 96%, 97%, 98%, or 99% identical thereto.

This composite promoter/5′ UTR construct comprises SP0246 operably linked to the 5′ UTR from the CMV-IE gene and the GCCACC (SEQ ID NO: 153) Kozak sequence. This (composite) promoter is referred to as SP0246-UTR. Again, in this promoter, a short sequence downstream of the TSS in the CRE0052 promoter element have been replaced with sequences from the 5′ UTR from the CMV-IE. Thus, this promoter actually comprises a minor variant of SP0246 with a modification to CRE0052 whereby some sequence has been removed. SP0246-UTR is preferred in some embodiments. As discussed above, the 5′ UTR suitably comprises a nucleic acid motif that functions as the protein translation initiation site, e.g. sequences that define a Kozak sequence. In the sequence above, the 5′ UTR comprises the sequence motif GCCACC (SEQ ID NO: 153) at its 3′ end, but this sequence motif can be omitted or alternative sequences can be used.

In some embodiments, the SP0131_A1 promoter, or variants thereof, as discussed above is linked to a sequence encoding a 5′ UTR to provide a composite promoter.

In some embodiments, the composite promoter comprises or consists of SEQ ID NO: 150 (SP0131 A1-UTR), or a functional variant thereof. In some embodiments, functional variants may have a sequence that is at least 60%, 65%, 70%, 75%, 80%, 85%, 90%, 95%, 96%, 97%, 98%, or 99% identical thereto.

This composite promoter/5′ UTR construct comprises SP0131 operably linked to the 5′ UTR from the CMV-IE gene and the GCCACC (SEQ ID NO: 153) Kozak sequence. This (composite) promoter is referred to as SP0131-UTR. Again, in this promoter, a short sequence downstream of the TSS in the CRE0052 promoter element have been replaced with sequences from the 5′ UTR from the CMV-IE. Thus, this promoter actually comprises a minor variant of SP0131 with a modification to CRE0052 whereby some sequence has been removed. SP0131-UTR is preferred in some embodiments. As discussed above, the 5′ UTR suitably comprises a nucleic acid motif that functions as the protein translation initiation site, e.g. sequences that define a Kozak sequence. In the sequence above, the 5′ UTR comprises the sequence motif GCCACC (SEQ ID NO: 153) at its 3′ end, but this sequence motif can be omitted or alternative sequences can be used.

In some embodiments, the liver-specific promoter is SP0412 (SEQ ID NO: 91) and comprises the following components: CRE0051 (SEQ ID NO: 97), CRE0067 (SEQ ID NO: 152), CRE0059 (SEQ ID NO: 110) and a Kozak sequence (SEQ ID NO: 153); or functional variants may have a sequence that is at least 60%, 65%, 70%, 75%, 80%, 85%, 90%, 95%, 96%, 97%, 98%, or 99% identical thereto.

In some embodiments, the liver-specific promoter is SP0422 (SEQ ID NO: 9) and comprises the following components: CRE0051 (SEQ ID NO: 97), CRE0067 (SEQ ID NO: 152), CRE0059 (SEQ ID NO: 110), CMV-IE 5′UTR (SEQ ID NO: 153) and a Kozak sequence (SEQ ID NO: 153, or functional variants may have a sequence that is at least 60%, 65%, 70%, 75%, 80%, 85%, 90%, 95%, 96%, 97%, 98%, or 99% identical thereto.

(iii) Functional Variants of the Synthetic Liver-Specific Promoters

In some embodiments, a functional variant of a liver-specific promoter can be viewed as a promoter element which, when substituted in place of a reference promoter element in a promoter, substantially retains its activity. For example, a functional variant of liver-specific promoter which comprises a functional variant of a given promoter in Table 4 herein, or any promoter listed from SEQ ID NOS: 86 (CRM 0412), SEQ ID NO: 91 (SP0412) or SEQ ID NO: 92 (SP0422), SEQ ID NOS: 93 (SP0239), SEQ ID NO: 94 (SP0265, also referred to SP131_A1), SEQ ID NO: 95 (SP0240) or SEQ ID NO: 96 (SP0246), or SEQ ID NO: 146 (SP0265-UTR), SEQ ID NO: 147 (SP0239-UTR), SEQ ID NO: 148 (SP0240-UTR), SEQ ID NO: 149 (SP0246-UTR) or SEQ ID NO: 150 (SP0131-A1-UTR), or a functional fragment or variant thereof, or any LSP selected from SEQ ID NO: 270-341 or 342-430, or a functional fragment or variant thereof where a functional variant or functional fragments preferably retains at least 35%, or at least 40%, or at least 45%, or at least 50%, or at least 55%, or at least 60%, or at least 70% or at least 80% of its activity, more preferably at least 90% of its activity, more preferably at least 95% of the activity of the unchanged promoter, and yet more preferably 100% of the activity (as compared to the unchanged promoter sequence comprising the unmodified promoter element). Suitable assays for assessing liver-specific promoter activity are disclosed herein, e.g. in Examples 12 and 13.

In some embodiments, a functional variant or a functional fragment of a liver-specific promoter disclosed in Table 4 herein, or any promoter listed from SEQ ID NOS: 86 (CRM 0412), SEQ ID NO: 91 (SP0412) or SEQ ID NO: 92 (SP0422), SEQ ID NOS: 93 (SP0239), SEQ ID NO: 94 (SP0265, also referred to SP131 A1), SEQ ID NO: 95 (SP0240) or SEQ ID NO: 96 (SP0246), or SEQ ID NO: 146 (SP0265-UTR), SEQ ID NO: 147 (SP0239-UTR), SEQ ID NO: 148 (SP0240-UTR), SEQ ID NO: 149 (SP0246-UTR) or SEQ ID NO: 150 (SP0131-A1-UTR), or any LSP selected from SEQ ID NO: 270-341 or 342-430, has at least about 75% sequence identity to, or at least about 80% sequence identity to, at least about 90% sequence identity to, at least about 95% sequence identity to, at least about 98% sequence identity to the original unmodified sequence, and also at least 35% of the promoter activity, or at least about 45% of the promoter activity, or at least about 50% of the promoter activity, or at least about 60% of the promoter activity, or at least about 75% of the promoter activity, or at least about 80% of the promoter activity, or at least about 85% of the promoter activity, or at least about 90% of the promoter activity, or at least about 95% of the promoter activity of the corresponding unmodified promoter sequence.

For example, a functional variant or a functional fragment of SEQ ID NO: 258 (SP0412) or SEQ ID NO: 92 (SP0422) has at least about 75% sequence identity to SEQ ID NO: 91 (SP0412) or SEQ ID NO: 92 (SP0422), or at least about 80% sequence identity to SEQ ID NO: 91 (SP0412) or SEQ ID NO: 92 (SP0422), at least about 90% sequence identity to SEQ ID NO: 91 (SP0412) or SEQ ID NO: 92 (SP0422), at least about 95% sequence identity to SEQ ID NO: 91 (SP0412) or SEQ ID NO: 92 (SP0422), at least about 98% sequence identity to SEQ ID NO: SEQ ID NO: 91 (SP0412) or SEQ ID NO: 92 (SP0422), or the original unmodified sequence, and also at least 35% of the promoter activity, or at least about 45% of the promoter activity, or at least about 50% of the promoter activity, or at least about 60% of the promoter activity, or at least about 75% of the promoter activity, or at least about 80% of the promoter activity, or at least about 85% of the promoter activity, or at least about 90% of the promoter activity, or at least about 95% of the promoter activity of the corresponding unmodified promoter sequence of SEQ ID NO: 91 (SP0412) or SEQ ID NO: 92 (SP0422), respectively.

In some embodiments, a functional variant of a liver-specific promoter disclosed herein retains a significant level of sequence identity to the unmodified promoter sequence. Suitably functional variants comprise a sequence that is at least 60% identical to the unmodified promoter sequence, more preferably at least 70%, 80%, 90%, 95% or 99% identical to the unmodified liver-specific promoter sequence.

In some embodiments, a functional fragment of a liver-specific promoter disclosed herein retains a significant level of sequence identity to the unmodified promoter sequence. Suitable functional fragments comprise a sequence that is at least 60% identical to the unmodified promoter sequence, more preferably at least 70%, 80%, 90%, 95% or 99% identical to the unmodified liver-specific promoter sequence.

In some embodiments, a functional variant of a promoter element can be viewed as a promoter element which, when substituted in place of a reference promoter element in a promoter, substantially retains its activity. For example, a liver-specific promoter which comprises a functional variant of a given promoter element preferably retains at least 80% of its activity, more preferably at least 90% of its activity, more preferably at least 95% of its activity, and yet more preferably 100% of its activity (compared to the reference promoter comprising the unmodified promoter element). Suitable assays for assessing liver-specific promoter activity are disclosed herein, e.g. in Examples 12 and 13.

It should be noted that the sequences of a liver-specific promoter as disclosed herein in Table 4, or any LSP selected from SEQ ID NO: 270-341 or 342-430 can be altered without causing a substantial loss of activity. Thus, functional variants of a liver-specific promoter are discussed below can be prepared by modifying the sequence of a liver-specific promoter disclosed in Table 4 herein, or any or any LSP selected from SEQ ID NO: 270-341 or 342-430, provided that modifications which are significantly detrimental to activity of the liver-specific promoter are avoided. In view of the information provided in the present disclosure, modification of a liver-specific promoter disclosed herein in Table 4, or any LSP selected from SEQ ID NO: 270-341 or 342-430 to provide functional variants is straightforward. Moreover, the present disclosure provides methodologies for simply assessing the functionality of any given liver-specific promoter variant. Functional variants for each liver-specific promoter are discussed below.

In some embodiments of the invention the synthetic liver-specific promoter comprises a sequence from the group consisting of: any promoter listed from SEQ ID NOS: 86 (CRM 0412), SEQ ID NO: 91 (SP0412) or SEQ ID NO: 92 (SP0422), SEQ ID NOS: 93 (SP0239), SEQ ID NO: 94 (SP0265, also referred to SP131_A1), SEQ ID NO: 95 (SP0240) or SEQ ID NO: 96 (SP0246), or SEQ ID NO: 146 (SP0265-UTR), SEQ ID NO: 147 (SP0239-UTR), SEQ ID NO: 148 (SP0240-UTR), SEQ ID NO: 149 (SP0246-UTR) or SEQ ID NO: 150 (SP0131-A1-UTR), or any LSP selected from SEQ ID NO: 270-341 or 342-430, or a functional variant of any thereof. Suitably the functional variant of any of said liver-specific promoter comprises a sequence that is at least 70% identical to the reference synthetic liver-specific promoter, more preferably at least 80%, 90%, 95% or 99% identical to the reference synthetic liver-specific promoter.

In some embodiments, the functional variant thereof may suitably comprise a sequence that is at least 60%, 70%, 80%, 90%, 95% or 99% identical to any one of the sequences listed in Table 4. Additionally or alternatively, a functional variant of any one of the sequences listed in Table 4, suitably comprises a sequence which hybridizes under stringent conditions to the reference sequence. Functional variants of any one of the sequences listed in Table 4 include variants in which one or more of the sequence provided therein has been replaced with a functional variant thereof as defined above, and/or where the order of the sequences provided therein has been altered.

In some embodiments, a functional variant of any one of the liver-specific promoter sequences listed in Table 4 can be viewed as a liver-specific promoter, when at least one or more nucleotides are substituted and it substantially retains its activity. For example, a liver-specific promoter which comprises a functional variant of any one of the liver-specific promoter sequences listed in Table 4 preferably retains 80% of its activity, more preferably 90% of its activity, more preferably 95% of its activity, and yet more preferably 100% of its activity (compared to the reference promoter sequence). For example, if a LSP comprises a nucleic acid sequence comprising SP0412 (SEQ ID NO: 91) as an example, a portion of nucleotides in SP0412 (e.g., SEQ ID NO:91) in can be replaced with a functional variant of thereof, and the liver specific SP0412 promoter substantially retains its activity. Retention of activity can be assessed by comparing expression of a suitable reporter under the control of the reference promoter with an otherwise identical promoter comprising the substituted nucleic acids under equivalent conditions. Suitable assays for assessing liver-specific promoter activity are disclosed herein, e.g. in examples 12 and 13.

In some embodiments of the compositions and methods disclosed herein, a synthetic liver-specific promoter disclosed herein in Table 4, or any promoter listed from SEQ ID NOS: 86 (CRM 0412), SEQ ID NO: 91 (SP0412) or SEQ ID NO: 92 (SP0422), SEQ ID NOS: 93 (SP0239), SEQ ID NO: 94 (SP0265, also referred to SP131 A1), SEQ ID NO: 95 (SP0240) or SEQ ID NO: 96 (SP0246), or SEQ ID NO: 146 (SP0265-UTR), SEQ ID NO: 147 (SP0239-UTR), SEQ ID NO: 148 (SP0240-UTR), SEQ ID NO: 149 (SP0246-UTR) or SEQ ID NO: 150 (SP0131-A1-UTR), or any LSP selected from SEQ ID NO: 270-341 or 342-430 is a functional variant thereof that has length of 700, 600, 500, 450, 400, 350, 300, 250 or 200 or 150 or fewer nucleotides.

In some embodiments of the compositions and methods disclosed herein, the synthetic liver-specific promoter disclosed herein in Table 4, any promoter listed from SEQ ID NOS: 86 (CRM 0412), SEQ ID NO: 91 (SP0412) or SEQ ID NO: 92 (SP0422), SEQ ID NOS: 93 (SP0239), SEQ ID NO: 94 (SP0265, also referred to SP131_A1), SEQ ID NO: 95 (SP0240) or SEQ ID NO: 96 (SP0246), or SEQ ID NO: 146 (SP0265-UTR), SEQ ID NO: 147 (SP0239-UTR), SEQ ID NO: 148 (SP0240-UTR), SEQ ID NO: 149 (SP0246-UTR) or SEQ ID NO: 150 (SP0131-A1-UTR), or any LSP selected from SEQ ID NO: 270-341 or 342-430, comprises a synthetic liver-specific cis-regulatory element (CRE) or cis-regulatory module (CRM) operably linked to a minimal promoter. Examples of suitable minimal promoters for use in the present invention include, but are not limited to, the CMV-minimal promoter, MinTk minimal promoter, and the LVR_CRE0052_G6PC minimal promoter (SEQ ID NO: 126). In particular embodiments, the minimal promoter is the CMV-IE promoter comprising the sequence of SEQ ID NO: 145, a sequence that is at least 60%, 70%, 80%, 90%, 95% or 99% identical to SEQ ID NO: 145 Exemplary promoters comprising a CMV-IE for use in the methods and compositions disclosed herein can be selected from, but are not limited to, SEQ ID NO: 92 (SP0422), SEQ ID NO: 146 (SP0265-UTR), SEQ ID NO: 147 (SP0239-UTR), SEQ ID NO: 148 (SP0240-UTR), SEQ ID NO: 149 (SP0246-UTR) or SEQ ID NO: 150 (SP0131-A1-UTR).

In some embodiments of the compositions and methods disclosed herein, a synthetic liver-specific promoter disclosed herein in Table 4, e.g., any promoter listed from SEQ ID NOS: 86 (CRM 0412), SEQ ID NO: 91 (SP0412) or SEQ ID NO: 92 (SP0422), SEQ ID NOS: 93 (SP0239), SEQ ID NO: 94 (SP0265, also referred to SP131 A1), SEQ ID NO: 95 (SP0240) or SEQ ID NO: 96 (SP0246), or SEQ ID NO: 146 (SP0265-UTR), SEQ ID NO: 147 (SP0239-UTR), SEQ ID NO: 148 (SP0240-UTR), SEQ ID NO: 149 (SP0246-UTR) or SEQ ID NO: 150 (SP0131-A1-UTR), or any LSP selected from SEQ ID NO: 270-341 or 342-430 is able to increase expression of gene in the liver of a subject or in a liver cell by at least 20%, at least 40%, at least 60%, at least 80%, at least 100%, at least 200%, at least 300%, at least 500%, at least 1000% or more relative to the LP-1 promoter (SEQ ID NO: 432).

In some embodiments of the compositions and methods disclosed herein, the synthetic liver-specific promoter disclosed herein in Table 4,or any LSP promoter selected from SEQ ID NOS: 86, 91-96, 146-150, or any LSP selected from SEQ ID NO: 270-341 or 342-430, a synthetic liver-specific promoter is able to promote liver-specific transgene expression and has an activity in liver cells which is at least 15%, 20%, 30%, 40%, 50%, 60%, 70%, 80%, 90%, 100%, 125%, 150%, 175%, 200%, 250%, 300%, 350% or 400% of the activity of the TTR promoter (SEQ ID NO: 431).

In some embodiments of the compositions and methods disclosed herein, the synthetic liver-specific promoter disclosed herein in Table 4, or any LSP promoter selected from SEQ ID NOS: 86, 91-96, 146-150, or any LSP selected from SEQ ID NO: 270-341 or 342-430, a synthetic liver-specific promoter is able to promote liver-specific transgene expression and has an activity in liver cells which is at least 15%, 20%, 30%, 40%, 50%, 60%, 70%, 80%, 90%, 100%, 125%, 150%, 175%, 200%, 250%, 300%, 350% or 400% of the activity of the TBG promoter (SEQ ID NO: 435).

### G. Intron Sequence

In some embodiments, the rAAV genotype comprises an intron sequence located 3′ of the promoter sequence and 5′ of the secretory signal peptide. Intron sequences serve to increase one or more of: mRNA stability, mRNA transport out of nucleus and/or expression and/or regulation of the expressed GAA fusion polypeptide (e.g., SS-GAA fusion polypeptide or SS-IGF2-GAA polypeptide). In alternative embodiments, a rAAV genotype does not comprise an intron sequence.

In some embodiments, the intron sequence is a MVM intron sequence, for example, but not limited to and intron sequence of SEQ ID NO: 13 or nucleic acid sequence having at least 80%, 85%, 90%, 95%, 96%, 97%, 98% or 99% nucleotide sequence identity thereto.

In some embodiments, the intron sequence is a HBB2 intron sequence, for example, but not limited to and intron sequence of SEQ ID NO: 14 or nucleic acid sequence having at least 80%, 85%, 90%, 95%, 96%, 97%, 98% or 99% nucleotide sequence identity thereto.

In some embodiments of the methods and compositions disclosed herein, a recombinant AAV vector comprises a heterologous nucleic acid sequence that further comprises an intron sequence located 5′ of the sequence encoding the secretory signal peptide, and 3′ of the promoter. In some embodiments, the intron sequence comprises a MVM sequence or a HBB2 sequence, wherein the MVM sequence comprises the nucleic acid sequence of SEQ ID NO: 13, or a nucleic acid sequence at least about 75%, or 80%, or 85%, or 90%, or 95%, or 98%, or 99% sequence identity to SEQ ID NO: 13, and the HBB2 sequence comprises the nucleic acid sequence of SEQ ID NO: 14, or a nucleic acid sequence at least about 75%, or 80%, or 85%, or 90%, or 95%, or 98%, or 99% sequence identity to SEQ ID NO: 14.

In some embodiments, the rAAV genotype comprises an intron sequence selected in the group consisting of a human beta globin b2 (or HBB2) intron, a FIX intron, a chicken beta-globin intron, and a SV40 intron. In some embodiments, the intron is optionally a modified intron such as a modified HBB2 intron (see, e.g., SEQ ID NO: 17 in of WO2018046774A1): a modified FIX intron (see., e.g., SEQ ID NO: 19 in WO2018046774A1), or a modified chicken beta-globin intron (e.g., see SEQ ID NO: 21 in WO2018046774A1), or modified HBB2 or FIX introns disclosed in WO2015/162302, which are incorporated herein in their entirety by reference.

### H. Poly-A

In some embodiments, an rAAV vector genome includes at least one poly-A tail that is located 3′ and downstream from the heterologous nucleic acid gene encoding the in one embodiment, a GAA fusion polypeptide (e.g., SS-GAA fusion polypeptide or SS-IGF2-GAA polypeptide). In some embodiments, the polyA signal is 3′ of a stability sequence or CS sequence as defined herein. Any polyA sequence can be used, including but not limited to hGH poly A, synpA polyA and the like. In some embodiments, the polyA is a synthetic polyA sequence. In some embodiments, the rAAV vector genome comprises two poly-A tails, e.g., a hGH poly A sequence and another polyA sequence, where a spacer nucleic acid sequence is located between the two poly A sequences. In some embodiments, the rAAV genome comprises 3′ of the nucleic acid encoding the GAA fusion polypeptide (e.g., SS-GAA fusion polypeptide or SS-IGF2-GAA polypeptide), or alternatively, 3′ of the CS sequence the following elements; a first polyA sequence, a spacer nucleic acid sequence (of between 100-400 bp, or about 250 bp), a second poly A sequence, a spacer nucleic acid sequence, and the 3′ ITR. In some embodiments, the first and second poly A sequence is a hGH poly A sequence, and in some embodiments, the first and second poly A sequences are a synthetic poly A sequence. In some embodiments, the first poly A sequence is a hGH poly A sequence and the second poly A sequence is a synthetic sequence, or vice versa—that is, in alternative embodiments, the first poly A sequence is a synthetic poly A sequence and the second poly A sequence is a hGH polyA sequence. An exemplary poly A sequence is, for example, SEQ ID NO: 15 (hGH poly A sequence), or a poly A nucleic acid sequence having at least 80%, 85%, 90%, 95%, 96%, 97%, 98% or 99% nucleotide sequence identity to SEQ ID NO: 15. In some embodiments, the hGH poly sequence encompassed for use is described in Anderson et al. J. Biol. Chem 264(14); 8222-8229, 1989 (See, e.g. p. 8223, 2nd column, first paragraph) which is incorporated herein in its entirety by reference.

In some embodiments, a poly-A tail can be engineered to stabilize the RNA transcript that is transcribed from an rAAV vector genome, including a transcript for a heterologous gene, which in one embodiment is a GAA, and in alternative embodiments, the poly-A tail can be engineered to include elements that are destabilizing.

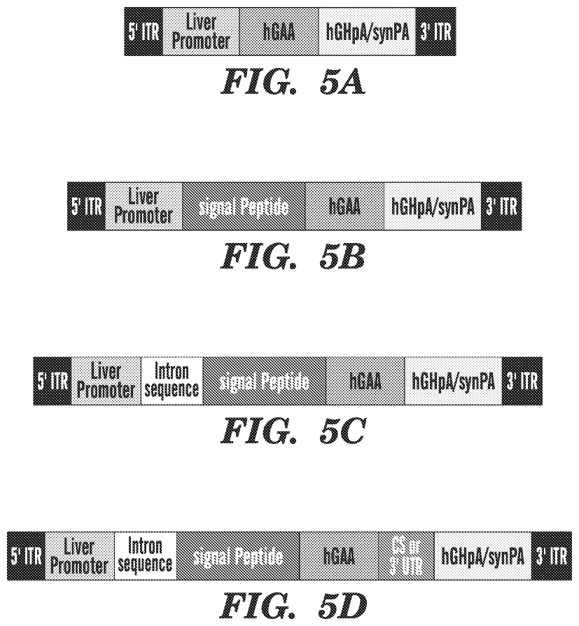

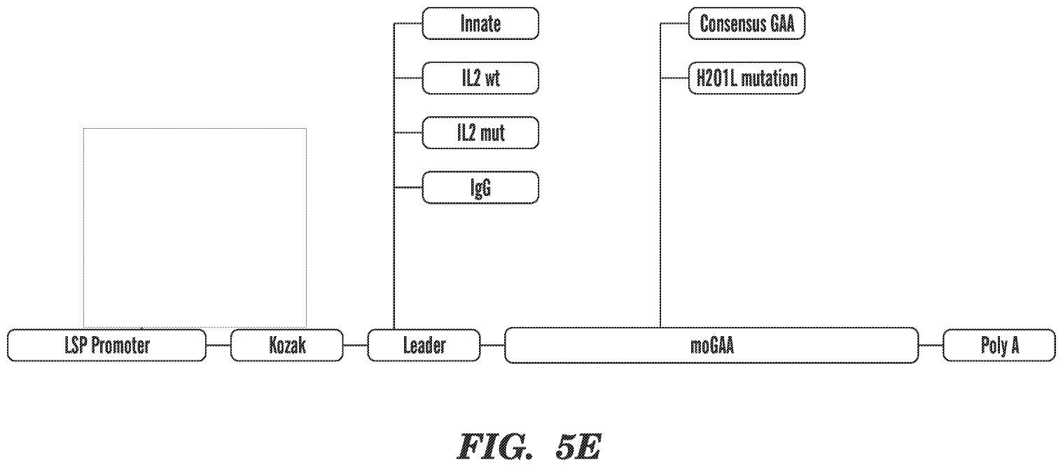

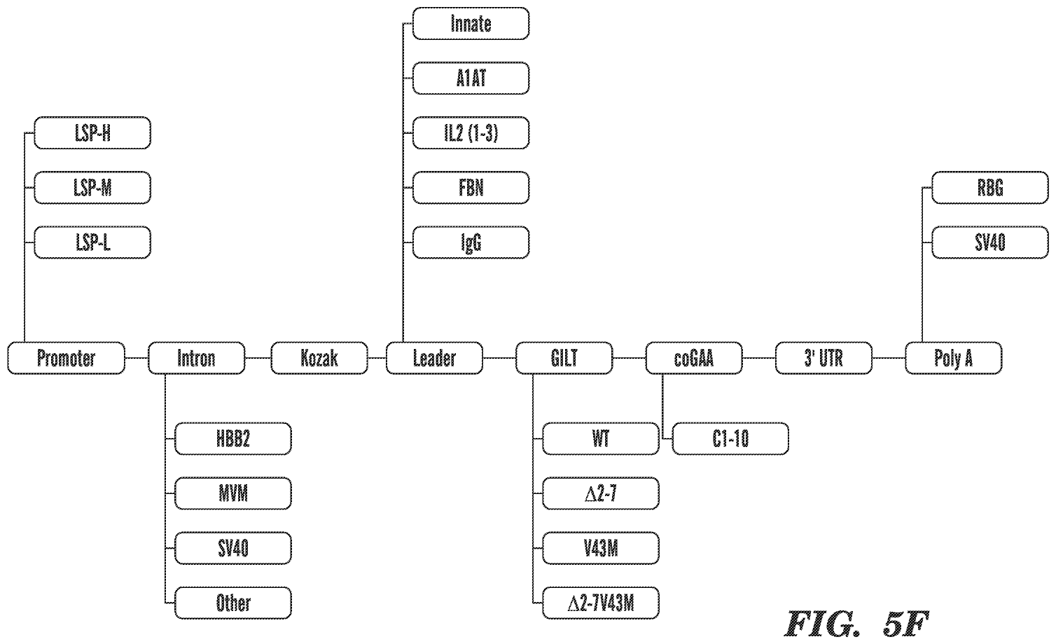

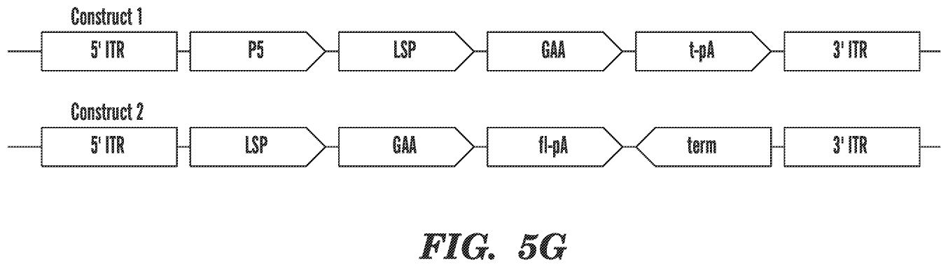

In some embodiments of the methods and compositions disclosed herein, a recombinant AAV vector comprises at least one polyA sequence located 3′ of the nucleic acid encoding the GAA gene and 5′ of the 3′ ITR sequence. In some embodiments, the poly A is a full length poly A (fl-polyA) sequence. In some embodiments, the polyA is a truncated polyA sequence, see. e.g., FIG. 5G.

In an embodiment, a poly-A tail can be engineered to become a destabilizing element by altering the length of the poly-A tail. In an embodiment, the poly-A tail can be lengthened or shortened. In some embodiments, the 3′ untranslated region comprises GAA 3′ UTR (SEQ ID NO: 85) or a 3′ UTR (SEQ ID NO: 77).

In another embodiment, a destabilizing element is a microRNA (miRNA) that has the ability to silence (repress translation and promote degradation) the RNA transcripts the miRNA binds to that encode a heterologous gene. In an embodiment, addition or deletion of seed regions within the poly-A tail can increase or decrease expression of a protein, such as the GAA protein or modified GAA polypeptide.

In another embodiment, seed regions can also be engineered into the 3′ untranslated regions located between the heterologous gene and the poly-A tail. In a further embodiment, the destabilizing agent can be an siRNA. The coding region of the siRNA can be included in an rAAV vector genome and is generally located downstream, 3′ of the poly-A tail.

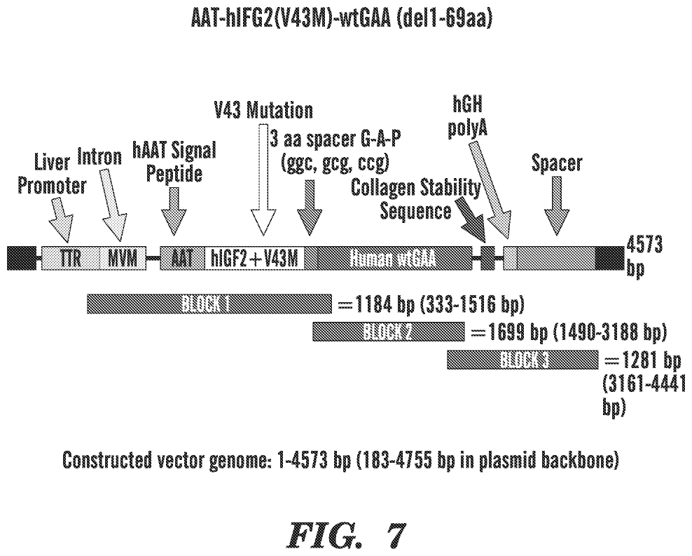

In all aspects of the methods and compositions as disclosed herein, the rAAV genome may also comprise a Stuffer DNA nucleic sequence. An exemplary stuffer DNA sequence is SEQ ID NO: 71, or a nucleic acid sequence having at least 80%, 85%, 90%, 95%, 96%, 97%, 98% or 99% nucleotide sequence identity thereto. As shown in FIGS. 7-8 and FIGS. 9A-9E, the stuffer sequence is located 3 of the poly A tail, for example, and is located 5‘ of the’3 ITR sequence. In some embodiments, the stuffer DNA sequence comprises a synthetic polyadenylation signal in the reverse orientation.

In some embodiments, a stuffer nucleic acid sequence (also referred to as a “spacer” nucleic acid fragment, see FIGS. 7-8) can be located between the poly A sequence and the 3′ ITR (i.e., a stuffer nucleic acid sequence is located 3′ of the polyA sequence and 5′ of the 3′ ITR) (see, e.g., FIG. 7-8). Such a stuffer nucleic acid sequence can be about 30 bp, 50pb, 75 bp, 100 bp, 150 bp, 200 bp, 250 bp, 300 bp or longer than 300 bp. In some embodiments of the methods and compositions as disclosed herein, a stuffer nucleic acid fragment is between 20-50 bp, 50-100 bp, 100-200 bp, 200-300 bp, 300-500 bp, or any integer between 20-500 bp. Exemplary stuffer (or spacer) nucleic acid sequence comprise SEQ ID NO: 16, SEQ ID NO: 71 or SEQ ID NO: 78, or a nucleic acid sequence at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99%, identical to SEQ ID NO: 16 or SEQ ID NO: 71 or SEQ ID NO: 78.

### I. AAV ITRs

The rAAV genome as disclosed here comprises AAV ITRs that have desirable characteristics and can be designed to modulate the activities of, and cellular responses to vectors that incorporate the ITRs. In another embodiment, the AAV ITRs are synthetic AAV ITRs that has desirable characteristics and can be designed to manipulate the activities of and cellular responses to vectors comprising one or two synthetic ITRs, including, as set forth in U.S. Pat. No. 9,447,433, which is incorporated herein by reference.

In another embodiment, an ITR exhibits modified transcription activity relative to a naturally occurring ITR, e.g., ITR2 from AAV2. It is known that the ITR2 sequence inherently has promoter activity. It also inherently has termination activity, similar to a poly(A) sequence. The minimal functional ITR of the present invention exhibits transcription activity as shown in the examples, although at a diminished level relative to ITR2. Thus, in some embodiments, the ITR is functional for transcription. In other embodiments, the ITR is defective for transcription. In certain embodiments, the ITR can act as a transcription insulator, e.g., preventing transcription of a transgenic cassette present in the vector when the vector is integrated into a host chromosome.

One aspect of the invention relates to an rAAV vector genome comprising at least one synthetic AAV ITR, wherein the nucleotide sequence of one or more transcription factor binding sites in the ITR is deleted and/or substituted, relative to the sequence of a naturally occurring AAV ITR such as ITR2. In some embodiments, it is the minimal functional ITR in which one or more transcription factor binding sites are deleted and/or substituted. In some embodiments at least 1 transcription factor binding site is deleted and/or substituted, e.g., at least 5 or more or 10 or more transcription factor binding sites, e.g., at least 1, 2, 3, 4, 5, 6, 7, 8, 9, 10, 11, 12, 13, 14, 15, 16, 17, 18, 19, 20, or 21 transcription factor binding sites.

Another embodiment, a rAAV vector, including an rAAV vector genome as described herein comprises a polynucleotide comprising at least one synthetic AAV ITR, wherein one or more CpG islands (a cytosine base followed immediately by a guanine base (a CpG) in which the cytosines in such arrangement tend to be methylated) that typically occur at, or near the transcription start site in an ITR are deleted and/or substituted. In an embodiment, deletion or reduction in the number of CpG islands can reduce the immunogenicity of the rAAV vector. This results from a reduction or complete inhibition in TLR-9 binding to the rAAV vector DNA sequence, which occurs at CpG islands. It is also well known that methylation of CpG motifs results in transcriptional silencing. Removal of CpG motifs in the ITR is expected to result in decreased TLR-9 recognition and/or decreased methylation and therefore decreased transgene silencing. In some embodiments, it is the minimal functional ITR in which one or more CpG islands are deleted and/or substituted. In an embodiment, AAV ITR2 is known to contain 16 CpG islands of which one or more, or all 16 can be deleted.

In some embodiments, at least 1 CpG motif is deleted and/or substituted, e.g., at least 4 or more or 8 or more CpG motifs, e.g., at least 1, 2, 3, 4, 5, 6, 7, 8, 9, 10, 11, 12, 13, 14, 15, or 16 CpG motifs.

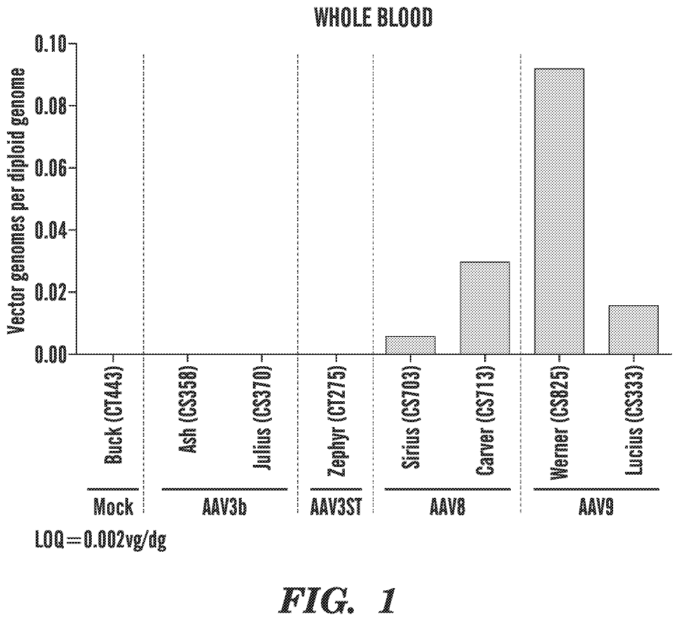

In another embodiment, the synthetic ITR comprises, consists essentially of, or consists of one of the nucleotide sequences listed in Table 7. In other embodiments, the synthetic ITR comprises, consist essentially of, or consist of a nucleotide sequence that is at least 80% identical, e.g., at least 85%, 90%, 95%, 96%, 97%, 98%, or 99% identical to any one of the nucleotide sequences listed in Table 7. In some embodiments, the ITR is a sequence is disclosed in FIG. 1 of Samulski et al., 1983, Cell, 33; 135-143 (referred to “Samulski et al, 1983” as which is incorporated herein in its entirety by reference), which discloses modified ITR sequences in FIG. 1. In some embodiments, the ITR sequence comprises, or consists of a nucleotide sequence that is at least 80% identical, e.g., at least 85%, 90%, 95%, 96%, 97%, 98%, or 99% identical to one of the ITR sequences in FIG. 1 as disclosed in Samulski et al, 1993. In some embodiments, the ITR comprises, or consists of a nucleotide sequence that is at least 80% identical, e.g., at least 85%, 90%, 95%, 96%, 97%, 98%, or 99% or 99.5% identical to the ITR sequence of pSM 609 right disclosed in the middle panel of FIG. 1 (that lacks the 9 bp) disclosed in Samulski et al, 1983. In some embodiments, the ITR comprises a nucleotide sequence that is at least 80% identical, e.g., at least 85%, 90%, 95%, 96%, 97%, 98%, or 99% or 99.5% identical to the ITR sequence of any of SEQ ID NOs: 441-444. In some embodiments, the ITR sequence, e.g., Right ITR (or 3′ ITR) is SEQ ID NO: 442 or a nucleotide sequence that is at least 80% identical, e.g., at least 85%, 90%, 95%, 96%, 97%, 98%, or 99% or 99.5% identical to SEQ ID NO: 442. In some embodiments, the ITR sequence, e.g., left ITR (or 5′ ITR) is SEQ ID NO: 441 or a nucleotide sequence that is at least 80% identical, e.g., at least 85%, 90%, 95%, 96%, 97%, 98%, or 99% or 99.5% identical to SEQ ID NO: 441.

TABLE 7
Exemplary synthetic ITR sequences

| | |
|---|---|
| MH-257 | AGGAACCCCTAGTGATGGAGTTGGCCACTCCCTCTCTGCGCGCTCGCTCG |
| | CTCACTGAGGCAATTTGATAAAAATCGTCAAATTATAAACAGGCTTTGCC |
| | TGTTTAGCCTCAGTGAGCGAGCGAGCGCGCAGAGAGGGAGTGGCCAACT |
| | CCATCACTAGGGGTTCCT (SEQ ID NO: 36) |
| MH-258 | AGGAACCCCTAGTGATGGAGTTGGCCACTCCCTCTCTGCGCGCTCGCTCG |
| | CTCACTGAGGGATAAAAATCCAGGCTTTGCCTGCCTCAGTGAGCGAGCG |
| | AGCGCGCAGAGAGGGAGTGGCCAACTCCATCACTAGGGGTTCCT (SEQ |
| | ID NO: 37) |
| MH Delta | AGGAACCCCTAGTGATGGAGTTGGCCACTCCCTCTCTGCGCGCTCGCTCG |
| 258 | CTCACTGAGGGATAAAAATCCAGGCTTTGCCTGCCTCAGTGAGCGAGCG |
| | AGCGCGCAGAGAGGGAGTGGCCAACTCCATCACTAGGGGTTCCT (SEQ |
| | ID NO: 38) |
| MH Telomere-1 | AGGAACCCCTAGTGATGGAGTTGGCCACTCCCTCTCTGGGATTGGGATTG |
| ITR | CGCGCTCGCTCGCGGGATTGGGATTGGGATTGGGATTGGGATTGGGATTG |
| | ATAAAAATCAATCCCAATCCCAATCCCAATCCCAATCCCAATCCCGCGAG |
| | CGAGCGCGCAATCCCAATCCCAGAGAGGGAGTGGCCAACTCCATCACTA |
| | GGGGTTCCT (SEQ ID NO: 39) |
| MH Telomere-2 | AGGAACCCCTAGTGATGGAGTTGGCCACTCCCTCTCTGCGCGCTCGCTCG |
| ITR | CTCGGGATTGGGATTGGGATTGGGATTGGGATTGGGATTGATAAAAATC |
| | AATCCCAATCCCAATCCCAATCCCAATCCCAATCCCGCGAGCGAGCGCGC |
| | AGGAGAGGGAGTGGCCAACTCCATCACTAGGGGTTCCTAAGCTTATTAT |
| | A (SEQ ID NO: 40) |
| MH PoIII 258 | AGGAACCCCTAGTGATGGAGTTGGCCACTCCCTCTCTGCGCGCTCGCTCG |
| ITR | CTCACTGAGGGCGCCTATAAAGATAAAAATCCAGGCTTTGCCTGCCTCAG |
| | TTAGCGAGCGAGCGCGCAGAGAGGGAGTGGCCAACTCCATCACTAGGGG |
| | TTCCT(SEQ ID NO: 41) |
| MH 258 | CTAGTGATGGAGTTGGCCACTCCCTCTCTGCGCGCTCGCTCGCTCACTGA |
| Delta D | GGGATAAAAATCCAGGCTTTGCCTGCCTCAGTGAGCGAGCGAGCGCGCA |
| conservative | GAGAGGGAGTGGCCAACTCCATCACTAG (SEQ ID NO: 42) |
| 5′ ITR | TTGGCCACTCCCTCTCTGCGCGCTCGCTCGCTCACTGAGGCCGGGCGACC |
| | AAAGGTCGCCCGACGCCCGGGCTTTGCCCGGGCGGCCTCAGTGAGCGAG |
| | CGAGCGCGCAGAGAGGGAGTGGCCAACTCCATCACTAGGGGTTCCT (SEQ |
| | ID NO: 161) |
| 3′ ITR | AGGAACCCCTAGTGATGGAGTTGGCCACTCCCTCTCTGCGCGCTCGCTCG |
| | CTCACTGAGGCCGGGCGACCAAAGGTCGCCCGACGCCCGGGCTTTGCCC |
| | GGGCGGCCTCAGTGAGCGAGCGAGCGCGCAGAGAGGGAGTGGCCAA |
| | (SEQ ID NO: 165) |
| L-ITR | CTGCGCGCTCGCTCGCTCACTGAGGCCGCCCGGGCAAAGCCCGGGCGTC |
| | GGGCGACCTTTGGTCGCCCGGCCTCAGTGAGCGAGCGAGCGCGCAGAGA |
| | GGGAGTGGCCAACTCCATCACTAGGGGTTCCT (SEQ ID NO: 441) |
| R-ITR | AGGAACCCCTAGTGATGGAGTTGGCCACTCCCTCTCTGCGCGCTCGCTCG |
| | CTCACTGAGGCCGGGCGACCAAAGGTCGCCCGACGCCCGGGCTTTGCCC |
| | GGGCGGCCTCAGTGAGCGAGCGAGCGCGCAG (SEQ ID NO: 442) |
| ITR (145 bp) | AGGAACCCCTAGTGATGGAGTTGGCCACTCCCTCTCTGCGCGCTCGCTCG |
| | CTCACTGAGGCCGGGCGACCAAAGGTCGCCCGACGCCCGGGCTTTGCCC |
| | GGGCGGCCTCAGTGAGCGAGCGAGCGCGCAGAGAGGGAGTGGCCAA |
| | (SEQ ID NO: 443) |
| ITR (145 bp- | AGGAACCCCTAGTGATGGAGTTGGCCACTCCCTCTCTGCGCGCTCGCTCG |
| 1983, lacking | CTCACTGAGGCCGGGCGACCAAAGGTCGCCCGACGCCCGGGCTTTGCCC |
| 9 bp) | GGGCGGCCTCAGTGAGCGAGCGAGCGCGCAGAGAGGG (SEQ ID NO: 444) |

J. Exemplary rAAV Genome Elements:

### (i) AAV-LVR412

In some embodiments, the rAAV comprises in its genome, a construct comprising in a 5′ to 3′ direction, a 5′ AAV2-ITR (SEQ ID NO: 161), stuffer DNA (SEQ ID NO: 162), SP0412 LSP (SEQ ID NO: 91), Kozak sequence for Glue (SEQ ID NO: 153), a nucleic acid sequence encoding GAA polypeptide (SEQ ID NO: 182), a 3′ UTR (SEQ ID NO: 77), poly A sequence (SEQ ID NO: 164), a 3′ AAV-ITR sequence (SEQ ID NO: 165). An exemplary construct is shown as SEQ ID NO: 154 (LVR412_AskBioEU). One can readily change the ITRs sequences of SEQ ID NO: 161 and 165 for any ITR sequences for different AAV serotypes, or for ITRs selected from any of SEQ ID Nos: 441-444, as well as use a different UTR sequence or polyA sequence instead of SEQ ID NO: 77 and SEQ ID NO: 164, respectively.

In some embodiments, the rAAV comprises in its genome, a construct comprising in a 5′ to 3′ direction, a 5′ AAV2-ITR (SEQ ID NO: 161), stuffer DNA (SEQ ID NO: 162), SP0412 LSP (SEQ ID NO: 91), a nucleic acid sequence encoding GAA polypeptide (SEQ ID NO: 182), a 3′ UTR (SEQ ID NO: 77), a poly A sequence (SEQ ID NO: 166), a 3′ AAV-ITR sequence (SEQ ID NO: 165). An exemplary construct is shown as SEQ ID NO: 155 (ssAAV_LVR412WT-hGAA_AskBio_CHATHAM_Backbone Ask). One can readily change the ITRs sequences of SEQ ID NO: 161 and 165 for any ITR sequences for different AAV serotypes, or for ITRs selected from any of SEQ ID Nos: 441-444, as well as use a different UTR sequence or polyA sequence instead of SEQ ID NO: 77 and SEQ ID NO: 164, respectively.

AAV2-LVP422:

In some embodiments, the rAAV comprises in its genome, a construct comprising in a 5′ to 3′ direction, a 5′ AAV2-ITR (SEQ ID NO: 161), stuffer DNA, SP0422 LSP (SEQ ID NO: 92), Kozak sequence for Glue (SEQ ID NO: 153), a nucleic acid sequence encoding GAA polypeptide (SEQ ID NO: 55), a collagen stability sequence (SEQ ID NO: 65), a poly A sequence (SEQ ID NO: 164), a 3′ AAV-ITR sequence (SEQ ID NO: 165). An exemplary construct is shown as SEQ ID NO: 156 (LVR412Stuffer). One can readily change the ITRs sequences of SEQ ID NO: 161 and 165 for any ITR sequences for different AAV serotypes, or for ITRs selected from any of SEQ ID Nos: 441-444 as well as use a different collagen stability sequence or polyA sequence instead of SEQ ID NO: 65 and SEQ ID NO: 164, respectively.

In some embodiments, the rAAV comprises in its genome, a construct comprising in a 5′ to 3′ direction, a 5′ AAV2-ITR (SEQ ID NO: 161), stuffer DNA, SP0422 LSP (SEQ ID NO: 92), Kozak sequence for Glue (SEQ ID NO: 153), a nucleic acid sequence encoding GAA polypeptide (SEQ ID NO: 182), a 3′ UTR sequence (SEQ ID NO: 77), a poly A sequence (SEQ ID NO: 164), optionally comprising a AATAA stop signal, a 3′ AAV-ITR sequence (SEQ ID NO: 165). An exemplary construct is shown as SEQ ID NO: 157 (LVR422AskBio EU construct). One can readily change the ITRs sequences of SEQ ID NO: 161 and 165 for any ITR sequences for different AAV serotypes or for ITRs selected from any of SEQ ID Nos: 441-444, as well as use a different UTR sequence or polyA sequence instead of SEQ ID NO: 77 and SEQ ID NO: 164, respectively

One can readily change the ITRs sequences in the rAAV genomes of SEQ ID Nos: 160, 159, 155, 158, 156 for any ITR sequences for different AAV serotypes or for ITRs of SEQ ID NOs: 441-444, as well as use a different UTR sequence or polyA sequence, as disclosed herein.

In some embodiments, the rAAV vector or rAAV genome comprises a heterologous nucleic acid sequence encoding a GAA polypeptide comprising SEQ ID NO: 170 (GAA polypeptide with a cognate GAA signal sequence and H199R, R223H modifications), or SEQ ID NO: 171 (GAA polypeptide with a cognate GAA signal sequence and H199R, H201L and R223H modifications). The GAA polypeptide of SEQ ID NO: 170 is encoded by the nucleic acid sequence of SEQ ID NO: 182. Accordingly, in some embodiments, the rAAV vector comprises a nucleic acid of SEQ ID NO: 182 encoding a modified GAA polypeptide comprising H199R, R223H modifications. The GAA polypeptide of SEQ ID NO: 171 is encoded by the nucleic acid sequence of SEQ ID NO: 182 where basepairs (bp) 667-669 of SEQ ID NO: 182 are changed from CAC to any of: UUA, UUG, CUU, CUC CUA, CUG (resulting in a Histadine (H) to Leucine (L) amino acid change); or where bp 668 of SEQ ID NO: 182 is changed from A to U (resulting in a Histadine (H) to Leucine (L) amino acid change). Accordingly, in some embodiments, the rAAV vector comprises a nucleic acid of SEQ ID NO: 182, where bp 667-669 of SEQ ID NO: 182 are changed from CAC to any of: UUA, UUG, CUU, CUC CUA, CUG); or where bp 668 of SEQ ID NO: 182 is changed from A to U, which encodes a modified GAA polypeptide comprising H199R, H201L and R223H modifications.

In some embodiments, the rAAV vector or rAAV genome comprises a heterologous nucleic acid sequence encoding a GAA polypeptide selected from any of: SEQ ID NO: 172 (GAA polypeptide where cognate signal peptide is replaced with a IgG signal sequence and H199R, R223H modifications), or a sequence at least 85% sequence identity to SEQ ID NO: 172, or SEQ ID NO: 173 (GAA polypeptide where cognate signal peptide is replaced with a wtIL2 signal sequence and H199R and R223H modifications), or a sequence at least 85% sequence identity to SEQ ID NO: 173, or SEQ ID NO: 174 (GAA polypeptide where cognate signal peptide is replaced with a mutIL3 signal sequence and H199R and R223H modifications) or a sequence at least 85% sequence identity to SEQ ID NO: 174.

In some embodiments, the rAAV vector or rAAV genome comprises a heterologous nucleic acid sequence comprising SEQ ID NO: 182 where bp 1-81 of SEQ ID NO: 182 is replaced with the nucleic acid of SEQ ID NO: 177 (IgG signal sequence), which encodes a GAA polypeptide of SEQ ID NO: 172 (IgG leader-GAA with H199R and R223H modifications). In some embodiments, the rAAV vector comprises a heterologous nucleic acid sequence comprising SEQ ID NO: 182, where bp 668 of SEQ ID NO: 182 is changed from A to U and where bp 1-81 of SEQ ID NO: 182 is replaced with the nucleic acid of SEQ ID NO: 177 (IgG signal peptide), or a sequence at least 85% sequence identity thereto, which encodes a GAA polypeptide of SEQ ID NO: 172 (IgG leader-GAA with H199R, H201L and R223H modifications).

In some embodiments, the rAAV vector or rAAV genome comprises a heterologous nucleic acid sequence comprising SEQ ID NO: 182 where bp 1-81 of SEQ ID NO: 182 is replaced with the nucleic acid of SEQ ID NO: 179 (wt IL2 signal peptide), or a sequence at least 85% sequence identity thereto, which encodes a GAA polypeptide of SEQ ID NO: 173 (wt IL2 signal peptide-GAA with H199R, R223H modifications). In some embodiments, the rAAV vector comprises a heterologous nucleic acid sequence comprising SEQ ID NO: 182, where bp 668 of SEQ ID NO: 182 is changed from A to U and where bp 1-81 of SEQ ID NO: 182 is replaced with the nucleic acid of SEQ ID NO: 179 (wt IL2 signal peptide), or a sequence at least 85% sequence identity thereto, which encodes a GAA polypeptide of SEQ ID NO: 173 (wt IL2 signal peptide-GAA with H199R, H201L R223H modifications).

In some embodiments, the rAAV vector comprises a heterologous nucleic acid sequence comprising SEQ ID NO: 182 where bp 1-81 of SEQ ID NO: 182 is replaced with the nucleic acid of SEQ ID NO: 181 (mutIL2 signal peptide), or a sequence at least 85% sequence identity thereto, which encodes a GAA polypeptide of SEQ ID NO: 174 (mutIL2 signal peptide-GAA with H199R, R223H modifications). In some embodiments, the rAAV vector comprises a heterologous nucleic acid sequence comprising SEQ ID NO: 182, where bp 668 of SEQ ID NO: 182 is changed from A to U and where bp 1-81 of SEQ ID NO: 182 is replaced with the nucleic acid of SEQ ID NO: 181 (mut IL2 signal peptide), or a sequence at least 85% sequence identity thereto, which encodes a GAA polypeptide of SEQ ID NO: 174 (mut IL2 signal peptide-GAA with H199R, H201L and R223H modifications).

### III. Vectors and Virions

In one embodiment, the rAAV vector (also referred to as a rAAV virion) as disclosed herein comprises a capsid protein, and a rAAV genome in the capsid protein. A rAAV capsid of the rAAV virion used to treat Pompe Disease is any of those listed in Table 1 disclosed in U.S. Pat. No. 62,937,556, filed on Nov. 19, 2019, which is incorporated herein in its entirety by reference, or any combination thereof.

In one embodiment, a rAAV capsid of the rAAV virion used to treat Pompe Disease is any of those listed in Table 1 as disclosed in International Applications WO2020/102645, and WO2020/102667, each of which are incorporated herein in their entirety. In one embodiment, a rAAV capsid of the rAAV virion used to treat Pompe Disease is an AAV8 capsid. In one embodiment, a rAAV vector is an rAAV8 vector.

In one embodiment, the AAV vector (also referred to as a rAAV virion) as disclosed herein comprises a capsid protein from any of those disclosed in WO2019/241324, which is specifically incorporated herein in its entirety by reference. In some embodiments, the rAAV vector comprises a liver specific capsid, e.g., a liver specific capsid selected from XL32 and XL32.1, as disclosed in WO2019/241324, which is incorporated herein in its entirety by reference. In some embodiments, the rAAV vector is a AAVXL32 or AAVXL32.1 as disclosed in WO2019/241324, which is incorporated herein in its entirety by reference.

Exemplary chimeric or variant capsid proteins that can be used as the AAV capsid in the rAAV vector described herein can be selected from Table 2 from U.S. provisional application 62,937,556, filed on Nov. 19, 2019, which is specifically incorporated herein in its reference, or can be used with any combination with wild type capsid proteins and/or other chimeric or variant capsid proteins now known or later identified and each is incorporated herein. In some embodiments, the rAAV vector encompassed for use is a chimeric vector, e.g., as disclosed in 9,012,224 and U.S. Pat. No. 7,892,809, which are incorporated herein in their entirety by reference.

In some embodiments, the rAAV vector is a haploid rAAV vector, as disclosed in US application US2018/0371496 and PCT/US18/22725, or polyploid rAAV vector, e.g., as disclosed in PCT/US2018/044632 filed on Jul. 31, 2018 and in U.S. application Ser. No. 16/151,110, each of which are incorporated herein in their entirety by reference. In some embodiments, the rAAV vector is a rAAV3 vector, as disclosed in 9,012,224 and WO 2017/106236 which are incorporated herein in their entirety by reference.

In a particular embodiment, the rAAV is a AAVXL32 or AAVXL32.1 AAV vector as disclosed in WO2019/241324, which is incorporated herein in its entirety by reference. In some embodiments, the rAAV vector comprises a capsid disclosed in WO2019241324A1, or International Patent application PCT/US2019/036676, which are incorporated herein in their entirety by reference. In some embodiments, the AAV vector is a AAV8 vector or a rational haploid comprising an AAV8 capsid protein. In some embodiments, the recombinant AAV vector is a chimeric AAV vector, haploid AAV vector, a hybrid AAV vector or polyploid AAV vector. In some embodiments, the recombinant AAV vector is a rational haploid vector, a mosaic AAV vector, a chemically modified AAV vector, or a AAV vector from any AAV serotypes, for example, from any AAV serotype disclosed in Table 1 as disclosed in International Applications WO2020/102645, and WO2020/102667, each of which are incorporated herein in their entirety.

In some embodiments, the AAV vector comprises a capsid which is encoded by a nucleic acid AAV capsid coding sequence that is at least 90% identical to a nucleotide sequence of any one of SEQ ID NOs: 1-3 as disclosed in WO2019241324A1; or (b) a nucleotide sequence encoding any one of SEQ ID NOS:4-6 as disclosed in WO2019241324A1. In some embodiments, an AAV capsid comprises an amino acid sequence at least 90% identical to any one of SEQ ID NOS:4-6 as disclosed in WO2019241324A1, along with AAV particles comprising an AAV vector genome and the AAV capsid of the invention.

In one embodiment, the rAAV vector as disclosed herein comprises a capsid protein, associated with any of the following biological sequence files listed in the file wrappers of USPTO issued patents and published applications, which describe chimeric or variant capsid proteins that can be incorporated into the AAV capsid of this invention in any combination with wild type capsid proteins and/or other chimeric or variant capsid proteins now known or later identified (for demonstrative purposes, 11486254 corresponds to U.S. patent application Ser. No. 11/486,254 and the other biological sequence files are to be read in a similar manner): U.S. Ser. Nos. 11/486,254, 11/932,017, 12/172,121, 12/302,206, 12/308,959, 12/679,144, 13/036,343, 13/121,532, 13/172,915, 13/583,920, 13/668,120, 13/673,351, 13/679,684, 14/006,954, 14/149,953, 14/192,101, 14/194,538, 14/225,821, 14/468,108, 14/516,544, 14/603,469, 14/680,836, 14/695,644, 14/878,703, 14/956,934, 15/191,357, 15/284,164, 15/368,570, 15/371,188, 15/493,744, 15/503,120, 15/660,906, and 15/675,677.

In an embodiment, the AAV capsid proteins and virus capsids of this invention can be chimeric in that they can comprise all or a portion of a capsid subunit from another virus, optionally another parvovirus or AAV, e.g., as described in international patent publication WO 00/28004, which is incorporated by reference. In some embodiments, an rAAV vector genome is single stranded or a monomeric duplex as described in U.S. Pat. No. 8,784,799, which is incorporated herein.

As a further embodiment, the AAV capsid proteins and virus capsids of this invention can be polyploid (also referred to as haploid) in that they can comprise different combinations of VP1, VP2 and VP3 AAV serotypes in a single AAV capsid as described in US application US2018/0371496, which is incorporated by reference.

In an embodiment, an rAAV vector useful in the treatment of Pompe Disease as disclosed herein is an AAV3b capsid. AAV3b capsids encompassed for use are described in 2017/106236, and 9,012,224 and 7,892,809, and International application PCT/US19/61653, filed Nov. 15, 2019, and International Applications WO2020/102645, and WO2020/102667, each of which are incorporated herein in their entirety.

In some embodiments, the AAV3b capsid comprises SEQ ID NO: 44. In an embodiment, the AAV capsid used in the treatment of Pompe Disease can be a modified AAV capsid that is derived in whole or in part from the AAV capsid set forth in SEQ ID NO: 44. In some embodiments, the amino acids from an AAV3b capsid as set forth in SEQ ID NO: 44 can be, or are substituted with amino acids from another capsid of a different AAV serotype, wherein the substituted and/or inserted amino acids can be from any AAV serotype, and can include either naturally occurring or partially or completely synthetic amino acids.

In another embodiment, an AAV capsid used in the treatment of Pompe Disease is an AAV3b265D capsid. In this particular embodiment, an AAV3b265D capsid comprises a modification in the amino acid sequence of the two-fold axis loop of an AAV3b capsid via replacement of amino acid G265 of the AAV3b capsid with D265. In some embodiments, an AAV3b265D capsid comprises SEQ ID NO: 46. However, the modified virus capsids of the invention are not limited to AAV capsids set forth in SEQ ID NO: 46. In some embodiments, the amino acids from AAV3b265D as set forth in SEQ ID NO. 46 can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment an rAAV vector useful in the treatment of Pompe Disease as disclosed herein is an AAV3b265D549A capsid. In this particular embodiment, an AAV3b265D549A capsid comprises a modification in the amino acid sequence of the two-fold axis loop of an AAV3b capsid via replacement of amino acid G265 of the AAV3b capsid with D265 and replacement of amino acid T549 of the AAV3b capsid with A549. In some embodiments, an AAV3b265D549A capsid comprises SEQ ID NO: 50. However, the modified virus capsids of the invention are not limited to AAV capsids set forth in SEQ ID NO: 50. In some embodiments, the amino acids from AAV3b265D549A as set forth in SEQ ID NO: 50 can be, or are substituted with amino acids from a capsid from an AAV of a different serotype, wherein the substituted and/or inserted amino acids can be from any AAV serotype, and can include either naturally occurring or partially or completely synthetic amino acids. In some embodiments, the amino acids from AAV3bSASTG (i.e., a AAV3b capsid comprising Q263A/T265 mutations) can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment, an rAAV vector useful in the treatment of Pompe Disease as disclosed herein is an AAV3b549A capsid. In this particular embodiment, an AAV3b549A capsid comprises a modification in the amino acid sequence of the two-fold axis loop of an AAV3b capsid via replacement of amino acid T549 of the AAV3b capsid with A549. In some embodiments, an AAV3b549A capsid comprises SEQ ID NO: 52. However, the modified virus capsids of the invention are not limited to AAV capsids set forth in SEQ ID NO: 52. In some embodiments, the amino acids from AAV3b549A as set forth in SEQ ID NO: 52 can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment, an rAAV vector useful in the treatment of Pompe Disease as disclosed herein is an AAV3bQ263Y capsid. In this particular embodiment, an AAV3bQ263Y capsid comprises a modification in the amino acid sequence of the two-fold axis loop of an AAV3b capsid via replacement of amino acid Q263 of the AAV3b capsid with Y263. In some embodiments, an AAV3b549A capsid comprises SEQ ID NO: 54. However, the modified virus capsids of the invention are not limited to AAV capsids set forth in SEQ ID NO: 54. In some embodiments, the amino acids from AAV3bQ263Y as set forth in SEQ ID NO: 54 can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment, an rAAV vector useful in the treatment of Pompe Disease as disclosed herein is AAV3bSASTG serotype or comprises a AAV3bSASTG capsid. In this particular embodiment, an AAV3bSASTG capsid comprises a modification in the amino acid sequence to comprise a SASTG mutation, in particular, the AAV3b capsid was modified to resemble AAV2 Q263A/T265 subvariant by introducing these modifications at similar positions in the AAV3b capsid (as disclosed in Messina E L, et al., Adeno-associated viral vectors based on serotype 3b use components of the fibroblast growth factor receptor signaling complex for efficient transduction. Hum. Gene Ther. 2012 Oct.: 23(10):1031-4, Piacentino III, Valentino, et al. “X-linked inhibitor of apoptosis protein-mediated attenuation of apoptosis, using a novel cardiac-enhanced adeno-associated viral vector.” Human gene therapy 23.6 (2012): 635-646. which are both incorporated herein in their entirety by reference). Accordingly, in some embodiments, an rAAV vector useful in the treatment of Pompe Disease as disclosed herein is AAV3bSASTG serotype or comprises a AAV3bSASTG capsid comprising a AAV3b Q263A/T265 capsid. In some embodiments, the amino acids from AAV3bSASTG can be, or are substituted with amino acids from a capsid from an AAV of a different serotype, wherein the substituted and/or inserted amino acids can be from any AAV serotype, and can include either naturally occurring or partially or completely synthetic amino acids.

In order to facilitate their introduction into a cell, an rAAV vector genome useful in the invention are recombinant nucleic acid constructs that include (1) a heterologous sequence to be expressed (in one embodiment, a polynucleotide encoding a GAA polypeptide) and (2) viral sequence elements that facilitate integration and expression of the heterologous genes. The viral sequence elements may include those sequences of an AAV vector genome that are required in cis for replication and packaging (e.g., functional ITRs) of the DNA into an AAV capsid. In an embodiment, the heterologous gene encodes GAA, which is useful for correcting a GAA-deficiency in a patient suffering from Pompe Disease. In an embodiment, such an rAAV vector genome may also contain marker or reporter genes. In an embodiment, an rAAV vector genome can have one or more of the AAV3b wild-type (WT) cis genes replaced or deleted in whole or in part, but retain functional flanking ITR sequences.

In one embodiment, an rAAV vector as disclosed herein useful in the treatment of Pompe Disease comprises a rAAV genome as disclosed herein, encapsulated by an AAV3b capsid. In some embodiments, an rAAV vector as disclosed herein useful in the treatment of Pompe Disease comprises a rAAV genome as disclosed herein, encapsulated by any AAV3b capsid selected from: AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54), or a AAV3bSASTG (i.e., Q263A/T265) capsid.

In some embodiments of the methods and compositions as disclosed herein, the rAAV vector as disclosed herein comprises the nucleic acid sequences of any of: AAV_LVR412_EU (SEQ ID NO: 154), ssAAV_LVR412WT-hGAA_AskBio_CHATHAM (SEQ ID NO: 155), AAV-LVR412Stuffer (SEQ ID NO: 156), AAV_LVR422_EU (SEQ ID NO: 157), AAV-LVR422_Stuffer (SEQ ID NO: 158), ssAAV_LVR412_WT-hGAA CHATHAM (SEQ ID NO: 159), ssAAV_LSP_WT-hGAA-CHATHAM (SEQ ID NO: 160), or a nucleic acid sequence having at least 80%, 85%, 90%, 95% or 98% identity thereto. In some embodiments of the methods and compositions as disclosed herein, the rAAV vector that comprises a nucleic acid sequence of any of: SEQ ID NO: 154-160 can have the wtGAA sequence replaced by a modified GAA nucleic acid sequence as disclosed herein.

In some embodiments of the methods and compositions as disclosed herein, the rAAV vector as disclosed herein comprises the nucleic acid sequences of any of: SEQ ID NO: 57 (AAT-V43M-wtGAA (delta1-69aa)); SEQ ID NO: 58 (ratFN1-IGF2V43M-wtGAA (delta1-69aa)); SEQ ID NO: 59 (hFN1-IGF2V43M-wtGAA (delta1-69aa)); SEQ ID NO: 60 (AAT-IGF2-Δ2-7-wtGAA (delta 1-69)); SEQ ID NO: 61 (FN1rat-IGFΔ2-7-wtGAA (delta 1-69)); SEQ ID NO: 62 (hFN1-IGFΔ2-7-wtGAA (delta 1-69)), or a nucleic acid sequence having at least 80%, 85%, 90%, 95% or 98% identity thereto. In some embodiments of the methods and compositions as disclosed herein, the rAAV vector comprises a nucleic acid sequence of any of: AAT_hIGF2-V43M_wtGAA_del1-69_Stuffer.V02 (SEQ ID NO: 79); FIBrat_hIGF2-V43M_wtGAA_del1-69_Stuffer.V02 (SEQ ID NO: 80); FIBhum_hIGF2-V43M_wtGAA_del1-69_Stuffer.V02 (SEQ ID NO: 81); AAT_GILT_wtGAA_del1-69__Stuffer.V02 (SEQ ID NO: 82); FIBrat_GILT_wtGAA_del1-69_Stuffer.V02 (SEQ ID NO: 83); FIBhum_GILT_wtGAA_del1-69_Stuffer.V02 (SEQ ID NO: 84) or a nucleic acid sequence having at least 80%, 85%, 90%, 95% or 98% identity thereto.

In some embodiments of the methods and compositions as disclosed herein, the rAAV vector as disclosed herein comprises the nucleic acid sequences of any of: AAV_LVR412_EU (SEQ ID NO: 154), ssAAV_LVR412WT-hGAA_AskBio_CHATHAM (SEQ ID NO: 155), AAV-LVR412Stuffer (SEQ ID NO: 156), AAV_LVR422_EU (SEQ ID NO: 157), AAV-LVR422_Stuffer (SEQ ID NO: 158), ssAAV_LVR412_WT-hGAA CHATHAM (SEQ ID NO: 159), ssAAV_LSP_WT-hGAA-CHATHAM (SEQ ID NO: 160), or a nucleic acid sequence having at least 80%, 85%, 90%, 95% or 98% identity thereto. In some embodiments of the methods and compositions as disclosed herein, the rAAV vector that comprises a nucleic acid sequence of any of: SEQ ID NO: 154-160 can have the wtGAA sequence replaced by a modified GAA nucleic acid sequence as disclosed herein

### IV. Optimized rAAV Vector Genome

In some embodiments of the methods and compositions as disclosed herein, an optimized rAAV vector genome is created from any of the elements disclosed herein and in any combination, including nucleic acid sequences encoding a promoter, an ITR, a poly-A tail, elements capable of increasing or decreasing expression of a heterologous gene, and in one embodiment, a nucleic acid sequence that is codon optimized for expression of GAA protein in vivo (i.e., coGAA or codon optimized GAA) and optionally, one or more element to reduce immunogenicity. Such an optimized rAAV vector genome can be used with any AAV capsid that has tropism for the tissue and cells in which the rAAV vector genome is to be transduced and expressed.

In some embodiments, rAAV genome lacks the AAV P5 promoter, which is normally located upstream of the liver-specific promoter as disclosed herein. Normally, the P5 promoter controls expression of the AAV rep/cap proteins during AAV replication. In some embodiments, this P5 promoter fragment is present in the rAAV vector as disclosed herein which contains predicted transcription factor binding sites, e.g., cyclic AMP-responsive element-binding protein 3 (CREB3), which can be activated by endoplasmic reticulum (ER)/Golgi stress (Sampieri 2019), activating transcription factor 2 (ATF2), which is also involved in stress response (Watson 2017), Nuclear Receptor Subfamily 1 Group I Member 2 (NR1I2) (also known as Pregnane X receptor [PXR]) is known to be enriched in liver, and is activated by pregnane steroids, rifampin and other molecules including dexamethasone (NR1I2_HGNC) (Xing 2020). Accordingly, in some embodiments, a fragment of the AAV P5 promoter in the rAAV genome is removed without affecting the intended performance of the GAA cassette. In some embodiments, the rAAV vector also comprises a RNA polymerase II termination sequence located between the polyA signal and the 3′ ITR. An exemplary terminal sequence is SEQ ID NO: 439 which introduces two termination codons and one restriction site (e.g., XhoI) replaces TAG, and is located immediately downstream of the last coding amino acids of hGAA, and immediately located upstream of the 3′ UTR.

### AAV3b Capsid Modifications

In some embodiments of the methods and compositions as disclosed herein, an AAV3b capsid for use in a rAAV vector as disclosed herein, has an amino acid identity in the range of, e.g., about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to any of AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54) or a AAV3bSASTG capsid (i.e., a AAV3b capsid comprising Q263A/T265 mutations) disclosed in Nienaber et al., Hum. Gen Ther, 2012, 23(10); 1031-42 and Piacentino III, Valentino, et al. “X-linked inhibitor of apoptosis protein-mediated attenuation of apoptosis, using a novel cardiac-enhanced adeno-associated viral vector.” Human gene therapy 23.6 (2012): 635-646, both of which are incorporated herein in their entirety by reference. In yet other aspects of this embodiment, an AAV derived from AAV3b has an amino acid identity in the range of, e.g., about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to any of the amino acid sequence for AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54) a AAV3bSASTG capsid (i.e., a AAV3b capsid comprising Q263A/T265 mutations) as disclosed in Nienaber et al., Hum. Gen Ther, 2012, 23(10); 1031-42, and Piacentino III, Valentino, et al. “X-linked inhibitor of apoptosis protein-mediated attenuation of apoptosis, using a novel cardiac-enhanced adeno-associated viral vector.” Human gene therapy 23.6 (2012): 635-646. but the capsid still is a functionally active AAV protein.

In some embodiments of the methods and compositions as disclosed herein, the AAV serotype (e.g. AAV3b) comprises an SASTG mutation as described in Messina E L, et al., Adeno-associated viral vectors based on serotype 3b use components of the fibroblast growth factor receptor signaling complex for efficient transduction. Hum. Gene Ther. 2012 Oct.: 23(10):1031-42, which is incorporated herein in its entirety by reference.

In some embodiments of the methods and compositions as disclosed herein, an AAV3b capsid for use in a rAAV vector as disclosed herein, has, e.g., at least 1, 2, 3, 4, 5, 6, 7, 8, 9, 10, 15, 20, 25, 30, 35, 40, 45, or 50 contiguous amino acid deletions, additions, and/or substitutions relative to any of the amino acid sequence for AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54), or a AAV3bSASTG capsid (i.e., a AAV3b capsid comprising Q263A/T265 mutations) (as disclosed in Nienaber et al., Hum. Gen Ther, 2012, 23(10); 1031-42); or at most 1, 2, 3, 4, 5, 6, 7, 8, 9, 10, 15, 20, 25, 30, 35, 40, 45, or 50 contiguous amino acid deletions, additions, and/or substitutions relative to any of the amino acid sequence for AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54) a AAV3bSASTG capsid (i.e., a AAV3b capsid comprising Q263A/T265 mutations) (as disclosed in Nienaber et al., Hum. Gen Ther, 2012, 23(10); 1031-42). In yet another embodiment, an AAV3b capsid for use in a rAAV vector as disclosed herein, has, e.g., at least 1, 2, 3, 4, 5, 6, 7, 8, 9, 10, 15, 20, 25, 30, 35, 40, 45, or 50 contiguous amino acid deletions, additions, and/or substitutions relative to any of the amino acid sequence for AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54), or a AAV3bSASTG capsid (i.e., a AAV3b capsid comprising Q263A/T265 mutations) (as disclosed in Nienaber et al., Hum. Gen Ther, 2012, 23(10); 1031-42); or at most 1, 2, 3, 4, 5, 6, 7, 8, 9, 10, 15, 20, 25, 30, 35, 40, 45, or 50 contiguous amino acid deletions, additions, and/or substitutions relative to any of the amino acid sequence for AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54), or a AAV3bSASTG capsid (i.e., a AAV3b capsid comprising Q263A/T265 mutations) (as disclosed in Nienaber et al., Hum. Gen Ther, 2012, 23(10); 1031-42), but is still a functionally active AAV.

In some embodiments of the methods and compositions as disclosed herein, an AAV3b capsid for use in a rAAV vector as disclosed herein, has an amino acid identity in the range of, e.g., about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to any of the amino acid sequence for AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54), a AAV3bSASTG capsid(i.e., a AAV3b capsid comprising Q263A/T265 mutations) (as disclosed in Nienaber et al., Hum. Gen Ther, 2012, 23(10); 1031-42). In yet a further embodiment, an AAV3b capsid for use in a rAAV vector as disclosed herein has an amino acid identity in the range of, e.g., about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to any of the amino acid sequence for AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54), a AAV3bSASTG capsid (i.e., a AAV3b capsid comprising Q263A/T265 mutations) (as disclosed in Nienaber et al., Hum. Gen Ther, 2012, 23(10); 1031-42), but is still a functionally active AAV.

### V. Methods of Treatment

### A. Pompe Disease

The recombinant AAV expressing GAA protein as disclosed herein can be used in methods to treat Pompe disease and other glycogen storage diseases (GSD). Pompe disease is a rare genetic disorder caused by a deficiency in the enzyme acid alpha-glucosidase (GAA), which is needed to break down glycogen, a stored form of sugar used for energy. Pompe disease is also known as glycogen storage disease type II, GSD II, type II glycogen storage disease, glycogenosis type II, acid maltase deficiency, alpha-1,4-glucosidase deficiency, cardiomegalia glycogenic diffusa, and cardiac form of generalized glycogenosis. The build-up of glycogen causes progressive muscle weakness (myopathy) throughout the body and affects various body tissues, particularly in the heart, skeletal muscles, liver, respiratory and nervous system.

The presenting clinical manifestations of Pompe disease can vary widely depending on the age of disease onset and residual GAA activity. Residual GAA activity correlates with both the amount and tissue distribution of glycogen accumulation as well as the severity of the disease. Infantile-onset Pompe disease (less than 1% of normal GAA activity) is the most severe form and is characterized by hypotonia, generalized muscle weakness, and hypertrophic cardiomyopathy, and massive glycogen accumulation in cardiac and other muscle tissues. Death usually occurs within one year of birth due to cardiorespiratory failure. Juvenile-onset (1-10% of normal GAA activity) and adult-onset (10-40% of normal GAA activity) Pompe disease are more clinically heterogeneous, with greater variation in age of onset, clinical presentation, and disease progression. Juvenile- and adult-onset Pompe disease are generally characterized by lack of severe cardiac involvement, later age of onset, and slower disease progression, but eventual respiratory or limb muscle involvement results in significant morbidity and mortality. While life expectancy can vary, death generally occurs due to respiratory failure.

In any embodiment of the methods and compositions as disclosed herein, a GAA enzyme suitable for treating Pompe disease includes a wild-type human GAA, or a fragment or sequence variant thereof which retains the ability to cleave al-4 linkages in linear oligosaccharides. In some embodiments of the methods and compositions as disclosed herein, the GAA protein encoded by a wild type GAA nucleic acid sequence, e.g., SEQ ID NO: 11 or SEQ ID NO: 72. In some embodiments of the methods and compositions as disclosed herein, the GAA protein is encoded by a codon optimized GAA nucleic acid sequence, for example, for any one or more of: (1) enhanced expression in vivo, (2) to reduce CpG islands or (3) reduce the innate immune response. In some embodiments of the methods and compositions as disclosed herein, the GAA protein is encoded by a codon optimized GAA nucleic sequence, for example, any nucleic acid sequence selected from any of: SEQ ID NO: 73, SEQ ID NO: 74, SEQ ID NO: 75 and SEQ ID NO: 76 or SEQ ID NO: 182, or a nucleic acid sequence having at least 60%, or 70%, or 80%, 85% or 90% or 95%, or 98%, or 99% sequence identity to SEQ ID NO: 73, SEQ ID NO: 74, SEQ ID NO: 75, SEQ ID NO: 76 or SEQ ID NO: 182.

In some embodiments of the methods and compositions as disclosed herein, a rAAV vector as described herein transduces the liver of a subject and secretes the hGAA polypeptide into the blood, which perfuses patient tissues where the hGAA polypeptide, with the assistance of the fused IGF2-sequence, is taken up by cells and transported to the lysosome, where the enzyme acts to eliminate material that has accumulated in the lysosomes due to the enzyme deficiency. For lysosomal enzyme replacement therapy to be effective, the therapeutic enzyme must be delivered to lysosomes in the appropriate cells in tissues where the storage defect is manifest.

### B. Modulating GAA Levels in a Cell Ex Vivo

The nucleic acids, vector, and virions as described herein can be used to modulate levels of GAA in a cell. The method includes the step of administering to the cell a composition including a nucleic acid that includes a polynucleotide encoding GAA interposed between two AAV ITRs. The cell can be from any animal into which a nucleic acid of the invention can be administered. Mammalian cells (e.g., humans, dogs, cats, pigs, sheep, mice, rats, rabbits, cattle, goats, etc.) from a subject with GAA deficiency are typical target cells for use in the invention. In some embodiments, the cell is a liver cell or a myocardial cell e.g., a myocardiocyte.

In an embodiment ex vivo delivery of cells transduced with rAAV vector is disclosed herein. In a further embodiment, ex vivo gene delivery may be used to transplant cells transduced with a rAAV vector as disclosed herein back into the host. In a further embodiment, ex vivo stem cell (e.g., mesenchymal stem cell) therapy may be used to transplant cells transduced with a rAAV vector as disclosed herein cells back into the host. In another embodiment, a suitable ex vivo protocol may include several steps.

In some embodiments, a segment of target tissue (e.g., muscle, liver tissue) may be harvested from the subject, and the rAAV vector described herein used to transduce a GAA-encoding nucleic acid into a host's cells. These genetically modified cells may then be transplanted back into the host. Several approaches may be used for the reintroduction of cells into the host, including intravenous injection, intraperitoneal injection, subcutaneous injection, or in situ injection into target tissue. Microencapsulation of modified ex vivo cells transduced or infected with an rAAV vector as described herein is another technique that may be used within the invention. Autologous and allogeneic cell transplantation may be used according to the invention.

In yet another embodiment, disclosed herein is a method of treating a deficiency of GAA in a subject, comprising administering to the subject a cell expressing GAA as disclosed herein, in a pharmaceutically acceptable carrier and in a therapeutically effective amount. In some embodiments, the subject is a human.

### C. Increasing GAA Activity in a Subject

The nucleic acids, vectors, and virions as described herein can be used to modulate levels of functional GAA polypeptide in a subject, e.g., a human subject, or subject with Pompe disease or at risk of having Pompe disease. The method includes administering to the subject a composition comprising the rAAV vector, comprising the rAAV genome as described herein, comprising a heterologous nucleic acid encoding GAA interposed between two AAV ITRs, where the hGAA is linked to a signal peptide as described herein, and optionally a IGF2 targeting peptide as disclosed herein. The subject can be any animal, e.g., mammals (e.g., human beings, dogs, cats, pigs, sheep, mice, rats, rabbits, cattle, goats, etc.) are suitable subjects. The methods and compositions of the invention are particularly applicable to GAA-deficient human subjects.

Furthermore, the nucleic acids, vectors, and virions described herein may be administered to animals including human beings in any suitable formulation by any suitable method. For example, in any embodiment of the methods and compositions as disclosed herein, an rAAV vector, or rAAV genome as disclosed herein can be directly introduced into an animal, including through administration by oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain) or other parenteral route depending on the desired route of administration and the tissue that is being targeted.

In some embodiments, as the an rAAV vector, or rAAV genome comprises a nucleic acid sequence encoding GAA under the control of, or operatively linked to a liver specific promoter, the methods and compositions as disclosed herein can be administered via intravenous or intramuscular injection, where the rAAV vector, or rAAV genome will travel to the liver and express the GAA protein.

In any embodiment of the methods and compositions as disclosed herein, administration to a muscle can be by any suitable method including intravenous administration, intra-arterial administration, and/or intra-peritoneal administration. Exemplary modes of administration include oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). The most suitable route in any given case will depend on the nature and severity of the condition being treated and/or prevented and on the nature of the particular vector that is being used.

In any embodiment of the methods and compositions as disclosed herein, administration to skeletal muscle according to the present invention includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. Suitable skeletal muscles that can be injected are disclosed in U.S. provisional application 62,937,556, filed on Nov. 19, 2019, which is incorporated herein its entirety by reference.

In any embodiment of the methods and compositions as disclosed herein, the rAAV vectors and/or rAAV genome as disclosed herein are administered to the skeletal muscle, liver, diaphragm, costal, and/or cardiac muscle cells of a subject. For example, a conventional syringe and needle can be used to inject a rAAV virion suspension into an animal. Parenteral administration of a the rAAV vectors and/or rAAV genome, by injection can be performed, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain agents for a pharmaceutical formulation, such as suspending, stabilizing and/or dispersing agents. Alternatively, the rAAV vectors and/or rAAV genome as disclosed herein can be in powder form (e.g., lyophilized) for constitution with a suitable vehicle, for example, sterile pyrogen-free water, before use.

In particular embodiments, more than one administration (e.g., two, three, four, five, six, seven, eight, nine, 10, etc., or more administrations) may be employed to achieve the desired level of gene expression over a period of various intervals, e.g., hourly, daily, weekly, monthly, yearly, etc. Dosing can be single dosage or cumulative (serial dosing), and can be readily determined by one skilled in the art. For instance, treatment of a disease or disorder may comprise a one-time administration of an effective dose of a pharmaceutical composition virus vector disclosed herein. Alternatively, treatment of a disease or disorder may comprise multiple administrations of an effective dose of a virus vector carried out over a range of time periods, such as, e.g., once daily, twice daily, trice daily, once every few days, or once weekly.

The timing of administration can vary from individual to individual, depending upon such factors as the severity of an individual's symptoms. For example, an effective dose of a virus vector disclosed herein can be administered to an individual once every six months for an indefinite period of time, or until the individual no longer requires therapy. A person of ordinary skill in the art will recognize that the condition of the individual can be monitored throughout the course of treatment and that the effective amount of a virus vector disclosed herein that is administered can be adjusted accordingly.

Injectables can be prepared in conventional forms, either as liquid solutions or suspensions, solid forms suitable for solution or suspension in liquid prior to injection, or as emulsions. Alternatively, one may administer the virus vector and/or virus capsids of the invention in a local rather than systemic manner, for example, in a depot or sustained-release formulation. Further, the virus vector and/or virus capsid can be delivered adhered to a surgically implantable matrix (e.g., as described in U.S. Patent Publication No. US-2004-0013645-A1). The virus vectors and/or virus capsids disclosed herein can be administered to the lungs of a subject by any suitable means, optionally by administering an aerosol suspension of respirable particles comprised of the virus vectors and/or virus capsids, which the subject inhales. The respirable particles can be liquid or solid. Aerosols of liquid particles comprising the virus vectors and/or virus capsids may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the virus vectors and/or capsids may likewise be produced with any solid particulate medicament aerosol generator, by techniques known in the pharmaceutical art.

In some embodiments, the rAAV vectors and/or rAAV genome as disclosed herein can be formulated in a solvent, emulsion or other diluent in an amount sufficient to dissolve an rAAV vector disclosed herein. In other aspects of this embodiment, the rAAV vectors and/or rAAV genome as disclosed herein can herein may be formulated in a solvent, emulsion or a diluent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In other aspects, the rAAV vectors and/or rAAV genome as disclosed herein can disclosed herein may comprise a solvent, emulsion or other diluent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

In any embodiment of the methods and compositions as disclosed herein, the rAAV vectors and/or rAAV genome as disclosed herein, of any serotype, including but not limited to encapsulated by any AAV3b capsid selected from: AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54) or AAV3bSASTG capsid (i.e., a AAV3b capsid comprising Q263A/T265 mutations) can comprise a therapeutic compound in a therapeutically effective amount. In an embodiment, as used herein, without limitation, the term “effective amount” is synonymous with “therapeutically effective amount”, “effective dose”, or “therapeutically effective dose.” In an embodiment, the effectiveness of a therapeutic compound disclosed herein to treat Pompe Disease can be determined, without limitation, by observing an improvement in an individual based upon one or more clinical symptoms, and/or physiological indicators associated with Pompe Disease. In an embodiment, an improvement in the symptoms associated with Pompe Disease can be indicated by a reduced need for a concurrent therapy.

To facilitate delivery of a rAAV vector and/or rAAV genome as disclosed herein, it can be mixed with a carrier or excipient. Carriers and excipients that might be used include saline (especially sterilized, pyrogen-free saline) saline buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of virions to human subjects.

In addition to the formulations described previously, a rAAV vector and/or rAAV genome as disclosed herein can also be formulated as a depot preparation. Such long acting formulations may be administered by implantation (for example subcutaneously or intramuscularly) or by IM injection. Thus, for example, a rAAV vector and/or rAAV genome as disclosed herein may be formulated with suitable polymeric or hydrophobic materials (for example as an emulsion in an acceptable oil) or ion exchange resins, or as sparingly soluble derivatives.

In any embodiment of the methods and compositions as disclosed herein, the method is directed to treating Pompe Disease that results from a deficiency of GAA in a subject, wherein a rAAV vector and/or rAAV genome as disclosed herein is administered to a patient suffering from Pompe Disease, and following administration, GAA is secreted from cells in the liver and there is uptake of the secreted GAA by cells in skeletal muscle tissue, cardiac muscle tissue, diaphragm muscle tissue or a combination thereof, wherein uptake of the secreted GAA results in a reduction in lysosomal glycogen stores in the tissue(s). In some embodiments, the rAAV vector and/or rAAV genome as disclosed herein is encapsulated in a capsid, e.g., encapsulated by any AAV3b capsid selected from: AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54).

In a particular embodiment, at least about 10² to about 108 cells or at least about 103 to about 10⁶ cells will be administered per dose in a pharmaceutically acceptable carrier. In a further embodiment, dosages of the virus vector and/or capsid to be administered to a subject depend upon the mode of administration, the disease or condition to be treated and/or prevented, the individual subject's condition, the particular virus vector or capsid, the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10¹³, 10¹⁴, 10¹⁵ transducing units, optionally about 10⁸-10¹³ transducing units.

In another aspect, disclosed herein is a method of administering a nucleic acid encoding a GAA to a cell, comprising contacting the cell with a rAAV vector and/or rAAV genome as disclosed herein, under conditions for the nucleic acid to be introduced into the cell and expressed to produce GAA. In some embodiments, the cell is a cultured cell. In some embodiments, the cell is a cell in vivo. In some embodiments, the cell is a mammalian cell. In some embodiments, method of administering a nucleic acid encoding a GAA to a cell further comprises collecting the GAA secreted into a cell culture medium.

### D. Increasing Motoneuron Function in a Mammal

In any embodiment of the methods and compositions as disclosed herein, a rAAV vector and/or rAAV genome as disclosed herein is useful in compositions and methods to increase phrenic nerve activity in a mammal having Pompe disease and/or insufficient GAA levels. For example, a rAAV vector and/or rAAV genome as disclosed herein, e.g., a rAAV vector and/or rAAV genome encapsulated in a capsid, e.g., encapsulated by any AAV3b capsid selected from: AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54), can be administered to the central nervous system (e.g., neurons). In another embodiment, retrograde transport of rAAV vector and/or rAAV genome as disclosed herein encoding GAA from the diaphragm (or other muscle) to the phrenic nerve or other motor neurons can result in biochemical and physiological correction of Pompe disease. These same principles could be applied to other neurodegenerative disease.

In an embodiment, a rAAV GAA construct of any serotype as described in Table 1, including AAV8 or AAV3, or AAV3b (including but not limited to AAV3b serotypes AAV3b265D, AAV3b265D549A, AAV3b549A, AAV3bQ263Y, AAV3bSASTG (i.e., a AAV3b capsid comprising Q263A/T265 mutations) serotypes) is capable of reducing any one or more of the symptoms of (i) the feeling of weakness in a patient's lower extremities, including, the legs, trunk and/or arms, (ii) a shortness of breath, a hard time exercising, lung infections, a big curve in the spine, trouble breathing while sleeping, an enlarged liver, an enlarged tongue and/or a stiff joint, (iii) in a patient suffering from Pompe Disease by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, an AAV GAA of any serotype is capable of reducing any one or more of the systems of (i) the feeling of weakness in a patient's lower extremities, including, the legs, trunk and/or arms, ii) a shortness of breath, a hard time exercising, lung infections, a big curve in the spine, trouble breathing while sleeping, an enlarged liver, an enlarged tongue and/or a stiff joint, (iii) in a patient suffering from Pompe Disease by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In any embodiment of the methods and compositions as disclosed herein, at least one symptom associated with Pompe Disease, or at least one adverse side effect associated with Pompe Disease are reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, and the severity of at least one symptom associated with Pompe Disease, or at least one adverse side effect is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In another embodiment, at least one symptom associated with Pompe Disease, or at least one adverse side effect associated with Pompe Disease is reduced by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

E. Immunosuppression

In any embodiment of the methods and compositions as disclosed herein, a subject being administered a rAAV vector or rAAV genome as disclosed herein is also administered an immunosuppressive agent. Various methods are known to result in the immunosuppression of an immune response of a patient being administered AAV. Methods known in the art include administering to the patient an immunosuppressive agent, such as a proteasome inhibitor. One such proteasome inhibitor known in the art, for instance as disclosed in U.S. Pat. No. 9,169,492 and U.S. patent application Ser. No. 15/796,137, both of which are incorporated herein by reference, is bortezomib. In another embodiment, an immunosuppressive agent can be an antibody, including polyclonal, monoclonal, scfv or other antibody derived molecule that is capable of suppressing the immune response, for instance, through the elimination or suppression of antibody producing cells. In a further embodiment, the immunosuppressive element can be a short hairpin RNA (shRNA). In such an embodiment, the coding region of the shRNA is included in the rAAV cassette and is generally located downstream, 3′ of the poly-A tail. The shRNA can be targeted to reduce or eliminate expression of immunostimulatory agents, such as cytokines, growth factors (including transforming growth factors β1 and β2, TNF and others that are publicly known).

VI. Replacing GAA with other lysosomal enzymes.

In some embodiments, in any of the methods and compositions as disclosed herein, the nucleic acid sequence encoding a GAA polypeptide can be substituted for the nucleic acid sequence of a lysosomal enzyme. A lysosomal enzyme suitable to be expressed by the rAAV vectors or rAAV genomes as disclosed herein includes any enzyme that is capable of reducing accumulated materials in mammalian lysosomes or that can rescue or ameliorate one or more lysosomal storage disease symptoms. Suitable lysosomal enzymes include both wild-type or modified lysosomal enzymes and can be produced using recombinant or synthetic methods or purified from nature sources. Exemplary lysosomal enzymes are listed in Table 5A or Table 6A.

TABLE 5A
Exemplary Lysosomal Storage Diseases (LSD) and associated enzyme defects

| | | |
|---|---|---|
| Disease | Enzyme defect | Substance stored |

A. Glycogenosis Disorders

| | | |
|---|---|---|
| Pompe Disease | Acid-a1,4-Glucosidase | Glycogen α1-4 linked |
| | | Oligosaccharides |

B. Glycolipidosis Disorders

| | | |
|---|---|---|
| GM1 Gangliodsidosis | β-Galactosidase | GM₁ Gangliosides |
| Tay-Sachs Disease | β-Hexosaminidase A | GM₂ Ganglioside |
| AB Variant | Protein | |
| Sandhoff Disease | β-Hexosaminidase A & B | GM₂ Ganglioside |
| Fabry Disease | α-Galactosidase A | Globosides |
| Gaucher Disease | Glucocerebrosidase | Glucosylcerami |
| Metachromatic | Arylsulfatase A | Sulphatides |
| Leukodystrophy | | |
| Krabbe Disease | Galactosylceramidase | Galactocerebroside |
| Niemann-Pick, Types A & B | Acid Sphingomyelinase | Sphingomyelin |
| Niemann-Pick, Type D | Unknown | Sphingomyelin |
| Farber Disease | Acid Ceramidase | Ceramide |
| Wolman Disease | Acid Lipase | Cholesteryl Esters |

C. Mucopolysaccharide disorders

| | | |
|---|---|---|
| Hurler Syndrome (MPS IH) | α-L-Iduronidase | Heparan & Dermatan Sulfates |
| Scheie Syndrome (MPS IS) | α-L-Iduronidase | Heparan & Dermatan Sulfates |
| Hurler-Scheie (MPS IH/S) | α-L-Iduronidase | Heparan & Dermatan Sulfates |
| Hunter Syndrome (MPS II) | Iduronate Sulfatase | Heparan & Dermatan Sulfates |
| Sanfilippo A (MPS IIIA) | Heparan N-Sulfatase | Heparan Sulfate |
| Sanfilippo B (MPS IIIB) | α-N-Acetylglucosaminidase | Heparan Sulfate |
| Sanfilippo C (MPS IIIC) | Acetyl-CoA-Glucosaminide | Heparan Sulfate |
| | Acetyltransferase | |
| Sanfilippo D (MPS IIID) | N-Acetylglucosamine- | Heparan Sulfate |
| | 6-Sulfatase | |
| Morquio A (MPS IVA) | Galactosamine-6-Sulfatase | Keratan Sulfate |
| Morquio B (MPS IVB) | β-Galactosidase | Keratan Sulfate |
| Maroteaux-Lamy (MPS VI) | Arylsulfatase B | Dermatan Sulfate |
| Sly Syndrome (MPS VII) | β-Glucuronidase | |

D. Oligosaccharide/Glycoprotein Disorders

| | | |
|---|---|---|
| α-Mannosidosis | α-Mannosidosis | Mannose/Oligosacharides |
| β-Mannosidosis | β-Mannosidosis | Mannose/Oligosacharides |
| Fucosidosis | α-L-Fucosidase | Fucosyl Oligosaccharides |
| Aspartylglucosaminuria | N-Aspartyl-3- | Aspartylglucosamine |
| | Glucosaminidase | Asparagines |
| Sialidosis (Mucolipidosis 1) | α-Neuraminidase | Sialyloligosaccharides |
| Galactosialidosis | Lysosomal Protective Protein | Sialyloligosaccharides |
| (Goldberg Syndrome) | Deficiency | |
| Schindler Disease | α-N-Acetyl- | |
| | Galactosaminidase | |

E. Lysosomal Enzyme Transport Disorders

| | | |
|---|---|---|
| Mucolipidosis II | N-Acetylglucosamine-1- | Heparan Sulfate |
| (I-Cell Disease) | Phosphotransferase | |
| Mucolipidosis III (Pseudo- | Same as MLII | |
| Hurler Polydystrophy) | | |

F. Lysosomal Membrane Transport Disorders

| | | |
|---|---|---|
| Cystinosis | Cystine Transport Protein | Free Cystine |
| Salla Disease | Sialic Acid Transport Protein | Free Sialic Acid and |
| | | Glucuronic Acid |
| Infantile Sialic Acid | Sialic Acid Transport Protein | Free Sialic Acid and |
| Storage Disease | | Glucuronic Acid |

G. Other

| | | |
|---|---|---|
| Batten Disease (Juvenile | Unknown | Lipofuscins |
| Neuronal Ceroid | | |
| Lipofuscinosis) | | |
| Infantile Neuronal | Palmitoyl-Protein | Lipofuscins |
| Ceroid Lipofuscinosis | Thioesterase | |
| Mucolipidosis IV | Unknown | Gangliosides & |
| | | Hyaluronic Acid |
| Prosaposin | Saposins A, B, C or D | |

In some embodiments of the composition and methods disclosed herein, one particularly preferred lysosomal enzyme is glucocerebrosidase, which is currently recombinantly produced and manufactured by Genzyme and used in enzyme replacement therapy for Gaucher's Disease. Currently, the recombinant enzyme is prepared with exposed mannose residues, which targets the protein specifically to cells of the macrophage lineage. Although the primary pathology in type 1 Gaucher patients are due to macrophage accumulating glucocerebroside, there can be therapeutic advantage to delivering glucocerebrosidase to other cell types. Targeting glucocerebrosidase to lysosomes using the present invention would target the agent to multiple cell types and can have a therapeutic advantage compared to other preparations. In some embodiments of the composition and methods disclosed herein, the lysosomal disease treated in the methods disclosed herein is not Pompe. In some embodiments of the composition and methods disclosed herein, the lysosomal enzyme encoded by the nucleic acid in the targeting vector or rAAV vector is not GAA.

While methods and compositions of the invention are useful for producing and delivering any therapeutic agent to a subcellular compartment, the invention is particularly useful for delivering gene products for treating metabolic diseases.

In some embodiments, a lysosomal enzyme for treating lysosomal storage diseases (LSD) are shown in Table 5A. In some embodiments, the lysosomal enzyme is associated with Golgi or ER defects, which are shown in Table 6A. In a preferred embodiment, a viral vector encoding a lysosomal enzyme as described herein is delivered to a patient suffering from a defect in the same lysosomal enzyme gene. In alternative embodiments, a functional sequence or species variant of the lysosomal enzyme gene is used. In further embodiments, a gene coding for a different enzyme that can rescue a lysosomal enzyme gene defect is according to methods of the invention.

TABLE 6A
Diseases of the Golgi and ER
Diseases of the Golgi and ER

| | Gene and | |
|---|---|---|
| Disease Name | Enzyme Defect | Features |
| Ehlers-Danlos | PLOD1 lysyl | Defect in lysyl hydroxylation |
| Syndrome | hydroxylase | of Collagen; located in |
| Type VI | | ER lumen |
| Type Ia glycogen | glucose6 phosphatase | Causes excessive |
| storage disease | | accumulation of Glycogen |
| | | in the liver, kidney, and |
| | | Intestinal mucosa; enzyme |
| | | is transmembrane but |
| | | active site is ER lumen |

Congenital Disorders of Glycosylation

| | | |
|---|---|---|
| CDG Ic | ALG6 | Defects in N-glycosylation |
| | α1,3 | ER lumen |
| | glucosyltransferase | |
| CDG Id | ALG3 | Defects in N-glycosylation |
| | α1,3 | ER transmembrane protein |
| | mannosyltransferase | |
| CDG IIa | MGAT2 | Defects in N-glycosylation |
| | N-acetylglucosaminyl- | golgi transmembrane protein |
| | transferase II | |
| CDG IIb | GCS1 | Defect in N glycosylation |
| | α1,2-Glucosidase I | ER membrane bound with |
| | | lumenal catalytic domain |
| | | releasable by proteolysis |

In some embodiments of the methods and compositions as disclosed herein, a targeting vector or rAAV expresses a protein of any of the sequences in Table 5B or in Table 6B.

TABLE 5B
Exemplary Lysosomal Storage Diseases (LSD) and proteins to be expressed by
targeting vectors or rAAV vectors, and nucleic acid sequences encoding the proteins.

| | | | |
|---|---|---|---|
| | | Nucleic acid | |
| | | sequence for | |
| | | expression by a | Protein sequence |
| | | targeting vector | encoding the |
| Disease | Enzyme defect | or rAAV | enzyme defect |

A. Glycogenosis Disorders

| | | | |
|---|---|---|---|
| Pompe Disease | Acid-a1,4-Glucosidase | SEQ ID NO: 11 >> wt | SEQ ID NO: 10 (aa |
| | (GAA)(WT) | full length hGAA | wt full length hGAA |
| | | (non-codon | aa (non-codon |
| | | optimized); | optimized); |
| | | NP_000143.2 | NM_000152.4), |
| | | | or SEQ ID |
| | | | Nos 170, 171, 172, |
| | | | 173, 174 |
| | Acid-a1,4-Glucosidase | SEQ ID NO: 72 or | |
| | (hGAA) | SEQ ID NO: 182 >>> | |
| | | hGAA | |
| | Acid-a1,4-Glucosidase | SEQ ID NO: 73 > | |
| | (hGAA 3X) | hGAA 3X | |
| | hGAA | SEQ ID NO: 74 > | |
| | hGAA_Codon_ | hGAA_Codon_ | |
| | Optimized_No1 > | Optimized_No1 | |
| | hGAA_Codon_ | SEQ ID NO: 75 > | |
| | Optimized_No2 | hGAA_Codon_ | |
| | | Optimized_No2 | |
| | >hGAA_Codon_ | SEQ ID NO: 76 > | |
| | Optimized_No3 | hGAA_Codon_ | |
| | | Optimized_No3 | |

B. Glycolipidosis Disorders

| | | | |
|---|---|---|---|
| GM1 Gangliodsidosis | β-Galactosidase (GLB1) | SEQ ID NO: 227 | SEQ ID NO: 185 |
| (GLB1 deficiency) | | GLB1 | NP_000395.3 |
| | | (NM 000404.4) | |
| Tay-Sachs Disease | β-Hexosaminidase A | SEQ ID NO: 228 | SEQ ID NO: 186 |
| | (HEXA) | NM_000520.6 | NP_000511.2 |
| | | (HEXA) | (HEXA) |
| GM2-gangliosidosis, AB | β-Hexosaminidase A | SEQ ID NO: 229 | SEQ ID NO: 186 |
| variant | (HEXA) and HEXB | NM_000520.6 | NP_000511.2 |
| | | (HEXA) | (HEXA) |
| Sandhoff Disease | β-Hexosaminidase A & | SEQ ID NO:229; | SEQ ID NO: 186 |
| | β-Hexosaminidase B | NM_000520.6 | NP 000511.2 |
| | (HEXA and HEXB) | (HEXA) | (HEXA) |
| | | SEQ ID NO: 230; | SEQ ID NO: 187; |
| | | NM_000521.4 | NP_000512.2 |
| | | (HEXB) | (HEXB) |
| Fabry Disease | α-Galactosidase A | SEQ ID NO: 231 | SEQ ID NO: 188; |
| | (GLA) | NM_000169.3 (GLA) | NP_000160.1 |
| | | | (GLA) |
| Gaucher Disease | Glucocerebrosidase | SEQ ID NO: 232 | SEQ ID NO: 189; |
| | (GBA) | NM_000157.4 (GBA) | NP_000148.2 |
| | | | (GBA) |
| Metachromatic | Arylsulfatase A (ARSA) | SEQ ID NO: 233 | SEQ ID NO: 190 |
| Leukodystrophy | | NM_000487.6(ARSA) | |
| Krabbe Disease (also | Galactosylceramidase | SEQ ID NO: 234 | SEQ ID NO: 191; |
| called globoid cell | (GALC) | | NP_000144.2 |
| leukodystrophy) | | | |
| Niemann-Pick, | Acid Sphingomyelinase | SEQ ID NO: 235 | SEQ ID NO: 192; |
| Types A & B | (SMPD1) | NM_000543.5 | NP_000534.3 |
| | | (SMPD1) | (SMPD1) |
| Niemann-Pick, Type C1 | NPC intracellular | SEQ ID NO: 236 | SEQ ID NO: 193; |
| | cholesterol transporter 1 | NM_000271.5 | NP_000262.2 |
| | (NPC1) | (NPC1) | (NPC1) |
| Niemann-Pick, Type C2 | NPC intracellular | SEQ ID NO: 237 | SEQ ID NO: 194; |
| | cholesterol transporter 2 | NM_006432.5 | NP_006423.1 |
| | (NPC2) | (NPC2) | (NPC2) |
| Farber Disease (Farber | Acid Ceramidase | SEQ ID NO: 238 | SEQ ID NO: 195; |
| lipogranulomatosis) | (ASAH1)(also known as | NM_004315.6 | NP_004306.3 |
| | N-acylsphingosine | (ASAH1) | (ASAH1) |
| | amidohydrolase) | | |
| Wolman Disease (also | Lysomal Acid Lipase | SEQ ID NO: 239 | SEQ ID NO: 196; |
| known as Lysosomal acid | (LIPA) (also known | NM_000235.4 | NP_000226.2 |
| lipase deficiency) | as Lipase A) | (LIPA) | (LIPA) |

C. Mucopolysaccharide disorders

| | | | |
|---|---|---|---|
| Mucopolysaccharidosis | α-L-Iduronidase | SEQ ID NO: 240; | SEQ ID NO: 197; |
| type 1 (MPS 1) (includes | (IDUA) | NM_000203.5 | NP_000194.2 |
| 3 MPS I types: Hurler | | (IDUA) | (IDUA) |
| Syndrome (MPS IH); | | | |
| Scheie Syndrome (MPS | | | |
| IS) Hurler-Scheie | | | |
| (MPS IH/S) | | | |
| Mucopolysaccharidosis | Iduronate Sulfatase | SEQ ID NO: 241; | SEQ ID NO: 198; |
| type II (MPS II), (also | (IDS) | NM_000202.8 | NP_000193.1 |
| known as Hunter | | (IDS) | (IDS) |
| syndrome) | | | |
| Sanfilippo A | Heparan N-Sulfatase | SEQ ID NO: 242; | SEQ ID NO: 199; |
| (MPS IIIA) | (also referred to as | NM_000199.5 | NP_000190.1 |
| | N-sulfoglucosamine | (SGSH) | (SGSH) |
| | sulfohydrolase) | | |
| | (SGSH) | | |
| Sanfilippo B | α-N- | SEQ ID NO: 243; | SEQ ID NO: 200 |
| (MPS IIIB) | Acetylglucosaminidase | NM_000263.4 | NP_000254.2 |
| | (NAGLU) | (NAGLU) | (NAGLU) |
| Sanfilippo C | Acetyl-CoA- | SEQ ID NO: 244; | SEQ ID NO: 201; |
| (MPS IIIC) | Glucosaminide | NM_152419.3 | NP_689632.2 |
| | Acetyltransferase (also | (HGSNAT) | (HGSNAT) |
| | referred to as heparan- | | |
| | alpha-glucosaminide N- | | |
| | acetyltransferase) (often | | |
| | shortened to N- | | |
| | acetyltransferase) | | |
| | HGSNAT) | | |
| Sanfilippo D | N-Acetylglucosamine- | SEQ ID NO: 245; | SEQ ID NO: 202; |
| (MPS IIID) | 6-Sulfatase (GNS) | NM_002076.4 | NP_002067.1 |
| | (also referred to | (GNS) | (GNS) |
| | as glucosamine | | |
| | (N-acetyl)-6-sulfatase) | | |
| Mucopolysaccharidosis | Galactosamine-6- | SEQ ID NO: 246; | SEQ ID NO: 203; |
| type IVA (MPS IVA) | Sulfatase | NM_000512.5 | NP_000503.1 |
| also known as | (GALNS) | (GALNS) | (GALNS) |
| Morquio A syndrome) | | | |
| Mucopolysaccharidosis | β-Galactosidase | SEQ ID NO: 227; | SEQ ID NO: 185 |
| type IVB (MPS IVB), | (GLB1) | GLB1 | NP_000395.3 |
| (also known as | | (NM_000404.4) | |
| Morquio B syndrome) | | | |
| Mucopolysaccharidosis | Arylsulfatase B | SEQ ID NO: 247; | SEQ ID NO: 204 |
| type VI (MPS VI), (also | (ARSB) | NM_000046.5 | NP_000037.2 |
| known as Maroteaux- | | (ARSB) | (ARSB) |
| Lamy syndrome) | | | |
| Mucopolysaccharidosis | β-Glucuronidase | SEQ ID NO: 248 | SEQ ID NO: 205 |
| type VII (MPS VII), (also | (GUSB) | NM_000181.4 | NP_000172.2 |
| known as Sly syndrome) | | (GUSB) | (GUSB) |

D. Oligosaccharide,Glycoprotein Disorders

| | | | |
|---|---|---|---|
| α-Mannosidosis | α-Mannosidosis | SEQ ID NO: 249 | SEQ ID NO: 206 |
| | (MAN2B1) | | |
| | | NM_000528.4 | NP_000519.2 |
| | | (MAN2B1) | (MAN2B1) |
| α-Mannosidosis | α-Mannosidosis | SEQ ID NO: 250 | SEQ ID NO: 207 |
| | (MANBA) | NM_005908.4 | NP_005899.3 |
| | | (MANBA) | (MANBA) |
| Fucosidosis | α-L-Fucosidase | SEQ ID NO: 251 | SEQ ID NO: 208 |
| | (FUCA1) | NM_000147.4 | NP_000138.2 |
| | | (FUCA1) | (FUCA1) |
| Aspartylglucosaminuria | N-Aspartyl-β- | SEQ ID NO: 252 | SEQ ID NO: 209 |
| | Glucosaminidase | NM_000027.4 | NP_000018.2 |
| | (AGA) (also known as | (AGA) | (AGA) |
| | aspartylglucosaminidase | | |
| | (ASRG)or N(4)-(beta- | | |
| | N-acetylglucosaminyl)- | | |
| | L-asparaginase or | | |
| | glycosylasparaginase | | |
| | (AGU) | | |
| Sialidosis | neuraminidase 1 | SEQ ID NO: 253 | SEQ ID NO: 210 |
| (Mucolipidosis 1) | (also referred to as | NM_000434.4 | NP_000425.1 |
| | α-Neuraminidase) | (NEU1) | (NEU1) |
| | (NEU1) | | |
| Galactosialidosis (also | Cathepsin A (CTSA) | SEQ ID NO: 254 | SEQ ID NO: 211 |
| known as Goldberg | (also known | NM_000308.4 | NP_000299.3 |
| Syndrome or Lysosomal | as protective | (CTSA) | (CTSA) |
| Protective Protein | protein/cathepsin | | |
| Deficiency or PPCA | A or PPCA or | | |
| deficiency or | PPGB, or GSL) | | |
| neuraminidase | | | |
| deficiency with | | | |
| beta-galactosidase | | | |
| deficiency) | | | |
| Schindler Disease (also | α-N-Acetyl- | SEQ ID NO: 255 | SEQ ID NO: 212 |
| referred to as NAGA | Galactosaminidase | NM_000262.3 | NP_000253.1 |
| deficiency or alpha- | (NAGA) (also | (NAGA) | (NAGA) |
| galactosidase B | referred to a | | |
| deficiency) | alpha-galactosidase | | |
| | B or GALB) | | |

E. Lysosomal Enzyme Transport Disorders

| | | | |
|---|---|---|---|
| Mucolipidosis II | N-Acetylglucosamine- | SEQ ID NO: 256 | SEQ ID NO: 213 |
| (I-Cell Disease) | 1-Phosphotransferase | NM_024312.5 | NP_077288.2 |
| | (GNPTAB) | (GNPTAB) | (GNPTAB) |
| | (also referred | | |
| | to as GlcNAc- | | |
| | 1-phosphotransferase) | | |
| Mucolipidosis III | Same as MLII | SEQ ID NO: 256 | SEQ ID NO: 213 |
| (Pseudo-Hurler | | | |
| Polydystrophy) | | | |

F. Lysosomal Membrane

| | | | |
|---|---|---|---|
| Transport Disorders | | | |
| Cystinosis | cystinosin (also referred | SEQ ID NO: 257 | SEQ ID NO: 214 |
| | to as lysosomal cystine | NM_004937.3 | NP_004928.2 |
| | transporter) (CTNS) | (CTNS) | (CTNS) |
| Sialic acid storage disease | solute carrier family 17 | SEQ ID NO: 258 | SEQ ID NO: 2015 |
| (Salla Disease is a less | member 5 (SLC17A5) | NM_012434.5 | NP_036566.1 |
| severe form) | (also known as Sialin or | (SLC17A5) | (SLC17A5) |
| | Sialic Acid Transport | | |
| | Protein or SAISD, SD, | | |
| | ISSD, NSD) | | |
| Infantile Sialic Acid | solute carrier family 17 | SEQ ID NO: 1258 | SEQ ID NO: 215 |
| Storage Disease (ISSD) | member 5 (SLC17A5) | | |
| | (also known as Sialin or | | |
| | Sialic Acid Transport | | |
| | Protein or SAISD, SD, | | |
| | ISSD, NSD) | | |

G. Other

| | | | |
|---|---|---|---|
| CLN3 disease | Battenin (also | SEQ ID NO: 259 | SEQ ID NO: 216 |
| (including Batten | referred to as CLN3 | NM_000086.2 | NP_000077.1 |
| Disease (Juvenile | lysosomal/endosomal | (CLN3) | (CLN3) |
| Neuronal Ceroid | transmembrane protein | | |
| Lipofuscinosis) | or Batten) (CLN3) | | |
| Infantile Neuronal | Palmitoyl-Protein | SEQ ID NO: 260 | SEQ ID NO: 217 |
| Ceroid Lipofuscinosis | Thioesterase 1 | NM_000310.3 | NP_000301.1 |
| (or infantile Batten | (PPT1 gene) | (PPT1) | (PPT1) |
| disease) | | | |
| (CLN1 disease) | | | |
| Mucolipidosis IV | mucolipin-1 | SEQ ID NO: 261 | SEQ ID NO: 218 |
| | (MCOLN1) | NM_020533.3 | NP_065394.1(MCO |
| | | (MCOLN1) | LN1) |
| Prosaposin Deficiency | Prosaposin (PSAP). | SEQ ID NO: 262 | SEQ ID NO: 219 |
| (associated with | PSAP protein is the | PSAP | NP_002769.1 |
| metachromatic | precursor of four smaller | (NM_002778.4) | (PSAP) |
| leukodystrophy or | proteins called saposin | | |
| PSAP mutation) | A, B, C, and D) | | |

TABLE 6B
Exemplary Lysosomal Storage Diseases (LSD) and proteins to be expressed by
targeting vectors or rAAV vectors, and nucleic acid sequences encoding the proteins.

| Disease | Enzyme defect | Nucleic acid sequence | Protein sequence |
|---|---|---|---|
| Ehlers-Danlos | PLOD1 lysyl | SEQ ID NO: 263 | SEQ ID NO: 220 |
| Syndrome Type | Hydroxylase (PLOD1) | NM_000302.4 | NP_000293.2 |
| VI | (procollagen-lysine, | | |
| | 2-oxoglutarate | | |
| | 5-dioxygenase 1) | | |
| Type Ia glycogen | glucose6 phosphatase | SEQ ID NO: 264 | SEQ ID NO: 221 |
| storage disease | catalytic subunit | NM_000151.4 | NP_000142.2 |
| (GSDIa) | (G6PC) | (G6PC) | (G6PC) |
| Type Ib glycogen | solute carrier family 37 | SEQ ID NO: 265 | SEQ ID NO: 222 |
| storage disease | member 4 (also known | NM_001467.6 | NP_001458.1 |
| (GSDIb) | as glucose 6-phosphate | (SLC37A4) | (SLC37A4) |
| | translocase protein or | | |
| | G6PT1 or GSDIb) | | |
| | (SLC37A4) | | |

Congenital Disorders of Glycosylation

| | | | |
|---|---|---|---|
| ALG6-congenital | ALG6 α1,3 | SEQ ID NO: 266 | SEQ ID NO: 223 |
| disorder of | glucosyltransferase | NM_013339.4 | NP_037471.2 |
| glycosylation | (ALG6) | (ALG6) | (ALG6) |
| (ALG6-CDG) | | | |
| (also known as | | | |
| congenital disorder | | | |
| of glycosylation | | | |
| type Ic or CDG 1c) | | | |
| congenital disorder | ALG3 | SEQ ID NO: 267 | SEQ ID NO: 224 |
| of glycosylation | α1,3 | NM_005787.6 | NP_005778.1 |
| type Id (CDG-Id) | mannosyltransferase | (ALG3) | (ALG3) |
| characterized by | (ALG3) | | |
| abnormal | | | |
| N-glycosylation | | | |
| Congenital disorder | MGAT2 (encodes | SEQ ID NO: 268 | SEQ ID NO: 225 |
| of glycosylation | N-acetylglucosaminyl- | NM_002408.4 | NP_002399.1 |
| 2A (CDG2A or | transferase II | (MGAT2) | (MGAT2) |
| CDG IIa) | (also referred to as | | |
| | alpha-1,6-mannosyl- | | |
| | glycoprotein 2-beta- | | |
| | N-acetylglucosaminyl- | | |
| | transferase | | |
| | or GNT-II) | | |
| Type IIb | GCSI | SEQ ID NO: 269 | SEQ ID NO: 226 |
| congenital | α1,2-Glucosidase 1 | NM_006302.3 | NP_006293.2 |
| disorder of | (GDG2B or GCS1) | (MOGS) | (MOGS) |
| glycosylation | (also known as | | |
| (CDG IIb) | mannosyl- | | |
| | oligosaccharide | | |
| | glucosidase or MOGS) | | |

### VII. Administration

In some embodiments, the technology described herein relates to methods and compositions for administering a rAAV vector or rAAV genome as disclosed herein to a subject with a lysosomal disease or disorder, e.g., to a subject with Pompe disease. In some embodiments, the method comprises administering a composition comprising a homogenous population of rAAV vector comprising a rAAV vector, where the rAAV vector is a AAV3 or AAV8 vector, or a haploid rAAV vector comprising at least one capsid protein from AAV3 or AAV8 as disclosed herein. In some embodiments, the subject is administered a cocktail of different rAAV vectors as disclosed herein, e.g., a composition comprising a rAAV vector targeting or transduces liver cells and a rAAV vector that targets or transduces muscle cells. In an exemplary embodiment, a subject is administered a composition comprising cocktail of two or more different rAAV vectors as disclosed herein, e.g., a compostions comprising a AAV3 vector and AAV8 vector as disclosed herein, where each AAV vector comprises a nucleic acid encoding a GAA polypeptide operatively linked to a LSP as disclosed herein.

In some embodiments, the subject is co-administered, for example at the same time, or subsequently, two or more different rAAV vectors as disclosed herein. For exemplary purposes only, a subject can be administered a composition comprising a rAAV vector that targets or transduces liver cells and where the subject is also co-administered a composition comprising a rAAV vector that targets or transduces muscle cells. In an exemplary embodiment, a subject is co-administered a composition comprising a rAAV vector as disclosed herein, e.g., a composition comprising a AAV3 vector or variant thereof as disclosed herein, which comprises a nucleic acid encoding a GAA polypeptide operatively linked to a LSP as disclosed herein, and where the subject is also co-administered a composition comprising a different rAAV vector as disclosed herein, e.g., a compostions comprising a AAV8 vector or haploid AAV vector which comprises a nucleic acid encoding a GAA polypeptide operatively linked to a LSP as disclosed herein, or alternatively, where the LSP is replaced with a different promoter, e.g., a muscle specific promoter.

Dosages of the a rAAV vector or rAAV genome as disclosed herein to be administered to a subject depend upon the mode of administration, the severity of the Pompe disease or other condition to be treated and/or prevented, the individual subject's condition, the particular virus vector or capsid, and the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10¹³, 10¹⁴, 10¹⁵ transducing units, optionally about 10⁸ to about 10¹³ transducing units.

In a further embodiment, administration of rAAV vector or rAAV genome as disclosed herein to a subject results in production of a GAA protein with a circulatory half-life of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a rAAV vector or rAAV genome as disclosed herein to a subject is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In another embodiment, administration of a rAAV vector or rAAV genome as disclosed herein for the treatment of Pompe Disease results in an increase in weight by, e.g., at least 0.5 pounds, at least 1 pound, at least 1.5 pounds, at least 2 pounds, at least 2.5 pounds, at least 3 pounds, at least 3.5 pounds, at least 4 pounds, at least 4.5 pounds, at least 5 pounds, at least 5.5 pounds, at least 6 pounds, at least 6.5 pounds, at least 7 pounds, at least 7.5 pounds, at least 8 pounds, at least 8.5 pounds, at least 9 pounds, at least 9.5 pounds, at least 10 pounds, at least 10.5 pounds, at least 11 pounds, at least 11.5 pounds, at least 12 pounds, at least 12.5 pounds, at least 13 pounds, at least 13.5 pounds, at least 14 pounds, at least 14.5 pounds, at least 15 pounds, at least 20 pounds, at least 25 pounds, at least 30 pounds, at least 50 pounds.

In another embodiment, an AAV GAA of any serotype, as disclosed herein for the treatment of Pompe Disease results in an increase in weight by, e.g., from 0.5 pounds to 50 pounds, from 0.5 pounds to 30 pounds, from 0.5 pounds to 25 pounds, from 0.5 pounds to 20 pounds, from 0.5 pounds to 15 pounds, from 0.5 pounds to ten pounds, from 0.5 pounds to 7.5 pounds, from 0.5 pounds to 5 pounds, from 1 pound to 15 pounds, from 1 pound to 10 pounds, from 1 pound to 7.5 pounds, form 1 pound to 5 pounds, from 2 pounds to ten pounds, from 2 pounds to 7.5 pounds.

A. Pharmaceutical Compositions

The rAAV vectors as disclosed herein for use in the methods of administration as disclosed herein can be formulated in a pharmaceutical composition with a pharmaceutically acceptable excipient, i.e., one or more pharmaceutically acceptable carrier substances and/or additives, e.g., buffers, carriers, excipients, stabilizers, etc. The pharmaceutical composition may be provided in the form of a kit. Pharmaceutical compositions comprising the rAAV vectors as disclosed herein for use in the methods of administration as disclosed herein and uses thereof are known in the art.

Accordingly, a further aspect of the invention provides a pharmaceutical composition comprising a rAAV vector as disclosed herein for use in the methods of administration as disclosed herein. Relative amounts of the active ingredient (e.g. a rAAV vectors aa disclosed herein), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1 percent and 99 percent (w/w) of the active ingredient. By way of example, the composition may comprise between 0.1 percent and 100 percent, e.g., between 0.5 and 50 percent, between 1-30 percent, between 5-80 percent, at least 80 percent (w/w) active ingredient.

The pharmaceutical compositions can be formulated using one or more excipients or diluents to (1) increase stability; (2) increase cell transfection or transduction; (3) permit the sustained or delayed release of the payload; (4) alter the biodistribution (e.g., target the viral particle to specific tissues or cell types); (5) increase the translation of encoded protein; (6) alter the release profile of encoded protein and/or (7) allow for regulatable expression of the payload of the invention. In some embodiments, a pharmaceutically acceptable excipient may be at least 95 percent, at least 96 percent, at least 97 percent, at least 98 percent, at least 99 percent, or 100 percent pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia. Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21 st Edition, A. R. Gennaro, Lippincott, Williams and Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

The rAAV vectors as disclosed herein for use in the methods of administration as disclosed herein may be used in combination with one or more other therapeutic, prophylactic, research or diagnostic agents. By “in combination with,” it is not intended to imply that the agents must be administered at the same time and/or formulated for delivery together, although these methods of delivery are within the scope of the present invention. Compositions can be administered concurrently with, prior to, or subsequent to, one or more other desired therapeutics or medical procedures. In some embodiments, the delivery of one treatment (e.g., gene therapy vectors) is still occurring when the delivery of the second (e.g., one or more therapeutic) begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The composition described herein and the at least one additional therapy can be administered simultaneously, in the same or in separate compositions, or sequentially. For sequential administration, the gene therapy vectors described herein can be administered first, and the one or more therapeutic can be administered second, or the order of administration can be reversed. The gene therapy vectors and the one or more therapeutic can be administered during periods of active disorder, or during a period of remission or less active disease. The gene therapy vectors can be administered before another treatment, concurrently with the treatment, post-treatment, or during remission of the disorder.

When administered in combination, the rAAV vectors as disclosed herein for use in the methods of administration as disclosed herein and the one or more therapeutic (e.g., second or third therapeutic), or all, can be administered in an amount or dose that is higher, lower or the same as the amount or dosage of each used individually, e.g., as a monotherapy. In certain embodiments, the administered amount or dosage of a rAAV vector as disclosed herein for use in the methods of administration as disclosed herein and the one or more therapeutic (e.g., second or third agent), or all, is lower (e.g., at least 20%, at least 30%, at least 40%, or at least 50%) than the amount or dosage of each used individually. In other embodiments, the amount or dosage of the rAAV vector as disclosed herein for use in the methods of administration as disclosed herein and the one or more therapeutic (e.g., second or third agent), or all, that results in a desired effect (e.g., treatment of a cardiovascular disease or heart disease) is lower (e.g., at least 20%, at least 30%, at least 40%, or at least 50% lower) than the amount or dosage of each individually required to achieve the same therapeutic effect.

In some embodiments, the methods of administration of a rAAV vector as disclosed herein can deliver a rAVV vector disclosed herein alone, or in combination with an additional agent, for example, an immune modulator as disclosed herein.

B. Immune Modulator:

In some embodiments, the methods and compositions using the AAV vectors and AAV genomes as described herein, for treating lysosomal disease, e.g., Pompe, further comprises administering an immune modulator. In some embodiments, the immune modulator can be administered at the time of rAAV vector administration, before rAAV vector administration or, after the rAAV vector administration.

In some embodiments, the immune modulator is an immunoglobulin degrading enzyme such as IdeS, IdeZ, IdeS/Z, Endo S, or, their functional variant. Non-limiting examples of references of such immunoglobulin degrading enzymes and their uses as described in U.S. Pat. Nos. 7,666,582, 8,133,483, US 20180037962, US 20180023070, US 20170209550, U.S. Pat. No. 8,889,128, WO2010/057626, U.S. Pat. Nos. 9,707,279, 8,323,908, US 20190345533, US 20190262434, and WO2020/016318, each of which are incorporated in their entirety by reference.

In some embodiments, the immune modulator is Proteasome inhibitor. In certain aspects, the proteasome inhibitor is Bortezomib. In some aspects of the embodiment, the immune modulator comprises bortezomib and anti CD20 antibody, Rituximab. In other aspects of the embodiment, the immune modulator comprises bortezomib, Rituximab, methotrexate, and intravenous gamma globulin. Non-limiting examples of such references, disclosing proteasome inhibitors and their combination with Rituximab, methotrexate and intravenous gamma globulin, as described in U.S. Pat. Nos. 10,028,993, 9,592,247, and, U.S. Pat. No. 8,809,282, each of which are incorporated in their entirety by reference.

In alternative embodiments, the immune modulator is an inhibitor of the NF-kB pathway. In certain aspects of the embodiment, the immune modulator is Rapamycin or, a functional variant. Non-limiting examples of references disclosing rapamycin and its use described in U.S. Pat. No. 10,071,114, US 20160067228, US 20160074531, US 20160074532, US 20190076458, U.S. Pat. No. 10,046,064, are incorporated in their entirety. In other aspects of the embodiment, the immune modulator is synthetic nanocarriers comprising an immunosuppressant. Non limiting examples of references of immunosuppressants, immunosuppressants coupled to synthetic nanocarriers, synthetic nanocarriers comprising rapamycin, and/or, tolerogenic synthetic nanocarriers, their doses, administration and use as described in US20150320728, US 20180193482, US 20190142974, US 20150328333, US20160243253, U.S. Pat. No. 10,039,822, US 20190076522, US 20160022650, U.S. Pat. Nos. 10,441,651, 10,420,835, US 20150320870, US 2014035636, U.S. Pat. Nos. 10,434,088, 10,335,395, US 20200069659, U.S. Pat. No. 10,357,483, US 20140335186, U.S. Pat. Nos. 10,668,053, 10,357,482, US 20160128986, US 20160128987, US 20200038462, US 20200038463, each of which are incorporated in their entirety by reference.

In some embodiments, the immune modulator is synthetic nanocarriers comprising rapamycin (ImmTOR™ nanoparticles) (Kishimoto, et al., 2016, Nat Nanotechnol, 11(10): 890-899; Maldonado, et al., 2015, PNAS, 112(2): E156-165), as disclosed in US20200038463, U.S. Pat. No. 9,006,254 each of which is incorporated herein in its entirety. In some embodiments, the immune modulator is an engineered cell, e.g., an immune cell that has been modified using SQZ technology as disclosed in WO2017192786, which is incorporated herein in its entirety by reference.

In some embodiments, the immune modulator is selected from the group consisting of poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila's QS21 stimulon. In another further embodiment, the immunomodulator or adjuvant is poly-ICLC

In some embodiments, the immune modulator is a small molecule that inhibit the innate immune response in cells, such as chloroquine (a TLR signaling inhibitor) and 2-aminopurine (a PKR inhibitor), can also be administered in combination with the composition comprising at least one rAAV as disclosed herein. Some non-limiting examples of commercially available TLR-signaling inhibitors include BX795, chloroquine, CLI-095, OxPAPC, polymyxin B, and rapamycin (all available for purchase from INVIVOGEN™). In addition, inhibitors of pattern recognition receptors (PRR) (which are involved in innate immunity signaling) such as 2-aminopurine, BX795, chloroquine, and H-89, can also be used in the compositions and methods comprising at least one rAAV vector as disclosed herein for in vivo protein expression as disclosed herein.

In some embodiments, a rAAV vector can also encode a negative regulators of innate immunity such as NLRX1. Accordingly, in some embodiments, a rAAV vector can also optionally encode one or more, or any combination of NLRX1, NS1, NS3/4A, or A46R. Additionally, in some embodiments, a composition comprising at least one rAAV vector as disclosed herein can also comprise a synthetic, modified-RNA encoding inhibitors of the innate immune system to avoid the innate immune response generated by the tissue or the subject.

In some embodiments, an immune modulator for use in the administration methods as disclosed herein is an immunosuppressive agent. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Patent Pub. No 2002/0182211. In one embodiment, an immunosuppressive agent is cyclosporine A. Other examples include myophenylate mofetil, rapamicin, and anti-thymocyte globulin. In one embodiment, the immunosuppressive drug is administered in a composition comprising at least one rAAV vector as disclosed herein, or can be administered in a separate composition but simultaneously with, or before or after administration of a composition comprising at least one rAAV vector according to the methods of administration as disclosed herein. An immunosuppressive drug is administered in a formulation which is compatible with the route of administration and is administered to a subject at a dosage sufficient to achieve the desired therapeutic effect. In some embodiments, the immunosuppressive drug is administered transiently for a sufficient time to induce tolerance to the rAAV vector as disclosed herein.

In any embodiment of the methods and compositions as disclosed herein, a subject being administered a rAAV vector or rAAV genome as disclosed herein is also administered an immunosuppressive agent. Various methods are known to result in the immunosuppression of an immune response of a patient being administered AAV. Methods known in the art include administering to the patient an immunosuppressive agent, such as a proteasome inhibitor. One such proteasome inhibitor known in the art, for instance as disclosed in U.S. Pat. No. 9,169,492 and U.S. patent application Ser. No. 15/796,137, both of which are incorporated herein by reference, is bortezomib. In some embodiments, an immunosuppressive agent can be an antibody, including polyclonal, monoclonal, scfv or other antibody derived molecule that is capable of suppressing the immune response, for instance, through the elimination or suppression of antibody producing cells. In a further embodiment, the immunosuppressive element can be a short hairpin RNA (shRNA). In such an embodiment, the coding region of the shRNA is included in the rAAV cassette and is generally located downstream, 3′ of the poly-A tail. The shRNA can be targeted to reduce or eliminate expression of immunostimulatory agents, such as cytokines, growth factors (including transforming growth factors 31 and 02, TNF and others that are publicly known).

The use of such immune modulating agents facilitates the ability to for one to use multiple dosing (e.g., multiple administration) over numerous months and/or years. This permits using multiple agents as discussed below, e.g., a rAAV vector encoding multiple genes, or multiple administrations to the subject.

All aspects of the compositions and methods of the technology disclosed herein can be defined in any one or more of the following numbered paragraphs:
1. A recombinant adenovirus associated (AAV) vector comprising in its genome: (a) 5′ and 3′ AAV inverted terminal repeats (ITR) sequences, and (b) located between the 5′ and 3′ ITRs, a heterologous nucleic acid sequence encoding an alpha-glucosidase (GAA) polypeptide, wherein the heterologous nucleic acid is operatively linked to a liver-specific promoter.
2. The recombinant AAV vector of paragraph 1, wherein the heterologous nucleic acid sequence encoding the GAA polypeptide further comprises a signal peptide located 5′ of the nucleic acid encoding the alpha-glucosidase (GAA) polypeptide, or wherein the heterologous nucleic acid sequence encoding the GAA polypeptide further comprises a IGF2 targeting peptide at the N-terminus of GAA polypeptide.
3. The recombinant AAV vector of paragraph 1, wherein the heterologous nucleic acid sequence encoding the GAA polypeptide further comprises a IGF2 targeting peptide located between the secretory signal peptide and the alpha-glucosidase (GAA) polypeptide, or wherein the heterologous nucleic acid sequence encoding the GAA polypeptide further comprises a IGF2 targeting peptide at the N-terminus of GAA polypeptide.
4. The recombinant AAV vector of any of paragraphs 1-3, wherein the AAV genome comprises, in the 5′ to 3′ direction: (a) a 5′ ITR, (b) a liver-specific promoter sequence, (c) an intron sequence, (d) a nucleic acid encoding a secretory signal peptide, (e) a nucleic acid encoding an alpha-glucosidase (GAA) polypeptide, (f) a poly A sequence, and (g) a 3′ ITR. In some embodiments, the intron sequence (c) is absent.
5. The recombinant AAV vector of any of paragraphs 1-3, wherein the AAV genome comprises, in the 5′ to 3′ direction: (a) a 5′ ITR, (b) a liver-specific promoter sequence, (c) an intron sequence, (d) a nucleic acid encoding an IGF2 targeting peptide, (e) a nucleic acid encoding an alpha-glucosidase (GAA) polypeptide, (f) a poly A sequence, and (g) a 3′ ITR. In some embodiments, the intron sequence (c) is absent.
6. The recombinant AAV vector of any of paragraphs 1-6, wherein the AAV genome comprises, in the 5′ to 3′ direction: (a) a 5′ ITR, (b) a liver-specific promoter sequence, (c) an intron sequence, (d) a nucleic acid encoding a secretory signal peptide, (e) a nucleic acid encoding an IGF2 targeting peptide, (f) a nucleic acid encoding an alpha-glucosidase (GAA) polypeptide, (g) a poly A sequence, and (h) a 3′ ITR. In some embodiments, the intron sequence is absent.
7. The recombinant AAV vector of any of paragraphs 1-6, wherein the secretory signal peptide is selected from an AAT signal peptide, a fibronectin signal peptide (FN1), a GAA signal peptide, or an active fragment thereof having secretory signal activity. For example, the nucleic acid encoding a secretory signal peptide, can be selected from an AAT signal peptide (e.g., SEQ ID NO: 17), a fibronectin signal peptide (FN1) (e.g., SEQ ID NO: 18-21), a cognate GAA signal peptide (SEQ ID NO: 175), an hIGF2 signal peptide (e.g., SEQ ID NO: 22), a IgG1 leader peptide (SEQ ID NO: 177), wtIL2 leader peptide (SEQ ID NO: 179), mutant IL2 leader peptide (SEQ ID NO: 181) or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NOs: 17-22, 175, 177, 179 or 181.
8. The recombinant AAV vector of any of paragraphs 1-7, wherein the IGF2 targeting peptide binds human cation-independent mannose-6-phosphate receptor (CI-MPR) or the IGF2 receptor.
9. The recombinant AAV vector of any of paragraphs 1-5, wherein the IGF2 targeting peptide comprises SEQ ID NO: 5 or comprises at least one amino modification in SEQ ID NO: 5 that does not affect the binding to the CI-MPR receptor, or that reduces its binding to one or more IGF binding proteins (IGFBPs, such as IGFBP1-6).
10. The recombinant AAV vector of any of paragraphs 1-9, wherein the at least one amino modification in SEQ ID NO: 5 is a V43M amino acid modification (SEQ ID NO: 8 or SEQ ID NO: 9) or Δ2-7 (SEQ ID NO: 6) or Δ1-7 (SEQ ID NO: 7).
11. The recombinant AAV vector of any of paragraphs 1-10, wherein the liver-specific promoter is a promoter of any of the sequence listed in Table 4 herein, or Table 4A or 4B provisional application 62,937,556, filed on Nov. 19, 2019, or a functional variant thereof or functional fragment thereof.
12. The recombinant AAV vector of any of paragraphs 1-10, wherein the liver-specific promoter comprises CRM_SP0412 (SEQ ID NO: 86) or SP0412 (SEQ ID NO: 91) or a functional variant or functional fragment thereof having at least 60% activity to SEQ ID NO: 86 or SEQ ID NO: 91.
13. The recombinant AAV vector of any of paragraphs 1-12, wherein the liver specific promoter is selected from any of: (i) SP0422 (SEQ ID NO: 92) or a functional variant or functional fragment thereof having at least 60% activity to SEQ ID NO: 92; (ii) CRM_SP0239 (SEQ ID NO: 87) or SP0239 (SEQ ID NO: 93) or SP0238-UTR (SEQ ID NO: 147) or a functional variant or functional fragment thereof having at least 60% activity to SEQ ID NO: 87, SEQ ID NO: 93 or SEQ ID NO: 147; (iii) CRM_SP0265 (SP0131_A1) (SEQ ID NO: 88) or SP0265 (LVR SP0131 A1) (SEQ ID NO: 94) or SP0265-UTR (SEQ ID NO: 146) or a functional variant or functional fragment thereof having at least 60% activity to SEQ ID NO: 88, SEQ ID NO: 94 or SEQ ID NO: 146; (iv) CRM_SP0240 (SEQ ID NO: 89) or SP0240 (SEQ ID NO: 95) or SP0240-UTR (SEQ ID NO: 148) or a functional variant or functional fragment thereof having at least 60% activity to SEQ ID NO: 89, SEQ ID NO: 95 or SEQ ID NO: 148; (v) CRM_SP0246 (SEQ ID NO: 90) or SP0246 (SEQ ID NO: 96) or SP0246-UTR (SEQ ID NO: 149) or a functional variant or functional fragment thereof having at least 60% activity to SEQ ID NO: 90, SEQ ID NO: 96 or SEQ ID NO: 149.
14. The recombinant AAV vector of any of paragraphs 1-13, wherein the nucleic acid sequence encodes a wild-type GAA polypeptide or a modified GAA polypeptide.
15. The recombinant AAV vector of any of paragraphs 1-14, wherein the nucleic acid sequence encoding the GAA polypeptide is the human GAA gene or a human codon optimized GAA gene (coGAA) or a modified GAA nucleic acid sequence.
16. The recombinant AAV vector of any of paragraphs 1-15, wherein the nucleic acid sequence encoding the GAA polypeptide is codon optimized for enhanced expression in vivo.
17. The recombinant AAV vector of any of paragraphs 1-16, wherein the nucleic acid sequence encoding the GAA polypeptide is codon optimized to reduce CpG islands.
18. The recombinant AAV vector of any of paragraphs 1-17, wherein the nucleic acid sequence encoding the GAA polypeptide is codon optimized to reduce the innate immune response or to reduce CpG islands, or to reduce the innate immune response and reduce the innate immune response.
19. The recombinant AAV vector of any of paragraphs 1-18, wherein the nucleic acid sequence encodes a GAA polypeptide which comprises at least one, at least 2 or at least all three amino acid modifications selected from; H201L, H199R or R233H of SEQ ID NO: 10.
20. The recombinant AAV vector of any of paragraphs 1-19, wherein the encoded polypeptide further comprising a spacer comprising a nucleotide sequence for at least 1 amino acids located amino-terminal to the GAA polypeptide, and C-terminal to the IGF2 targeting peptide.
21. The recombinant AAV vector of any of paragraphs 1-20, further comprising a nucleic acid encoding a spacer of at least 1 amino acids located between the nucleic acid encoding the IGF2 targeting peptide and the nucleic acid encoding the GAA polypeptide.
22. The recombinant AAV vector of any of paragraphs 1-21, further comprising at least one polyA sequence located 3′ of the nucleic acid encoding the GAA gene and 5′ of the 3′ ITR sequence.
23. The recombinant AAV vector of any of paragraphs 1-22, wherein the heterologous nucleic acid sequence further comprises at collagen stability (CS) sequence located 3′ of the nucleic acid encoding the GAA polypeptide and 5′ of the 3′ ITR sequence.
24. The recombinant AAV vector of any of paragraphs 1-23, further comprising a nucleic acid encoding a collagen stability (CS) sequence located between the nucleic acid encoding the GAA polypeptide and the poly A sequence

25. The recombinant AAV vector of any of paragraphs 1-24, further comprising an intron sequence located 5′ of the sequence encoding the secretory signal peptide, and 3′ of the promoter.
26. The recombinant AAV vector of any of paragraphs 1-25, wherein the intron sequence comprises a MVM sequence, SV40 sequence or a HBB2 sequence, wherein the MVM sequence comprises the nucleic acid sequence of SEQ ID NO: 13, or a nucleic acid sequence at least about 75%, or 80%, or 85%, or 90%, or 95%, or 98%, or 99% sequence identity to SEQ ID NO: 13, and the HBB2 sequence comprises the nucleic acid sequence of SEQ ID NO: 14, or a nucleic acid sequence at least about 75%, or 80%, or 85%, or 90%, or 95%, or 98%, or 99% sequence identity to SEQ ID NO: 14.
27. The recombinant AAV vector of any of paragraphs 1-26, wherein the ITR comprises an insertion, deletion or substitution.
28. The recombinant AAV vector of any of paragraphs 1-27, wherein one or more CpG islands in the ITR are removed.
29. The recombinant AAV vector of any of paragraphs 1-28, wherein:
   a. the heterologous nucleic acid sequence encodes a secretory signal peptide selected from:
      1. a fibronectin signal peptide (FN1) or an active fragment thereof having secretory signal activity (e.g., a FN1 signal peptide has the sequence of any of SEQ ID NO: 18-21, or an amino acid sequence at having at least about 75%, or 80%, or 85%, or 90%, or 95%, or 98%, or 99% sequence identity to any of SEQ ID NOs: 18-21),
      2. an AAT signal peptide (e.g., SEQ ID NO: 17), or an amino acid sequence at having at least about 75%, or 80%, or 85%, or 90%, or 95%, or 98%, or 99% sequence identity to any of SEQ ID NO: 17;
      3. an hIGF2 signal peptide (e.g., SEQ ID NO: 22), or an amino acid sequence at having at least about 75%, or 80%, or 85%, or 90%, or 95%, or 98%, or 99% sequence identity to any of SEQ ID NO: 22;
      4. a IgG1 leader peptide (SEQ ID NO: 177), or an amino acid sequence at having at least about 75%, or 80%, or 85%, or 90%, or 95%, or 98%, or 99% sequence identity to any of SEQ ID NO: 177;
      5. a wtIL2 leader peptide (SEQ ID NO: 179), or an amino acid sequence at having at least about 75%, or 80%, or 85%, or 90%, or 95%, or 98%, or 99% sequence identity to any of SEQ ID NO: 179;
      6. a mutant IL2 leader peptide (SEQ ID NO: 181) or an amino acid sequence at having at least about 75%, or 80%, or 85%, or 90%, or 95%, or 98%, or 99% sequence identity to any of SEQ ID NO: 181, and
   b. the heterologous nucleic acid sequence encodes a GAA polypeptide is selected from any of the group consisting of: SEQ ID NO: 11, SEQ ID NO: 72 or SEQ ID NO: 182 or a nucleic acid sequence having at least 60%, or 70%, or 80%, 85% or 90% or 95%, or 98%, or 99% sequence identity to SEQ ID NO: 11, SEQ ID NO: 72, or SEQ ID NO: 182, and optionally, the nucleic acid sequence encodes a GAA polypeptide with at least one, at least 2 or at least all three amino acid modifications selected from; H201L, H199R or R233H of SEQ ID NO: 10.
30. The recombinant AAV vector of paragraph 29, wherein the heterologous nucleic acid also encodes a IGF2 targeting peptide selected from any of: SEQ ID NO: 5, SEQ ID NO: 6, SEQ ID NO: 7, SEQ ID NO: 8 or SEQ ID NO: 9, or a IGF2 peptide having at least about 75%, or 80%, or 85%, or 90%, or 95%, or 98%, or 99% sequence identity to SEQ ID NOs: 5-9.
31. The recombinant AAV vector of any of paragraphs 1-30, wherein the encoded secretory signal peptide is AAT signal peptide or an active fragment thereof having secretory signal activity, (e.g., a AAT signal peptide has the sequence of SEQ ID NO: 17, or an amino acid sequence at having at least about 75%, or 80%, or 85%, or 90%, or 95%, or 98%, or 99% sequence identity to SEQ ID NO: 17), and the heterologous nucleic acid sequence encodes a IGF2 targeting peptide selected from any of: SEQ ID NO: 5, SEQ ID NO: 6, SEQ ID NO: 7, SEQ ID NO: 8 or SEQ ID NO: 9, or a IGF2 peptide having at least about 75%, or 80%, or 85%, or 90%, or 95%, or 98%, or 99% sequence identity to SEQ ID NOs: 5-9.
32. The recombinant AAV vector of any of paragraphs 1-31, wherein the IGF2 targeting peptide is SEQ ID NO: 8 or SEQ ID NO: 9, or a IGF2 peptide having at least about 75%, or 80%, or 85%, or 90%, or 95%, or 98%, or 99% sequence identity to SEQ ID NO: 8 or 9.
33. The recombinant AAV vector of any of paragraphs 1-32, wherein the recombinant AAV vector is selected from any of: a chimeric AAV vector, a haploid AAV vector, a hybrid AAV vector, a polyploid AAV vector, a rational haploid vector, a mosaic AAV vector, a chemically modified AAV vector, or a AAV vector from any AAV serotypes, e.g., any serotype listed in Table 1 of disclosed in International Applications WO2020/102645, and WO2020/102667.
34. The recombinant AAV vector of any of paragraphs 1-33, wherein the recombinant AAV vector comprises a capsid protein selected from any AAV serotype in the group consisting of those listed in Table 1 of U.S. provisional application 62,937,556, filed on Nov. 19, 2019, or in International Applications WO2020/102645, and WO2020/102667, or and any combination thereof.
35. The recombinant AAV vector of any of paragraphs 1-34, wherein the AAV vector is selected from a serotype from the group consisting of: a AAV3 vector, a AAVXL32 vector, a AAVXL32.1 vector, a AAV8 vector, or a haploid AAV8 vector comprising at least one AAV8 capsid protein.
36. The recombinant AAV vector of any of paragraphs 1-35, wherein the AAV3b serotype comprises one or mutations in a capsid protein selected from any of: 265D, 549A, Q263Y
37. The recombinant AAV vector of any of paragraphs 1-36, wherein the AAV3b serotype is selected from any of: AAV3b265D, AAV3b265D549A, AAV3b549A or AAV3bQ263Y, or AAV3bSASTG. 38. A recombinant adenovirus associated (AAV) vector comprising in its genome:
   a. 5′ and 3′ AAV inverted terminal repeats (ITR) sequences, and
   b. located between the 5′ and 3′ ITRs, a heterologous nucleic acid sequence encoding an alpha-glucosidase (GAA) polypeptide, wherein the heterologous nucleic acid is operatively linked to a liver specific promoter, and
   wherein the recombinant AAV vector comprises a capsid protein of the AAV8 serotype or AAV3b serotype.
39. The recombinant AAV vector of paragraph 38, wherein the GAA polypeptide further comprises a secretory signal peptide located at the N-terminal of the GAA polypeptide.
40. The recombinant AAV vector of paragraph 38-39, wherein the heterologous nucleic acid sequence encoding the GAA polypeptide further comprises a IGF2 targeting peptide located N-terminal of the GAA polypeptide, or located between the secretory signal peptide and the an alpha-glucosidase (GAA) polypeptide.
41. The recombinant AAV vector of paragraph 38, wherein the AAV genome comprises, in the 5′ to 3′ direction: a 5′ ITR, a liver specific promoter sequence, a nucleic acid encoding an alpha-glucosidase (GAA) polypeptide, a poly A sequence, and a 3′ ITR.
42. The recombinant AAV vector of paragraph 38, wherein the AAV genome comprises, in the 5′ to 3′ direction: a 5′ ITR, a liver specific promoter sequence, a nucleic acid encoding a secretory signal peptide, a nucleic acid encoding an alpha-glucosidase (GAA) polypeptide, a poly A sequence, and a 3′ ITR.
43. The recombinant AAV vector of paragraph 38, wherein the AAV genome comprises, in the 5′ to 3′ direction: a 5′ ITR, a liver specific promoter sequence, an intron sequence, a nucleic acid encoding a secretory signal peptide, a nucleic acid encoding an alpha-glucosidase (GAA) polypeptide, a poly A sequence, and a 3′ ITR.
44. The recombinant AAV vector of paragraph 38, wherein the AAV genome comprises, in the 5′ to 3′ direction: a 5′ ITR, a liver specific promoter sequence, an intron sequence, a nucleic acid encoding a secretory signal peptide, a nucleic acid encoding a IGF2 targeting peptide, a nucleic acid encoding an alpha-glucosidase (GAA) polypeptide, a poly A sequence, and a 3′ ITR.
45. The recombinant AAV vector of paragraph 38, wherein the AAV genome comprises, in the 5′ to 3′ direction: a 5′ ITR, a liver specific promoter sequence, an intron sequence, a nucleic acid encoding a secretory signal peptide, a nucleic acid encoding a IGF2 targeting peptide, a nucleic acid encoding an alpha-glucosidase (GAA) polypeptide, a 3′ ITR sequence, a poly A sequence, and a 3′ ITR.
46. The recombinant AAV vector of any of paragraphs 34-37, wherein the secretory signal peptide is selected from nucleic acid encoding a secretory signal peptide, can be selected from an AAT signal peptide (e.g., SEQ ID NO: 17), a fibronectin signal peptide (FN1) (e.g., SEQ ID NO: 18-21), a cognate GAA signal peptide (SEQ ID NO: 175), an hIGF2 signal peptide (e.g., SEQ ID NO: 22), a IgG1 leader peptide (SEQ ID NO: 177), wtIL2 leader peptide (SEQ ID NO: 179), mutant IL2 leader peptide (SEQ ID NO: 181) or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NOs: 17-22, 175, 177, 179 or 181.
47. The recombinant AAV vector of any of paragraphs 38-46, wherein the IGF2 targeting peptide binds human cation-independent mannose-6-phosphate receptor (CI-MPR) or the IGF2 receptor.
48. The recombinant AAV vector of any of paragraphs 38-47, wherein the IGF2 targeting peptide comprises SEQ ID NO: 5 or comprises at least one amino modification in SEQ ID NO: 5 that does not affect binding to the IGF2 receptor or reduces its binding to one or more of IGF Binding proteins IGFBP1-6.
49. The recombinant AAV vector of paragraph 48, wherein the at least one amino modification in SEQ ID NO: 5 is a V43M amino acid modification (SEQ ID NO: 8 or SEQ ID NO: 9) or Δ2-7 (SEQ ID NO: 6) or Δ1-7 (SEQ ID NO: 7).
50. The recombinant AAV vector of any of paragraphs 38-49, wherein the liver specific promoter is selected from any of the sequences listed in Table 4, or Table 4A or 4B provisional application 62,937,556, filed on Nov. 19, 2019, or a functional variant or functional fragment thereof.
51. The recombinant AAV vector of any of paragraphs 38-50, wherein the liver specific promoter is selected from any of: CRM_SP0412 (SEQ ID NO: 86) or SP0412 (SEQ ID NO: 91) or a functional variant or functional fragment thereof having at least 60% activity to SEQ ID NO: 86 or SEQ ID NO: 91.
52. The recombinant AAV vector of any of paragraphs 38-50, wherein the liver specific promoter is selected from any of: (i) SP0422 (SEQ ID NO: 92) or a functional variant or functional fragment thereof having at least 60% activity to SEQ ID NO: 92; (ii) CRM_SP0239 (SEQ ID NO: 87) or SP0239 (SEQ ID NO: 93) or SP0238-UTR (SEQ ID NO: 147) or a functional variant or functional fragment thereof having at least 60% activity to SEQ ID NO: 87, SEQ ID NO: 93 or SEQ ID NO: 147; (iii) CRM_SP0265 (SP0131_A1) (SEQ ID NO: 88) or SP0265 (LVR_SP0131 A1) (SEQ ID NO: 94) or SP0265-UTR (SEQ ID NO: 146) or a functional variant or functional fragment thereof having at least 60% activity to SEQ ID NO: 88, SEQ ID NO: 94 or SEQ ID NO: 146; (iv) CRM_SP0240 (SEQ ID NO: 89) or SP0240 (SEQ ID NO: 95) or SP0240-UTR (SEQ ID NO: 148) or a functional variant or functional fragment thereof having at least 60% activity to SEQ ID NO: 89, SEQ ID NO: 95 or SEQ ID NO: 148; (v) CRM_SP0246 (SEQ ID NO: 90) or SP0246 (SEQ ID NO: 96) or SP0246-UTR (SEQ ID NO: 149) or a functional variant or functional fragment thereof having at least 60% activity to SEQ ID NO: 90, SEQ ID NO: 96 or SEQ ID NO: 149.
53. The recombinant AAV vector of paragraphs 38-52, wherein the nucleic acid sequence encodes a wild-type GAA polypeptide or a modified GAA polypeptide.
54. The recombinant AAV vector of any of paragraphs 38-53, wherein the nucleic acid sequence encoding the GAA polypeptide is the human GAA gene or a human codon optimized GAA gene (coGAA) or a modified GAA nucleic acid sequence.
55. The recombinant AAV vector of any of paragraphs 38-54, wherein the nucleic acid sequence encoding the GAA polypeptide is codon optimized for enhanced expression in vivo.
56. The recombinant AAV vector of any of paragraphs 38-55, wherein the nucleic acid sequence encoding the GAA polypeptide is codon optimized to reduce CpG islands.
57. The recombinant AAV vector of any of paragraphs 38-56, wherein the nucleic acid sequence encoding the GAA polypeptide is codon optimized to reduce the innate immune response, or reduce the CpG islands, or reduce the innate immune response and reduce the CpG islands.
58. The recombinant AAV vector of any of paragraphs 38-57, wherein the nucleic acid sequence encoding the GAA polypeptide encodes a GAA polypeptide comprising at least one, at least 2 or at least all three amino acid modifications selected from; H201L, H199R or R233H of SEQ ID NO: 10.
59. The recombinant AAV vector any of paragraphs 38-58, wherein the intron sequence is selected from any of the MVM, HBB2 or SV40 intron sequence, wherein the MVM sequence comprises the nucleic acid sequence of SEQ ID NO: 13, or a nucleic acid sequence at least about 75%, or 80%, or 85%, or 90%, or 95%, or 98%, or 99% sequence identity to SEQ ID NO: 13, and the HBB2 sequence comprises the nucleic acid sequence of SEQ ID NO: 14, or a nucleic acid sequence at least about 75%, or 80%, or 85%, or 90%, or 95%, or 98%, or 99% sequence identity to SEQ ID NO: 14.
60. The recombinant AAV vector any of paragraphs 38-59, wherein the ITR comprises an insertion, deletion or substitution.
61. The recombinant AAV vector of paragraph 60, wherein one or more CpG islands in the ITR are removed.
62. The recombinant AAV vector of any of paragraphs 38-61, wherein the 3′ ITR comprises or consist of SEQ ID NO: 442, or a nucleotide sequence that is at least 80% identical, e.g., at least 85%, 90%, 95%, 96%, 97%, 98%, or 99% or 99.5% identical to SEQ ID NO: 442, and the 5′ ITR comprises, or consists of SEQ ID NO: 441 or a nucleotide sequence that is at least 80% identical, e.g., at least 85%, 90%, 95%, 96%, 97%, 98%, or 99% or 99.5% identical to SEQ ID NO: 441.
63. The recombinant AAV vector of any of paragraphs 38-61, wherein the 3′ ITR comprises or consist of SEQ ID NO: 165, or a nucleotide sequence that is at least 80% identical, e.g., at least 85%, 90%, 95%, 96%, 97%, 98%, or 99% or 99.5% identical to SEQ ID NO: 165, and the 5′ ITR comprises, or consists of SEQ ID NO: 161 or a nucleotide sequence that is at least 80% identical, e.g., at least 85%, 90%, 95%, 96%, 97%, 98%, or 99% or 99.5% identical to SEQ ID NO: 161.
64. The recombinant AAV vector of any of paragraphs 38-63, wherein the secretory signal peptide is a fibronectin signal peptide (FN1) or an active fragment thereof having secretory signal activity, (e.g., a FN1 signal peptide has the sequence of any of SEQ ID NO: 18-21, or an amino acid sequence at having at least about 75%, or 80%, or 85%, or 90%, or 95%, or 98%, or 99% sequence identity to any of SEQ ID NOs: 18-21), and the heterologous nucleic acid sequence encodes a IGF2 targeting peptide selected from any of: SEQ ID NO: 5, SEQ ID NO: 6, SEQ ID NO: 7, SEQ ID NO: 8 or SEQ ID NO: 9, or a IGF2 peptide having at least about 75%, or 80%, or 85%, or 90%, or 95%, or 98%, or 99% sequence identity to SEQ ID NOs: 5-9.
65. The recombinant AAV vector of any of paragraphs 38-64, wherein the encoded secretory signal peptide is AAT signal peptide or an active fragment thereof having secretory signal activity, (e.g., a AAT signal peptide has the sequence of SEQ ID NO: 17, or an amino acid sequence at having at least about 75%, or 80%, or 85%, or 90%, or 95%, or 98%, or 99% sequence identity to SEQ ID NO: 17), and the heterologous nucleic acid sequence encodes a IGF2 targeting peptide selected from any of: SEQ ID NO: 5, SEQ ID NO: 6, SEQ ID NO: 7, SEQ ID NO: 8 or SEQ ID NO: 9, or a IGF2 peptide having at least about 75%, or 80%, or 85%, or 90%, or 95%, or 98%, or 99% sequence identity to SEQ ID NOs: 5-9.
66. The recombinant AAV vector of any of paragraphs 38-65, wherein the IGF2 targeting peptide is SEQ ID NO: 8 or SEQ ID NO: 9, or a IGF2 peptide having at least about 75%, or 80%, or 85%, or 90%, or 95%, or 98%, or 99% sequence identity to SEQ ID NO: 8 or 9.
67. The recombinant AAV vector of any of paragraphs 38-66, wherein the liver specific promoter is CRM_SP0412 (SEQ ID NO: 86) or SP0412 (SEQ ID NO: 91) or a functional variant or functional fragment thereof having at least 60% activity to SEQ ID NO: 86 or SEQ ID NO: 91.
68. The recombinant AAV vector of any of paragraphs 38-66, wherein the liver specific promoter is SP0422 (SEQ ID NO: 92) or a functional variant or functional fragment thereof having at least 60% activity to SEQ ID NO: 92.
69. The recombinant AAV vector of any of paragraphs 38-66, wherein the liver specific promoter is CRM_SP0239 (SEQ ID NO: 87) or SP0239 (SEQ ID NO: 93) or SP0238-UTR (SEQ ID NO: 147) or a functional variant or functional fragment thereof having at least 60% activity to SEQ ID NO: 87, SEQ ID NO: 93 or SEQ ID NO: 147.
70. The recombinant AAV vector of any of paragraphs 38-66, wherein the liver specific promoter is CRM_SP0265 (SP0131_A1) (SEQ ID NO: 88) or SP0265 (LVR_SP0131_A1) (SEQ ID NO: 94) or SP0265-UTR (SEQ ID NO: 146) or a functional variant or functional fragment thereof having at least 60% activity to SEQ ID NO: 88, SEQ ID NO: 94 or SEQ ID NO: 146.
71. The recombinant AAV vector of any of paragraphs 38-66, wherein the liver specific promoter is CRM_SP0240 (SEQ ID NO: 89) or SP0240 (SEQ ID NO: 95) or SP0240-UTR (SEQ ID NO: 148) or a functional variant or functional fragment thereof having at least 60% activity to SEQ ID NO: 89, SEQ ID NO: 95 or SEQ ID NO: 148.
72. The recombinant AAV vector of any paragraphs 38-66, wherein the liver specific promoter is CRM_SP0246 (SEQ ID NO: 90) or SP0246 (SEQ ID NO: 96) or SP0246-UTR (SEQ ID NO: 149) or a functional variant or functional fragment thereof having at least 60% activity to SEQ ID NO: 90, SEQ ID NO: 96 or SEQ ID NO: 149
73. A pharmaceutical composition comprising the recombinant AAV vector of any one of the previous paragraphs in a pharmaceutically acceptable carrier.
74. A nucleic acid sequence comprising:
   a liver specific promoter operatively linked to a heterologous nucleic acid sequence, the heterologous nucleic acid sequence encoding a GAA polypeptide, wherein the liver specific promoter is selected from any one of the sequences disclosed in Table 4, or Table 4A or 4B of U.S. provisional application 62,937,556, filed on Nov. 19, 2019, or a functional variant or functional fragment thereof.
75. The nucleic acid sequence of paragraph 74, wherein the heterologous nucleic acid sequence comprises in the following order: a nucleic acid encoding a secretory signal peptide, and a nucleic acid encoding a GAA polypeptide.
76. The nucleic acid sequence of paragraph 74, wherein the heterologous nucleic acid sequence comprises in the following order: a nucleic acid encoding a secretory signal peptide, a nucleic acid encoding a IGF2 targeting peptide and a nucleic acid encoding a GAA polypeptide.
77. A nucleic acid sequence for a recombinant adenovirus associated (rAAV) vector genome comprising:
   a. 5′ and 3′ AAV inverted terminal repeats (ITR) nucleic acid sequences, and
   b. located between the 5′ and 3′ ITR sequence, a heterologous nucleic acid sequence encoding a polypeptide comprising an alpha-glucosidase (GAA) polypeptide, wherein the heterologous nucleic acid is operatively linked to a liver-specific promoter, wherein the liver specific promoter is selected from any one of the sequences disclosed in Table 4, or Table 4A or 4B of U.S. provisional application 62,937,556, filed on Nov. 19, 2019, or a functional variant or functional fragment thereof.
78. A nucleic acid sequence of paragraph 75, wherein the nucleic acid sequence encodes a fusion polypeptide comprising a secretory signal and an alpha-glucosidase (GAA) polypeptide.
79. A nucleic acid sequence of paragraph 75, wherein the nucleic acid sequence encodes a fusion polypeptide comprising a secretory signal, an IGF2 targeting peptide and an alpha-glucosidase (GAA) polypeptide.
80. A nucleic acid sequence comprising:
   a liver specific promoter operatively linked to a heterologous nucleic acid sequence comprising, a nucleic acid encoding a GAA polypeptide, wherein the liver specific promoter is selected from any of:
   i. CRM_SP0412 (SEQ ID NO: 86) or SP0412 (SEQ ID NO: 91) or a functional variant or functional fragment thereof having at least 60% activity to SEQ ID NO: 86 or SEQ ID NO: 91,
   ii. SP0422 (SEQ ID NO: 92) or a functional variant or functional fragment thereof having at least 60% activity to SEQ ID NO: 92,
   iii. CRM_SP0239 (SEQ ID NO: 87) or SP0239 (SEQ ID NO: 93) or SP0238-UTR (SEQ ID NO: 147) or a functional variant or functional fragment thereof having at least 60% activity to SEQ ID NO: 87, SEQ ID NO: 93 or SEQ ID NO: 147;
   iv. CRM_SP0265 (SP0131_A1) (SEQ ID NO: 88) or SP0265 (LVR_SP0131_A1) (SEQ ID NO: 94) or SP0265-UTR (SEQ ID NO: 146) or a functional variant or functional fragment thereof having at least 60% activity to SEQ ID NO: 88, SEQ ID NO: 94 or SEQ ID NO: 146;
   v. CRM_SP0240 (SEQ ID NO: 89) or SP0240 (SEQ ID NO: 95) or SP0240-UTR (SEQ ID NO: 148) or a functional variant or functional fragment thereof having at least 60% activity to SEQ ID NO: 89, SEQ ID NO: 95 or SEQ ID NO: 148; or
   vi. CRM_SP0246 (SEQ ID NO: 90) or SP0246 (SEQ ID NO: 96) or SP0246-UTR (SEQ ID NO: 149) or a functional variant or functional fragment thereof having at least 60% activity to SEQ ID NO: 90, SEQ ID NO: 96 or SEQ ID NO: 149.
81. A nucleic acid sequence of paragraph 80, wherein the heterologous nucleic acid sequence comprises in the following order: a nucleic acid encoding a secretory signal and nucleic acid encoding an alpha-glucosidase (GAA) polypeptide.
82. A nucleic acid sequence of paragraph 75, wherein the heterologous nucleic acid sequence comprises in the following order: a nucleic acid encoding a secretory signal, a nucleic acid sequence encoding an IGF2 targeting peptide and nucleic acid encoding an alpha-glucosidase (GAA) polypeptide.
83. A nucleic acid sequence for a recombinant adenovirus associated (rAAV) vector genome comprising: (a) 5′ and 3′ AAV inverted terminal repeats (ITR) nucleic acid sequences, and (b) located between the 5′ and 3′ ITR sequence, a heterologous nucleic acid sequence encoding an alpha-glucosidase (GAA) polypeptide, or encoding a fusion polypeptide comprising a secretory signal peptide and an alpha-glucosidase (GAA) polypeptide, wherein the heterologous nucleic acid is operatively linked to a liver-specific promoter, wherein the liver specific promoter is selected from any one of:
   i. CRM_SP0412 (SEQ ID NO: 86) or SP0412 (SEQ ID NO: 91) or a functional variant or functional fragment thereof having at least 60% activity to SEQ ID NO: 86 or SEQ ID NO: 91,
   ii. SP0422 (SEQ ID NO: 92) or a functional variant or functional fragment thereof having at least 60% activity to SEQ ID NO: 92,
   iii. CRM_SP0239 (SEQ ID NO: 87) or SP0239 (SEQ ID NO: 93) or SP0238-UTR (SEQ ID NO: 147) or a functional variant or functional fragment thereof having at least 60% activity to SEQ ID NO: 87, SEQ ID NO: 93 or SEQ ID NO: 147;
   iv. CRM_SP0265 (SP0131_A1) (SEQ ID NO: 88) or SP0265 (LVR_SP0131_A1) (SEQ ID NO: 94) or SP0265-UTR (SEQ ID NO: 146) or a functional variant or functional fragment thereof having at least 60% activity to SEQ ID NO: 88, SEQ ID NO: 94 or SEQ ID NO: 146;
   v. CRM_SP0240 (SEQ ID NO: 89) or SP0240 (SEQ ID NO: 95) or SP0240-UTR (SEQ ID NO: 148) or a functional variant or functional fragment thereof having at least 60% activity to SEQ ID NO: 89, SEQ ID NO: 95 or SEQ ID NO: 148; or
   vi. CRM_SP0246 (SEQ ID NO: 90) or SP0246 (SEQ ID NO: 96) or SP0246-UTR (SEQ ID NO: 149) or a functional variant or functional fragment thereof having at least 60% activity to SEQ ID NO: 90, SEQ ID NO: 96 or SEQ ID NO: 149.
84. The nucleic acid sequence of any of paragraphs 74-83, wherein the heterologous nucleic acid sequence encoding the GAA polypeptide, or the fusion polypeptide further comprises a IGF2 targeting peptide located between the secretory signal peptide and the an alpha-glucosidase (GAA) polypeptide.
85. The nucleic acid sequence of any of paragraphs 74-84, wherein the nucleic acid encoding the secretory signal is selected from any of SEQ ID NO: 17, 22-26, 175, 177, 179 or 1810r a nucleic acid sequence at least about 75%, or 80%, or 85%, or 90%, or 95%, or 98%, or 99% sequence identity to any of SEQ ID NOs: 17 or 22-26, or 175, 177, 179 or 181.
86. The nucleic acid sequence of any of paragraphs 74-85, wherein the nucleic acid encoding the IGF2 targeting peptide is selected from any of SEQ ID NO: 2 (IGF2-Δ2-7), SEQ ID NO: 3 (IGF2-A1-7), or SEQ ID NO: 4 (IGF2 V43M), or a nucleic acid sequence at least about 75%, or 80%, or 85%, or 90%, or 95%, or 98%, or 99% sequence identity to any of SEQ ID NOs: 2, 3 or 4.
87. The nucleic acid sequence of any of paragraphs 74-86, wherein the nucleic acid sequence encoding the GAA polypeptide is the human GAA gene or a human codon optimized GAA gene (coGAA) or a modified GAA nucleic acid sequence.
88. The nucleic acid sequence of any of paragraphs 74-87, wherein the nucleic acid sequence encoding the GAA polypeptide is codon optimized for enhanced expression in vivo.
89. The nucleic acid sequence of any of paragraphs 74-88, wherein the nucleic acid sequence encoding the GAA polypeptide is codon optimized to reduce CpG islands.
90. The nucleic acid sequence of any of paragraphs 74-89, wherein the nucleic acid sequence encoding the GAA polypeptide is codon optimized to reduce the innate immune response, or reduce the CpG islands, or to reduce the innate immune response and reduce the CpG islands.
91. The nucleic acid sequence of any of paragraphs 74-90, wherein the nucleic acid sequence encoding the GAA polypeptide encodes a GAA polypeptide comprising a H201L modification of SEQ ID NO: 10.
92. The nucleic acid sequence of any of paragraphs 74-91, wherein the nucleic acid encoding the GAA polypeptide is selected from any of SEQ ID NO: 11 (full length hGAA), SEQ ID NO: 55 (Dwight cDNA), SEQ ID NO: 56 (hGAA Δ1-66) or a nucleic acid sequence at least about 75%, or 80%, or 85%, or 90%, or 95%, or 98%, or 99% sequence identity to any of SEQ ID NOs: 11, 55 or 56.
93. The nucleic acid sequence of paragraph 74-92, wherein the nucleic acid encoding the GAA polypeptide is selected from any of SEQ ID NO: 74 (codon optimized 1), SEQ ID NO: 75 (codon optimized 2), and SEQ ID NO: 76 (codon optimized 3), or a nucleic acid sequence at least about 75%, or 80%, or 85%, or 90%, or 95%, or 98%, or 99% sequence identity to any of SEQ ID NOs: 74, 75 or 76.
94. The nucleic acid sequence of paragraph 74-93, wherein the nucleic acid is selected from any of: SEQ ID NO: 57 (AAT-V43M-wtGAA (delta1-69aa)); SEQ ID NO: 58 (ratFN1-IGF2V43M-wtGAA (delta1-69aa)); SEQ ID NO: 59 (hFN1-IGF2V43M-wtGAA (delta1-69aa)); SEQ ID NO: 60 (AAT-IGF2-Δ2-7-wtGAA (delta 1-69)); SEQ ID NO: 61 (FN1rat-IGFΔ2-7-wtGAA (delta 1-69)); SEQ ID NO: 62 (hFN1-IGFΔ2-7-wtGAA (delta 1-69)), SEQ ID NO: 79 (AAT_hIGF2-V43M_wtGAA_del1-69_Stuffer.V02); SEQ ID NO: 80 (FIBrat_hIGF2-V43M_wtGAA_del1-69_Stuffer.V02); SEQ ID NO: 81 (FIBhum_hIGF2-V43M_wtGAA_del1-69_Stuffer.V02); SEQ ID NO: 82 (AAT_GILT_wtGAA_del1-69__Stuffer.V02); SEQ ID NO: 83 (FIBrat_GILT_wtGAA_del1-69_Stuffer.V02); SEQ ID NO: 84 (FIBhum_GILT_wtGAA_del1-69_Stuffer.V02) or a nucleic acid sequence having at least 80%, 85%, 90%, 95% or 98% identity to SEQ ID Nos: 57, 58, 59, 60, 61, 62, 79, 80, 81, 82, 83 or 84.
95. A method to treat a subject with a glycogen storage disease type II (GSD II, Pompe Disease, Acid Maltase Deficiency) or having a deficiency in alpha-glucosidase (GAA) polypeptide, comprising administering any of the recombinant AAV vector, or the rAAV genome or the nucleic acid sequence of any one of the previous paragraphs 1-95 to the subject.
96. The method of paragraph 95, wherein GAA polypeptide is secreted from the subject's liver and there is uptake of the secreted GAA by skeletal muscle tissue, cardiac muscle tissue, diaphragm muscle tissue or a combination thereof, wherein uptake of the secreted GAA results in a reduction in lysosomal glycogen stores in the tissue(s).
97. The method of any of paragraphs 95-96, wherein the administering to the subject is selected from any of: intramuscular, sub-cutaneous, intraspinal, intracisternal, intrathecal, intravenous administration.
98. The method of any of paragraphs 95-97, wherein the recombinant AAV vector is a chimeric AAV vector, haploid AAV vector, a hybrid AAV vector or polyploid AAV vector.
99. The method of any of paragraphs 95-97, wherein the recombinant AAV vector is a rational haploid vector, a mosaic AAV vector, a chemically modified AAV vector, or a AAV vector from any AAV serotypes.
100. The method of any of paragraphs 95-97, wherein the recombinant AAV vector is a AAVXL32 vector or a AAVXL32.1 vector or a AAV8 vector, or a haploid AAV8 vector comprising at least one AAV8 capsid protein.
101. The method of paragraph 100, wherein the recombinant AAV vector is a AAV8 vector.
102. A method to treat a subject with a lysosomal storage disease (LSD), comprising administering any of: the recombinant AAV vector, or the rAAV genome or the nucleic acid sequences of any one of the previous paragraphs to the subject, wherein the AAV vector expresses a polypeptide selected from any polypeptide in Table 5B or Table 6B.
103. The method of paragraph 102, wherein the lysosomal storage disease (LSD) is selected from any of those listed in Table 5A or Table 6A.
104. The method of paragraph 102, wherein the recombinant AAV vector is a chimeric AAV vector, haploid AAV vector, a hybrid AAV vector or polyploid AAV vector.
105. The method of paragraph 102, wherein the recombinant AAV vector is a rational haploid vector, a mosaic AAV vector, a chemically modified AAV vector, or a AAV vector from any AAV serotypes.
106. The method of paragraph 102, wherein the recombinant AAV vector is a AAVXL32 vector or a AAVXL32.1 vector or a AAV8 vector, or a haploid AAV8 vector comprising at least one AAV8 capsid protein.
107. A cell comprising the nucleic acid sequence of any of paragraphs 74-94.
108. The cell of paragraph 107, wherein the cell is a human cell.
109. The cell of any of paragraphs 107-108, wherein the cell is a non-human cell mammalian cell.
110. The cell of any of paragraphs 107-109, wherein the cell is an insect cell.
111. A cell comprising the recombinant AAV vector of any of paragraphs 1-72.
112. A host animal comprising the recombinant AAV vector of any of paragraphs 1-72.
113. The host animal of paragraph 112, wherein the host animal is a mammal.
114. The host animal of paragraph 112 or 113, wherein the host animal is a non-human mammal.
115. The host animal of paragraph 113, wherein the host animal is a human.
116. The pharmaceutical composition of paragraph 73, for use in the method of any of paragraphs 95-101.
117. A host animal comprising a cell of any of paragraphs 107-111.
118. A host animal comprising the recombinant AAV vector of any of paragraphs 1-72.
119. The host animal of paragraph 118, wherein the host animal is a mammal.
120. The host animal of paragraph 118-119, wherein the host animal is a non-human mammal.
121. The host animal of paragraph 119, wherein the host animal is a human.

### Examples

The following non-limiting examples are provided for illustrative purposes only in order to facilitate a more complete understanding of representative embodiments now contemplated. These examples are intended to be a mere subset of all possible contexts in which the AAV virions and rAAV vectors may be utilized. Thus, these examples should not be construed to limit any of the embodiments described in the present specification, including those pertaining to AAV virions and rAAV vectors and/or methods and uses thereof. Ultimately, the AAV virions and vectors may be utilized in virtually any context where gene delivery is desired.

### Example 1: Construction of the rAAV Genome

Numerous rAAV genomes were constructed using Gibson cloning methodology. The following rAAV genomes were generated: AAV_LVR412_EU (SEQ ID NO: 154), ssAAV_LVR412WT-hGAA_AskBio_CHATHAM (SEQ ID NO: 155), AAV-LVR412Stuffer (SEQ ID NO: 156), AAV_LVR422_EU (SEQ ID NO: 157), AAV-LVR422_Stuffer (SEQ ID NO: 158), ssAAV_LVR412_WT-hGAA CHATHAM (SEQ ID NO: 159), ssAAV_LSP_WT-hGAA-CHATHAM (SEQ ID NO: 160), SEQ ID NO: 57 (AAT-V43M-wtGAA (delta1-69aa)); SEQ ID NO: 58 (ratFN1-IGF2V43M-wtGAA (delta1-69aa)); SEQ ID NO: 59 (hFN1-IGF2V43M-wtGAA (delta1-69aa)); SEQ ID NO: 60 (AAT-IGF2-Δ2-7-wtGAA (delta 1-69)); SEQ ID NO: 61 (FN1rat-IGFΔ2-7-wtGAA (delta 1-69)); SEQ ID NO: 62 (hFN1-IGFΔ2-7-wtGAA (delta 1-69)). While some rAAV vectors comprises a nucleic acid sequence encoding the wtGAA polypeptide, one can readily replace the wtGAA with a nucleic acid sequence encoding a modified GAA nucleic acid sequence as disclosed herein.

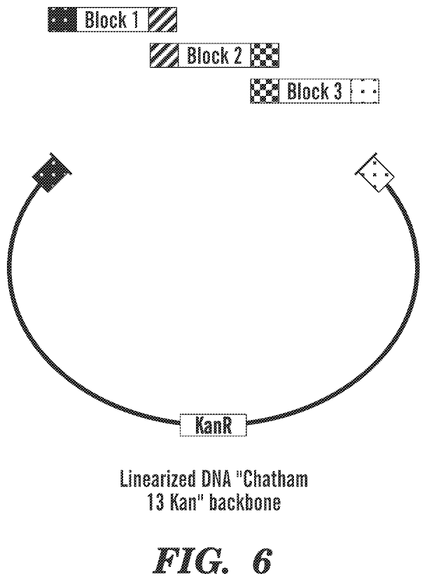

Gibson cloning involves cloning blocks (e.g., 3 blocks) of nucleic acid sequences together. The general protocol is as follows: the following reagents are combined into a single-tube reaction (i) Gibson Assembly Master Mix (Exonuclease, DNA polymerase, DNA Ligase, buffer) (ii) DNA inserts (Blocks 1-3) with 15-25 bp of homologous ends (see, FIG. 6) (iii) Linearized DNA backbone with 15-25 bp of homologous ends to the outermost DNA inserts (see, FIG. 6). The reaction is incubated at 50° C. for 15-60 minutes. The reaction mix is transformed into competent cells and plated on Kanamycin agar plates. Minipreps of fully-assembled plasmid DNA are screened via restriction digestion and/or colony PCR analysis and verified by DNA sequencing analysis. Verified clone is expanded for maxiprep production and transiently transfected in a rAAV producer cell line alongside the Adenovirus helper, XX680 Kan, and the appropriate Rep/Cap helper to produce rAAV. FIG. 6 show the cloning nucleic blocks to generate exemplary rAAV genomes.

While FIGS. 7-9 show wtGAA(A1-69) is an exemplary GAA enzyme, this nucleic acid sequence can easily be replaced by one of ordinary skill with a nucleic acid sequence encoding GAA that has been codon optimized for enhanced expression in vivo, and/or to reduce immune response, and/or to reduce CpG islands, and/or has a H201L modification. Accordingly, while FIG. 9A-9E show exemplary constructs with wild type GAA (wtGAA), one can readily replace the nucleic acid encoding the wtGAA with a nucleic acid sequence encoding a modified GAA nucleic acid sequence as disclosed herein, e.g., SEQ ID NO: 182. Also shown in the cloning blocks exemplified in FIGS. 7-8 is a generation of a rAAV genome a 3 amino acid (3aa) spacer nucleic acid sequence located 3′ of the nucleic acid sequence encoding the IGF2(V43M) or IGF2-Δ2-7 targeting peptide and 5′ of the nucleic acid encoding a GAA enzyme, and a stuffer nucleic acid sequence a stuffer sequence (referred to in FIGS. 7-8 as a “spacer” sequence) which is located 3′ of the polyA sequence and 5′ of the 3′ITR sequence.

### Example 2: Generating rAAV Vectors

The rAAV genomes were packed into capsids to generate rAAV vectors using a rAAV producing cell line. Solely for proof of principal of rAAV vector construction, the capsids used were AAV3b capsids.

Making rAAV in the rAAV producing cell line: triple transfection technique was used to make rAAV in a suspension rAAV producer cell line, which can be scaled up for making clinical grade vector. Alternatively, different plasmids can be used, e.g., 1) pXX680-ad helper and 2) pXR3 the Rep and Cap 3) and the Transgene plasmid (ITR-transgene-ITR).

The rAAV genomes generated in Example 1 are used to generate rAVV vectors using a rAAV producing cell line, according to the methods as described in U.S. Pat. No. 9,441,206, which is incorporated herein in its entirety by reference. In particular, rAAV vectors or rAAV virions are produced using a method comprising: (a) providing a rAAV producing cell line an AAV expression system; (b) culturing the cells under conditions in which AAV particles are produced; and (c) optionally isolating the AAV particles. Ratios of triple transfection of the plasmid and transfection cocktail volumes can be optimized, with varying plasmid ratios of XX680, AAV rep/cap helper and TR plasmid to determine the optimal plasmid ratio for rAAV vector production.

In some instances, the cells are cultured in suspension under conditions in which AAV particles are produced. In another embodiment, the cells are cultured in animal component-free conditions. The animal component-free medium can be any animal component-free medium (e.g., serum-free medium) compatible with the rAAV producer cell line. Examples include, without limitation, SFM4Transfx-293 (Hyclone), Ex-Cell 293 (JRH Biosciences), LC-SFM (Invitrogen), and Pro293-S(Lonza). Conditions sufficient for the replication and packaging of the AAV particles can be, e.g., the presence of AAV sequences sufficient for replication of an rAAV genome described herein and encapsidation into AAV capsids (e.g., AAV rep sequences and AAV cap sequences) and helper sequences from adenovirus and/or herpesvirus.

Bacterial DNA sequences from the plasmid backbone can be packaged into AAV capsids during manufacturing of the recombinant AAV vectors leading to activations of the innate immune system through its interaction with TLR9 (Akira, 2006; Chadeuf, 2005; Wright, 2014). Various technologies can be used to eliminate plasmid backbone sequences in recombinant AAV preparations, for example minicircles which have limited scalability (Schnodt, 2016). Another method to avoid bacterial DNA sequence in the plasmid backbone is to use closed ended linear duplex DNA, which includes a range of DNA replication technology, including but not limited to doggy bone DNA (dbDNA™) for specifically manufacturing of recombinant AAV vectors. Using closed ended linear duplex DNA, such as dbDNA™ eliminates the bacterial backbone and has been used to produce vaccines and lentivirus (Walters et al, 2014; Scott et al, 2015; Karda et al, 2019) and was shown to be unable to trigger TLR9 responses by DNA vaccine developers.

Accordingly, in alternative embodiments, generation of rAAV vectors for use in the methods and compositions as disclosed herein can be performed using closed ended linear duplex DNA, including but not limited to Doggybone technology (dbDNA™), as disclosed in US Application 2018/0037943 and Karbowniczek et al., Bioinsights, 2017, which is incorporated herein in its entirety by reference. In brief, a plasmid for AAV production using a closed ended linear duplex DNA technology can comprise the ITRs, promoter and gene of interest, e.g., GAA as disclosed herein, is flanked by a 56 bp palindromic protelomerase recognition sequence. The plasmid is denatured, and in the presence of a Phi29 DNA polymerase, and appropriate primers, Phi29 initiates rolling circle amplification (RCA), creating a double stranded cancatameric repeats of the original construct. When protelomerase is added, binding of the palindromic protelomerase recognition sequences occurs and cleavage-joining reaction occurs to result in a monomeric double stranded (ds) linear covalently closed DNA construct. Addition of common restriction enzymes remove the undesired DNA plasmid backbone sequence and digestion with exonuclease activity, resulting in dbDNA which can be size fractionated to isolate the dbDNA sequence encoding the ITRs, promoter and gene of interest. An exemplary plasmid for generation of rAAV vectors using closed ended linear duplex DNA such as dbDNA™ technology, comprises in the following 5′ to 3′ direction: 5′-protelomerase RS, 5′ITR, LSP promoter, hGAA, 3′UTR, hGH poly(A), 3′ ITR, 3′-protelomerase RS (sense strand), where the sense strand is linked to the complementary antisense strand for a stranded (ds) linear covalently closed DNA construct. The use of closed ended linear duplex DNA, e.g., doggy bone DNA (dbDNA™) as a starting material for the manufacturing of an AAV vector for use in the methods and composition as disclosed herein eliminates the bacterial backbone used to propagate the plasmid containing AAV vector with an inability for the product to trigger Toll-like receptor 9 (TLR9) responses. Use of closed ended linear duplex DNA technology for manufacturing further reduce the risk for liver enzyme elevations observed at a dose of 1.6E13 vg/kg in patients with Pompe disease.

### Example 3: Assessing rAAV Vectors

Whole Blood Clearance. FIG. 1 shows the results derived from an experiment where 3×10¹² vg/kg of different AAV serotypes (AAV3b, AAV3ST, AAV8, AAV9) were injected intravenously into 3 kg seronegative male macaques. The macaques were euthananized 60 days post administration of the different AAV serotypes. Vector genomes were searched in whole blood and results indicated that AAV3b was cleared within a week and was undetectable at sacrifice, whereas AAV8 and AAV9 were still detectable in whole blood when the macaques were sacrificed.

Liver Specific Vector Potency: FIG. 2 shows the results derived from an experiment where 3×10¹² vg/kg of different AAV serotypes (AAV3b, AAV3ST, AAV8, AAV9) were injected intravenously into 3 kg seronegative male macaques. The macaques were euthananized 60 days post administration of the different AAV serotypes. Vector genomes were quantified in each of the three lobes of the liver from each of the macaques. The limit of quantitation was 0.002 vg/dg. Based on the results presented in FIG. 2, AAV3b was found to be a potent liver vector. AAV3b is more liver specific than AAV8 and cleared from the blood more rapidly than AAV9. The AAV3ST mutant did not provide any significant beneficial affect.

### Example 4: Measuring Secretion of GAA into the Supernatant and GAA Uptake Assays

Measuring GAA in Supernatant.

Accordingly, the rAAV genomes generated in Example 1 are tested for secretion of GAA polypeptide into the supernatant. Measurement of GAA in the supernatant can be assessed using a 4-methyl-umbelliferyl-alpha-D-glucoside (4-MU) substrate (4-MU assay), as described in Kikuchi et al. (Kikuchi, Tateki, et al. “Clinical and metabolic correction of Pompe disease by enzyme therapy in acid maltase-deficient quail.” The Journal of clinical investigation 101.4 (1998): 827-833.).

In brief, a rAAV producer cell line can be transfected with rAAV genomes AAV_LVR412_EU (SEQ ID NO: 154), ssAAV_LVR412WT-hGAA_AskBio_CHATHAM (SEQ ID NO: 155), AAV-LVR412Stuffer (SEQ ID NO: 156), AAV_LVR422_EU (SEQ ID NO: 157), AAV-LVR422_Stuffer (SEQ ID NO: 158), ssAAV_LVR412_WT-hGAA CHATHAM (SEQ ID NO: 159), ssAAV_LSP_WT-hGAA-CHATHAM (SEQ ID NO: 160), SEQ ID NO: 57 (AAT-V43M-wtGAA (delta1-69aa)); SEQ ID NO: 58 (ratFN1-IGF2V43M-wtGAA (delta1-69aa)); SEQ ID NO: 59 (hFN1-IGF2V43M-wtGAA (delta1-69aa)); SEQ ID NO: 60 (AAT-IGF2-Δ2-7-wtGAA (delta 1-69)); SEQ ID NO: 61 (FN1rat-IGFΔ2-7-wtGAA (delta 1-69)); SEQ ID NO: 62 (hFN1-IGFΔ2-7-wtGAA (delta 1-69)). GAA activity is measured based on the % of initial activity (t=0) over 24 hours. Samples were assayed for GAA enzyme activity based on the hydrolysis of the fluorogenic substrate 4-MU-α-glucose at 0, 3, 6 and 24 hours. The GAA activity was expressed as % of initial activity, i.e. residual activity.

Alternatively, after harvest, culture supernatants were partially purified by HIC chromatography. All samples were treated with PNGase prior to electrophoresis. The expression of GAA polypeptides by the cells can be assessed using SDS-PAGE and immunoblotting.

GAA Uptake Assays & Measuring Uptake of GAA in Tissues.

Next, the rAAV genomes generated in Examples 1 and 2 are tested for retention of uptake activity into cells. For example, a rAAV producer cell line can be transfected with rAAV genomes AAV_LVR412_EU (SEQ ID NO: 154), ssAAV_LVR412WT-hGAA_AskBio_CHATHAM (SEQ ID NO: 155), AAV-LVR412Stuffer (SEQ ID NO: 156), AAV_LVR422_EU (SEQ ID NO: 157), AAV-LVR422_Stuffer (SEQ ID NO: 158), ssAAV_LVR412_WT-hGAA CHATHAM (SEQ ID NO: 159), ssAAV_LSP_WT-hGAA-CHATHAM (SEQ ID NO: 160), SEQ ID NO: 57 (AAT-V43M-wtGAA (delta1-69aa)); SEQ ID NO: 58 (ratFN1-IGF2V43M-wtGAA (delta1-69aa)); SEQ ID NO: 59 (hFN1-IGF2V43M-wtGAA (delta1-69aa)); SEQ ID NO: 60 (AAT-IGF2-Δ2-7-wtGAA (delta 1-69)); SEQ ID NO: 61 (FN1rat-IGFΔ2-7-wtGAA (delta 1-69)); SEQ ID NO: 62 (hFN1-IGFΔ2-7-wtGAA (delta 1-69)).

A 4-MU assay (as described above) can be to assess uptake of rhGAA into mammalian cells is described in US patent Application US2009/0117091A1, which is incorporated herein in its entirety by reference. rAAV vectors or rAAV genomes generated in Examples 1 and 2 are incubated in 20 μl reaction mixtures containing 123 mM sodium acetate pH 4.0 with 10 mM 4-methylumbelliferyl α-D-glucosidase substrate (Sigma, catalog #M-9766). Reactions were incubated at 37° C. for 1 hour and stopped with 200 μl of buffer containing 267 mM sodium carbonate, 427 mM glycine, pH 10.7. Fluorescence was measured with 355 nm excitation and 460 nm filters in 96-well microtiter plates and compared to standard curves derived from 4-methylumbelliferone (Sigma, catalog #M1381). 1 GAA 4 MU unit is defined as 1 nmole 4-methylumbelliferone hydrolyzed/hour. Specific activities of exemplary rAAV genomes in fibroblast cells are assessed, e.g., AAV_LVR412_EU (SEQ ID NO: 154), ssAAV_LVR412WT-hGAA_AskBio_CHATHAM (SEQ ID NO: 155), AAV-LVR412Stuffer (SEQ ID NO: 156), AAV_LVR422_EU (SEQ ID NO: 157), AAV-LVR422_Stuffer (SEQ ID NO: 158), ssAAV_LVR412_WT-hGAA CHATHAM (SEQ ID NO: 159), ssAAV_LSP_WT-hGAA-CHATHAM (SEQ ID NO: 160), SEQ ID NO: 57 (AAT-V43M-wtGAA (delta1-69aa)); SEQ ID NO: 58 (ratFN1-IGF2V43M-wtGAA (delta1-69aa)); SEQ ID NO: 59 (hFN1-IGF2V43M-wtGAA (delta1-69aa)); SEQ ID NO: 60 (AAT-IGF2-Δ2-7-wtGAA (delta 1-69)); SEQ ID NO: 61 (FN1rat-IGFΔ2-7-wtGAA (delta 1-69)); SEQ ID NO: 62 (hFN1-IGFΔ2-7-wtGAA (delta 1-69)). The enzymatic activity of IGF2-GAA fusion polypeptides and/or SS-IGF2-GAA double fusion polypeptide are assessed and compared to a GAA (wtGAA) polypeptide by itself (i.e., without a heterologous signal peptide or IGF2 targeting peptide).

Cell-based uptake assays can also be performed to demonstrate the ability of IGF2-tagged or untagged GAA to enter the target cell. Rat L6 myoblasts are plated at a density of 1×105 cells per well in 24-well plates 24 hours prior to uptake. At the start of the experiment, media is removed from the cells and replaced with 0.5 ml of uptake media which contains the rAAV vectors generated in Examples 1 and 2. In order to demonstrate specificity of uptake, some wells additionally contained the competitors M6P (5 mM final concentration) and/or IGF2 (18 μg/ml final concentration). After 18 hours, media is aspirated off of cells, and cells are washed 4 times with PBS. Then, cells are lysed with 200 μl CelLytic MTM lysis buffer. The lysate is assayed for GAA activity as described above using the 4 MU substrate. Protein is determined using the Pierce BCA™ Protein Assay Kit.

A typical uptake experiment is performed in CHO cells, although other cell lines and myoblast cell lines can be used. It is expected that uptake of the GAA polypeptides into Rat L6 myoblasts will be virtually unaffected by the addition of a large molar excess of M6P, whereas uptake is expected to be significantly abolished by excess IGF2. In contrast, it is expected that uptake of wtGAA to be significantly abolished by addition of excess M6P but virtually unaffected by competition with IGF2. In addition, it is expected that uptake of IGF2V43M-wtGAA and IGFdelta2-7wtGAA will be unaffected significantly by excess IGF2.

### Example 5 Half-Life of GAA in Rat L6 Myoblasts

An uptake experiment was performed as described above (see Example 3 & 4) with the rAAV vectors produced in Example 1 and 2 in rat L6 myoblasts. After 18 hours, media from cells transfected with the rAAV vectors was aspirated off and the cells were washed 4 times with PBS. At this time, duplicate wells were lysed (Time 0) and lysates were frozen at −80. Each day thereafter, duplicate wells were lysed and stored for analysis. After 14 days, all of the lysates were assayed for GAA activity, to assess the half-lives, and assess if, once inside cells, the IGF2-tagged GAA enzyme persists with similar kinetics to untagged GAA.

### Example 6: Processing of GAA after Uptake

Mammalian GAA typically undergoes sequential proteolytic processing in the lysosome as described by Moreland et al. (2005) J. Biol. Chem., 280:6780-6791 and references contained therein. The processed protein gives rise to a pattern of peptides of 70 kDa, 20 kDa, 10 kDa and some smaller peptides. To determine whether IGF2-GAA fusion polypeptide and/or SS-IGF2-GAA double fusion polypeptide is processed similarly to the untagged GAA, aliquots of lysates from the above uptake experiment were analyzed by Western blot using a monoclonal antibody that recognizes the 70 kDa IGF2 peptide and larger intermediates with the IGF2 tag. A similar profile of polypeptides identified in this experiment indicates that once entering the cell, the IGF2 targeting peptide is lost and the IGF2-GAA polypeptide is processed similarly to untagged GAA, which demonstrates that the IGF2 targeting peptide has little or no impact on the behavior of GAA once it is inside the cell.

### Example 7: Pharmacokinetics

Pharmacokinetics of IGF2-GAA fusion polypeptide and/or SS-IGF2-GAA double fusion polypeptide produced by the rAAV vectors can be measured in wild-type 129 mice. 129 mice are injected with the rAAV vectors generated in Example 1 and 2. Serum samples are taken preinjection and at 15 min, 30 min, 45 min, 60 min, 90 min, 120 min, 4 hours, and 8 hours post injection. The animals are then sacrificed. Serum samples are assayed by quantitative western blot. The half-lives for the GAA from rAAV vectors expressing IGF2-GAA fusion polypeptide or SS-IGF2-GAA double fusion polypeptide are assessed to determine if the IGF2 fused GAA polypeptide is cleared from the circulation excessively rapidly.

### Example 8: Tissue Half-Life of GAA

The objective of this experiment was to determine the rate at which GAA activity is lost once the IGF2-GAA fusion polypeptide or SS-IGF2-GAA double fusion polypeptide expressed from the rAAV vector reaches its target tissue. In the Pompe mouse model, MYOZYME® appears to have a tissue half-life of about 6-7 days in various muscle tissues (Application Number 125141/0 to the Center for Drug Evaluation and Research and Center for Biologics Evaluation and Research, Pharmacology Reviews).

Pompe mice (Pompe mouse model 6neo/6neo as described in Raben (1998) JBC, 273:19086-19092, the disclosure of which is hereby incorporated by reference) are injected in the jugular vein with the rAAV vectors generated in Examples 1 and 2. Mice are then sacrificed at 1, 5, 10, and 15 days post injection. Tissue samples were homogenized and GAA activity measured according to standard procedures. The tissue half-life of GAA activity from IGF2-GAA fusion polypeptide and/or SS-IGF2-GAA double fusion polypeptide and the untagged GAA are calculated from the decay curves in different tissues (e.g., quadriceps tissue; heart tissue; diaphragm tissue; and liver tissue), and the half-life in each tissue calculated. This can be compared to the half-life in rat L6 myoblasts to determine if, once inside cells in Pompe mice, IGF2-GAA fusion polypeptide and/or SS-IGF2-GAA double fusion polypeptide expressed from the rAAV vectors described herein appears to persist with kinetics similar to the untagged GAA. Furthermore, the knowledge of the decay kinetics of the IGF2-GAA fusion polypeptide and/or SS-IGF2-GAA double fusion polypeptide can help in the design of appropriate dosing intervals.

### Example 9: Uptake of IGF2-GAA Fusion Polypeptide and/or SS-IGF2-GAA Double Fusion Polypeptide into Lysosomes of C2C12 Mouse Myoblasts

C2C12 mouse myoblasts grown on poly-lysine coated slides (BD Biosciences) are transduced with the rAAV vectors produced in Examples 1 and 2. After washing the cells, the cells are then incubated in growth media for 1 hour, then washed four times with D-PBS before fixing with methanol at room temperature for 15 minutes. The following incubations were all at room temperature, each separated by three washes in D-PBS. Slides are permeabilized with 0.1% triton X-100 for 15 minutes, then blocked with blocking buffer (10% heat-inactivated horse serum (Invitrogen) in D-PBS). Slides are incubated with primary mouse monoclonal anti-GAA antibody 3A6-1F2 (1:5,000 in blocking buffer), then with secondary rabbit anti-mouse IgG AF594 conjugated antibody (Invitrogen A11032, 1:200 in blocking buffer). A FITC-conjugated rat anti-mouse LAMP-1 (BD Pharmingen 553793, 1:50 in blocking buffer) is the incubated. Slides are mounted with DAPI-containing mounting solution (Invitrogen) and viewed with a Nikon Eclipse 80i microscope equipped with fluorescein isothiocyanate, texas red and DAPI filters (Chroma Technology). Images can be captured with a photometric Cascade camera controlled by MetaMorph software (Universal Imaging), and merged using Photoshop software (Adobe). Co-localization of signal detected by anti-GAA antibody with signal detected by antibody directed against a lysosomal marker, LAMP1 can be assessed to demonstrates that IGF2-tagged GAA is delivered to lysosomes.

### Example 10: Assessing the Treatment of the rAAV Vectors in a Pompe Mouse Model and Reversing Pompe Pathology

The rAAV vectors generated in Example 1 can be assessed in Pompe mouse mode, e.g., according to the methods described in Peng et al., “Reveglucosidase alfa (BMN 701), an IGF2-Tagged rhAcid α-Glucosidase, Improves Respiratory Functional Parameters in a Murine Model of Pompe Disease.” Journal of Pharmacology and Experimental Therapeutics 360.2 (2017): 313-323), which is incorporated herein in its entirety by reference.

Any Pompe mouse model can be used to assess the effect of the rAAV vectors at treating Pomoe disease. One mouse model of Pompe is described in Raben et al., JBC, 1998; 273(30); 19086-19092, which describes a disrupted GAA mouse model, and recapitulates critical features of both the infantile and the adult forms of the disease. In other instances, a Pompe mouse model (Sidman et al., 2008) can be used, as well as a strain of mice with a disrupted acid α-glucosidase gene (B6;129-GAAtm1Rabn/J; Pompe) (Jackson Laboratory, Bar Harbor, Me.). The Pompe mice develop the same cellular and clinical characteristics as in human adult Pompe disease (Raben et al., 1998). Animals are maintained in a 12-hour light/dark cycle, provided with fresh water and standard rodent chow ad libitum.

4.5-5 month old Pompe mice can be administered the rAAV vectors described herein, and evaluated for glycogen clearance after administration for 4 or more weeks. Following a macroscopic assessment, the heart (left ventricle), quadriceps, diaphragm, psoas, and soleus muscles were collected, weighed, snap-frozen in liquid nitrogen, and stored at −60 to −90° C. prior to a quantitative analysis of glycogen-derived glucose. Muscles were homogenized in buffer (0.2 M NaOAc/0.5% NP40) on ice using ceramic spheres. Amyloglucosidase was added to clarified lysates at 37° C. to digest glycogen into glucose for subsequent colorimetric detection (430 nm, SpectraMax; Molecular Devices, Sunnyvale, Calif.) using a peroxidase-glucose oxidase enzyme reaction system (Sigma-Aldrich, St. Louis, Mo.). Paired samples are also measured without amyloglucosidase to correct for endogenous tissue glucose that was not in glycogen form at harvest. Glucose values were extrapolated from a six-point standard curve. The measured glucose concentration (mg/ml) is proportional to the glycogen concentration of the sample and is converted to mg glycogen/g tissue by adjusting for the homogenization step (5 μl buffer added per gram of tissue).

The effect of rAAV vectors described herein on individual mouse muscle glycogen levels can be evaluated using Phoenix-WinNonlin classic PD modeling (Phoenix build version 6.4; Certara, L. P., Princeton, N.J.). Results can be obtained for hGAA in heart, diaphragm, quadriceps, psoas, and soleus muscles. For pharmacokinetic analysis, WT mice can be administered the rAAV vectors generated in Example 1 and blood samples collected as terminal cardiac punctures at predose, 0.083, 0.5, 1, 2, and 4 hours postdose. Plasma hGAA concentrations can be quantified using a bridging electrochemiluminescent method with an LOQ of 100 ng/ml. Briefly, 0.5 μg/ml ruthenium-labeled anti-rhGAA (affinity purified goat polyclonal) and 0.5 μg/ml biotin-labeled anti-IGF2 (MAB792; R&D Systems, Minneapolis, Minn.) can be combined with K2EDTA plasma samples diluted 1:10 in buffer [Starting Block T20 (PBS); ThermoFisher Scientific, Sunnyvale, Calif.] and incubated for 1 hour before transfer to a blocked streptavidin assay plate (Meso Scale Diagnostics, Rockville, Md.). After a 30-minute incubation, the plate is washed, 1× Read Buffer T (Meso Scale Diagnostics) was added, and the electrochemiluminescent signal read on an SECTOR Imager 2400 (Meso Scale Diagnostics). hGAA concentrations can be extrapolated from a standard curve.

Alternatively, Heart and Diaphragm tissue homogenates can be harvested and rhGAA activity measured using the fluorogenic substrate (4-MUG).

The therapeutic effect of the GAA polypeptide produced using rAAV vectors generated in Examples 1 and 2 herein can be compared wt GAA in vivo. A study can be performed to compare the ability of a rAAV vector disclosed in Example 1 to that expressing a non-tagged wt GAA to clear glycogen from skeletal muscle tissue in Pompe mice (e.g., Pompe mouse model 6neo/6neo animals were used (Raben (1998) JBC 273:19086-19092)). Groups of Pompe mice (5/group) received IV injections of one of two doses of wt GAA or a rAAV vector generated in Example 1 or vehicle. Five untreated animals can be used as control, and receive four weekly injection of saline solution. Animals receive oral diphenhydromine, 5 mg/kg one hour prior to injections 2, 3, and 4. Mice were sacrificed one week after the injection, and tissues (diaphragm, heart, lung, liver, soleus, quadriceps, gastrocnemius, TA, EDL, tongue) are harvested for histological and biochemical analysis. Glycogen content in the tissue homogenates can be measured using A. niger amyloglucosidase and the Amplex Red Glucose assay kit, and GAA enzyme levels assessed in different tissue homogenates using standard procedures.

Glycogen content in tissue homogenates can be measured using A. niger amyloglucosidase and the Amplex® Red Glucose assay kit (Invitrogen) essentially as described by Zhu et al. (2005) Biochem J., 389:619-628.

It is expected that the rAAV vector ss-IGF2-GAA rAAV as described herein and produced by the methods of Examples 1 and 2 will have more secretion followed by uptake into muscle and greater therapeutic effect in the Pompe mouse model as compared to a IGF2-GAA rAAV (i.e., without the secretory signal sequence), which is expected to be greater than wtGAA rAAV vector (i.e., without either of the heterologous secretory signal and the IGF2 targeting peptide), and/or MYOZYME®. It is further expected that the rAAV vector comprising modified GAA as disclosed herein e.g., comprising GAA with H201L mutation is therapeutically more effective in the Pompe mouse model than the rAAV comprising unmodified GAA e.g., wtGAA. Given the established Pompe model, these results are expected to translate into the clinic and correlate with therapeutic effect for the treatment of Pompe disease.

### Example 11: Clearance of Glycogen In Vivo

The objective of this experiment is to determine the rate at which glycogen is cleared from heart tissue of Pompe mice after a single injection of rAAV vector expressing IGF2-GAA fusion polypeptide and/or SS-IGF2-GAA double fusion polypeptide produced in Examples 1 and 2.

Pompe mice (Pompe mouse model 6neo/6neo as described in Raben (1998) JBC, 273:19086-19092, the disclosure of which is hereby incorporated by reference) are injected in the jugular vein with a rAAV vector expressing produced in Examples 1 and 2. Mice were sacrificed at 1, 5, 10, and 15 days post injection. Heart tissue samples are homogenized according to standard procedures and analyzed for glycogen content. Glycogen content in these tissue homogenates is measured using A. niger amyloglucosidase and the Amplex® Red Glucose assay kit (Invitrogen) essentially as described by Zhu et al. (2005) Biochem J., 389:619-628. Assessment of the heart tissue from mice can determine if there is almost complete clearance of glycogen in the mice administered rAAV vector expressing IGF2-GAA fusion polypeptide and/or SS-IGF2-GAA double fusion polypeptide produced in Examples 1 and 2 as compared to mice administered a rAAV where GAA was not fused to a IGF2 targeting peptide and/or SS as described herein, where only a small change in glycogen content would indicate minimal clearance.

### Example 12: Exemplary Liver Specific Promoters

Exemplary liver-specific promoters selected from Table 4 herein, or selected from those disclosed in Tables 4 herein, were cloned upstream of the luciferase reporter gene followed by SV40 late PolyA signal into a vector with a backbone having properties essentially identical to pUC19. In particular, experiments assessing promoters SP0412 and SP0422 were cloned into constructs for generation of rAAV. DNA preparations of the plasmids were transfected into either Huh7 (a hepato-cellular carcinoma cell line), HeLa (an immortal cell line derived from cervical cancer) or HEK293 (human embryonic kidney cells) to assess transcriptional activity. Huh-7 cells were sourced from JCRB Cell Bank (JCRB0403), HeLa and HEK293 were sourced from ECACC cell bank. All cell lines were grown and maintained according to the cell banks' recommendations.

Transfections were performed in 48 well plates in triplicate using FuGene HD Transfection Reagent (Promega #E2311) at a DNA:FuGene HD ratio of 1:1.1. Luciferase activity was measured 24 hours after transfection. Cells were washed with phosphate buffered saline (PBS), lysed in 100 μl Passive Lysis Buffer (Promega #E194A) and stored at −80° C. overnight. Luciferase activity was quantified using the Luciferase Reporter 1000 assay system (Promega #E4550) following manufacturer's guidelines in 10 μl of lysate using 96 well flat bottom solid white Microplate FluoroNunc plates (ThermoFisher #236105) and luminescence quantified in a FLUOstar Omega plate reader (BMG Labtech) machine.

The above luciferase methods are conventional in the art, and similar techniques have been described extensively in the literature, e.g. in Alam and Cook, “Reporter Genes: Application to the Study of Mammalian Gene Transcription”, ANALYTICAL BIOCHEMISTRY 188, 245-254 (1990).

Data

The sequences of exemplary liver-specific promoters used are shown in Tables 4 herein. The ability of these synthetic liver-specific promoters to drive expression in liver cells was benchmarked against the ubiquitous CMV_IE and CBA promoters, and also against the known liver specific promoter LP1. All of the synthetic promoters according to the invention showed higher activity than the LP1 promoter in Huh7 cells (data not shown). When these promoters were counter-screened in non-liver-derived HEK293 and HeLa cells, they showed negligible activity compared to the ubiquitously active promoters CMV_IE and CBA (data not shown).

The activity of the liver-specific promoters (i.e. all of the promoters set out in Tables 4) were also tested in Huh7 cells using the materials and methods essentially as described in above. However, in this case the activity of the liver-specific promoters was compared to the activity of the promoter TBG (SEQ ID NO: 435), as TBG was found to have higher and more consistent in vitro expression than LP1. It should be noted that TBG is an extremely powerful liver-specific promoter, and thus a promoter which shows expression which is less than TBG may still be extremely useful. In particular, liver-specific promoters disclosed in Table 4, or functional variants thereof, which are shorter than TBG, but which still demonstrate high levels of activity (e.g. 15%, more preferably 25%, 50%, or 75% of the activity of TBG of SEQ ID NO: 435 or higher) are of particular interest.

The specificity of the liver-specific promoters for liver cells was also tested using non-liver HEK293 cells, using the materials and methods described in Example 2. The activity of the liver-specific promoters is expressed compared to the activity of CMV-IE (SEQ ID NO: 433) (TBG and LP1 are liver-specific and thus not particularly active in HEK293 cells). ‘Relative activity’ in the graphs showing the specificity of the liver-specific promoters tested in HEK293 cells is the activity of the named promoter expressed as a ratio to the activity of CMV-IE, wherein 1 is the same activity as the CMV-IE promoter (SEQ ID NO: 433), more than 1 is higher activity compared to CMV-IE and lower than 1 is lower activity compared to the CMV-IE promoter of SEQ ID NO: 433.

### Example 13: Expression from Exemplary Liver Specific Promoters In Vivo

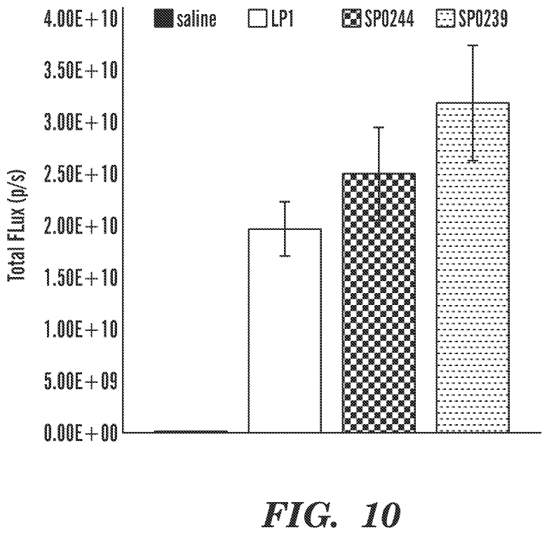

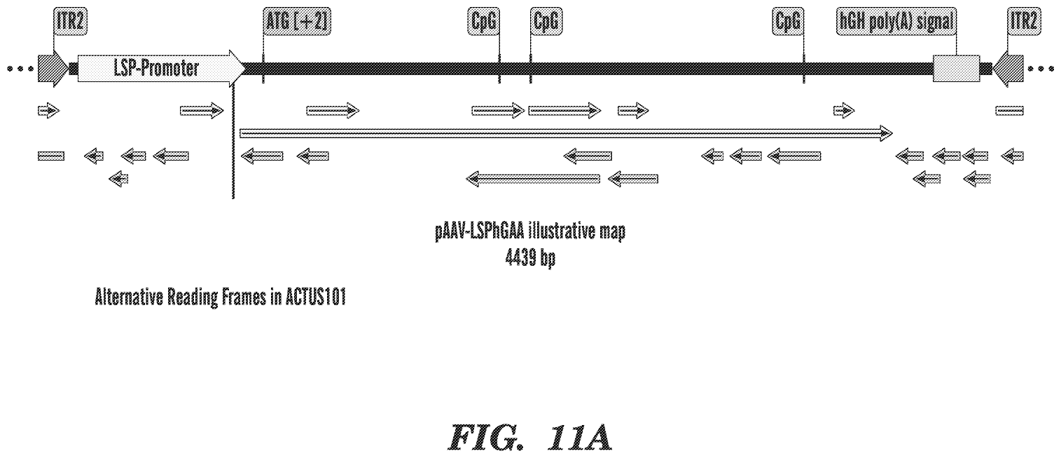

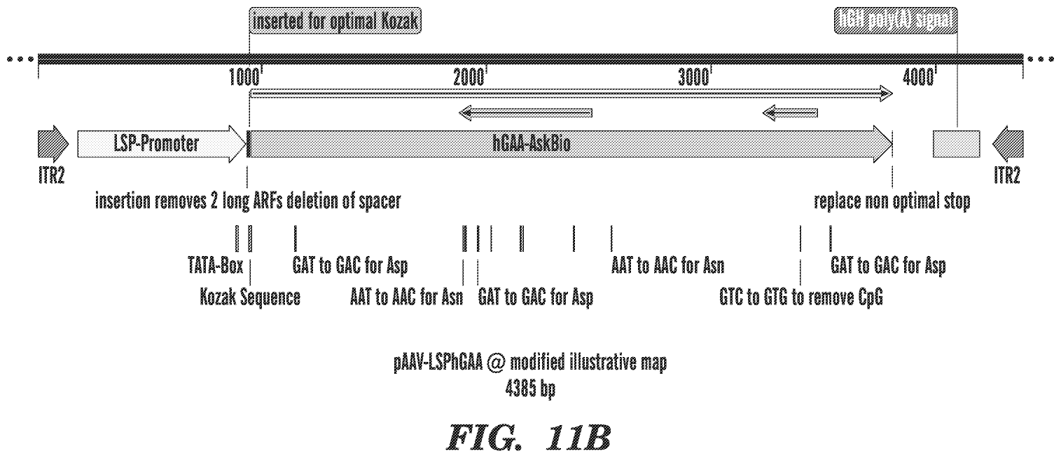

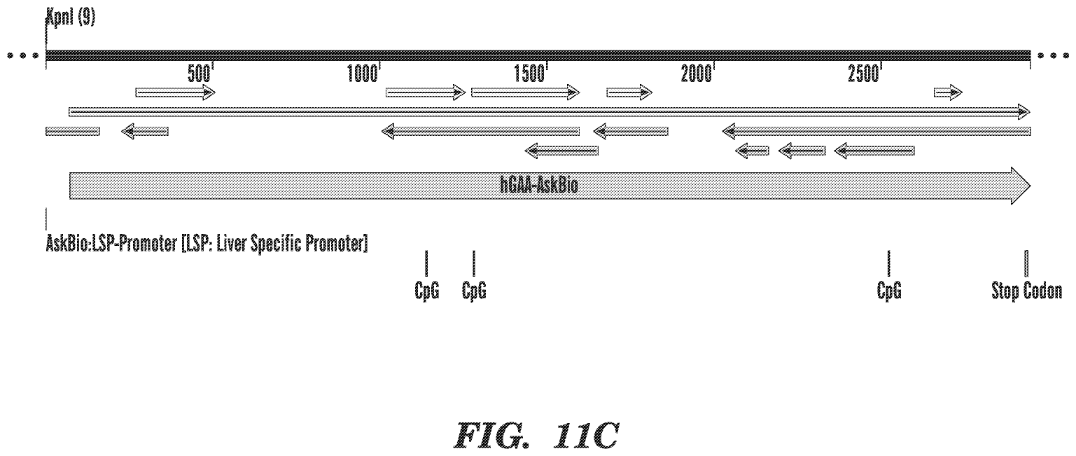

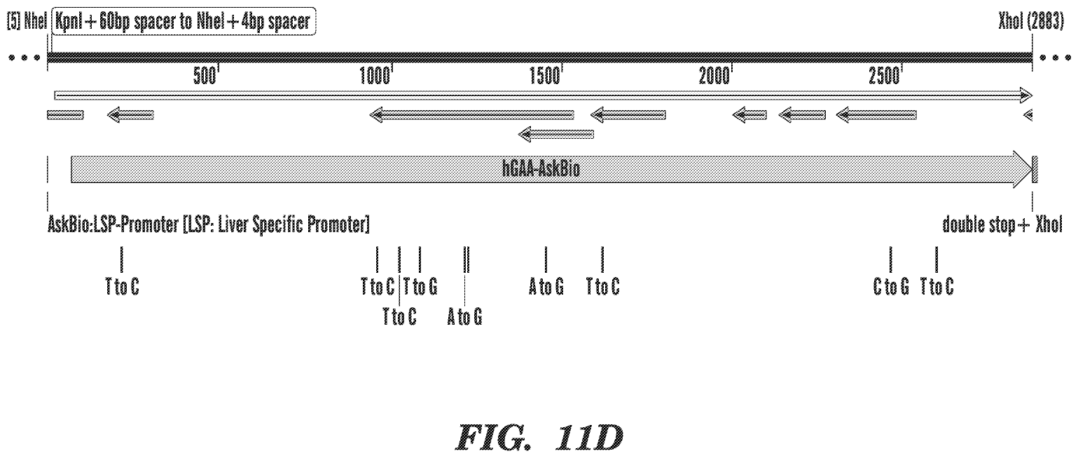

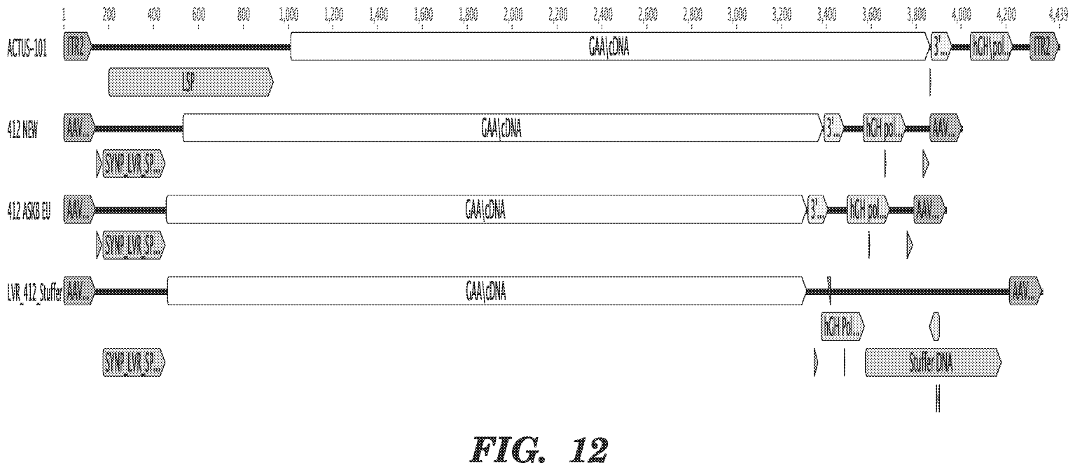

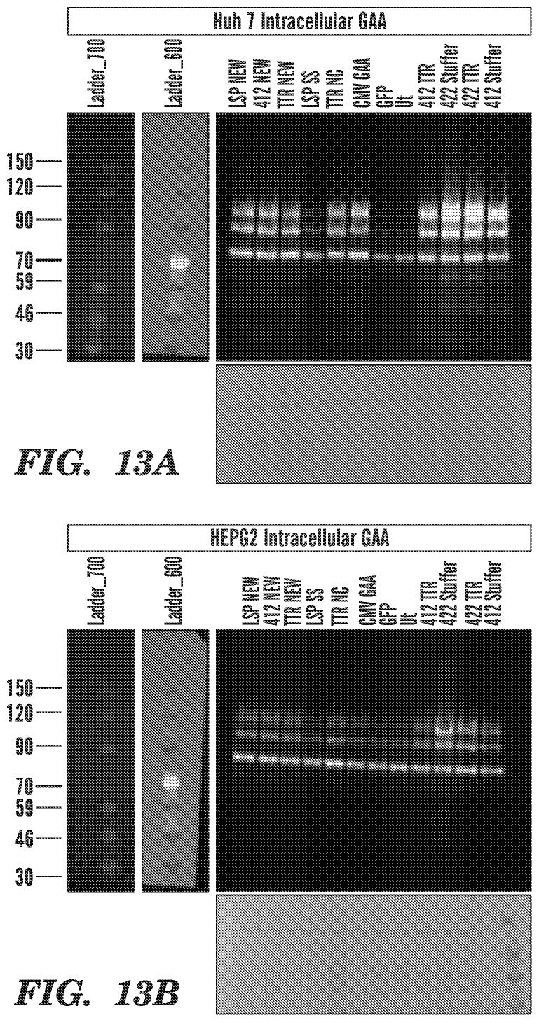

Certain liver-specific promoters were selected as exemplary liver-specific promoters for assessment in vivo. In particular, minimal promoter CRM_SP0239 (SEQ ID NO: 87) and CRM-_SP0412 (SEQ ID NO: 86) and synthetic liver-specific promoters SP0239 (SEQ ID NO: 93), SP0412 (SEQ ID NO: 91) and SP0422 (SEQ ID NO: 92) were assessed in vivo (see FIG. 10 and FIG. 13A-13B).

Aav Production:

The activity of a subset of the promoters according to this invention were tested in vivo. The synthetic promoters included in this study were SP0239, SP0244, SP0412 and the positive control LP1. The reporter gene used was fLUC-T2A-EGFP, i.e. fLUC (firefly Luciferase) fused to mEGFP (mutant green fluorescent protein) via T2A signal (two-way self-cleaving peptides). pAAV_SYNP_Luc-T2A-GFP destination vector is derived from pAAV ZsGreen1 (purchased from Clontech) in which the ZsGreen1 reporter was replaced by the Luc-T2A-GFP dual reporter. All DNA plasmids were prepared using QIAGEN Plasmid Mega Kit (Qiagen #12181, Germany) according to manufacturer instructions.

A rAAV producer cell line was cultured in Culture dish, Tissue culture treated, 145 mm (Greiner Bio-One Ltd #639160, UK) in Dulbecco's modified Eagle's medium, high glucose, GlutaMAX supplement (Gibco (Life Technologies) #61965-059, UK) supplemented with 10% (v/v) fetal bovine serum (Sigma #F7524,UK), and incubated at 37° C. and 5% CO₂. Other reagents for cell culture were purchased from Invitrogen-UK and plasticware form Life Technologies.

All AAV vectors that were used in this study were pseudotyped in AAV9 capsid. A rAAV producer cell line was co-transfected with plasmids wherein the reporter gene was controlled by different promoters alongside a plasmid encoding the helper functions to allow virus propagation (pDG9). The rAAV producer cell line were transfected using Polyethylenimine (PEI) (Sigma-Aldrich #764604, UK) at stock concentration of (1ug/ul) using molar ratio of 1:3 (DNA:PEI).

AAV Purification and Titration:

After 72 hr of transfection, cells were lysed and crude lysate was filtered then purified by HPLC columns containing POROS™ CaptureSelect™ AAV Resin (Thermo Scientific, #A36739) and using AKTAprime plus (High performance liquid chromatography-HPLC system (GE Healthcare, #11001313).

The number of vector genomes was determined by qPCR titration to target LUC cassettes with forward primer (ACGCTGGGCTACTTGATC—SEQ ID NO: 445), reverse primer (CGAGGAGGAGCTATTCTTG—SEQ ID NO: 446) and probe (TTTCGGGTCGTGCTCATG—SEQ ID NO: 447) following manufacturer instructions of Luna® Universal qPCR Master Mix (NEB #M3003, UK) in QuantStudio™ 3 System Real-Time PCR (Thermo Fisher Scientific, UK) and data analysed by QuantStudio design and analysis software V1.4.1.

Animal Procedures:

Outbred 6 weeks old CD1 male mice were purchased from Charles River-UK. They were kept in quarantine for one week and then moved to their closed ventilation cages and maintained in minimal-disease facilities. They were caged at 5 mice/cage and normalized into their weights with food and water ad libitum. Newly housed mice were given another week for acclimatization before carrying out any experiments. This study was conducted under statutory Home Office recommendation; regulatory, ethics, and licensing procedures; and the Animals (Scientific Procedures) Act 1986 and following the institutional guidelines at University College London.

Animal Injections:

AAV was administered to 8-week-old young adult male CD1 mice anaesthetised with 2%-4% isoflurane supplied in medical air (21% oxygen) (Abbotts Laboratories, UK) in warm chamber (Thermo Fisher Scientific, UK). The mice were injected intravenously into lateral tail vein using an Insulin syringe: 27 G 1/2 in., 1.0 ml (Fisher Scientific, UK). Each mouse is injected with AAV vectors dose of 8E+10 AAV viral genome per mouse in a final volume of 200 μl of physiological saline solution. The mice were then allowed to return to normal temperature before placing them back into their cages.

Bioluminescence Imaging

Mice were subjected to weekly whole-body bioluminescence imaging. Where appropriate, mice were anaesthetised with 2%-4% isoflurane supplied in medical air (21% oxygen) and received an intraperitoneal injection of 300 μl of 15 mg/mL of D-luciferin potassium salt (Syd Labs #MB000102, US) using an Insulin syringe (Fisher Scientific, UK). D-luciferin stock was prepared in physiological saline (Gibco #14190-094, UK). Mice were imaged after 5 minutes using a cooled charged-coupled device camera, (IVIS Lumina II machine, Perkin Elmer, UK) for between 1 second and 10 seconds. The regions of interest (ROI) were measured using IVIS Lumina Living image 4.5.5 (Perkin Elmer) and expressed as photons per second per centimetre squared per steradian (photons/second/cm²/sr).

Data

The results of this study are shown in FIG. 10. The results are expressed as the mean of the luciferase bioluminescence intensity, total flux (in photons per second), for all tested animals in each group. Error bars are standard error of the mean. In group ‘Saline’ (n=10), the animals were injected with saline only and no luciferase bioluminescence is detected. This group is a negative control and indicates that no luciferase bioluminescence is detected if no luciferase operably linked to a promoter is injected. In group ‘LP1’ (n=9), the animals were injected with luciferase operably linked to the LP1 promoter (SEQ ID NO: 432) and luciferase bioluminescence is detected. This group is a positive control and indicates that luciferase is expressed under the control of the LP1 promoter and can be detected.

To test the activity of the liver-specific promoters according to this invention, animals were injected with a construct comprising luciferase under operably linked to two promoters. In group ‘SP0244’ (n=8), luciferase is operably linked to the SP0244 promoter. In group ‘SP0239’ (n=10), luciferase is operably linked to the SP0239 promoter (SEQ ID NO: 93).

As can be seen from FIG. 10, expression was highest from promoters ‘SP0244’ (SEQ ID NO: 366) and ‘SP0239’ (SEQ ID NO: 93), as shown by higher bioluminescence intensity than the generic liver specific promoter ‘LP1’ (SEQ ID NO: 432). Accordingly, promoters SP0244 and SP0239 show high activity in vivo and their activity is higher than the activity of LP 1.

In FIGS. 13A and 13B, the expression of GAA was detected from different rAAV vectors comprising different LSP; LSP NEW (SEQ ID NO: 160); 412 NEW (SEQ ID NO: 159); TTR NEW (SEQ ID NO: 155); 412-TTR, 422 Stuffer (SEQ ID NO: 158); 422 TTR; 412 Stuffer (SEQ ID NO: 156) intracellularly in Huh7 cells and HEPH2 cells, demonstrating that hGAA can be expressed from rAAV at significant levels using SP0412 and SP0422 promoter. Furthermore, as shown in FIGS. 13A and 13B, the GAA expression by any of the above AAV vectors is significantly higher than the expression of GAA from an AAV vector comprising a generic liver specific promoter. Importantly, FIGS. 13A and 13B show that expression of hGAA from rAAV vectors comprising promoters 412 (SEQ ID NO: 91) and 422 (SEQ ID NO: 92) leads to high expression of hGAA in Huh7 and HepG2 cells, as compared to the level of hGAA expression from a rAAV comprising the LP1 promoter (SEQ ID NO: 432) which is referred to as “LSP SS” in FIGS. 13A and 13B.

This experiment demonstrates confirms that the in vitro results obtained in Example 12 can be achieved with significant GAA protein expression in vivo. A range of liver-specific promoters with varying strength can be used in the methods and compositions disclosed herein, which can be very useful to provide the desired level of liver-specific expression in a therapeutic setting for the treatment of a disease, e.g., Pompe disease.

### Example 14: Clinical Study of Pompe Patients

Two cohorts of 3 subjects each have been dosed with rAAV-LSP-hGAA in clinical study ACT-CS101, which recruited adult subjects with LOPD. Cohort 1 subjects received 1.6E12 vg/kg and Cohort 2 subjects received 1.6E13 vg/kg. All subjects have been withdrawn from enzyme replacement therapy (ERT) (alglucosidase alfa [LUMIZYME®]); Cohort 1 subjects remained on ERT for 6 months after rAAV-LSP-hGAA administration, whereas Cohort 2 subjects were withdrawn from ERT at the time of rAAV-LSP-hGAA administration. The construct for the AAV8-LSPhGAA is shown in FIG. 3B, and is an infectious, non-replicating recombinant adeno-associated virus (AAV) serotype 8 vecto comprising AAV2 ITRs, or a haploid AAV vector comprising capsid 8 and another capsid protein e.g., from AAV2 (e.g., AAV2/8-LSPhGAA), expressing human acid alpha-glucosidase (hGAA).

Preliminary data indicate no serious adverse events as of 1 Jun. 2020 that met the expedited reporting rule. Reported adverse events were mostly expected/anticipated and have been mild to moderate in severity, transient and reversible.

Cohort 1 received 1.6E12 vg/kg which has been well tolerated by all 3 subjects, all of which have now reached 12 months post-dose with biopsy data being available shortly. No elevations in aspartate transaminase (AST) or alanine transaminase (ALT) have been observed in any subject enrolled in Cohort 1 (1.6E12 vg/kg). All subjects received immune prophylaxis with prednisone starting at 60 mg/day for 4 weeks followed by a tapering regimen (5 mg/week for 11 weeks). No subject in Cohort 1 exhibited positive enzyme-linked immune absorbent spot (ELISPOT) reactivity for capsid or acid alfa-glucosidase (GAA). All subjects exhibited serum GAA concentrations above their respective baseline values; however, serum GAA concentrations are decreasing over time. None have needed to receive rescue with ERT.

Cohort 2 received 1.6E13 vg/kg (dosed mid-August, mid-September and early October 2019). Asymptomatic elevations in AST and ALT have been observed in all subjects in Cohort 2 beginning on Day 46 (004), Day 41 (005), and Day 55 (006). All subjects received immune prophylaxis with prednisone starting at 60 mg/day; however, all subjects received augmented doses of prednisone in response to elevations in AST and ALT. Two subjects (004 and 005) received IV methylprednisolone. Two subjects in Cohort 2 had CTCAE Grade 3 laboratory elevations. Subject 004 experienced one Grade 3 elevation in ALT (348 U/L; ULN=63; 5×ULN=315) and one Grade 3 elevation in AST (222 U/L; ULN=41; 5×ULN=205). Both Grade 3 elevations occurred on the same day and were considered definitely/probably related to ACTUS-101 by the Principal Investigator. Subject 5 experienced one Grade 3 elevation in gamma-glutamyl transferase (GGT) (311 U/L; ULN=50; 5× ULN=250) which the Principal Investigator considered possibly related to ACTUS-101, although more probably to prednisone. All Grade 3 elevations were transient and asymptomatic. Subject 004 had a liver biopsy within 10 days following the Grade 3 laboratory elevations, the results of which indicated mild and non-specific inflammation. This subject also reported a lipoma at Week 24, which has been attributed to the high and long duration of steroid administration, which has since been tapered. All subjects exhibited positive but varying levels of ELISPOT T cell responses to AAV8 capsid which is not always concordant in time with the noted liver enzyme changes.

Aalytical comparability studies were conducted to determine the effects, or absence thereof, of proposed manufacturing changes on the identity, strength, quality, purity, or potency of the AAV8-LSPhGAA.

Analytical comparability of AAV8-LSPhGAA can be assessed using a qualified in vitro potency assay run at Absorption Systems Boston, LLC (ASB, Medford, Mass.) under Method standard operating procedure (SOP), which is an in vitro assay for determination of the potency of AAV8-LSPhGAA test samples relative to a reference standard. In brief, a 6-day assay in 96-well HuH-7 cell culture plate format (human hepatocyte cell line, Sekisui XenoTech, Cat. No JCRB0403) is performed, transduced with AAV8-LSPhGAA using a 9-point dilution scheme, and incubated for 96±2 hours after which GAA is measured in the cell supernatant by the 4-MU bioassay. Relative potency is calculated with the parallel line assay model using PLA Software 3.0 (Stegmann Systems).

Dose Ranging Studies:

Dose-ranging studies in GAA knockout (KO) mice, which were used to support the pharmacodynamic effects of AAV8-LSPhGAA. Male and female GAA KO Pompe mice (B6;129-Gaatm1Rabn/J, stock no. 004154, 6neo) are be injected at 8 to 12 weeks (modification 1 proposed 9 to 12 week prior to feedback from the agency) of age by tail vein (intravenous) injection (bolus). A minimum of 6 males (M) and 6 females (F) are be randomly assigned to receive one of the test products (AAV8-LSPhGAA) dosed at 2E11, 2E12 and 2E13 vg/kg based on Han et al (2017 and 2019) and a control group will receive the formulation buffer only (vehicle). Animals will be followed for 8 weeks and then sacrificed for tissue and blood collection. Experimental and control groups will remain blinded to the operator(s).

In life monitoring will consist of daily checks for mortality. Moribund animals will be removed from the study (with gross necropsy examination and tissue sampling for histopathology performed). Serum GAA will be measured in all groups every 2 weeks until termination at 8 weeks after dosing.

Measurements and tissue collection at necropsy will include, Liver, heart, quadriceps, Tibialis A., Diaphram, Brain, Soleus, kidney, spleen, lung, and other clinical measurements, including: (1) Terminal body weight, (2) Organ weights for liver and heart, (3) Serum (GAA activity) and storage at −20° C. for subsequent measurement of anti-GAA antibodies (ADA), (4) Liver, heart, soleus, quadricep, tibialis anterior, tongue, diaphragm, brain to be split in several fragments. Additional measurements includes: (4) GAA activity in serum and tissue and glycogen content in tissues, as measured by qualified assays (tissue will be snap frozen in liquid nitrogen and stored at −80° C.). A target of ˜50 mg of each specified tissue will be collected for each assay, and (5) Histopathological examination analysis fixed in 10% neutral buffered formalin for Haemotoxylin and Eosin (H&E) staining on tissues as indicated above as well as any observed gross lesions at necropsy. A subsection of tissues will be fixed in glutaraldehyde for glycogen staining as needed (Periodic acid-Schiff staining—PAS), Assessment of immune responses to the transgene product by measuring anti-GAA antibody responses (ADA), Total DNA for vector genome copy number per diploid genome determination—vg/ug of DNA (snap frozen in liquid nitrogen and then stored at −80° C.).

Dose-ranging studies in GAA KO mice will support in vivo comparability between AAV8-LSPhGAA and a modified AAV vector, modified according to the disclosure herein (e.g., different LSP, optimization of hGAA sequence, if no significant differences are observed by the following measurements: 1. Histopathology by certified veterinary pathologist in all collected tissues (safety); 2. GAA activity and glycogen content in heart, diaphragm and quadriceps (efficacy) with a relative activity of the modified AAV vector between 0.80 to 1.25 (80-125%) of AAV8-LSPhGAA; 3. Vector genome DNA per ug DNA in collected tissues (biodistribution) with a relative biodistribution of a modified AAV vector between 0.80 to 1.25 (80-125%) of AAV8-LSPhGAA; 4. No change of anti-GAA ADA.

While the present inventions have been described and illustrated in conjunction with a number of specific embodiments, those skilled in the art will appreciate that variations and modifications may be made without departing from the principles of the inventions as herein illustrated, as described and claimed. The present inventions may be embodied in other specific forms without departing from their spirit or essential characteristics. The described embodiments are considered in all respects to be illustrative and not restrictive.

In closing, regarding the exemplary embodiments of the present invention as shown and described herein, it will be appreciated that a genomic construct, comprising an AAV (adeno-associated virus) viral virion is disclosed and configured for delivery of AAV vectors. Because the principles of the invention may be practiced in a number of configurations beyond those shown and described, it is to be understood that the invention is not in any way limited by the exemplary embodiments, but is generally directed to a genomic construct, comprising an AAV (adeno-associated virus) viral virion apparatus and is able to take numerous forms to do so without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of” As such, embodiments described herein or so claimed with the phrase “comprising” are expressly or inherently unambiguously described, enabled and supported herein for the phrases “consisting essentially of” and “consisting of.”

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

### REFERENCES

The references disclosed in the specification and Examples, including but not limited to patents and patent applications, and international patent applications are all incorporated herein in their entirety by reference.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### REFERENCES

1. L Lisowski, A P Dane, K Chu, Y Zhang, S C Cunninghamm, E M Wilson, et al. Selection and evaluation of clinically relevant AAV variants in a xenograft liver model Nature, 506 (2014), pp. 382-386 (LK03 and others LKO-19)
2. Grimm D, Lee J S, Wang L, Desai T, Akache B Storm T A, Kay M A. In vitro and in vivo gene therapy vector evolution via multispecies interbreeding and retargeting of adeno-associated viruses. J Virol. 2008 June: 82(12):5887-911. (AAV-DJ)
3. Powell S K, Khan N, Parker C L, Samulski R J, Matsushima G, Gray S J, McCown T J. Characterization of a novel adeno-associated viral vector with preferential oligodendrocyte tropism. Gene Ther. 2016 Nov.: 23(1 1):807-814. (Olig001)
4. Tervo D G, Hwang B Y, Viswanathan S, Gaj T, Lavzin M, Ritola K D, Lindo S, Michael S, Kuleshova E, Ojala D, Huang C C, Gerfen C R, Schiller J, Dudman J T, Hantman A W, Looger L L, Schaffer D V, Karpova A Y. A Designer AAV Variant Permits Efficient Retrograde Access to Projection Neurons. Neuron. 2016 Oct. 19: 92(2):372-382. (rAAV2-retro)
5. Marsic D, Govindasamy L, Currlin S, Markusic D M, Tseng Y S, Herzog R W, Agbandje-McKenna M, Zolotukhin S. Vector design Tour de Force: integrating combinatorial and rational approaches to derive novel adeno-associated virus variants. Mol Ther. 2014 Nov.: 22(11):1900-9. (AAV-LiC)
6. Sallach J, Di Pasquale G, Larcher F, Niehoff N, Rubsam M, Huber A, Chiarini J, Almarza D, Eming S A, Ulus H, Nishimura S, Hacker U T, Ballek M, Niessen C M, Buning H. Tropism-modified AAV vectors overcome barriers to successful cutaneous therapy. Mol Ther. 2014 May: 22(5):929-39. (AAV-Keral, AAV-Kera2, and AAV-Kera3)
7. Dalkara D, Byrne L C, Klimczak R R, Visel M, Yin L, Merigan W H, Flannery J G, Schaffer D V. In vivo-directed evolution of a new adeno-associated virus for therapeutic outer retinal gene delivery from the vitreous. Sci Transl Med. 2013 June 12:5(189):189ra76. (AAV 7m8)
8. Asuri P, Bartel M A, Vazin T, Jang J H, Wong T B, Schaffer D V. Directed evolution of adeno-associated virus for enhanced gene delivery and gene targeting in human pluripotent stem cells. Mol Ther. 2012 Feb.: 20(2):329-38. (AAV1.9)
9. Jang J H, Koerber J T, Kim J S, Asuri P, Vazin T, Bartel M, Keung A, Kwon I, Park K I, Schaffer D V. An evolved adeno-associated viral variant enhances gene delivery and gene targeting in neural stem cells. Mol Ther. 2011 Apr.: 19(4):667-75. doi: 10.1038/mt.2010.287. (AAV r3.45)
10. Gray S J, Blake B L, Criswell H E, Nicolson S C, Samulski R J, McCown T J, Li W. Directed evolution of a novel adeno-associated virus (AAV) vector that crosses the seizure-compromised blood-brain barrier (BBB). Mol Ther. 2010 Mar.: 18(3):570-8 (AAV clone 32 and 83)
11. Maguire C A, Gianni D, Meijer D H, Shaket L A, Wakimoto H, Rabkin S D, Gao G, Sena-Esteves M. Directed evolution of adeno-associated virus for glioma cell transduction. J Neurooncol. 2010 Feb.: 96(3):337-47. (AAV-U87R7-C5)
12. Koerber J T, Klimczak R, Jang J H, Dalkara D, Flannery J G, Schaffer D V. Molecular evolution of adeno-associated virus for enhanced glial gene delivery. Mol Ther. 2009 Dec.: 17(12):2088-95. (AAV ShH13, AAV ShH19, AAV Ll-12)
13. Li W, Zhang L, Johnson J S, Zhijian W, Grieger J C, Ping-Jie X, Drouin L M, Agbandje-McKenna M, Pickles R J, Samulski R J. Generation of novel AAV variants by directed evolution for improved CFTR delivery to human ciliated airway epithelium. Mol Ther. 2009 Dec.: 17(12):2067-77. (AAV HAE-1, AAV HAE-2)
14. Klimczak R R, Koerber J T, Dalkara D, Flannery J G, Schaffer D V. A novel adeno-associated viral variant for efficient and selective intravitreal transduction of rat Muller cells. PLoS One. 2009 Oct. 14:4(10):e7467. (AAV variant ShH10)
15. Excoffon K J, Koerber J T, Dickey D D, Murtha M, Keshavjee S, Kaspar B K, Zabner J, Schaffer D V. Directed evolution of adeno-associated virus to an infectious respiratory virus. Proc Natl Acad Sci USA. 2009 Mar. 10:106(10):3865-70. (AAV2.5T)
16. Sellner L, Stiefelhagen M, Kleinschmidt J A, Laufs S, Wenz F, Fruehauf S, Zeller W J, Veldwijk M R. Generation of efficient human blood progenitor-targeted recombinant adeno-associated viral vectors (AAV) by applying an AAV random peptide library on primary human hematopoietic progenitor cells. Exp Hematol. 2008 August: 36(8):957-64. (AAV LS1-4, AAV Lsm)
17. Li W, Asokan A, Wu Z, Van Dyke T, DiPrimio N, Johnson J S, Govindaswamy L, Agbandje-McKenna M, Leichtle S, Redmond D E Jr, McCown T J, Petermann K B, Sharpless N E, Samulski R J. Engineering and selection of shuffled AAV genomes: a new strategy for producing targeted biological nanoparticles. Mol Ther. 2008 Jul.:16(7):1252-60. (AAV1289)
18. Charbel Issa P, De Silva S R, Lipinski D M, Singh M S, Mouravlev A, You Q. Assessment of tropism and effectiveness of new primate-derived hybrid recombinant AAV serotypes in the mouse and primate retina. PLoS ONE. 2013:8:e60361. (AAVHSC 1-17)
19. Huang W, McMurphy T, Liu X, Wang C, Cao L. Genetic Manipulation of Brown Fat Via Oral Administration of an Engineered Recombinant Adeno-associated Viral Serotype Vector. Mol Ther. 2016 Jun.: 24(6):1062-9. (AAV2 Rec 1-4)
20. Cronin T, Vandenberghe L H, Hantz P, et al. Efficient transduction and optogenetic stimulation of retinal bipolar cells by a synthetic adeno-associated virus capsid and promoter. EMBO Mol Med 2014:6:1175-1190 (AAV8BP2)
21. Choudhury S R, Fitzpatrick Z, Harris A F, Maitland S A, Ferreira J S, Zhang Y, Ma S, Sharma R B, Gray-Edwards H L, Johnson J A, Johnson A K, Alonso L C, Punzo C, Wagner K R, Maguire C A, Katin R M, Martin D R, Sena-Esteves M. In Vivo Selection Yields AAV-Bl Capsid for Central Nervous System and Muscle Gene Therapy. Mol Ther. 2016 Aug.: 24(7):1247-57. (AAV-B1)
22. Deverman B E, Pravda P L, Simpson B P, Kumar S R, Chan K Y, Banerjee A, Wu W L, Yang B, Huber N, Pasca S P, Gradinaru V. Cre-dependent selection yields AAV variants for widespread gene transfer to the adult brain. Nat Biotechnol. 2016 February: 34(2):204-9. doi: 10.1038/nbt.3440. (AAV-PHP.B)
23. Pulicherla N, Shen S, Yadav S, Debbink K, Govindasamy L, Agbandje-McKenna M, Asokan A. Engineering liver-detargeted AAV9 vectors for cardiac and musculoskeletal gene transfer. Mol Ther. 2011 Jun.: 19(6):1070-8. (AAV9 derived mutants-AAV9.45, AAV9.61, AAV9.47)
24. Yang L, Jiang J, Drouin L M, Agbandje-McKenna M, Chen C, Qiao C, Pu D, Hu X, Wang D Z, Li J, Xiao X. A myocardium tropic adeno-associated virus (AAV) evolved by DNA shuffling and in vivo selection. Proc Natl Acad Sci USA. 2009 Mar. 10:106(10):3946-51. (AAVM41)
25. Korbelin J, Sieber T, Michelfelder S, Lunding L, Spies E, Hunger A, Alawi M, Rapti K, Indenbirken D, Muller O J, Pasqualini R, Arap W, Kleinschmidt J A, Trepel M. Pulmonary Targeting of Adena-associated Viral Vectors by Next-generation Sequencing-guided Screening of Random Capsid Displayed Peptide Libraries. Mol Ther. 2016 Jun. 24(6):1050-61. (AAV2 displayed peptides)
26. Geoghegan J C, Keiser N W, Okulist A, Martins I, Wilson M S, Davidson B L. Chondroitin Sulfate is the Primary Receptor for a Peptide-Modified AAV That Targets Brain Vascular Endothelium In Vivo. Mol Ther Nucleic Acids. 2014 Oct 14:3:e202. (AAV2-GMN)
27. Varadi K, Michelfelder S, Korff T, Hecker M, Trepel M, Katus H A, Kleinschmidt J A, Muller O J. Novel random peptide libraries displayed on AAV serotype 9 for selection of endothelial cell-directed gene transfer vectors. Gene Ther. 2012 Aug. 19(8):800-9. (AAV9-peptide displayed)
28. Michelfelder S, Varadi K, Raupp C, Hunger A, Korbelin J, Pahrmann C, Schrepfer S, Muller O J, Kleinschmidt J A, Trepel M. Peptide ligands incorporated into the threefold spike capsid domain to re-direct gene transduction of AAV8 and AAV9. in vivo. PLoS One. 2011:6(8):e23101. (AAV8 and AAV9 peptide displayed)
29. Yu C Y, Yuan Z, Cao Z, Wang B, Qiao C, Li J, Xiao X. A muscle-targeting peptide displayed on AAV2 improves muscle tropism on systemic delivery. Gene Ther. 2009 Aug. 16(8):953-62.
30. Michelfelder S, Lee M K, deLima-Hahn E, Wilmes T, Kaul F, Muller O, Kleinschmidt J A, Trepel M. Vectors selected from adeno-associated viral display peptide libraries for leukemia cell-targeted cytotoxic gene therapy. Exp Hematol. 2007 Dec: 35(12): 1766-76.
31. Muller O J, Kaul F, Weitzman M D, Pasqualini R, Arap W, Kleinschmidt J A, Trepel M. Random peptide libraries displayed on adeno-associated virus to select for targeted gene therapy vectors. Nat Biotechnol. 2003 Sep. 21(9):1040-6.
32. Grifman M, Trepel M, Speece P, Gilbert L B, Arap W, Pasqualini R, Weitzman M D. Incorporation of tumor-targeting peptides into recombinant adeno-associated virus capsids. Mol Ther. 2001 Jun. 3(6):964-75.
33. Anne Girod, Martin Ried, Christiane Wobus, Harald Lahm, Kristin Leike, Jurgen Kleinschmidt, Gilbert Deleage & Michael Ballek. Genetic capsid modifications allow efficient retargeting of adeno-associated virus type 2. Nature Medicine, 1052-1056 (1999)
34. Bello A, Chand A, Aviles J, Soule G, Auricchio A, Kobinger G P. Novel adeno-associated viruses derived from pig tissues transduce most major organs in mice. Sci Rep. 2014 Oct 22:4:6644. (AAVpo2.1, -po4, -poS, and -po6).
35. Gao G, Vandenberghe L H, Alvira M R, Lu Y, Calcedo R, Zhou X, Wilson J M. Clades of Adena-associated viruses are widely disseminated in human tissues. J Virol. 2004 Jun. 78(12):6381-8. (AAV rh and AAV Hu)
36. Arbetman A E, Lochrie M, Zhou S, Wellman J, Scallan C, Doroudchi M M, et al. Novel caprine adeno-associated virus (AAV) capsid (AAV-Go.1) is closely related to the primate AAV-5 and has unique tropism and neutralization properties. J Virol. 2005:79:15238-15245. (AAV-Go.1)
37. Lochrie M A, Tatsuno G P, Arbetman A E, Jones K, Pater C, Smith P H, et al. Adena-associated virus (AAV) capsid genes isolated from rat and mouse liver genomic DNA define two new AAV species distantly related to AAV-5. Virology. 2006:353:68-82. (AAV-mo.1)
38. Schmidt M, Katano H, Bossis I, Chiarini J A. Cloning and characterization of a bovine adeno-associated virus. J Virol. 2004:78:6509-6516. (BAAV)
39. Bossis I, Chiarini J A. Cloning of an avian adeno-associated virus (AAAV) and generation of recombinant AAAV particles. J Virol. 2003:77:6799-6810. (AAAV)
40. Chen C L, Jensen R L, Schnepp B C, Connell M J, Shell R, Sferra T J, Bartlett J S, Clark K R, Johnson P R. Molecular characterization of adeno-associated viruses infecting children. J Virol. 2005 Dec: 79(23):14781-92. (AAV variants)
41. Sen D, Gadkari R A, Sudha G, Gabriel N, Kumar Y S, Selot R, Samuel R, Rajalingam S, Ramya V, Nair S C, Srinivasan N, Srivastava A, Jayandharan G R. Targeted modifications in adeno-associated virus serotype 8 capsid improves its hepatic gene transfer efficiency in vivo. Hum Gene Ther Methods. 2013 Apr: 24(2):104-16. (AAV8 K137R)
42. Li B, Ma W, Ling C, Van Vliet K, Huang L Y, Agbandje-McKenna M, Srivastava A, Aslanidi G V. Site-Directed Mutagenesis of Surface-Exposed Lysine Residues Leads to Improved Transduction by AAV2, But Not AAV8, Vectors in Murine Hepatocytes In Vivo. Hum Gene Ther Methods. 2015 Dec: 26(6):211-20.
43. Gabriel N, Hareendran S, Sen D, Gadkari R A, Sudha G, Selot R, Hussain M, Dhaksnamoorthy R, Samuel R, Srinivasan N, et al. Bioengineering of AAV2 capsid at specific serine, threonine, or lysine residues improves its transduction efficiency in vitro and in vivo. Hum Gene Ther Methods. 2013 Apr: 24(2):80-93.
44. Zinn E, Pacouret S, Khaychuk V, Turunen H T, Carvalho L S, Andres-Mateos E, Shah S, Shelke R, Maurer A C, Plovie E, Xiao R, Vandenberghe L H. In Silico Reconstruction of the Viral Evolutionary Lineage Yields a Potent Gene Therapy Vector. Cell Rep. 2015 Aug. 11:12(6):1056-68. (AAV Anc80L65)
45. Shen S, Horowitz E D, Troupes A N, Brown S M, Pulicherla N, Samulski R J, Agbandje-McKenna M, Asokan A. Engraftment of a galactose receptor footprint onto adeno-associated viral capsids improves transduction efficiency. J Biol Chem. 2013 Oct 4:288(40):28814-23. (AAV2G9)
46. Li C, Diprirnio N, Bowles D E, Hirsch M L, Monahan P E, Asokan A, Rabinowitz J, Agbandje-McKenna M, Samulski R J. Single amino acid modification of adeno-associated virus capsid changes transduction and humoral immune profiles. J Virol. 2012 August 86(15):7752-9. (AAV2 265 insertion-AAV2/265D)
47. Bowles D E, McPhee S W, Li C, Gray S J, Sarnulski J J, Camp A S, Li J, Wang B, Monahan P E, Rabinowitz J E, et al. Phase 1 gene therapy for Duchenne muscular dystrophy using a translational optimized AAV vector. Mol Ther. 2012 Feb. 20(2):443-55 (AAV2.5)
48. Messina E L, Nienaber J, Daneshmand M, Villarnizar N, Samulski J, Milano C, Bowles D E. Adena-associated viral vectors based on serotype 3b use components of the fibroblast growth factor receptor signaling complex for efficient transduction. Hum. Gene Ther. 2012 Oct: 23(10):1031-42. (AAV3 SASTG)
49. Asokan A, Conway J C, Phillips J L, Li C, Hegge J, Sinnott R, Yadav S, DiPrimio N, Nam H J, Agbandje-McKenna M, McPhee S, Wolff J, Samulski R J. Reengineering a receptor footprint of adeno-associated virus enables selective and systemic gene transfer to muscle. Nat Biotechnol. 2010 Jan. 28(1):79-82. (AAV2i8)
50. Vance M, Llanga T, Bennett W, Woodard K, Murlidharan G, Chungfat N, Asokan A, Gilger B, Kurtzberg J, Sarnulski R J, Hirsch M L. AAV Gene Therapy for MPS1-associated Corneal Blindness. Sci Rep. 2016 Feb. 22:6:22131. (AAV8G9)
51. Zhong L, Li B, Mah C S, Govindasarny L, Agbandje-McKenna M, Cooper M, Herzog R W, Zolotukhin I, Warrington K H Jr, Weigel-Van Aken K A, Hobbs J A, Zolotukhin S, Muzyczka N, Srivastava A. Next generation of adeno-associated virus 2 vectors: point mutations in tyrosines lead to high-efficiency transduction at lower doses. Proc Natl Acad Sci USA. 2008 Jun. 3:105(22):7827-32. (AAV2 tyrosine mutants AAV2 Y-F)
52. Petrs-Silva H, Dinculescu A, Li Q, Min S H, Chiodo V, Pang J J, Zhong L, Zolotukhin S, Srivastava A, Lewin A S, Hauswirth W W. High-efficiency transduction of the mouse retina by tyrosine-mutant AAV serotype vectors. Mol Ther. 2009 Mar. 1 7(3):463-71. (AAV8 Y-F and AAV9 Y-F)
53. Qiao C, Zhang W, Yuan Z, Shin J H, Li J, Jayandharan G R, Zhong L, Srivastava A, Xiao X, Duan D. Adena-associated virus serotype 6 capsid tyrosine-to-phenylalanine mutations improve gene transfer to skeletal muscle. Hum Gene Ther. 2010 Oct: 21(10):1343-8 (AAV6 Y-F)
54. Carlon M, Toelen J, Van der Perren A, Vandenberghe L H, Reumers V, Sbragia L, Gijsbers R, Baekelandt V, Himmelreich U, Wilson J M, Deprest J, Debyser Z. Efficient gene transfer into the mouse lung by fetal intratracheal injection of rAAV2/6.2. Mol Ther. 2010 Dec: 18(12):2130-8. (AAV6.2) PCT Publication No. WO2013158879A1 (lysine mutants)
55. Piacentino III, Valentino, et al. “X-linked inhibitor of apoptosis protein-mediated attenuation of apoptosis, using a novel cardiac-enhanced adeno-associated viral vector.” Human gene therapy 23.6 (2012): 635-646.
56. Hirschhorn et al. (2001) “Glycogen Storage Disease Type II: Acid Alpha-glucosidase (Acid Maltase) Deficiency,” in Scriver et al., eds., The Metabolic and Molecular Basis of Inherited Disease, 8th Ed., New York: McGraw-Hill, 3389-3420.
57. Hermonat, P. L., Labow, M. A., Wright, R., Berns, K. I., & Muzyczka, N. (1984). Genetics of adeno-associated virus: isolation and preliminary characterization of adeno-associated virus type 2 mutants. Journal of Virology, 51(2), 329-339. https://doi.org/10.1128/jvi.51.2.329-339.1984
58. West, M. H., Trempe, J. P., Tratschin, J. D., & Carter, B. J. (1987). Gene expression in adeno-associated virus vectors: the effects of chimeric mRNA structure, helper virus, and adenovirus VA1 RNA. Virology, 160(1), 38-47. https://doi.org/10.1016/0042-6822(87)90041-9
59. Bohenzky, R. A., & Berns, K. I. (1989). Interactions between the termini of adeno-associated virus DNA. Journal of Molecular Biology, 206(1), 91-100. https://doi.org/10.1016/0022-2836(89)90526-3
60. Flotte, T. R., Solow, R., Owens, R. A., Afione, S., Zeitlin, P. L., & Carter, B. J. (1992). Gene expression from adeno-associated virus vectors in airway epithelial cells. American Journal of Respiratory Cell and Molecular Biology, 7(3), 349-356. https://doi.org/10.1165/ajrcmb/7.3.349
61. Flotte, T. R., Afione, S. A., Solow, R., Drumm, M. L., Markakis, D., Guggino, W. B., Zeitlin, P. L., & Carter, B. J. (1993). Expression of the cystic fibrosis transmembrane conductance regulator from a novel adeno-associated virus promoter. Journal of Biological Chemistry, 268(5), 3781-3790.
62. Flotte, T., Carter, B., Conrad, C., Guggino, W., Reynolds, T., Rosenstein, B., Taylor, G., Walden, S., & Wetzel, R. (1996). A phase I study of an adeno-associated Virus-CFTR gene vector in adult C F patients with mild lung disease. In Human Gene Therapy (Vol. 7, Issue 9, pp. 1145-1159). Mary Ann Liebert Inc. https://doi.org/10.1089/hum.1996.7.9-1145
63. Rubenstein, R. C., McVeigh, U., Flotte, T. R., Guggino, W. B., & Zeitlin, P. L. (1997). CFTR gene transduction in neonatal rabbits using an adeno-associated virus (AAV) vector. Gene Therapy, 4(5), 384-392. https://doi.org/10.1038/sj.gt.3300417
64. Haberman, R. P., McCown, T. J., & Samulski, R. J. (1998). Inducible long-term gene expression in brain with adeno-associated virus gene transfer. Gene Therapy, 5(12), 1604-1611. https://doi.org/10.1038/sj.gt.3300782
65. Wang, D., Fischer, H., Zhang, L., Fan, P., Ding, R. X., & Dong, J. (1999). Efficient CFTR expression from AAV vectors packaged with promoters—The second generation. Gene Therapy, 6(4), 667-675. https://doi.org/10.1038/sj.gt.3300856
66. Hsiao, C. D., Hsieh, F. J., & Tsai, H. J. (2001). Enhanced expression and stable transmission of transgenes flanked by inverted terminal repeats from adeno-associated virus in zebrafish. Developmental Dynamics: An Official Publication of the American Association of Anatomists,
220(4), 323-336. https://doi.org/10.1002/dvdy.1113
67. Davidoff, A. M., Ng, C. Y. C., Zhou, J., Spence, Y., & Nathwani, A. C. (2003). Sex significantly influences transduction of murine liver by recombinant adeno-associated viral vectors through an androgen-dependent pathway. Blood, 102(2), 480-488. https://doi.org/10.1182/blood-2002-09-2889
68. Farris, K. D., & Pintel, D. J. (2008). Improved splicing of adeno-associated viral (AAV) capsid protein-supplying pre-mRNAs leads to increased recombinant AAV vector production. Human Gene Therapy, 19(12), 1421-1427. https://doi.org/10.1089/hum.2008.118
69. Li, C., Hirsch, M., Carter, P., Asokan, A., Zhou, X., Wu, Z., & Samulski, R. J. (2009). A small regulatory element from chromosome 19 enhances liver-specific gene expression. Gene Therapy, 16(1), 43-51. https://doi.org/10.1038/gt.2008.134
70. Dean, J., Plante, J., Huggins, G. S., Snyder, R. O., & Aikawa, R. (2009). Role of cyclic AMP-dependent kinase response element-binding protein in recombinant adeno-associated virus-mediated transduction of heart muscle cells. Human Gene Therapy, 20(9), 1005-1012. https://doi.org/10.1089/hum.2009.054
71. Ling, C., Wang, Y., Lu, Y., Wang, L., Jayandharan, G. R., Aslanidi, G. V., Li, B., Cheng, B., Ma, W., Lentz, T., Ling, C., Xiao, X., Samulski, R. J., Muzyczka, N., & Srivastava, A. (2015). Enhanced Transgene Expression from Recombinant Single-Stranded D-Sequence-Substituted Adeno-Associated Virus Vectors in Human Cell Lines In Vitro and in Murine Hepatocytes In Vivo. Journal of Virology, 89(2), 952-961. https://doi.org/10.1128/jvi.02581-14
72. Wang, L., Yin, Z., Wang, Y., Lu, Y., Zhang, D., Srivastava, A., Ling, C., Aslanidi, G. V., & Ling, C. (2015). Productive life cycle of adeno-associated virus serotype 2 in the complete absence of a conventional polyadenylation signal. Journal of General Virology, 96(9), 2780-2787. https://doi.org/10.1099/jgv.0.000229
73. Powell, S. K., Rivera-Soto, R., & Gray, S. J. (2015). Viral expression cassette elements to enhance transgene target specificity and expression in gene therapy. Discovery Medicine, 19(102), 49-57. https://www.ncbi.nlm.nih.gov/pmc/articles/PMC4505817/
74. Stutika, C., Mietzsch, M., Gogol-D6ring, A., Weger, S., Sohn, M., Chen, W., & Heilbronn, R. (2016). Comprehensive Small RNA-Seq of Adeno-Associated Virus (AAV)-Infected Human Cells Detects Patterns of Novel, Non-Coding AAV RNAs in the Absence of Cellular miRNA Regulation. PloS One, 11(9), e0161454. https://doi.org/10.1371/journal.pone.0161454
75. Weger, S., Hammer, E., Gonsior, M., Stutika, C., & Heilbronn, R. (2016). A Regulatory Element Near the 3′ End of the Adeno-Associated Virus rep Gene Inhibits Adenovirus Replication in cis by Means of p40 Promoter-Associated Short Transcripts. Journal of Virology, 90(8), 3981-3993. https://doi.org/10.1128/jvi.03120-15
76. Logan, G. J., Dane, A. P., Hallwirth, C. V, Smyth, C. M., Wilkie, E. E., Amaya, A. K., Zhu, E., Khandekar, N., Ginn, S. L., Liao, S. H. Y., Cunningham, S. C., Sasaki, N., Cabanes-Creus, M., Tam, P. P. L., Russell, D. W., Lisowski, L., & Alexander, I. E. (2017). Identification of liver-specific enhancer-promoter activity in the 3′ untranslated region of the wild-type AAV2 genome. Nature Genetics, 49(8), 1267-1273. https://doi.org/10.1038/ng.3893
77. Lu, J., Williams, J. A., Luke, J., Zhang, F., Chu, K., & Kay, M. A. (2017). A 5′ noncoding exon containing engineered intron enhances transgene expression from recombinant AAV vectors in vivo. Human Gene Therapy, 28(1), 125-134. https://doi.org/10.1089/hum.2016.140
78. Xu, H., Hao, S., Zhang, J., Chen, Z., Wang, H., & Guan, W. (2017). The formation and modification of chromatin-like structure of human parvovirus B19 regulate viral genome replication and RNA processing. Virus Research, 232, 134-138. https://doi.org/10.1016/j.virusres.2017.03.001
79. Julien, L., Chassagne, J., Peccate, C., Lorain, S., Pidtri-Rouxel, F., Danos, O., & Benkhelifa-Ziyyat, S. (2018). RFX1 and RFX3 Transcription Factors Interact with the D Sequence of Adeno-Associated Virus Inverted Terminal Repeat and Regulate AAV Transduction. Scientific Reports, 8(1). https://doi.org/10.1038/s41598-017-18604-3
80. Wilmott, P., Lisowski, L., Alexander, I. E., & Logan, G. J. (2019). A User's Guide to the Inverted Terminal Repeats of Adeno-Associated Virus. Human Gene Therapy Methods, 30(6), 206-213. https://doi.org/10.1089/hgtb.2019.276
81. Lu, L., Zhang, F., Li, Y., Yang, A., Guan, C., Ding, X., Liu, Y., Liu, Y., Zhang, C.-Y., Li, L., & Zhang, Q. (2019). Dendritic targeted mRNA expression via a cis-acting RNA UTR element. Biochemical and Biophysical Research Communications, 509(2), 402-406. https://doi.org/10.1016/j.bbrc.2018.12.137
82. Earley, L. F., Conatser, L. M., Lue, V. M., Dobbins, A. L., Li, C., Hirsch, M. L., & Samulski, R. J. (2020). Adeno-Associated Virus Serotype-Specific Inverted Terminal Repeat Sequence Role in Vector Transgene Expression. Human Gene Therapy, 31(3-4), 151-162. https://doi.org/10.1089/hum.2019.274

1. A recombinant adenovirus associated (AAV) vector comprising in its genome:
a. 5′ and 3′ AAV inverted terminal repeats (ITR) sequences, and
b. located between the 5′ and 3′ ITRs, a heterologous nucleic acid sequence encoding a polypeptide comprising an alpha-glucosidase (GAA) polypeptide, wherein the heterologous nucleic acid is operatively linked to a liver-specific promoter selected from any one of:
i. CRM_SP0412 (SEQ ID NO: 86) or SP0412 (SEQ ID NO: 91) or a functional variant or functional fragment thereof having at least 60% activity to SEQ ID NO: 86 or SEQ ID NO: 91,
ii. SP0422 (SEQ ID NO: 92) or a functional variant or functional fragment thereof having at least 60% activity to SEQ ID NO: 92,
iii. CRM_SP0239 (SEQ ID NO: 87) or SP0239 (SEQ ID NO: 93) or SP0238-UTR (SEQ ID NO: 147) or a functional variant or functional fragment thereof having at least 60% activity to SEQ ID NO: 87, SEQ ID NO: 93 or SEQ ID NO: 147;
iv. CRM_SP0265 (SP0131_A1) (SEQ ID NO: 88) or SP0265 (LVR_SP0131_A1) (SEQ ID NO: 94) or SP0265-UTR (SEQ ID NO: 146) or a functional variant or functional fragment thereof having at least 60% activity to SEQ ID NO: 88, SEQ ID NO: 94 or SEQ ID NO: 146;
v. CRM_SP0240 (SEQ ID NO: 89) or SP0240 (SEQ ID NO: 95) or SP0240-UTR (SEQ ID NO: 148) or a functional variant or functional fragment thereof having at least 60% activity to SEQ ID NO: 89, SEQ ID NO: 95 or SEQ ID NO: 148;
vi. CRM_SP0246 (SEQ ID NO: 90) or SP0246 (SEQ ID NO: 96) or SP0246-UTR (SEQ ID NO: 149) or a functional variant or functional fragment thereof having at least 60% activity to SEQ ID NO: 90, SEQ ID NO: 96 or SEQ ID NO: 149.

2. The recombinant AAV vector of claim 1, wherein the heterologous nucleic acid sequence encodes a fusion protein comprising a secretory signal fused to the GAA polypeptide.

3. The recombinant AAV vector of claim 2, wherein the AAV genome comprises, in the 5′ to 3′ direction:
a. a 5′ ITR,
b. a liver-specific promoter sequence,
c. an intron sequence,
d. a nucleic acid encoding a secretory signal peptide,
e. a nucleic acid encoding an alpha-glucosidase (GAA) polypeptide,
f. a poly A sequence, and
g. a 3′ ITR.

4. The recombinant AAV vector of claim 2, wherein nucleic acid encoding the secretory signal peptide encodes a signal sequence selected from any of: an AAT signal peptide, a fibronectin signal peptide (FN1), a GAA leader sequence, a IL-2 wt leader sequence, modified IL-2 leader sequence, IL2(1-3) leader sequence, IgG leader sequence, a AAT leader sequence, or an active fragment thereof having secretory signal activity.

5.-7. (canceled)

8. The recombinant AAV vector of claim 1, wherein the nucleic acid sequence encodes a wild-type human GAA polypeptide or a modified human GAA polypeptide, or human codon optimized GAA gene (coGAA) or a modified GAA nucleic acid sequence.

9. (canceled)

10. The recombinant AAV vector of claim 1, wherein the nucleic acid sequence encoding the GAA polypeptide is modified from SEQ ID NO: 11 for any one or more of: (i) codon optimized for enhanced expression in vivo, (ii) reduce CpG islands, (iii) modification of STOP sequences, (iv) reduction of alternative reading frames, and (v) to reduce the innate immune response.

11. The recombinant AAV vector of claim 1, wherein the nucleic acid sequence encoding the GAA polypeptide encodes a GAA polypeptide which comprises at least one or at least 2 amino acid modifications selected from; H201L, H199R or R233H of SEQ ID NO: 10.

12.-17. (canceled)

18. The recombinant AAV vector of claim 3, wherein the intron sequence comprises a MVM sequence or a HBB2 sequence or a SV40 sequence.

19. The recombinant AAV vector of claim 1, wherein the ITR comprises an insertion, deletion or substitution or one or more CpG islands in the ITR are deleted.

20. (canceled)

21. The recombinant AAV vector of claim 2, wherein
a. the nucleic acid encoding the secretory signal peptide is selected from any of the group consisting of:
AAT signal peptide (e.g., SEQ ID NO: 17), or an active fragment thereof having secretory signal activity, e.g., a nucleic acid encoding an amino acid sequence that has at least about sequence identity to SEQ ID NO: 17-22;
a fibronectin signal peptide (FN1) (e.g., SEQ ID NO: 18-21), or an active fragment thereof having secretory signal activity, e.g., a nucleic acid encoding an amino acid sequence that has at least about 90% sequence identity to SEQ ID NO: 18-21;
a cognate GAA signal peptide (SEQ ID NO: 175), or an active fragment thereof having secretory signal activity, e.g., a nucleic acid encoding an amino acid sequence that has at least about sequence identity to SEQ ID NO: 175;
an hIGF2 signal peptide (e.g., SEQ ID NO: 22), or an active fragment thereof having secretory signal activity, e.g., a nucleic acid encoding an amino acid sequence that has at least about 90% sequence identity to SEQ ID NO: 22;
a IgG1 leader peptide (SEQ ID NO: 177), or an active fragment thereof having secretory signal activity, e.g., a nucleic acid encoding an amino acid sequence that has at least about 90% sequence identity to SEQ ID NO: 177;
wtIL2 leader peptide (SEQ ID NO: 179), or an active fragment thereof having secretory signal activity, e.g., a nucleic acid encoding an amino acid sequence that has at least about 90% sequence identity to SEQ ID NO: 179;
mutant IL2 leader peptide (SEQ ID NO: 181) or an active fragment thereof having secretory signal activity, e.g., a nucleic acid encoding an amino acid sequence that has at least about 90% sequence identity to SEQ ID NO: 181; and
b. the nucleic acid encoding the GAA polypeptide is selected from any of the group consisting of: SEQ ID NO: 11, SEQ ID NO: 72, SEQ ID NO: 74, SEQ ID NO: 75, SEQ ID NO: 76, SEQ ID NO: 82 or SEQ ID NO: 182 or a nucleic acid sequence having at least about 90% sequence identity to SEQ ID NO: 11, SEQ ID NO: 72, SEQ ID NO: 74, SEQ ID NO: 75, SEQ ID NO: 76, SEQ ID NO: 82 or SEQ ID NO: 182.

22. (canceled)

23. (canceled)

24. The recombinant AAV vector of claim 1, wherein the recombinant AAV vector is a chimeric AAV vector, a haploid AAV vector, a hybrid AAV vector or a polyploid AAV vector, or a rational haploid vector, a mosaic AAV vector, a chemically modified AAV vector, or a AAV vector from any AAV serotype.

25. (canceled)

26. The recombinant AAV vector of claim 1, wherein the recombinant AAV vector is selected from the group consisting of: a AAVXL32 vector, a AAVXL32.1 vector, a AAV3b vector, a AAV8 vector, or a haploid AAV8 vector comprising at least one AAV8 capsid protein.

27. The recombinant AAV vector of claim 24, wherein the recombinant vector comprises a capsid protein selected from the serotypes AAV3, is-AAV3b, AAV8.

28.-37. (canceled)

38. The recombinant AAV vector of claim 1, wherein the nucleic acid sequence encodes a wild-type GAA polypeptide of SEQ ID NO: 10, or a modified GAA polypeptide having at least about 85% sequence homology to SEQ ID NO: 10.

39. The recombinant AAV vector of claim 1, wherein the nucleic acid sequence encodes a GAA polypeptide which comprises at least all three amino acid modifications selected from; H201L, H199R or R233H of SEQ ID NO: 10.

40. The recombinant AAV vector of claim 39, wherein the nucleic acid sequence encoding the GAA polypeptide is the human GAA gene or a human codon optimized GAA gene (coGAA) or a modified GAA nucleic acid sequence, or a nucleic acid sequence encoding the GAA polypeptide is codon optimized to reduce CpG islands or to reduce the innate immune response, or to reduce the innate immune response and reduce the innate immune response.

41.-48. (canceled)

49. A pharmaceutical composition comprising the recombinant AAV vector of claim 1 in a pharmaceutically acceptable carrier.

50. A nucleic acid sequence comprising:
a liver specific promoter operatively linked to a nucleic acid sequence encoding a GAA polypeptide, wherein the liver specific promoter is selected from any one of the liver specific promoters of: wherein the liver specific promoter is selected from any of:
i. CRM_SP0412 (SEQ ID NO: 86) or SP0412 (SEQ ID NO: 91) or a functional variant or functional fragment thereof having at least 60% activity to SEQ ID NO: 86 or SEQ ID NO: 91, or
ii. SP0422 (SEQ ID NO: 92) or a functional variant or functional fragment thereof having at least 60% activity to SEQ ID NO: 92,
iii. CRM_SP0239 (SEQ ID NO: 87) or SP0239 (SEQ ID NO: 93) or SP0238-UTR (SEQ ID NO: 147) or a functional variant or functional fragment thereof having at least 60% activity to SEQ ID NO: 87, SEQ ID NO: 93 or SEQ ID NO: 147;
iv. CRM_SP0265 (SP0131_A1) (SEQ ID NO: 88) or SP0265 (LVR_SP0131_A1) (SEQ ID NO: 94) or SP0265-UTR (SEQ ID NO: 146) or a functional variant or functional fragment thereof having at least 60% activity to SEQ ID NO: 88, SEQ ID NO: 94 or SEQ ID NO: 146;
v. CRM_SP0240 (SEQ ID NO: 89) or SP0240 (SEQ ID NO: 95) or SP0240-UTR (SEQ ID NO: 148) or a functional variant or functional fragment thereof having at least 60% activity to SEQ ID NO: 89, SEQ ID NO: 95 or SEQ ID NO: 148; or
vi. CRM_SP0246 (SEQ ID NO: 90) or SP0246 (SEQ ID NO: 96) or SP0246-UTR (SEQ ID NO: 149) or a functional variant or functional fragment thereof having at least 60% activity to SEQ ID NO: 90, SEQ ID NO: 96 or SEQ ID NO: 149.

51. The nucleic acid sequence of claim 50, further comprising:
a. 5′ and 3′ AAV inverted terminal repeats (ITR) nucleic acid sequences, wherein the 5′ and the 3′ ITR sequences flank the heterologous nucleic acid sequence encoding a alpha-glucosidase (GAA) polypeptide operatively linked to the liver-specific promoter.

52. (canceled)

53. (canceled)

54. (canceled)

55. The nucleic acid sequence of claim 50, wherein the nucleic acid sequence encoding the GAA polypeptide is the human GAA gene or a human codon optimized GAA gene (coGAA) or a modified GAA nucleic acid sequence, or wherein the nucleic acid sequence encoding the GAA polypeptide is modified from SEQ ID NO: 11 for any one or more of: (i) codon optimized for enhanced expression in vivo, (ii) reduce CpG islands, (iii) modification of STOP sequences, (iv) reduction of alternative reading frames, and (v) to reduce the innate immune response.

56.-59. (canceled)

60. The nucleic acid sequence of claim 50, wherein the nucleic acid encoding the GAA polypeptide is selected from any of SEQ ID NO: 74 (codon optimized 1), SEQ ID NO: 75 (codon optimized 2), SEQ ID NO: 76 (codon optimized 3), and SEQ ID NO: 182 (mod hGAA) a nucleic acid sequence having at least 90%, identity thereto.

61. The nucleic acid sequence of claim 50, wherein the nucleic acid encodes a GAA polypeptide which comprises at least one, at least 2 or at least all three amino acid modifications selected from: H201L, H199R or R233H of SEQ ID NO: 10.

62. A method to treat a subject with a glycogen storage disease type II (GSD II, Pompe Disease, Acid Maltase Deficiency) or having a deficiency in alpha-glucosidase (GAA) polypeptide, comprising administering the recombinant AAV vector of claim 1 to the subject.

63. (canceled)

64. (canceled)

65. The method of claim 62, wherein the recombinant AAV vector is a chimeric AAV vector, haploid AAV vector, a hybrid AAV vector or polyploid AAV vector, a rational haploid vector, a mosaic AAV vector, a chemically modified AAV vector, or a AAV vector from any AAV serotypes.

66. (canceled)

67. The method of claim 62, wherein the recombinant AAV vector is selected from any of: a AAVXL32 vector, a AAVXL32.1 vector, a AAV8 vector and a haploid AAV8 vector comprising at least one AAV8 capsid protein.

68.-73. (canceled)
